SEQUENCE LISTING (429 sequences)

SEQ ID NO: 1 - DNA, 66 bp, Homo sapiens
aggtagcgggcagtagccgcttcagggagggacgaagagacccagcaacccacagagttgagaaat

SEQ ID NO: 2 - DNA, 684 bp, Homo sapiens
ttgactggcattcaagctgtccaatcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatgctatcaaagagatgattgagaactggtacggagggagtcgagccgggctcacttaagggctacgacttaacgggccgcgtcactcaatggcgcggacacgcctctttgcccgggcagaggcatgtacagcgcatgcccacaacggcggaggccgccgggttccctgacgtgccagtcaggccttctccttttccgcagaccgtgtgtttctttaccgctctcccccgagaccttttaagggttgtttggagtgtaagtggaggaatatacgtagtgttgtcttaatggtaccgttaactaagtaaggaagccacttaatttaaaattatgtatgcagaacatgcgaagttaaaagatgtataaaagcttaagatggggagaaaaaccttttttcagagggtactgtgttactgttttcttgcttttca

SEQ ID NO: 3 - DNA, 56 bp, Artificial Sequence
  synthetic construct
cgcccgccgcgccccgcgcccgccccgccgcccccgcccgcaatagctgccgctga

SEQ ID NO: 4 - DNA, 16 bp, Artificial Sequence
  synthetic construct
cgctggataacttccc

SEQ ID NO: 5 - DNA, 16 bp, Artificial Sequence
  synthetic construct
ggcgggggaagttatc

SEQ ID NO: 6 - DNA, 56 bp, Artificial Sequence
  synthetic construct
cgcccgccgcgccccgcgcccgccccgccgcccccgcccgcgcgccattgagtgac

SEQ ID NO: 7 - DNA, 59 bp, Artificial Sequence
  synthetic construct
cgcccgccgcgccccgcgcccgccccgccgcccccgcccgcaaagagatgattgagaac

SEQ ID NO: 8 - DNA, 15 bp, Artificial Sequence
  synthetic construct
catgcctctgcccgg

SEQ ID NO: 9 - DNA, 58 bp, Artificial Sequence
  synthetic construct
cgcccgccgcgccccgcgcccgccccgccgcccccgcccgggaagaacgtgagcacga

SEQ ID NO: 10 - DNA, 16 bp, Artificial Sequence
  synthetic construct
cattagctggccgctg

SEQ ID NO: 11 - DNA, 22 bp, Artificial Sequence
  synthetic construct
tctgcctttttcttccatcggg

SEQ ID NO: 12 - DNA, 20 bp, Artificial Sequence
  synthetic construct
tccccaaccccctaaagcga

SEQ ID NO: 13 - DNA, 42 bp, Artificial Sequence
  synthetic construct
tctgcctttttcttccatcggggcttcagggagggacgaaga

SEQ ID NO: 14 - DNA, 40 bp, Artificial Sequence
  synthetic construct
tccccaaccccctaaagcgatgcgctgtacatgcctctgc

SEQ ID NO: 15 - DNA, 1200 bp, Homo sapiens
gattctcctgccttagcctcctgagtagctgggattacaggcatgcgtcaccatgcctggctaattttgtatttttagtacaaatggggtttctccatgttggtcaggctggtctcaaactcctgacctcaggtgatccacccgccttggcctcccaaagtgctgggattatgggtgtgagccattgcgcctggccagaaaattcattgacttcctaaagatttattaactttctgcattacttttttttttcccctccatcgtaaatataaaagggaatagtagagaaaatcattcagaattttattttttagtgacattatttagtgacattttattagagtcacttaggaacctgaggctgaataaagttcaggtaaaagtaaaattagttgagaagagacatctgccaaaagaaatctatttttaacttcacttgctgtctttcctagaggaacagaaatagtgctgaatgtcctattagaaatgatggttgctctgcccgtctcttccctctctctcacacaatatgtaaactcatacagtgtatgagcctgtaagacaaaggaaaaacacgttaatgaggcactattgtttgtatttggagtttgttatcattgcttggctcatattaaaatatgtacattagagtagttgcagactgataaattattttctgtttgatttgccagtttagatgcaaaatccacaagtattcaagtgattgttaaagagggaggcctgaagttgattcagatccaagacaatggcaccgggatcagggtaagtaaaacctcaaagtagcaggatgtttgtgcgcttcatggaagagtcaggacctttctctgttctggaaactaggcttttgcagatgggattttttcactgaaaaattcaacaccaacaataaatatttattgagtacctattatttgctgggcactgttcaggggatgtgtcagtgaataaaatagattaaaatctattctcttctgatgcttacattatagtggtgggagacaaaatgggtataataaatattatattagatagcattaagtgctgtggagaaaactaaagcagggaggaagataggagtgtgcaagccagaaaggttgcaattaaattgagtagttcaggaaggcttcaatatggatgtgatatttgagagaccggtggaagtcaaggagcaagttgtga

SEQ ID NO: 16 - DNA, 56 bp, Artificial Sequence
  synthetic construct
cgcccgccgcgccccgcgcccgccccgccgcccccgcccgttatcattgcttggct

SEQ ID NO: 17 - DNA, 18 bp, Artificial Sequence
  synthetic construct
ttgtcttggatctgaatc

SEQ ID NO: 18 - DNA, 58 bp, Artificial Sequence
  synthetic construct
cgcccgccgcgccccgcgcccgccccgccgcccccgcccggcaaaatccacaagtatt

SEQ ID NO: 19 - DNA, 17 bp, Artificial Sequence
  synthetic construct
cctgactcttccatgaa

SEQ ID NO: 20 - DNA, 42 bp, Artificial Sequence
  synthetic construct
tctgcctttttcttccatcgggtgcccgtctcttccctctct

SEQ ID NO: 21 - DNA, 40 bp, Artificial Sequence
  synthetic construct
tccccaaccccctaaagcgacctgaacagtgcccagcaaa

SEQ ID NO: 22 - DNA, 1020 bp, Homo sapiens
acctgtaatcccagccactctggaggctgagacatgaaaattgcttgaacccgggaggcggaggttgcagtgagctgagatctcgccactgcacttcagcctgggtgacagagcaagactctgtctcaaaggaggttgcagtgagctgagatctcgccactgcacttcagcctgggtgacagagcaagactctgtctcaaaaaaaaaaaaaacaaaaaccaagaaaagaaaaaaaaactcttctaagaggatttttttttcctggattaaatcaagaaaatgggaattcaaagagatttggaaaaatgagtaacatgattatttactcatctttttggtatctaacagaaagaagatctggatattgtatgtgaaaggttcactactagtaaactgcagtcctttgaggatttagccagtatttctacctatggctttcgaggtgaggtaagctaaagattcaagaaatgtgtaaaatatcctcctgtgatgacattgtctgtcatttgttagtatgtatttctcaacatagataaataaggtttggtaccttttacttgttaaatgtatgcaaatctgagcaaacttaatgaactttaactttcaaagactgagaattgttcataaataaactattttacctgcagagacctctgatatatgtttcttgatggaagtacccagtaccacctatgaagttttcttgtcaaaaaatcaaatgtgaatctgatcattacttagatctaagtaccaatatatgaaaaatataggagacaaggaagcatggtaaatgatactgagattgggagactacatggaaaaagacttgttcccttcaacagatagacagcagggaaaaaagaatagagaaaggagtaaagaacctgtagattaaaagacatttaagggacatatgaaccaggtccagtgtatagatcttacctaaatcctgatggagcaaactataaaaaaatttttttgagacaaatgtttgaatacaggttgactatttgatggcat

SEQ ID NO: 23 - DNA, 56 bp, Artificial Sequence
  synthetic construct
cgcccgccgcgccccgcgcccgccccgccgcccccgcccggggaattcaaagagat

SEQ ID NO: 24 - DNA, 18 bp, Artificial Sequence
  synthetic construct
ttcttgaatctttagctt

SEQ ID NO: 25 - DNA, 18 bp, Artificial Sequence
  synthetic construct
ttcttgaatctttagctt

SEQ ID NO: 26 - DNA, 24 bp, Artificial Sequence
  synthetic construct
gcaccaaaccttatttatctatgt

SEQ ID NO: 27 - DNA, 46 bp, Artificial Sequence
  synthetic construct
tctgcctttttcttccatcgggcaagactctgtctcaaaggaggtt

SEQ ID NO: 28 - DNA, 42 bp, Artificial Sequence
  synthetic construct
tccccaaccccctaaagcgagacaatgtcatcacaggaggat

SEQ ID NO: 29 - DNA, 24 bp, Artificial Sequence
  synthetic construct
cctggattaaatcaagaaaatggg

SEQ ID NO: 30 - DNA, 45 bp, Artificial Sequence
  synthetic construct
tccccaaccccctaaagcgacattaagtttgctcagatttgcata

SEQ ID NO: 31 - DNA, 1320 bp, Homo sapiens
gagatgctgtcacacagaccccgtcatagcacagttcctgagttacatctttacatactgtagtatccttcttgtgaaaaaagatacagattccaaaggtctgagaaaccaatcttggttataaaggggaaaaatggtcatgggtttttaaaatttgttttgtcttaattgcatttcaaatttacatttctaaatgaataattgcttatataaagcagttttgattaacaatataaaacactatctatttggagtgattcctttacccatttctgaaggcaagttttaaaaattactagaagacacttcattgagaatattattaaacatgcctatagttctaccacctcaacacaattgcttattaacacattaatgttttggtgtgttttggactttttaatatgtatttttcacttgttctagtaattatgctacagattgatcatttctttttcaacatgtcatcaaagcaagtgagcaaagtgctcatcgttgccacatattaatacaaaatggaagcagcagttcagataacctttccctttggtgaggtgacagtgggtgacccagcagtgagtttttctttcagtctattttcttttcttccttaggctttggccagcataagccatgtggctcatgttactattacaacgaaaacagctgatggaaagtgtgcatacaggtatagtgctgacttcttttactcatatatattcattctgaaatgtattttttgcctaggtctcagagtaatcctgtctcaacaccagtgttatcttttttggcagagatcttgagtacgttttcttttctccttattgataaattgataatcctcaaggatgattattaggtgatactcttacttcatggattcttaaaagatatgatttaacatattacaagtgcctagcaaggtgtctgttacacgtaggtattttaagtaaatggtagctgctgatgtaatttctgcccctttgcccttcagttggggtattgctttggaccgattagagggctgtggctgggatgctaaaggttcatgtttccttagctggctcctgagccaccagctcccaccacctgtgtatacctgtgctagtttgccttcccacaagtagctgctggctatctgttatgctggtacagttttcagaaactgatgaatggcctttgaacagaacaaaaatgagattcagaataacaaaattgcacctttgtttttataagcactggccattcactagttgaagactggtaggaatacctaattcatgccaaaagaaagataatttttaaaaatcacacaggttg

SEQ ID NO: 32 - DNA, 18 bp, Artificial Sequence
  synthetic construct
ggtgaggtgacagtgggt

SEQ ID NO: 33 - DNA, 67 bp, Artificial Sequence
  synthetic construct
cgcccgccgcgccccgcgcccgccccgccgcccccgcccgtgaatatatatgagtaaaagaagtcag

SEQ ID NO: 34 - DNA, 23 bp, Artificial Sequence
  synthetic construct
tcatgttactattacaacgaaaa

SEQ ID NO: 35 - DNA, 61 bp, Artificial Sequence
  synthetic construct
cgcccgccgcgccccgcgcccgccccgccgcccccgcccggataacactggtgttgagaca

SEQ ID NO: 36 - DNA, 44 bp, Artificial Sequence
  synthetic construct
tctgcctttttcttccatcgggcatgtcatcaaagcaagtgagc

SEQ ID NO: 37 - DNA, 44 bp, Artificial Sequence
  synthetic construct
tccccaaccccctaaagcgatgagacaggattactctgagacct

SEQ ID NO: 38 - DNA, 1140 bp, Homo sapiens
catttgctggaagaacagatagtttttcaaatccaattcaaggactgggtatggtggctcatgcctgtaatcccagcactttgggaggccgaggcaggcgtatccaggagttcgagactagcctgaccaacatggtgaaactccgtctctactaaaaatacaaaattagccaggtgtggtggtgggcacctgtaatctcagctacttgggaggctgaggcaggagaatcgcttgaacctggtaggcggaggttgtagtgagctgagattgtgccattgctctccagcctgggaaacaagagcaaaactccgtctcaaaaaaaaaaaaaatccaattcaaatgattatggaagtagtggagaaataaacaggaaaatgataaataattaagataatatataatatggctatattttaatctattgttgatatgattttctcttttccccttgggattagtatctatctctctactggatattaatttgttatattttctcattagagcaagttactcagatggaaaactgaaagcccctcctaaaccatgtgctggcaatcaagggacccagatcacggtaagaatggtacatgggagagtaaattgttgaagctttgtttgtataaatattggaataaaaaataaaattgcttctaagttttcagggtaataataaaatgaatttgcactagttaatggaggtcccaagatatcctctaagcaagataaatgactattggcttttgtggcatggcagcctgccacgtccttgtcttttttaagggctaggagattctttattgggatggcaaaagtcaatggcagggtagttgtcattgaaagaagattaagcttgaccccagaaggcatgggttagagcccagccttgtcactcaatggttgtatgtccagaggcaagtcacttaacatcccttaaccccagttttctcatctgtcaaatgaagcaaagaatacttgccctcttgacttaaagggtgtctgatgagacatatgactgtatcattagctgggagaaagtccatcgtgctgcctatgtatagtgcctcaagttggtctctttcccttctatgattacacaaagcactccgctgtcatgttatccatcccgcccctccattc

SEQ ID NO: 39 - DNA, 59 bp, Artificial Sequence
  synthetic construct
cgcccgccgcgccccgcgcccgccccgccgcccccgcccggggattagtatctatctct

SEQ ID NO: 40 - DNA, 15 bp, Artificial Sequence
  synthetic construct
ggctttcagttttcc

SEQ ID NO: 41 - DNA, 16 bp, Artificial Sequence
  synthetic construct
ctgaaagcccctccta

SEQ ID NO: 42 - DNA, 60 bp, Artificial Sequence
  synthetic construct
cgcccgccgcgccccgcgcccgccccgccgcccccgcccgagcttcaacaatttactctc

SEQ ID NO: 43 - DNA, 17 bp, Artificial Sequence
  synthetic construct
caagggacccagatcac

SEQ ID NO: 44 - DNA, 62 bp, Artificial Sequence
  synthetic construct
cgcccgccgcgccccgcgcccgccccgccgcccccgcccgccaatatttatacaaacaaagc

SEQ ID NO: 45 - DNA, 63 bp, Artificial Sequence
  synthetic construct
cgcccgccgcgccccgcgcccgccccgccgcccccgcccgtttgttatattttctcattagag

SEQ ID NO: 46 - DNA, 18 bp, Artificial Sequence
  synthetic construct
attcttaccgtgatctgg

SEQ ID NO: 47 - DNA, 46 bp, Artificial Sequence
  synthetic construct
tctgcctttttcttccatcgggcccttgggattagtatctatctct

SEQ ID NO: 48 - DNA, 42 bp, Artificial Sequence
  synthetic construct
tccccaaccccctaaagcgaggacctccattaactagtgcaa

SEQ ID NO: 49 - DNA, 1020 bp, Homo sapiens
atgcgtcaccatgcccggctaatttttgtatttttagtagagacagggtttcaccatgttggccaggctggtctcgaactcctgacctcaggtgacccacccaccttggcctcccaaagttctgggattacagacgtgagccactgcacccagcctgaaaaatatctttgaatgccatgtgatactatacttgtcagtttacatgtgtgtcccactaaatcatgtactctcctgagcaggatcatgctttgtcttcatattttctgtacaaagcaaagactctgacacaaagctagcccccagtgcatagttgagaaatcagtgaatgaatgtgggaggcaggaaaaatgtcctttaattcttctgttaatgctgtcttatccctggccccagtcagtgcttagaactgtgctgttggtaaatataattggattcactatcttaagacctcgcttttgccaggacatcttgggttttattttcaagtacttctatgaatttacaagaaaaatcaatcttctgttcaggtggaggaccttttttacaacatagccacgaggagaaaagctttaaaaaatccaagtgaagaatatgggaaaattttggaagttgttggcaggtacagtccaaaatctgggagtgggtctctgagatttgtcatcaaagtaatgtgttctagtgctcatacattgaacagttgctgagctagatggtgaaaagtaaaactagcttacagatagtttctggtcaaggtttagccaccaattttgcagtttctctcatctccccaggaaagagcagttggtctttagatcaatgagagctcttttatggcagacaaaacaaagtgactctagccaacttgagctaaaaagaaatttagtggaaggctaggagttaccacatgaagtgtgtgcagctgccccttggagagaataagaaccagggtgcctctgggacttaacatcattactgtactccagttgttttcattcttttcctgactttgctctagagtca

SEQ ID NO: 50 - DNA, 63 bp, Artificial Sequence
  synthetic construct
cgcccgccgcgccccgcgcccgccccgccgcccccgcccgattcactatcttaagacctcgct

SEQ ID NO: 51 - DNA, 25 bp, Artificial Sequence
  synthetic construct
ctagaacacattactttgatgacaa

SEQ ID NO: 52 - DNA, 46 bp, Artificial Sequence
  synthetic construct
tctgcctttttcttccatcgggctgttaatgctgtcttatccctgg

SEQ ID NO: 53 - DNA, 43 bp, Artificial Sequence
  synthetic construct
tccccaaccccctaaagcgaccatctagctcagcaactgttca

SEQ ID NO: 54 - DNA, 1620 bp, Homo sapiens
aatccttcggttcacgagctctgtagagaaaagagaaataaccgccaaccaagaaaagattgggagatactagaataagacccaggggcaggaagaagccagtgagaaggagggcatgttgagagctctgagagagaataaaagcaggggttgttggagctagcttctcaagatgtccttgaggcaaaccagacctttgggacactctgaaaataaaactgaaagtgaagagattgtgggccgaatgtggtggctcacgcctgtaatcccagcactttgggaggtcgaggcgggtggatcacctgagatcaggagttcgataccagcctggccaacatggcgaaacgccatctctactaaaaatacaaaaaaaattagctgggcctggtggcaggcgcctataatcccagctactcgggaggctgaggcgggagaatcgcttgagtccaggaggcggaggttgcagtgagctgagatcgtgccattgcactccagcctgggcaacaagagcaaaactctgtctcaaaaataaataaaaataaataaaaaagagatagtggcgtgatatccttgattctatcagcaacctataaaagtagagaggagtctgtgttttgattcagtcacctttagcatttttatttccatgaagtttctgctggtttatttttctgtgggtaaaatattaataggctgtatggagatatttttctttatatgtacctttgtttagattactcaactccactaatttatttaactaaaagggggctctgacatctagtgtgtgtttttggcaactcttttcttactcttttgtttttcttttccaggtattcagtacacaatgcaggcattagtttctcagttaaaaaagtaagttcttggtttatgggggatggttttgttttatgaaaagaaaaaaggggatttttaatagtttgctggtggagataaggttatgatgtttcagtctcagccatgagacaataaatccttgtgtcttctgctgtttgtttatcagcaaggagagacagtagctgatgttaggacactacccaatgcctcaaccgtggacaatattcgctccatctttggaaatgctgttagtcggtatgtcgataacctatataaaaaaatcttttacatttattatcttggtttatcattccatcacattattttggaacctttcaagatattatgtgtgttaagagtttgctttagtcaaatacacaggcttgttttatgcttcagatttgttaatggagttcttatttcacgtaatcaacactttctaggtgtatgtaatctcctagattctgtggcgtgaatcatgtgttctttcaaggtcttagtcttgaaaatatttatagtgtagtagaactattttatcctccaatgctccttcttttccttgtatttccattatcatcactttaggatttcacttatttatcattcaacatttattaattgcctctcatattccaggctttgtgctagaagttagggatataaagacaaataagatatttcctgcccttaaagactagattcgtgttgctaagtcttcattatcaagaaaagcataagtggggaaaagtgcttgcattatgg

SEQ ID NO: 55 - DNA, 16 bp, Artificial Sequence
  synthetic construct
taactaaaagggggct

SEQ ID NO: 56 - DNA, 57 bp, Artificial Sequence
  synthetic construct
cgcccgccgcgccccgcgcccgccccgccgcccccgcccgtttattgtctcatggct

SEQ ID NO: 57 - DNA, 42 bp, Artificial Sequence
  synthetic construct
tctgcctttttcttccatcgggttccatgaagtttctgctgg

SEQ ID NO: 58 - DNA, 42 bp, Artificial Sequence
  synthetic construct
tccccaaccccctaaagcgaccttatctccaccagcaaacta

SEQ ID NO: 59 - DNA, 1020 bp, Homo sapiens
aaataaaaaagagatagtggcgtgatatccttgattctatcagcaacctataaaagtagagaggagtctgtgttttgattcagtcacctttagcatttttatttccatgaagtttctgctggtttatttttctgtgggtaaaatattaataggctgtatggagatatttttctttatatgtacctttgtttagattactcaactccactaatttatttaactaaaagggggctctgacatctagtgtgtgtttttggcaactcttttcttactcttttgtttttcttttccaggtattcagtacacaatgcaggcattagtttctcagttaaaaaagtaagttcttggtttatgggggatggttttgttttatgaaaagaaaaaaggggatttttaatagtttgctggtggagataaggttatgatgtttcagtctcagccatgagacaataaatccttgtgtcttctgctgtttgtttatcagcaaggagagacagtagctgatgttaggacactacccaatgcctcaaccgtggacaatattcgctccatctttggaaatgctgttagtcggtatgtcgataacctatataaaaaaatcttttacatttattatcttggtttatcattccatcacattattttggaacctttcaagatattatgtgtgttaagagtttgctttagtcaaatacacaggcttgttttatgcttcagatttgttaatggagttcttatttcacgtaatcaacactttctaggtgtatgtaatctcctagattctgtggcgtgaatcatgtgttctttcaaggtcttagtcttgaaaatatttatagtgtagtagaactattttatcctccaatgctccttcttttccttgtatttccattatcatcactttaggatttcacttatttatcattcaacatttattaattgcctctcatattccaggctttgtgctagaagttagggatataaagacaaataagatatttcctgcccttaaagactagattc

SEQ ID NO: 60 - DNA, 56 bp, Artificial Sequence
  synthetic construct
cgcccgccgcgccccgcgcccgccccgccgcccccgcccggctggtggagataagg

SEQ ID NO: 61 - DNA, 15 bp, Artificial Sequence
  synthetic construct
tgtccacggttgagg

SEQ ID NO: 62 - DNA, 21 bp, Artificial Sequence
  synthetic construct
gggggcaaggagagacagtag

SEQ ID NO: 63 - DNA, 60 bp, Artificial Sequence
  synthetic construct
cgcccgccgcgccccgcgcccgccccgccgcccccgcccgatataggttatcgacatacc

SEQ ID NO: 64 - DNA, 14 bp, Artificial Sequence
  synthetic construct
aaatgctgttagtc

SEQ ID NO: 65 - DNA, 56 bp, Artificial Sequence
  synthetic construct
cgcccgccgcgccccgcgcccgccccgccgcccccgcccgtcttgaaaggttccaa

SEQ ID NO: 66 - DNA, 43 bp, Artificial Sequence
  synthetic construct
tctgcctttttcttccatcgggggtttatgggggatggttttg

SEQ ID NO: 67 - DNA, 44 bp, Artificial Sequence
  synthetic construct
tccccaaccccctaaagcgacgccacagaatctaggagattaca

SEQ ID NO: 68 - DNA, 1260 bp, Homo sapiens
tattaaccttccctccccagtaaacactcctgggaacaacacacattgtagaaccacgttgtggtgctgttcagtatagcaagtaattcagcagagataagttcttggaatctcatctttgggatttagttactaagatacattcaagtttgagcaaaataaggtctcagagcttggattcattgttctgttccagcaattagagcagtacctggcacatagcacaagtgcttgaaaacactgactgagtagggtaggtgggtgagtgggtgggtgggtgggtgggtggatggatggatgggaggatgggtgggtgaatgggtgaacagacaaatggatggatgaatggacaggcacaggaggacctcaaatggaccaagtcttcggggccctcatttcacaaagttagtttatgggaaggaaccttgtgtttttaaattctgattcttttgtaatgtttgagttttgagtattttcaaaagcttcagaatctcttttctaatagagaactgatagaaattggatgtgaggataaaaccctagccttcaaaatgaatggttacatatccaatgcaaactactcagtgaagaagtgcatcttcttactcttcatcaaccgtaagttaaaaagaaccacatgggaaatccactcacaggaaacacccacagggaattttatgggaccatggaaaaatttctgatccataggtttgattaaacatggagaaacctcatggcaaagtttggttttattgggaagcatgtataatttttgtcctaagtctgtgctcagccctcccacatgtgctcattgctggttgactgttggagtctggttcttacctctaagaggaagcccaggagagggcataaagccagcacactgtcctcacctgatggtgtcagagtccttacgagtaagccctagccagaacattgctggaagagatcaagggccactgtttgaaattgcacagcaggatacggaaaaggggtaccttaggtataggcattgtcattaaagaaattgctaagatacttgagattttcctgtttaaggaatgagctttatgatacaaagagcagttctaaaaattagggagggaattaactaaattaattaggatatttctcaaattcctttacagtttttgtctctctgctgatatagtgtttacatgattgttatttactaaacaaatgctattttgtattgtgctccttataacttaattgtttattacaaggttttgatggtga

SEQ ID NO: 69 - DNA, 66 bp, Artificial Sequence
  synthetic construct
cgcccgccgcgccccgcgcccgccccgccgcccccgcccggtaatgtttgagttttgagtattttc

SEQ ID NO: 70 - DNA, 20 bp, Artificial Sequence
  synthetic construct
cagaaatttttccatggtcc

SEQ ID NO: 71 - DNA, 62 bp, Artificial Sequence
  synthetic construct
cgcccgccgcgccccgcgcccgccccgccgcccccgcccgcaaagttagtttatgggaagga

SEQ ID NO: 72 - DNA, 24 bp, Artificial Sequence
  synthetic construct
gaagagtaagaagatgcacttctt

SEQ ID NO: 73 - DNA, 63 bp, Artificial Sequence
  synthetic construct
cgcccgccgcgccccgcgcccgccccgccgcccccgcccgcttcaaaatgaatggttacatat

SEQ ID NO: 74 - DNA, 18 bp, Artificial Sequence
  synthetic construct
attccctgtgggtgtttc

SEQ ID NO: 75 - DNA, 42 bp, Artificial Sequence
  synthetic construct
tctgcctttttcttccatcgggggtgggtgaatgggtgaaca

SEQ ID NO: 76 - DNA, 40 bp, Artificial Sequence
  synthetic construct
tccccaaccccctaaagcgatttgccatgaggtttctcca

SEQ ID NO: 77 - DNA, 1080 bp, Homo sapiens
tgtctacaccttaagccgcggctcccgaagcacctagaaccggaagagttggctcactatttagcacacacacacgtctataatagtgctggccacttggggttggaattagtttatttatcagcatgttgtctcccagcacttggtgtgtgtgatatgcagtatgtatttgcagaatgaaaagtctgagggctgacatcatatttcccactgtgcccagaaagagcacagttagtccacatgagctaatgggggcaaagggaagtgaggagggagaatgtactgccttatcatgttttctattacttggctgaagtaaaacagtcccaagccgatagtaagatagtgggctggaaagtggcgacaggtaaaggtgcacctttcttcctggggatgtgatgtgcatatcactacagaaatgtctttcctgaggtgatttcatgactttgtgtgaatgtacacctgtgacctcacccctcaggacagttttgaactggttgctttctttttattgtttagatcgtctggtagaatcaacttccttgagaaaagccatagaaacagtgtatgcagcctatttgcccaaaaacacacacccattcctgtacctcaggtaatgtagcaccaaactcctcaaccaagactcacaaggaacagatgttctatcaggctctcctctttgaaagagatgagcatgctaatagtacaatcagagtgaatcccatacaccactggcaaaaggatgttctgtcccttcttacaggtacaaggcacagttttccttcatttattcactaatttagcagaacctcactaagagcctcctatatgccaggctctgcgttagcaataaaaggaatgccatgcctcaccccatcaggaggtgctgatagcttgtaggcggagtggaaacagatgtgctctagaggctctaaatattacttctgctggggtcagttgggaagccacaacagctactgttcatcttccataaaagacaatcagccgggcacagtggctcacacctgtaaatcccagcactttgggaggctgaggtgggtggatcacaaggtcaggtgt

SEQ ID NO: 78 - DNA, 58 bp, Artificial Sequence
  synthetic construct
cgcccgccgcgccccgcgcccgccccgccgcccccgcccgtgaatgtacacctgtgac

SEQ ID NO: 79 - DNA, 18 bp, Artificial Sequence
  synthetic construct
tagaacatctgttccttg

SEQ ID NO: 80 - DNA, 41 bp, Artificial Sequence
  synthetic construct
tctgcctttttcttccatcggggctggaaagtggcgacagg

SEQ ID NO: 81 - DNA, 41 bp, Artificial Sequence
  synthetic construct
tccccaaccccctaaagcgagccagtggtgtatgggattca

SEQ ID NO: 82 - DNA, 1140 bp, Homo sapiens
gatggagtcttgctctgtcgccaagctggagtgcagtggcacgatctcggcttactgcaacctctgactccctggttgaagggattctcctccctcagcctcccgagtacctgggattacaggcatgcgccaccacgcccagctaatttttgtatttttagtagagacgtggtttcatcatgttggccaggatggtctcgatctcctgaccttgtgatccacccgcctcggcctccccaaatgctgggattacaggcgtgagccaccacgcccggccacttggcatgaatttaattcccgccataaacctgtgagataggtaattctgttatatccactttacaaatgaagagactgaggcaaagaaagatgatgtaacttacgcaaagctacacagctcttaagtagcagtgccaatatttgaacacactcagactcgatcctgaggttttgaccactgtgtcatctggcctcaaatcttctggccaccacatacaccatatgtgggctttttctccccctcccactatctaaggtaattgttctctcttattttcctgacagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccaggtcagggcgcttctcatccagctacttctctggggcctttgaaatgtgcccggccagacgtgagagcccagatttttgcctgttatttaggaactttctttgcaagtattacctggatagttttaacattttcttctttgaacctagttataaaggtattgtgctgttgttcctaggcttagagtcataaggcctgagctcacttcctcactttgcctccatctggaaccttagaccaacttcctaggaaaacgagctgtctgaaaacagaatagggtgcctcttcaatgtgctcttcactggagatgttcaggaggaggctactcccacctacacagggtgcagtggagggtctgggccccagggaggcagcaggaagagtggaaagagcggaggctctactgttggacagacctgggtta

SEQ ID NO: 83 - DNA, 57 bp, Artificial Sequence
  synthetic construct
cgcccgccgcgccccgcgcccgccccgccgcccccgcccgttgaccactgtgtcatc

SEQ ID NO: 84 - DNA, 16 bp, Artificial Sequence
  synthetic construct
gtgcaggaagtgaact

SEQ ID NO: 85 - DNA, 59 bp, Artificial Sequence
  synthetic construct
cgcccgccgcgccccgcgcccgccccgccgcccccgcccgcagaatgtggatgttaatg

SEQ ID NO: 86 - DNA, 15 bp, Artificial Sequence
  synthetic construct
ggaggaattggagcc

SEQ ID NO: 87 - DNA, 17 bp, Artificial Sequence
  synthetic construct
cagcagcacatcgagag

SEQ ID NO: 88 - DNA, 58 bp, Artificial Sequence
  synthetic construct
cgcccgccgcgccccgcgcccgccccgccgcccccgcccgatctgggctctcacgtct

SEQ ID NO: 89 - DNA, 43 bp, Artificial Sequence
  synthetic construct
tctgcctttttcttccatcgggagactgaggcaaagaaagatg

SEQ ID NO: 90 - DNA, 39 bp, Artificial Sequence
  synthetic construct
tccccaaccccctaaagcgaaggcaaaaatctgggctct

SEQ ID NO: 91 - DNA, 1320 bp, Homo sapiens
aagatgaaaaagttctagagatagctggtggtgatggttgcgcaacaatgtaaatgccactgagctctcatttaaaaatggttaaaatggtaaattttatatatattttaccacaataaaaaaaagtcttcttctgggagcacccccccaagacaaaaatatgaaaattttacactgatacttccatttcaagataattttaagattataaggattttgcttaattcttgaattttatacctgtaaaccttttatacttcaaatttcgggcagaattgcttctataacaatgataattatacctcatactagcttctttcttagtactgctccatttggggacctgtatatctatacttcttattctgagtctctccactatatatatatatatatatatatatttttttttttttttttttttaatacagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccaggtatggccttttgggaaaagtacagcctacctcctttattctgtaataaaactgccttctaactttggcttttcatgaatcacttgcatcttctctctgcctgacttgccctctggaatggtgctggaatggtcctgtggccttgtccactgtctgcctttgaccataacttgaaagtcacccaccatagtgtcctttgaaataacttaaatgtccacagttccaagcatgagttaaaaacacttcagaatgtagagtagttgttcaattgaataaacacacacaccagaaaaaaaagcaagtttatcttttatttttagtaaagaattttgatagagcctcaacaccagaaatggctagagagagaagcctaacatatctggaggattatttttcatcctacttaaagctgctttcacttttttcaggaaaaaacacacgttctgaatctaatttataaaactccctggccgggtgctgtggctcacacctataatcccagcactttgggaggctga

SEQ ID NO: 92 - DNA, 61 bp, Artificial Sequence
  synthetic construct
cgcccgccgcgccccgcgcccgccccgccgcccccgcccgttttttttaatacagactttg

SEQ ID NO: 93 - DNA, 15 bp, Artificial Sequence
  synthetic construct
gtgacaatggcctgg

SEQ ID NO: 94 - DNA, 16 bp, Artificial Sequence
  synthetic construct
catttctgcagcctct

SEQ ID NO: 95 - DNA, 55 bp, Artificial Sequence
  synthetic construct
cgcccgccgcgccccgcgcccgccccgccgcccccgcccgtttttggcagccact

SEQ ID NO: 96 - DNA, 16 bp, Artificial Sequence
  synthetic construct
agcccctgctgaagtg

SEQ ID NO: 97 - DNA, 61 bp, Artificial Sequence
  synthetic construct
cgcccgccgcgccccgcgcccgccccgccgcccccgcccgagaaggcagttttattacaga

SEQ ID NO: 98 - DNA, 56 bp, Artificial Sequence
  synthetic construct
cgcccgccgcgccccgcgcccgccccgccgcccccgcccgtgtccagtcagcccca

SEQ ID NO: 99 - DNA, 17 bp, Artificial Sequence
  synthetic construct
ctctgatttttggcagc

SEQ ID NO: 100 - DNA, 41 bp, Artificial Sequence
  synthetic construct
tctgcctttttcttccatcgggtttcgggcagaattgcttc

SEQ ID NO: 101 - DNA, 43 bp, Artificial Sequence
  synthetic construct
tccccaaccccctaaagcgagcagagagaagatgcaagtgatt

SEQ ID NO: 102 - DNA, 24 bp, Artificial Sequence
  synthetic construct
cagactttgctaccaggacttgct

SEQ ID NO: 103 - DNA, 44 bp, Artificial Sequence
  synthetic construct
tctgcctttttcttccatcgggatagctggtggtgatggttgcg

SEQ ID NO: 104 - DNA, 42 bp, Artificial Sequence
  synthetic construct
tccccaaccccctaaagcgaccattccagcaccattccagag

SEQ ID NO: 105 - DNA, 1080 bp, Homo sapiens
gcctggaagacatagtgagactctctctcaaaaaaaaaaaaaaaaaaaaaggaagtaagcattgtgagggcaggtaccttctctgttttgttcattgctggatgtagttagtatacagcagtatctgatggatggatagatggaggaatgaatgaatgagacttcacaaattcagctcacttgctcaaggccctgcagctctacgggatgaagctatactccagagtcctgctacattggctgtgtggccagctgctgggatctgagggttgtcagataagcagtctaccagagaacagactgatcttgttggccttctgccagcacaggggttcattcacagctctgtagaaccagcacagagaagttgcttgctcctccaaaatgcaacccacaaaatttggctaagtttaaaaacaagaataataatgatctgcacttccttttcttcattgcagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgagggtacgtaaacgctgtggcctgcctgggatgcatagggcctcaactgccaaggttttggaaatggagaaagcagtcatgttgtcagagtggccactacagttttgctgggcaagctcctcttcctttactaacccacaatagcatcagcttaaagacaatttttgattgggagaaaagggagaaaaataatctctgtttattttaattagcattaattggtattcttgttaaaccataggagtcagagtaaatcagccatttcaccaattttcagtttgtttctgtcttagctaacagcagtgtaatggtcagcaaaattcttatcttgtgtactgaatggcatgtcctgttgctgaaagtgcacaggcttgggaggtagccatgagctcaaatcctggcactaccacctctcttgtgtgaccttagactcctgacctttctatgcctcagttctttcttacctataaaatgaa

SEQ ID NO: 106 - DNA, 57 bp, Artificial Sequence
  synthetic construct
cgcccgccgcgccccgcgcccgccccgccgcccccgcccgaatttggctaagtttaa

SEQ ID NO: 107 - DNA, 16 bp, Artificial Sequence
  synthetic construct
ggaatcatcttccacc

SEQ ID NO: 108 - DNA, 59 bp, Artificial Sequence
  synthetic construct
cgcccgccgcgccccgcgcccgccccgccgcccccgcccgcattgcagaaagagacatc

SEQ ID NO: 109 - DNA, 20 bp, Artificial Sequence
  synthetic construct
gtgaggttaatgatccttct

SEQ ID NO: 110 - DNA, 61 bp, Artificial Sequence
  synthetic construct
cgcccgccgcgccccgcgcccgccccgccgcccccgcccgtgattcccgaaaggaaatgac

SEQ ID NO: 111 - DNA, 28 bp, Artificial Sequence
  synthetic construct
caggccacagcgtttacgtaccctcatg

SEQ ID NO: 112 - DNA, 61 bp, Artificial Sequence
  synthetic construct
cgcccgccgcgccccgcgcccgccccgccgcccccgcccgattaacctcactagtgttttg

SEQ ID NO: 113 - DNA, 16 bp, Artificial Sequence
  synthetic construct
tgaggccctatgcatc

SEQ ID NO: 114 - DNA, 43 bp, Artificial Sequence
  synthetic construct
tctgcctttttcttccatcgggactgatcttgttggccttctg

SEQ ID NO: 115 - DNA, 40 bp, Artificial Sequence
  synthetic construct
tccccaaccccctaaagcgatggccactctgacaacatga

SEQ ID NO: 116 - DNA, 960 bp, Homo sapiens
tggtctcctattagactctccatttcaaaccattccatgattttgtcctccttttgccaccttccgagcctgtaaaaactaatgtttgtgattcctgaggtttctctaatgtcttttaataaagttgacctcagagatctcgttacctctctgagttcctgctttgtcttagattttgatccttgagtgttctttaatcttttagcaattccttgttgcatgttaaaagattagttatattttattcctcatttgtgttcgttttcaccaggaggctcaattcaggcttctttgcttacttggtgtctctagttctggtgcctggtgctttggtcaatgaagtggggttggtaggattctattacttacctgttttttggttttattttttgttttgcagttctccgggagatgttgcataaccactccttcgtgggctgtgtgaatcctcagtgggccttggcacagcatcaaaccaagttataccttctcaacaccaccaagcttaggtaaatcagctgagtgtgtgaacaagcagagctactacaacaatggtccagggagcacaggcacaaaagctaaggagagcagcatgaggtagttgggagggcacaggctttggagtcagacacatgtggtttcaaatccaagttcgaccatttcccatttatttgactgtagacaagttacattcctaaactatgtctcagatttctcatctgtaagttgtggtattactagttaacatgcaggggttttgtttgtttgtttgtttgtttgtttgtgagggtaagaaataacccaagaagcctagtccttggtagttgctcagtgccctataaatgttgtgaaccaggtggtgagggtttggtgctgctagagaattctggtatctgctctgtgcaacagagtactgtaggtgatgcaagagaaagaagacctgatgccttctttcctccc

SEQ ID NO: 117 - DNA, 56 bp, Artificial Sequence
  synthetic construct
cgcccgccgcgccccgcgcccgccccgccgcccccgcccgggtcaatgaagtgggg

SEQ ID NO: 118 - DNA, 17 bp, Artificial Sequence
  synthetic construct
ccacgaaggagtggtta

SEQ ID NO: 119 - DNA, 16 bp, Artificial Sequence
  synthetic construct
agttctccgggagatg

SEQ ID NO: 120 - DNA, 57 bp, Artificial Sequence
  synthetic construct
cgcccgccgcgccccgcgcccgccccgccgcccccgcccgtacctcatgctgctctc

SEQ ID NO: 121 - DNA, 42 bp, Artificial Sequence
  synthetic construct
tctgcctttttcttccatcgggtgttcgttttcaccaggagg

SEQ ID NO: 122 - DNA, 42 bp, Artificial Sequence
  synthetic construct
tccccaaccccctaaagcgatcgaacttggatttgaaaccac

SEQ ID NO: 123 - DNA, 1020 bp, Homo sapiens
tttaggaagactccctgcccttcctatacatttcacataatttttaataagttgtaaaaaagtgatttataggattctttgtaagtgggggaagttaagcagacaaaaagtttttaaatcttactgcagagtgtcaggaaccttttatagcaccagacaggtagggacagaacatgagtggcagcaagccagacttggtcttagtgctctaacctgtctgttagaggctggccagtcagacccctggttgaagacgttgggaatcccagctctttggaggggtaagagattttgttagactgttaaccagattccacagccaggcagaactatttctgtctcatccatgtttcagggattacttctcccattttgtcccaactggttgtatctcaagcatgaattcagcttttccttaaagtcacttcatttttattttcagtgaagaactgttctaccagatactcatttatgattttgccaattttggtgttctcaggttatcggtaagtttagatccttttcacttctgaaatttcaactgatcgtttctgaaaatagtagctctccactaatatcttatttgtagtatgttaaatttttctaaaacttctaaggatagttgctgtattgtatgatttgcatatggaggtatctataagaagttttatactttttagcaaaatagtcatttggtagccaacttaaacaaatgtttattaatatagaagttaataatatctactgatactcggccgggtgcggtggctcatgcctgtaatcccaccactttgggaggctgaggcgggcagatcatttgaggtcaggagttcaagaccagcctgaccaatatgatgaaaccctgtctctactaaattacaaatattagcagggtatggtggtgggcgcctgtaatcccagctactcaggaggctaaggcaggagaatcatttgaacccaggaggcagaggttgcaatgagctgagatcacgccactgcactccagcctgggcaacagag

SEQ ID NO: 124 - DNA, 17 bp, Artificial Sequence
  synthetic construct
ttcagggattacttctc

SEQ ID NO: 125 - DNA, 60 bp, Artificial Sequence
  synthetic construct
cgcccgccgcgccccgcgcccgccccgccgcccccgcccggaaaaatttaacatactaca

SEQ ID NO: 126 - DNA, 41 bp, Artificial Sequence
  synthetic construct
tctgcctttttcttccatcgggagattccacagccaggcag

SEQ ID NO: 127 - DNA, 44 bp, Artificial Sequence
  synthetic construct
tccccaaccccctaaagcgatacctccatatgcaaatcatacaa

SEQ ID NO: 128 - DNA, 960 bp, Homo sapiens
gcattagatgatttacctgaaatgtcattcaatttaacttactctccatcctcacccgcccagctttggttatgaggcagtagaaagaaatgatctgcctgtggttttctagaaatacgaaagttgagtccttaaggctacacagaaagaaagtacctccccagggcttcacccttcccatcctttcagcaggctttttgtctgtcgtatcttctctgttgaaatggccattgacaagaggaggaaaggggttttgttgtggattgttcaggcacttcctttggggtatatgggggatgagtgttacatttatggtttctcacctgccattctgatagtggattcttgggaattcaggcttcatttggatgctccgttaaagcttgctccttcatgttcttgcttcttcctaggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggtgtgacagccattcttatacttctgttgtattcttcaaataaaatttccagccgggtgcggtggctcatggctgtaatcccagcactttgggaggctgaggtgggcagataacttggggtcaggagttcaaaaccagctggccaacatgatgaaaccccgtctctactaaaaaaatagaaaaattagccaggcgtggtggcgggtacctgtaatccaagctgctcaggaggctgaggcagaagaatcacttaaacccaagaggtagaagttgcagtgagccgagattgcaccactgcactctagcctaggcgacagcgagactgcgtctcaaaaaaaaaaaaaaagaacgttccaaggtcaggactaggcctcccctcaga

SEQ ID NO: 129 - DNA, 57 bp, Artificial Sequence
  synthetic construct
cgcccgccgcgccccgcgcccgccccgccgcccccgcccggccattctgatagtgga

SEQ ID NO: 130 - DNA, 19 bp, Artificial Sequence
  synthetic construct
tctaaggcaagcatggcaa

SEQ ID NO: 131 - DNA, 14 bp, Artificial Sequence
  synthetic construct
gcaccgctctttga

SEQ ID NO: 132 - DNA, 58 bp, Artificial Sequence
  synthetic construct
cgcccgccgcgccccgcgcccgccccgccgcccccgcccggtataagaatggctgtca

SEQ ID NO: 133 - DNA, 17 bp, Artificial Sequence
  synthetic construct
ggctgagatgcttgcag

SEQ ID NO: 134 - DNA, 55 bp, Artificial Sequence
  synthetic construct
cgcccgccgcgccccgcgcccgccccgccgcccccgcccgcatgagccaccgcac

SEQ ID NO: 135 - DNA, 45 bp, Artificial Sequence
  synthetic construct
tctgcctttttcttccatcgggggttttgttgtggattgttcagg

SEQ ID NO: 136 - DNA, 40 bp, Artificial Sequence
  synthetic construct
tccccaaccccctaaagcgatgggattacagccatgagcc

SEQ ID NO: 137 - DNA, 900 bp, Homo sapiens
gagccgaatccctgcaggccattataaatgagattatgccatttgctcccatttcttcttattctttcatttttggggctctccatcttgatgtgttctttggatcgtgaacagatccaaagaaaaggttgttctgccgtgctgtttgtcaggatgaaaaactcttttttaagtgtttaggtctgcccccagtgcccagcccaatcaagtaacgtggtcacccagagtggcagataggagcacaaggcctgggaaagcactggagaaatgggatttgtttaaactatgacagcattatttcttgttcccttgtcctttttcctgcaagcaggaagggaacctgattggattaccccttctgattgacaactatgtgccccctttggagggactgcctatcttcattcttcgactagccactgaggtcagtgatcaagcagatactaagcatttcggtacatgcatgtgtgctggagggaaagggcaaatgaccaccctttgatctggaatgataaagatgataagggtgggatagctgaaggcctgctctcatccccactaatattcattcccagcaatattcagcagtcccatttacagttttaacgcctaaagtatcacatttcgttttttagctttaagtagtctgtgatctccgtttagaatgagaatgtttaaattcgtacctattttgaggtattgaatttctttggaccaggtgaattgggacgaagaaaaggaatgttttgaaagcctcagtaaagaatgcgctatgttctattccatccggaagcagtacatatctgaggagtcgaccctctcaggccagcaggtacagtggtgatgcacactggcaccccaggactaggacaggacctcatacaatctttaggagatgaa

SEQ ID NO: 138 - DNA, 59 bp, Artificial Sequence
  synthetic construct
cgcccgccgcgccccgcgcccgccccgccgcccccgcccgtgtttaaactatgacagca

SEQ ID NO: 139 - DNA, 15 bp, Artificial Sequence
  synthetic construct
tggtcatttgccctt

SEQ ID NO: 140 - DNA, 44 bp, Artificial Sequence
  synthetic construct
tctgcctttttcttccatcgggtttaagtgtttaggtctgcccc

SEQ ID NO: 141 - DNA, 43 bp, Artificial Sequence
  synthetic construct
tccccaaccccctaaagcgagctatcccacccttatcatcttt

SEQ ID NO: 142 - DNA, 1260 bp, Homo sapiens
gagccgaatccctgcaggccattataaatgagattatgccatttgctcccatttcttcttattctttcatttttggggctctccatcttgatgtgttctttggatcgtgaacagatccaaagaaaaggttgttctgccgtgctgtttgtcaggatgaaaaactcttttttaagtgtttaggtctgcccccagtgcccagcccaatcaagtaacgtggtcacccagagtggcagataggagcacaaggcctgggaaagcactggagaaatgggatttgtttaaactatgacagcattatttcttgttcccttgtcctttttcctgcaagcaggaagggaacctgattggattaccccttctgattgacaactatgtgccccctttggagggactgcctatcttcattcttcgactagccactgaggtcagtgatcaagcagatactaagcatttcggtacatgcatgtgtgctggagggaaagggcaaatgaccaccctttgatctggaatgataaagatgataagggtgggatagctgaaggcctgctctcatccccactaatattcattcccagcaatattcagcagtcccatttacagttttaacgcctaaagtatcacatttcgttttttagctttaagtagtctgtgatctccgtttagaatgagaatgtttaaattcgtacctattttgaggtattgaatttctttggaccaggtgaattgggacgaagaaaaggaatgttttgaaagcctcagtaaagaatgcgctatgttctattccatccggaagcagtacatatctgaggagtcgaccctctcaggccagcaggtacagtggtgatgcacactggcaccccaggactaggacaggacctcatacaatctttaggagatgaaacttgcccatctctaaaatttcgggatttctttgtacccaacaaggttcaaacacaacagtcagcttttattcatgatttttacttccatctgctgatgtagaacatacctccagagtgacctcagaaattgtcaaatgtgaaaacacaagccatcacagtgagaaatgggaggttgagttagattgtctaaggctggagagtccatatactcccactgttagctctgaagtgtgtagccagtcttcagattctgggtcagttgcctcagtctctcttagcttttgccttactctttatccgaccactgccctgccaggaaaacaaggctctataactcctcttacaggtcagcttgaca

SEQ ID NO: 143 - DNA, 58 bp, Artificial Sequence
  synthetic construct
cgcccgccgcgccccgcgcccgccccgccgcccccgcccgtgtgatctccgtttagaa

SEQ ID NO: 144 - DNA, 17 bp, Artificial Sequence
  synthetic construct
ctgagagggtcgactcc

SEQ ID NO: 145 - DNA, 59 bp, Artificial Sequence
  synthetic construct
cgcccgccgcgccccgcgcccgccccgccgcccccgcccgtgcgctatgttctattcca

SEQ ID NO: 146 - DNA, 31 bp, Artificial Sequence
  synthetic construct
gccgcccccgcccgctagtcctggggtgcca

SEQ ID NO: 147 - DNA, 44 bp, Artificial Sequence
  synthetic construct
tctgcctttttcttccatcgggaagatgataagggtgggatagc

SEQ ID NO: 148 - DNA, 40 bp, Artificial Sequence
  synthetic construct
tccccaaccccctaaagcgaccgaaattttagagatgggc

SEQ ID NO: 149 - DNA, 964 bp, Homo sapiens
tacttcctacagttgccatccaaatatcagtcaggatcagacatgatgttagctcctgctacaataaaaccattttctccctgaatgaaaacaaaggttccacaggagacagtcccacagagcagtggcttcttttcctccctttaaaacctcatgttggctggacacagtggctcacacctgtaatcccagcattttaggaggctgaggtgggaagatggcttaagcccaggagtttgaggctgtagagctatgatcacaccactgcccttcagcctgggtgacagagcaagaccttgtctctaaataaacaaacaaacaaaaaatcctcttgtgttcaggcctgtgggatcccctgagaggctagcccacaagatccacttcaaaagccctagataacaccaagtctttccagacccagtgcacatcccatcagccaggacaccagtgtatgttgggatgcaaacagggaggcttatgacatctaatgtgttttccagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacataatttcttatttaattttattatgtatatattgtgtcagttcagatgccaaaaagaggtcttgaacatgtcacaggctctgatggcactgaccatggagaaagct

SEQ ID NO: 150 - DNA, 17 bp, Artificial Sequence
  synthetic construct
caagtctttccagaccc

SEQ ID NO: 151 - DNA, 58 bp, Artificial Sequence
  synthetic construct
cgcccgccgcgccccgcgcccgccccgccgcccccgcccgtgtatagatcaggcaggt

SEQ ID NO: 152 - DNA, 61 bp, Artificial Sequence
  synthetic construct
cgcccgccgcgccccgcgcccgccccgccgcccccgcccgcagaagatggaaatatcctgc

SEQ ID NO: 153 - DNA, 42 bp, Artificial Sequence
  synthetic construct
gcgcgcgcgcgcgcgctgtatatcacactttgatacaacact

SEQ ID NO: 154 - DNA, 17 bp, Artificial Sequence
  synthetic construct
aagccttgcgctcacac

SEQ ID NO: 155 - DNA, 65 bp, Artificial Sequence
  synthetic construct
cgcccgccgcgccccgcgcccgccccgccgcccccgcccgaataaccatatttaacacctctcaa

SEQ ID NO: 156 - DNA, 43 bp, Artificial Sequence
  synthetic construct
tctgcctttttcttccatcggggctatgatcacaccactgccc

SEQ ID NO: 157 - DNA, 42 bp, Artificial Sequence
  synthetic construct
tccccaaccccctaaagcgacctctttttggcatctgaactg

SEQ ID NO: 158 - DNA, 568 bp, Homo sapiens
tgttttcgaatgagtgaatcatcaacgagtggatgaaacgataatgtggctaacaggcagcagtaaggaggctgtgtagaataaacccgtaatcccgatgttggcagtttgcttagaaagaaaaagggaggcagtcggagaggggcacacgttttaacaaaatactgggaggaggaggaaggctagttttttttttgttttcaagtttccttctgatgttactcccatgcttccgggcacattacgagctcagtgcctgccggaaatctcccacctggtggcaacctacccttgcatacaccccacccaggggcttcaagccttgcagctgagtaaacacagaaaggagctctactaaggatgcgcgtctgcgggtttccgcgcgacctaggcgcaggcatgcgcagtagctaaagtcaccagcgtgcgcgggaagctgggccgcgtctgcttatgattggttgccgcggcagactcccacccaccgaaacgcagccctggaagctgattgggtgtggtcgccgtggccggacgccgctcgggggacgtgggaggggaggcgggaaac

SEQ ID NO: 159 - DNA, 1020 bp, Homo sapiens
ggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggtgagggccgggacggcgcgtgctggggagggacccggggccttgtggcgcggctcctttcccgcctcagagagtgggcggtgagcagcctctccagtgcggaggcacgggggcggaacgttggtgcttgtgcggattccgccgtccccaggttctgcttggctccggagggacgcccccctcagccctgaaacccgtgcctctccagccgccccggatctgaacttgtgatcacggagtgtttacgtcgtgccaggcattttaatgcattgttctagttcattttccagcagtcgcattcctcgccttggccctacatgtagcgctcattacaaacacggccagaatctcttattaacaaacagcagccaggagtgagatttaaaatagactgggggtttaggagacccttttatgacacgtaattctgctcccacgacgctcccatttataccgccggtccagctaagggtctggtaatggagcgccgttgaagagcagtatgatgaagtggtcaggaccaacggactctggagctgggctgcttgggatcaagtcgctgcccctctgcttattaacgtgtgaccttgggccagtcatggacgctatctgcttcagctcagcattcagtgctctccgtcacccgaccccatctatccaggattatctctccctggaaagctacaaacgtctcaccctatgtgggccaaatgttctggataggcctagttaacctcttct

SEQ ID NO: 160 - DNA, 42 bp, Artificial Sequence
  synthetic construct
tctgcctttttcttccatcgggggcgggaaacagcttagtgg

SEQ ID NO: 161 - DNA, 40 bp, Artificial Sequence
  synthetic construct
tccccaaccccctaaagcgacgcactggagaggctgctca

SEQ ID NO: 162 - DNA, 44 bp, Artificial Sequence
  synthetic construct
tctgcctttttcttccatcggggcgcagtagctaaagtcaccag

SEQ ID NO: 163 - DNA, 40 bp, Artificial Sequence
  synthetic construct
tccccaaccccctaaagcgagaatccgcacaagcaccaac

SEQ ID NO: 164 - DNA, 1080 bp, Homo sapiens
gaattcccatgtattgtgggagggacctggtgggagatagttgaatcatggggatggatctttcccatgctgttgtgatagtgaataagcctcatgagatctgatggttttaaaaacggaagtctacctgcacaagctctttctttgcctgctgccatccatgtaagacatgacttgttcctccttgccttctgccatgattgtgagacctccccagccatgtggaactataagtccagtaagcctctttttcttcccagtctcgggtatgtctttatcagcagcatgaagtccagctaatacagtgcttgaacatgtaatatctcaaatctgtaatgtactttttttttttttaaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggtaattatcttcctttttaatttacttatttttttaagagtagaaaaataaaaatgtgaagaatttaattgtgttttagtattttaagtagattgtgatagtagaatggtttgagacactttaatagcaattagcatgtggtttttaaaaagttgcagtttggctggtcgcagtggctcatgcttgtaatcccagtattttgggaggctgaggcaggtaggttgcctgagcccaggagttcaagaccagcctgcccaacgtggtaaagccccatctctactgaagataaaaaaatttaaaaaaattagctggggctattggcacacacctgtggtcccagctaatcaagaggatgaggttagaggatcacttgagcccaggaggttgaggttacagtttaactttcagaggccaaggcaggaggattgcttgagtccaggagtttgagaccaccctggggaatgtagggagatcccatctctatagagggatagattagatagataatttctgaggggaggggagggggagggccagggaaggggagggaaaggggaggggagggcag

SEQ ID NO: 165 - DNA, 19 bp, Artificial Sequence
  synthetic construct
ataaggcatccaaggagaa

SEQ ID NO: 166 - DNA, 66 bp, Artificial Sequence
  synthetic construct
cgcccgccgcgccccgcgcccgccccgccgcccccgcccgatctacttaaaatactaaaacacaat

SEQ ID NO: 167 - DNA, 60 bp, Artificial Sequence
  synthetic construct
cgcccgccgcgccccgcgcccgccccgccgcccccgcccgggagcaaagaatctgcagag

SEQ ID NO: 168 - DNA, 25 bp, Artificial Sequence
  synthetic construct
taattaccttatatgccaaatacca

SEQ ID NO: 169 - DNA, 42 bp, Artificial Sequence
  synthetic construct
tctgcctttttcttccatcgggtgctgccatccatgtaagac

SEQ ID NO: 170 - DNA, 40 bp, Artificial Sequence
  synthetic construct
tccccaaccccctaaagcgaccagccaaactgcaactttt

SEQ ID NO: 171 - DNA, 43 bp, Artificial Sequence
  synthetic construct
tctgcctttttcttccatcgggttcctccttgccttctgccat

SEQ ID NO: 172 - DNA, 43 bp, Artificial Sequence
  synthetic construct
tccccaaccccctaaagcgagggattacaagcatgagccactg

SEQ ID NO: 173 - DNA, 1294 bp, Homo sapiens
ccctggttcaagcttttctcccgcctcagcctcccgagtagctgggattacaggtgcatgctgcaacacccggctaatttttgtatttttagtagagatggggtttcaccatgttggccaggacggtctcgatctcctgacctcgtgatccgcctgccttggcctcccaaagtgttgggattacaggcgtgagccacagcactcagccagttatttttttataagaaaacattttactggccaggcctggtggctcacacctgtaatcccagcactttgggaggccgaggcaggcggatcacgaggtcaggagttcgagaccagcctggccaacatggtgaaaccccatctctactaaaaatacaaaaattagccaggcgtggtggtgtgcgcctgtattcccagctactggggaggctgaagcaggagaatcgattgaacccttgaggcagaggttgcagtgagttgagatcgcaccattgcactctagcctgggtgacagagcaagacttcatctcaaaaaaaagagaaaacattttattaataaggttcatagagtttggatttttcctttttgcttataaaattttaaagtatgttcaagagtttgttaaatttttaaaattttatttttacttaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacaggtaagcaaattgagtctagtgatagaggagattccaggcctaggaaaggctctttaattgacatgatactgtttcatttaaggaaaaataataaaaaaactcttttttttgtatctaattaaaataatgttctgatgtttacagaaactttgtatatttaattggacattagaacaagctgtttgttgtgtaagatttattttacctcagatcttttctcccccctttcctttctgtcttgtgttccaaaagagtaattattacggtaaatattactgtaattatggatttatcaaataagatgcagttctttagcattttttgataaatcgagtggaactttagcctgttattttactatttgttttattttaa

SEQ ID NO: 174 - DNA, 61 bp, Artificial Sequence
  synthetic construct
cgcccgccgcgccccgcgcccgccccgccgcccccgcccgaacattttattaataaggttc

SEQ ID NO: 175 - DNA, 15 bp, Artificial Sequence
  synthetic construct
attgccaggagaagc

SEQ ID NO: 176 - DNA, 62 bp, Artificial Sequence
  synthetic construct
cgcccgccgcgccccgcgcccgccccgccgcccccgcccgatttttacttaggcttctcctg

SEQ ID NO: 177 - DNA, 18 bp, Artificial Sequence
  synthetic construct
cagtttccccatgtctcc

SEQ ID NO: 178 - DNA, 18 bp, Artificial Sequence
  synthetic construct
aatgtgttttacccggag

SEQ ID NO: 179 - DNA, 63 bp, Artificial Sequence
  synthetic construct
cgcccgccgcgccccgcgcccgccccgccgcccccgcccgcttaaatgaaacagtatcatgtc

SEQ ID NO: 180 - DNA, 46 bp, Artificial Sequence
  synthetic construct
tctgcctttttcttccatcgggggttcatagagtttggatttttcc

SEQ ID NO: 181 - DNA, 46 bp, Artificial Sequence
  synthetic construct
tccccaaccccctaaagcgaccttaaatgaaacagtatcatgtcaa

SEQ ID NO: 182 - DNA, 1020 bp, Homo sapiens
gtggcttgctcctgtaatcctagctacttgggaggctgaggcaggagaattgcttgaacctgggaggcagaggtagcagtgagccaagatcgtgtcaccgcattccatcctgggcgacagtgagactctgtctcaaaacaaaaaaagagttgttaccgttgggactattttttgaaagctttatgtgaacgtaattttatattttgatgaaaatttagtttattgatgtaaaaagtgtatcagtacatcatatcagtgtcttgcacattgtataaacatttaatgtaggtgaatctgttatcactatagttatcaatgttataattttcatttttgcttttcttattccttttctcatagtagtttaaactatttctttcaaaatagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggtacatggattataaatgtgaattacaatatatataatgtaaatatgtaatatataataaataatatgtaaactatagtgactttttagaaggatatttctgtcatatttatctcaaaacctaaactgtgtatcaatgatattaagcttttttttttttttgagacagagtttcacttttgttgcccaggctggagtacaatggcgcgatcttggctcaccacatcctctgcctcccaggttcaagtgatcctcctgccttggcctcctgagtagctgggattacaggcatgtgccaccacgcctggctcatcttttttgtatttttagtagagatggggtttctctatgttggtcaggctggtctcaaactcctgaacctcaggtgatccgcccgcctcgggcttccaaagcgctgagattgcaggcatgagccactgtgtctggcctatttttatagtttatgtacttggaattatataatata

SEQ ID NO: 183 - DNA, 61 bp, Artificial Sequence
  synthetic construct
cgcccgccgcgccccgcgcccgccccgccgcccccgcccgtccttttctcatagtagttta

SEQ ID NO: 184 - DNA, 18 bp, Artificial Sequence
  synthetic construct
ttgaggtcctgataaatg

SEQ ID NO: 185 - DNA, 62 bp, Artificial Sequence
  synthetic construct
cgcccgccgcgccccgcgcccgccccgccgcccccgcccgtttctttcaaaatagataattc

SEQ ID NO: 186 - DNA, 16 bp, Artificial Sequence
  synthetic construct
tttttgcctttcaaca

SEQ ID NO: 187 - DNA, 17 bp, Artificial Sequence
  synthetic construct
atttatcaggacctcaa

SEQ ID NO: 188 - DNA, 60 bp, Artificial Sequence
  synthetic construct
cgcccgccgcgccccgcgcccgccccgccgcccccgcccgtgtaattcacatttataatc

SEQ ID NO: 189 - DNA, 16 bp, Artificial Sequence
  synthetic construct
attgccagaaatggag

SEQ ID NO: 190 - DNA, 64 bp, Artificial Sequence
  synthetic construct
cgcccgccgcgccccgcgcccgccccgccgcccccgcccgacatatttacattatatatattgt

SEQ ID NO: 191 - DNA, 40 bp, Artificial Sequence
  synthetic construct
tctgcctttttcttccatcggggcattccatcctgggcga

SEQ ID NO: 192 - DNA, 40 bp, Artificial Sequence
  synthetic construct
tccccaaccccctaaagcgacagcctgggcaacaaaagtg

SEQ ID NO: 193 - DNA, 1020 bp, Homo sapiens
agagacggggtttcactatgttggctaggctggtctcaaactcctagcctcgagtcatccacccgcctcgtcctcccggagtgcttggattacagcatgagccactgcgcccggcccccattttagttttgatggacatttgggtaattttcttttttggctattctaaataatgctgcaattactgttaattttcaccttgtaaaaaccattttcaaatctcaagagattaacctttagttttcttggtttggattgggaaggaacaccaaggaaaatgagggacttcagaatttattttcattttgcatttgttttttaaaatctttagaactggatccagtggtatagaaatcttcgatttttaaattcttaattttaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcaggtaaaaaaaaaaaaaaaaaaaaaaaaaaagggttaaaaatgttgaatggttaaaaaatgttttcattgacatatactgaagaagcttataaaggagctaaaatattttgaaatattattatacttggattagataactagctttaaatggctgtatttttctctcccctcctccactccactttttaacttttttttttttaagtcagagtctcacttgttccctaggccagagtgcagtggcacaatctcagcccactctaacctccacctcccaagtagttgggattacagttgcctgccaccatgcctggttaatttttatatttttagtagggttgcggggacagggtttcaccatgttggccaggttggtctcaaacttctgaccttaggtgatcctcccacctcggcttcccaaagtgctgggattacaggcttgagccatcgtgcccagcctactttttacttttttagagactgggcttg

SEQ ID NO: 194 - DNA, 57 bp, Artificial Sequence
  synthetic construct
cgcccgccgcgccccgcgcccgccccgccgcccccgcccgttcattttgcatttgtt

SEQ ID NO: 195 - DNA, 16 bp, Artificial Sequence
  synthetic construct
cttgattaccgcagac

SEQ ID NO: 196 - DNA, 59 bp, Artificial Sequence
  synthetic construct
cgcccgccgcgccccgcgcccgccccgccgcccccgcccgatcttcgatttttaaattc

SEQ ID NO: 197 - DNA, 17 bp, Artificial Sequence
  synthetic construct
aaaggttaagggctctg

SEQ ID NO: 198 - DNA, 44 bp, Artificial Sequence
  synthetic construct
tctgcctttttcttccatcgggttcttggtttggattgggaagg

SEQ ID NO: 199 - DNA, 42 bp, Artificial Sequence
  synthetic construct
tccccaaccccctaaagcgaggggagagaaaaatacagccat

SEQ ID NO: 200 - DNA, 45 bp, Artificial Sequence
  synthetic construct
tctgcctttttcttccatcgggagttttgatggacatttgggtaa

SEQ ID NO: 201 - DNA, 43 bp, Artificial Sequence
  synthetic construct
tccccaaccccctaaagcgagttaaaaagtggagtggaggagg

SEQ ID NO: 202 - DNA, 1320 bp, Homo sapiens
atggggtttcatcttgttggctaggctggactctaactccaggtgatctgcctgcctcggcctcccaaattgatgggattacaggtgtaaaccactgggcctggcctagcaatttaaaatgacattctaagaagttttatgtctaaatctgcagtaagtggctgggtgacgtggctcatgcctgtaatcccaacgctttgggagtccagggtgggaggatgacttgaggccaggagttgagaccagcctgggcaacatagtgagactctgtctctacaaaagaaaaaattagcggggcttagtggcgtgcgcctgtagtctcagctactcgaaaggctgaagtgggaggattctttgagccccaagggttctggcttgccgtgagccaggatggcaccactgcactccagtctgggcaatagagtcagaccctgtctcaacaaataaaataaaactgtagtaattataaagtggttttggctgggggagaaatgtacagttgaacatacggattaagaggttgaaagttggtcttaggaagaggaactttttgtggaaatttcttaatatttgaagaatattatgttattgttcctctgtttttcatggcgtagtaaggttttcactaatgagcttgccattctttctattttattttttgtttactagggttctgttgaagataccactggctctcagtctctggctgccttgctgaataagtgtaaaacccctcaaggacaaagacttgttaaccagtggattaagcagcctctcatggataagaacagaatagaggagaggtatgttattagtttatactttcgttagttttatgtaacctgcagttacccacatgattataccacttattgtaatatgcagttttggaagtatatgttaccatttaactgtacagagtacatagtaatagagtggtaattatttagattgattaaagaactcatttttttaaataagttttttttttttcactataaaagtttattttatttgagatggtatggtatcgaacatgttcatattgtgtgtaatcgtgggtaaattactcaacctttatgtcatagtttcttcacctttaaaatgacattaataaaagagctacttaataggattataagcatgagatgatttaatatacataaaatacttacagtctgatatataggaagcacttaactctttatcctagaaaagatttaaggtgaccttaacatatatgtcagaaaatctttaaaattgtggaaataaaaggttgtataattctgctatcctaaaattactagtatttcaatatat

SEQ ID NO: 203 - DNA, 56 bp, Artificial Sequence
  synthetic construct
cgcccgccgcgccccgcgcccgccccgccgcccccgcccggtttttcatggcgtag

SEQ ID NO: 204 - DNA, 17 bp, Artificial Sequence
  synthetic construct
actgagagccagtggta

SEQ ID NO: 205 - DNA, 22 bp, Artificial Sequence
  synthetic construct
tttactagggttctgttgaaga

SEQ ID NO: 206 - DNA, 59 bp, Artificial Sequence
  synthetic construct
cgcccgccgcgccccgcgcccgccccgccgcccccgcccgatacctctcctctattctg

SEQ ID NO: 207 - DNA, 18 bp, Artificial Sequence
  synthetic construct
tcaaggacaaagacttgt

SEQ ID NO: 208 - DNA, 63 bp, Artificial Sequence
  synthetic construct
cgcccgccgcgccccgcgcccgccccgccgcccccgcccgcatattacaataagtggtataat

SEQ ID NO: 209 - DNA, 42 bp, Artificial Sequence
  synthetic construct
tctgcctttttcttccatcgggtgaacatacggattaagagg

SEQ ID NO: 210 - DNA, 40 bp, Artificial Sequence
  synthetic construct
tccccaaccccctaaagcgacatatacttccaaaactgca

SEQ ID NO: 211 - DNA, 960 bp, Homo sapiens
ttttttttgagacagagtcttgctcttgttgcccaggctggagtgccatggcatgatctcagtgcaccacaatctctgcttcccaggtttaagcgattctcctgcctcagcctcccaagtagatgggatcacaggcatgagccaccatgcctggctaattttgtattttttgtacagacggggtttctccatgttggtcaggccagtctcgaactccctacctcaggtgatctgcctgcctcggcctctcaaagtgctgggattacaggtgtgagccactgcgcccagcagattcaagctttttaaatggaattttgagctgatttagttgagacttacgtgcttagttgataaattttaattttatactaaaatattttacattaattcaagttaatttatttcagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggtaacaagtgattttgtttttttgttttccttcaactcatacaatatatacttggcaatgtgctgtcctcataaagttggtggtggtgactcactcttaggacacattcagatttcttttttttttttttttgagaaggagtcttgctccgttgccaaggctagagtgcagtggcacaatctcagctcactgcaacctctgcctcctgggttcaagcgattctcctgcctcagcttcctgagtggctgggattacaggcatgtgccaccatgcccggctaatttttgtacttttagttttaccatgttggccaggttcgtctggaactcccaatctcaggtgacccacctgcc

SEQ ID NO: 212 - DNA, 58 bp, Artificial Sequence
  synthetic construct
cgcccgccgcgccccgcgcccgccccgccgcccccgcccggttgagacttacgtgctt

SEQ ID NO: 213 - DNA, 21 bp, Artificial Sequence
  synthetic construct
caattctgcatcttctacaaa

SEQ ID NO: 214 - DNA, 61 bp, Artificial Sequence
  synthetic construct
cgcccgccgcgccccgcgcccgccccgccgcccccgcccgatttcagattgaatttagtgg

SEQ ID NO: 215 - DNA, 19 bp, Artificial Sequence
  synthetic construct
agtttgctgcttgtctttg

SEQ ID NO: 216 - DNA, 17 bp, Artificial Sequence
  synthetic construct
gacttgccaagaagttt

SEQ ID NO: 217 - DNA, 58 bp, Artificial Sequence
  synthetic construct
cgcccgccgcgccccgcgcccgccccgccgcccccgcccgtgagtcaccaccaccaac

SEQ ID NO: 218 - DNA, 47 bp, Artificial Sequence
  synthetic construct
tctgcctttttcttccatcggggctgatttagttgagacttacgtgc

SEQ ID NO: 219 - DNA, 40 bp, Artificial Sequence
  synthetic construct
tccccaaccccctaaagcgagaggacagcacattgccaag

SEQ ID NO: 220 - DNA, 840 bp, Homo sapiens
tataagaaatgaaattcatttagtcataattaatgtcatgtttctgcatctatattacttgttgggtttacagacgaggtagtgtattattagtgggaagctttgagtgctacatcatctccctttctataaaataaattgagtacgaaacaatttgaattaaaacacctgagtaaatagtaactttggagacctgctgtactatttgtaccttttggatcaaatgatgcttgtttatctcagtcaaaattttatgatttgtattctgtaaaatgagatctttttatttgtttgttttactactttcttttaggaaaacaccagaaattattgttggcagtttttgtgactcctcttactgatcttcgttctgacttctccaagtttcaggaaatgatagaaacaactttagatatggatcaggtatgcaatatactttttaatttaagcagtagttatttttaaaaagcaaaggccactttaagaaagtttgtagatttttctttttagtatctaattgtagcacctttgtggacagtggatgtaatattaagtgacagatgggaaaaggatttttaaaaaaatagcaactgtttcagtggatgaaataaagattattagcagagaaaatgaatattgggcataactgtcctggtgaaagacaatctcataaatgaacaatttcataatttcgtaaatgcaactgcattttattttcaaagagaaggaaaattatagtcactggaaacggaaagagaagttagaggtaaacataggacacacaagaaaactttcattttgtttattttcttgtttttcttttgagacagggtttccctc

SEQ ID NO: 221 - DNA, 57 bp, Artificial Sequence
  synthetic construct
cgcccgccgcgccccgcgcccgccccgccgcccccgcccgtttggatcaaatgatgc

SEQ ID NO: 222 - DNA, 19 bp, Artificial Sequence
  synthetic construct
atcagtaagaggagtcaca

SEQ ID NO: 223 - DNA, 18 bp, Artificial Sequence
  synthetic construct
ttgtgactcctcttactg

SEQ ID NO: 224 - DNA, 61 bp, Artificial Sequence
  synthetic construct
cgcccgccgcgccccgcgcccgccccgccgcccccgcccgaataactactgcttaaattaa

SEQ ID NO: 225 - DNA, 18 bp, Artificial Sequence
  synthetic construct
ctgacttctccaagtttc

SEQ ID NO: 226 - DNA, 21 bp, Artificial Sequence
  synthetic construct
gtgctacaattagatactaaa

SEQ ID NO: 227 - DNA, 20 bp, Artificial Sequence
  synthetic construct
agaaattattgttggcagtt

SEQ ID NO: 228 - DNA, 61 bp, Artificial Sequence
  synthetic construct
cgcccgccgcgccccgcgcccgccccgccgcccccgcccgattgcatacctgatccatatc

SEQ ID NO: 229 - DNA, 44 bp, Artificial Sequence
  synthetic construct
tctgcctttttcttccatcgggaatagtaactttggagacctgc

SEQ ID NO: 230 - DNA, 40 bp, Artificial Sequence
  synthetic construct
tccccaaccccctaaagcgacaggacagttatgcccaata

SEQ ID NO: 231 - DNA, 780 bp, Homo sapiens
cacattgaacgttatttggtaatttttagagaggacattttaaatgtttaggaaaaatataaataaaatgtagaatactattgggggcatatacatcatcagcactgtaactgtttcatatgaatcatttttgtacatatagaactctaaagtcctaatgaacagaattttacatttctataaatagaaagtccttaatagttgtgactgaataacttatggatagcaaattatttaactgaaaacagtaaaatttaagtgggaggaaatatttgctttataatttctgtctttacccattatttataggattttgtcactttgttctgtttgcaggtggaaaaccatgaattccttgtaaaaccttcatttgatcctaatctcagtgaattaagagaaataatgaatgacttggaaaagaagatgcagtcaacattaataagtgcagccagagatcttggtaagaatgggtcattggaggttggaataattcttttgtctatacactgtatagacaaaatattgatgccagaattattttataagttccctgtccccaagatgatgacttcacatctctgtcaaacagaaatcgcccaacaggcccttgtatgatgtcatttaaacaagccctattttaaatgtcacctccactggtaacaggatactcctaggaggatcaccaagcccaattcttctaggagtagtgcattgattaggctttggggtttccaagcagttcattaatgtcacttttggaaaaagtctgtctttcatacc

SEQ ID NO: 232 - DNA, 60 bp, Artificial Sequence
  synthetic construct
cgcccgccgcgccccgcgcccgccccgccgcccccgcccgaatatttgctttataatttc

SEQ ID NO: 233 - DNA, 17 bp, Artificial Sequence
  synthetic construct
agaattattccaacctc

SEQ ID NO: 234 - DNA, 46 bp, Artificial Sequence
  synthetic construct
tctgcctttttcttccatcggggaaagtccttaatagttgtgactg

SEQ ID NO: 235 - DNA, 44 bp, Artificial Sequence
  synthetic construct
tccccaaccccctaaagcgagggaacttataaaataattctggc

SEQ ID NO: 236 - DNA, 780 bp, Homo sapiens
tcatgcataactcctcgagggtggggttacaccttaatccatcctcaggtgctcatggtaattggggcaaatatgttgcccagtgctggtgctctgcagccttggatgggtttacccagaaagcagctttcaagtcagaaactaacattcataagggagttaaggattttataaatagatatccataattcatgtagttttcaagtaagtagtatttgaatcttttctggttagataataattgtgagtatgttgtcatataataacagtatgtttttcactatttaaataattttagaattacattgaaaaatggtagtaggtatttatggaatactttttcttttcttcttgattatcaaggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacaggtttgcaagtcgttattatatttttaaccctttattaattccctaaatgctctaacatgatgtgaatgttctatgataagttttactaatgtagtcatcaggtaagagtcaagctttcttccatagagcagtcagctgtcgcaacaccatttgttaaatagtccgtctgttctccattgactgaagtggtactttgggtctattttaaagactctacttttacctcgtctcaccattcttttgtctacacaaaatatattttatcg

SEQ ID NO: 237 - DNA, 59 bp, Artificial Sequence
  synthetic construct
cgcccgccgcgccccgcgcccgccccgccgcccccgcccggaattacattgaaaaatgg

SEQ ID NO: 238 - DNA, 17 bp, Artificial Sequence
  synthetic construct
ttaatctgtttgccagg

SEQ ID NO: 239 - DNA, 19 bp, Artificial Sequence
  synthetic construct
tcttcttgattatcaaggc

SEQ ID NO: 240 - DNA, 58 bp, Artificial Sequence
  synthetic construct
cgcccgccgcgccccgcgcccgccccgccgcccccgcccgacaccattcttctggata

SEQ ID NO: 241 - DNA, 21 bp, Artificial Sequence
  synthetic construct
tgcacagtttggatattactt

SEQ ID NO: 242 - DNA, 65 bp, Artificial Sequence
  synthetic construct
cgcccgccgcgccccgcgcccgccccgccgcccccgcccggtaaaacttatcatagaacattcac

SEQ ID NO: 243 - DNA, 43 bp, Artificial Sequence
  synthetic construct
tctgcctttttcttccatcgggtcataagggagttaaggattt

SEQ ID NO: 244 - DNA, 40 bp, Artificial Sequence
  synthetic construct
tccccaaccccctaaagcgactgctctatggaagaaagct

SEQ ID NO: 245 - DNA, 780 bp, Homo sapiens
gttctggggttacaggcgtgagccaccacgcccggctgtcttcaatcttaaataaggattccatttaaatattttgtaaaaggacacagatcacagttttactcaggggaatataattgttatagcaggaattgtgccattgcgctattccaaacagtgtaaaagaacattaataaattgaattctaactacatttgtccctaaggagttgttcgttttccacttgtatttccattttaattatcattatttggatgtttcataggatactttggatatgtttcacgtagtacacattgcttctagtacacattttaatatttttaataaaactgttatttcgatttgcagcaaattgacttctttaaatgaagagtataccaaaaataaaacagaatatgaagaagcccaggatgccattgttaaagaaattgtcaatatttcttcaggtaaacttaatagaactaataatgttctgaatgtcacctggcttttggtaacagaagaaaaatcatgatatttgaagtgtgttttgttattttcgcaagccattacattctgactatttaatatgttaggtttcctatataaaataaggcatggtatgttacagtaggacacataactggaagttactcttgcacatagaaacaaaaaatggcagaaaagcacaaaacttactatagttgtaacagggaaaggaaacactagggcctacaacgtactaatgtcttgggtcatctatgggctcatgaggctctaggttatggaagtaaatac

SEQ ID NO: 246 - DNA, 19 bp, Artificial Sequence
  synthetic construct
tttggatatgtttcacgta

SEQ ID NO: 247 - DNA, 18 bp, Artificial Sequence
  synthetic construct
ctttaacaatggcatcct

SEQ ID NO: 248 - DNA, 21 bp, Artificial Sequence
  synthetic construct
gcaaattgacttctttaaatg

SEQ ID NO: 249 - DNA, 18 bp, Artificial Sequence
  synthetic construct
atggcttgcgaaaataac

SEQ ID NO: 250 - DNA, 45 bp, Artificial Sequence
  synthetic construct
tctgcctttttcttccatcgggcatttgtccctaaggagttgttc

SEQ ID NO: 251 - DNA, 40 bp, Artificial Sequence
  synthetic construct
tccccaaccccctaaagcgacagaatgtaatggcttgcga

SEQ ID NO: 252 - DNA, 1260 bp, Homo sapiens
tgtggcgcaatctcagcttactgcaacttccaccttctgggttcatgcaattctggtgcctcagcctcccaagtatctgggtttacagacatgcaccaccatacctggctaatttttgtatttttggtagagatggggtttcgccgtgttaccaggctggtcttgaattcctggccccatgtgatcccccggcctcatgcgatctgcccgcctcagcctccctaagtgctgggattataggcgtgagccacccaacccagccagtactctgtttttgatagctattcacaatgggaaaggatgtagcaacacattttaaccctatgttgagttttaggtgggttcctttgaaattttgttaaggctaacttttgttaatttttttaaaaaagtgtaaattaggaaatgggttttgaattcccaaatggggggattaaatgtatttttacggcttatatctgtttattattcagtattcctgtgtacattttctgtttttatttttatacaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggtaaaaaacctggtttttgggctttgtgggggtaacgttttgtttttttttttttttttttaatcttggagtagaaatatatttaaaattgatggagaaaattcccagttcttaacattagaaagggaatatattattcttaccagttagtaatctattcacatttggtttagagggaagatttagaaggtgagataaaagcttgtgagagaatagtgtattcatgtgaaacttcttccatgggttcagagcatttagaaacaaacatcccttcacactcaaagcttacctttgagccagtcctccaatagtgaggtctttgaaggtcaggccaaattggctgtgggaggacctcaggttaggataggaattattttaagacatggcactatattcatgtgaaactcgcaaaaactagccttgcatataggctcatgtatcatgtctcagctgagatgtttgagagatcttaactagattctagaaaacaaaaaaggaagtag

SEQ ID NO: 253 - DNA, 57 bp, Artificial Sequence
  synthetic construct
cgcccgccgcgccccgcgcccgccccgccgcccccgcccgaggaaatgggttttgaa

SEQ ID NO: 254 - DNA, 20 bp, Artificial Sequence
  synthetic construct
gagctaacacatcattgagt

SEQ ID NO: 255 - DNA, 61 bp, Artificial Sequence
  synthetic construct
cgcccgccgcgccccgcgcccgccccgccgcccccgcccgatttttatacaggctatgtag

SEQ ID NO: 256 - DNA, 18 bp, Artificial Sequence
  synthetic construct
acatatggaacaggtgct

SEQ ID NO: 257 - DNA, 17 bp, Artificial Sequence
  synthetic construct
tggagcacctgttccat

SEQ ID NO: 258 - DNA, 57 bp, Artificial Sequence
  synthetic construct
cgcccgccgcgccccgcgcccgccccgccgcccccgcccgaacaaaacgttaccccc

SEQ ID NO: 259 - DNA, 19 bp, Artificial Sequence
  synthetic construct
cagctttgctcacgtgtca

SEQ ID NO: 260 - DNA, 62 bp, Artificial Sequence
  synthetic construct
cgcccgccgcgccccgcgcccgccccgccgcccccgcccgcatcttgaacttcaacacaagc

SEQ ID NO: 261 - DNA, 44 bp, Artificial Sequence
  synthetic construct
tctgcctttttcttccatcgggtgttgagttttaggtgggttcc

SEQ ID NO: 262 - DNA, 39 bp, Artificial Sequence
  synthetic construct
tccccaaccccctaaagcgatacccccacaaagcccaaa

SEQ ID NO: 263 - DNA, 780 bp, Homo sapiens
atgggcagtaactctgtccacatctttgggcaggctgtggttctgcctttatatgctatgtcagtgtaaacctacgcgattaatcatcagtgtacagtttaggactaacaatccatttattagtagcagaaagaagtttaaaatcttgctttctgatataatttgttttgtaggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtaagtgcatctcctagtcccttgaagatagaaatgtatgtctctgtcctgtgagaaggaaaagtatatttgcagattctcatgtaaaaacatctgagaatgtttgtcttagtttaatagttgttttcctgtggactttatatactttgtattgtcttaaaagagtgattgatggtagctacggaaaactttgatttttaaaattgtctctttaagtagacaatttataagctactggtacgagttcaccttataaatctccactaccatgtttttgcttggactgttcacacttcctggaatggtccttcttgccgtttatccaacttctttctaatttttaagtccctaatgatgggaattctatttctgtagtgatttttctggtcatacgaccgtaag

SEQ ID NO: 264 - DNA, 58 bp, Artificial Sequence
  synthetic construct
cgcccgccgcgccccgcgcccgccccgccgcccccgcccgtaggactaacaatccatt

SEQ ID NO: 265 - DNA, 16 bp, Artificial Sequence
  synthetic construct
tgggccatgagtacta

SEQ ID NO: 266 - DNA, 57 bp, Artificial Sequence
  synthetic construct
cgcccgccgcgccccgcgcccgccccgccgcccccgcccgatgggaggtaaatcaac

SEQ ID NO: 267 - DNA, 18 bp, Artificial Sequence
  synthetic construct
gactcctttcaattgact

SEQ ID NO: 268 - DNA, 20 bp, Artificial Sequence
  synthetic construct
ttgtggactgcatcttagcc

SEQ ID NO: 269 - DNA, 24 bp, Artificial Sequence
  synthetic construct
tcacaggacagagacatacatttc

SEQ ID NO: 270 - DNA, 45 bp, Artificial Sequence
  synthetic construct
tctgcctttttcttccatcggggctatgtcagtgtaaacctacgc

SEQ ID NO: 271 - DNA, 44 bp, Artificial Sequence
  synthetic construct
tccccaaccccctaaagcgacttctcacaggacagagacataca

SEQ ID NO: 272 - DNA, 1200 bp, Homo sapiens
ccgttgtttgttcatgttcatgacctttttttttttttcctattctcctccctccctccctccctccctcccttccttccttccctccttccctccttccctccctccctcccacacaaaggtgtgtgctaccatacctggctagtttttaatttttttttttttttttttttttagaggcaaggtctcactatgttgctcaggctggtctgggctcaagtgatcctcccacctccgccttccaaagtgctgggattacagacgtgagccatcatgcctggcccttgcccatttttctattgaagttttagtgctttttattgactttgtttatatattaagataatccattatgtttgtggcatatccttcccaatgtattgtcttaattttgtttttgtatgtgtatgttaccacattttatgtgatgggaaatttcatgtaattatgtgcttcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtatgtactattggagtactctaaattcagaacttggtaatgggaaacttactacccttgaaatcatcagtaattgccttattctaagttagtataaattattgatgttgttatagaacccatttaccccttaattcacagtctgggggtaggaacatgtacatcatatttctgtatctcatagtaggaccactcattctaaagcattcacagaaagaattatctgtactctttttgggacagaatctcgttctgttgcccaggctggagtgcgatctcggctcactgcaacctccgcctcccgggttcaagcgattctcctgcctcagcttcccgagtagctgggattacaggcgcctgccaccacacctggctaatttttatatttttagtagagacggggtttcaccatgctggccaggctggtctcgaattcctgacctcaggcaatccacccgtctcggcctcccaaagtgctgggattacaggtgtga

SEQ ID NO: 273 - DNA, 61 bp, Artificial Sequence
  synthetic construct
cgcccgccgcgccccgcgcccgccccgccgcccccgcccggtatgtgtatgttaccacatt

SEQ ID NO: 274 - DNA, 20 bp, Artificial Sequence
  synthetic construct
tagttaaggtctcttcagtg

SEQ ID NO: 275 - DNA, 20 bp, Artificial Sequence
  synthetic construct
ataatctacatgtcacagca

SEQ ID NO: 276 - DNA, 59 bp, Artificial Sequence
  synthetic construct
cgcccgccgcgccccgcgcccgccccgccgcccccgcccggaataaggcaattactgat

SEQ ID NO: 277 - DNA, 43 bp, Artificial Sequence
  synthetic construct
tctgcctttttcttccatcgggatgtttgtggcatatccttcc

SEQ ID NO: 278 - DNA, 45 bp, Artificial Sequence
  synthetic construct
tccccaaccccctaaagcgatagtaagtttcccattaccaagttc

SEQ ID NO: 279 - DNA, 960 bp, Homo sapiens
ccctcccttaccttcccatgaaatgagaaagcctcagagatagtggcttgattaatttttctttagattaagatatttgtctaagcctttaaggtttatctattgagcttttttgtctcctatttttatttttcctactatgtttgtcgaggataaaatacagcactgtgtgccaagtcataatcacttttcatttgagacttaattaaaatgcctttattttaatgatatatttggctaatgtatttgaagtaatccgaaattaagttttctaatgacaaggtgagaaggataaattccatttacataaattgctgtctcttctcatgctgtcccctcacgcttccccaaatttcttataggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagaggtttgtcagtttgttttcatagtttaacttagcttctctattattacataaacaggacactaagatgaaggttttttgttgttgtttgttttcctctgtgtttctagtgcttattttttaatcagtttttttgatggcaaagaatctatctctgtgttattttgatttctgcagtatatacatctgcatgatcaatattcgatttcaagtaccaaagtaggagtaaaggaatattaacctaggtttaaaattagtcatttcactaaaattagttattatggacgatagatgtctaggtatatctttgttcataaacgaatatatcaagttcagttattaaattacacattaggtaagaaaaggacaaagaaataaaaaagcatgattcataattcctgccctctatttgtctagaatttagt

SEQ ID NO: 280 - DNA, 18 bp, Artificial Sequence
  synthetic construct
gtctcttctcatgctgtc

SEQ ID NO: 281 - DNA, 61 bp, Artificial Sequence
  synthetic construct
cgcccgccgcgccccgcgcccgccccgccgcccccgcccgaatagagaagctaagttaaac

SEQ ID NO: 282 - DNA, 47 bp, Artificial Sequence
  synthetic construct
tctgcctttttcttccatcgggttggctaatgtatttgaagtaatcc

SEQ ID NO: 283 - DNA, 43 bp, Artificial Sequence
  synthetic construct
tccccaaccccctaaagcgaacacagaggaaaacaaacaacaa

SEQ ID NO: 284 - DNA, 1028 bp, Homo sapiens
gactcttttatgcaatctcttgtttccagttagaatagaagtcgtgtacttttgataacattaattataatatattttgagccctgtgaggttggtaacattattcccattttatgaatgaggaatgtgtgttaaggagtttgcccaagagtcacatagcaagtcatagtcatgctctctgaagcagcaataacttggcaataaaataaaaatgaagcatcttctgtatgtgttaacttttcagtgactgtttatgccttccagtattctttgtaaaccttgaattctttttttcacagatgattaaagtttatcaattgtaaaggtggaggaatttgggaactagacagtgcacacataaataataaatatgttcttcaaatattgggtgggctaatgtgggaggagtttgagaccagcctgggcaacatagtgagaccctcgtctctaaaaatatgaaaaataaaaaaaaaattttttaaatgtgtgatatgtttagatggaaatgaaacaatttgtcactgtctaacatgacttttagaaaagatattttaattactaatgggacattcacatgtgtttcagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtgg

SEQ ID NO: 285 - DNA, 22 bp, Artificial Sequence
  synthetic construct
ttactaatgggacattcacatg

SEQ ID NO: 286 - DNA, 65 bp, Artificial Sequence
  synthetic construct
cgcccgccgcgccccgcgcccgccccgccgcccccgcccgacaatagcttatcaatattaccttc

SEQ ID NO: 287 - DNA, 43 bp, Artificial Sequence
  synthetic construct
tctgcctttttcttccatcggggtaaaggtggaggaatttggg

SEQ ID NO: 288 - DNA, 44 bp, Artificial Sequence
  synthetic construct
tccccaaccccctaaagcgaggcactgacagttaacactatgga

SEQ ID NO: 289 - DNA, 22 bp, Artificial Sequence
  synthetic construct
tctgcctttttcttccatcggg

SEQ ID NO: 290 - DNA, 20 bp, Artificial Sequence
  synthetic construct
tccccaaccccctaaagcga

SEQ ID NO: 291 - DNA, 42 bp, Artificial Sequence
  synthetic construct
tctgcctttttcttccatcgggcagagcaagacttcatctca

SEQ ID NO: 292 - DNA, 24 bp, Artificial Sequence
  synthetic construct
ggagcaaagaatctgcagagtgtt

SEQ ID NO: 293 - DNA, 25 bp, Artificial Sequence
  synthetic construct
ctgaaaaaggttaagggctctgact

SEQ ID NO: 294 - DNA, 58 bp, Artificial Sequence
  synthetic construct
cgcccgccgcgccccgcgcccgccccgccgcccccgcccgggatgataattggaggca

SEQ ID NO: 295 - DNA, 20 bp, Artificial Sequence
  synthetic construct
taggagaagtgtgaataaag

SEQ ID NO: 296 - DNA, 60 bp, Artificial Sequence
  synthetic construct
cgcccgccgcgccccgcgcccgccccgccgcccccgcccgctgttctgtgatattatgtg

SEQ ID NO: 297 - DNA, 60 bp, Artificial Sequence
  synthetic construct
cgcccgccgcgccccgcgcccgccccgccgcccccgcccgtttgcttctccagttgaaca

SEQ ID NO: 298 - DNA, 60 bp, Artificial Sequence
  synthetic construct
cgcccgccgcgccccgcgcccgccccgccgcccccgcccgttgaagtgtccaccaaaatg

SEQ ID NO: 299 - DNA, 60 bp, Artificial Sequence
  synthetic construct
cgcccgccgcgccccgcgcccgcccctgccgcccccgcccggtactatccccaagtaacc

SEQ ID NO: 300 - DNA, 68 bp, Artificial Sequence
  synthetic construct
tcgcccgccgcgccccgcgcccgccccgccgcccccgcccgtacagtggatatagaaaggacaatttt

SEQ ID NO: 301 - DNA, 25 bp, Artificial Sequence
  synthetic construct
cagattctctacttcatagccatag

SEQ ID NO: 302 - DNA, 62 bp, Artificial Sequence
  synthetic construct
cgcccgccgcgccccgcgcccgccccgccgcccccgcccgctatttatggttttgcttgtgg

SEQ ID NO: 303 - DNA, 60 bp, Artificial Sequence
  synthetic construct
cgcccgccgcgccccgcgcccgccccgccgcccccgcccggctcagtataactgaggctg

SEQ ID NO: 304 - DNA, 59 bp, Artificial Sequence
  synthetic construct
cgcccgccgcgccccgcgcccgccccgccgcccccgcccgcaaaagttgatggcagagt

SEQ ID NO: 305 - DNA, 18 bp, Artificial Sequence
  synthetic construct
tgtcaggccaattacaga

SEQ ID NO: 306 - DNA, 58 bp, Artificial Sequence
  synthetic construct
cgcccgccgcgccccgcgcccgccccgccgcccccgcccgggggtgaggaattttgaa

SEQ ID NO: 307 - DNA, 59 bp, Artificial Sequence
  synthetic construct
cgcccgccgcgccccgcgcccgccccgccgcccccgcccgatacccttattccctgtgg

SEQ ID NO: 308 - DNA, 20 bp, Artificial Sequence
  synthetic construct
cactggttgggctagtatgt

SEQ ID NO: 309 - DNA, 58 bp, Artificial Sequence
  synthetic construct
cgcccgccgcgccccgcgcccgccccgccgcccccgcccgcccagtgttgagcctttg

SEQ ID NO: 310 - DNA, 21 bp, Artificial Sequence
  synthetic construct
gctcccagtagggtcagcatc

SEQ ID NO: 311 - DNA, 20 bp, Artificial Sequence
  synthetic construct
atggccaagtactaggttgg

SEQ ID NO: 312 - DNA, 20 bp, Artificial Sequence
  synthetic construct
ctaaccgattgaatatggag

SEQ ID NO: 313 - DNA, 60 bp, Artificial Sequence
  synthetic construct
cgcccgccgcgccccgcgcccgccccgccgcccccgcccgatactttgttacttgtctga

SEQ ID NO: 314 - DNA, 20 bp, Artificial Sequence
  synthetic construct
gttatcaagaattacaaggg

SEQ ID NO: 315 - DNA, 20 bp, Artificial Sequence
  synthetic construct
cgatcagaccctacaggaca

SEQ ID NO: 316 - DNA, 27 bp, Artificial Sequence
  synthetic construct
gatacccaatttcataaatagcattca

SEQ ID NO: 317 - DNA, 22 bp, Artificial Sequence
  synthetic construct
catagaatgacaggacaatagg

SEQ ID NO: 318 - DNA, 27 bp, Artificial Sequence
  synthetic construct
tgcttatttcatctcaatcctacgctt

SEQ ID NO: 319 - DNA, 67 bp, Artificial Sequence
  synthetic construct
cgcccgccgcgccccgcgcccgccccgccgcccccgcccgaatattcattttaaagatccaagatat

SEQ ID NO: 320 - DNA, 25 bp, Artificial Sequence
  synthetic construct
taaggggacatacactgagaatgaa

SEQ ID NO: 321 - DNA, 20 bp, Artificial Sequence
  synthetic construct
ctctgagtcagttaaacagt

SEQ ID NO: 322 - DNA, 18 bp, Artificial Sequence
  synthetic construct
atgactttatggcaggga

SEQ ID NO: 323 - DNA, 60 bp, Artificial Sequence
  synthetic construct
cgcccgccgcgccccgcgcccgccccgccgcccccgcccgtacggaaatggtataggaaa

SEQ ID NO: 324 - DNA, 19 bp, Artificial Sequence
  synthetic construct
ggtgaggggtgtaatggtt

SEQ ID NO: 325 - DNA, 21 bp, Artificial Sequence
  synthetic construct
atggctctatgtcatcttgtc

SEQ ID NO: 326 - DNA, 58 bp, Artificial Sequence
  synthetic construct
cgcccgccgcgccccgcgcccgccccgccgcccccgcccgtaggttgagggttgggac

SEQ ID NO: 327 - DNA, 21 bp, Artificial Sequence
  synthetic construct
cctcgtggtgtagagtgatgt

SEQ ID NO: 328 - DNA, 60 bp, Artificial Sequence
  synthetic construct
cgcccgccgcgccccgcgcccgccccgccgcccccgcccggagcccaggagcccagaaat

SEQ ID NO: 329 - DNA, 60 bp, Artificial Sequence
  synthetic construct
cgcccgccgcgccccgcgcccgccccgccgcccccgcccggagggccatagactatagca

SEQ ID NO: 330 - DNA, 20 bp, Artificial Sequence
  synthetic construct
tttctgtccctgctctggtc

SEQ ID NO: 331 - DNA, 60 bp, Artificial Sequence
  synthetic construct
cgcccgccgcgccccgcgcccgccccgccgcccccgcccgtcccacgagctccaattcca

SEQ ID NO: 332 - DNA, 58 bp, Artificial Sequence
  synthetic construct
cgcccgccgcgccccgcgcccgccccgccgcccccgcccgggtgaggtctgggaagtg

SEQ ID NO: 333 - DNA, 19 bp, Artificial Sequence
  synthetic construct
tgcctccttgagtatctgc

SEQ ID NO: 334 - DNA, 60 bp, Artificial Sequence
  synthetic construct
cgcccgccgcgccccgcgcccgccccgccgcccccgcccgcagccaagccctacctctcg

SEQ ID NO: 335 - DNA, 20 bp, Artificial Sequence
  synthetic construct
cttcatcaccccctccctgc

SEQ ID NO: 336 - DNA, 60 bp, Artificial Sequence
  synthetic construct
cgcccgccgcgccccgcgcccgccccgccgcccccgcccgcttgatgacttcctatccat

SEQ ID NO: 337 - DNA, 21 bp, Artificial Sequence
  synthetic construct
aacctccatccagtgcctagc

SEQ ID NO: 338 - DNA, 206 bp, Artificial Sequence
  synthetic construct
ggggggggggggggggggggggggggggggggggggggggggggggggggttatcattgcttggctcatattaaaatatgtacattagagtagttgcagactgataaattattttctgtttgatttgccagtttagatgcaaaatccacaagtattcaagtgattgttaaagagggaggcctgaagttgattcagatccaagacaa

SEQ ID NO: 339 - DNA, 188 bp, Artificial Sequence
  synthetic construct
gggggggggggggggggggggggggggggggggggggggggggggggggggcaaaatccacaagtattcaagtgattgttaaagagggaggcctgaagttgattcagatccaagacaatggcaccgggatcagggtaagtaaaacctcaaagtagcaggatgtttgtgcgcttcatggaagagtcagg

SEQ ID NO: 340 - DNA, 277 bp, Artificial Sequence
  synthetic construct
ggggggggggggggggggggggggggggggggggggggggggggggggggaatatttgctttataatttctgtctttacccattatttataggattttgtcactttgttctgtttgcaggtggaaaaccatgaattccttgtaaaaccttcatttgatcctaatctcagtgaattaagagaaataatgaatgacttggaaaagaagatgcagtcaacattaataagtgcagccagagatcttggtaagaatgggtcattggaggttggaataattct

SEQ ID NO: 341 - DNA, 314 bp, Artificial Sequence
  synthetic construct
gtctcttctcatgctgtcccctcacgcttccccaaatttcttataggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagaggtttgtcagtttgttttcatagtttaacttagcttctctattgggggggggggggggggggggggggggggggggggggggggggggggggg

SEQ ID NO: 342 - DNA, 236 bp, Artificial Sequence
  synthetic construct
gggggggggggggggggggggggggggggggggggggggggggggggggggggaattcaaagagatttggaaaaatgagtaacatgattatttactcatctttttggtatctaacagaaagaagatctggatattgtatgtgaaaggttcactactagtaaactgcagtcctttgaggatttagccagtatttctacctatggctttcgaggtgaggtaagctaaagattcaagaa

SEQ ID NO: 343 - DNA, 239 bp, Artificial Sequence
  synthetic construct
atattgtatgtgaaaggttcactactagtaaactgcagtcctttgaggatttagccagtatttctacctatggctttcgaggtgaggtaagctaaagattcaagaaatgtgtaaaatatcctcctgtgatgacattgtctgtcatttgttagtatgtatttctcaacatagataaataaggtttggtacgggggggggggggggggggggggggggggggggggggggggggggggggg

SEQ ID NO: 344 - DNA, 40 bp, Artificial Sequence
  synthetic construct
cgcccgccgcgccccgcgcccgccccgccgcccccgcccg

SEQ ID NO: 345 - DNA, 40 bp, Artificial Sequence
  synthetic construct
cgggcgggggcggcggggcgggcgcggggcgcggcgggcg

SEQ ID NO: 346 - DNA, 203 bp, Artificial Sequence
  synthetic construct
cgcccgccgcgccccgcgcccgccccgccgcccccgcccgggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggtaatta

SEQ ID NO: 347 - DNA, 174 bp, Artificial Sequence
  synthetic construct
ataaggcatccaaggagaatgattggtatttggcatataaggtaattatcttcctttttaatttacttatttttttaagagtagaaaaataaaaatgtgaagaatttaattgtgttttagtattttaagtagatcgggcgggggcggcggggcgggcgcggggcgcggcgggcg

SEQ ID NO: 348 - DNA, 164 bp, Artificial Sequence
  synthetic construct
cgcccgccgcgccccgcgcccgccccgccgcccccgcccgaacattttattaataaggttcatagagtttggatttttcctttttgcttataaaattttaaagtatgttcaagagtttgttaaatttttaaaattttatttttacttaggcttctcctggcaat

SEQ ID NO: 349 - DNA, 325 bp, Artificial Sequence
  synthetic construct
cgcccgccgcgccccgcgcccgccccgccgcccccgcccgatttttacttaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactg

SEQ ID NO: 350 - DNA, 175 bp, Artificial Sequence
  synthetic construct
aatgtgttttacccggaggagagactgctggagacatggggaaactgagacaggtaagcaaattgagtctagtgatagaggagattccaggcctaggaaaggctctttaattgacatgatactgtttcatttaagcgggcgggggcggcggggcgggcgcggggcgcggcgggcg

SEQ ID NO: 351 - DNA, 161 bp, Artificial Sequence
  synthetic construct
cgcccgccgcgccccgcgcccgccccgccgcccccgcccgtccttttctcatagtagtttaaactatttctttcaaaatagataattcaaagaggaggaattctgatcacagaaagaaaaaaagctgacttttccacaaaagacatttatcaggacctcaa

SEQ ID NO: 352 - DNA, 155 bp, Artificial Sequence
  synthetic construct
cgcccgccgcgccccgcgcccgccccgccgcccccgcccgtttctttcaaaatagataattcaaagaggaggaattctgatcacagaaagaaaaaaagctgacttttccacaaaagacatttatcaggacctcaaccggttgttgaaaggcaaaa

SEQ ID NO: 353 - DNA, 151 bp, Artificial Sequence
  synthetic construct
atttatcaggacctcaaccggttgttgaaaggcaaaaagggagagcagatgaatagtgctgtattgccagaaatggagaatcaggtacatggattataaatgtgaattacacgggcgggggcggcggggcgggcgcggggcgcggcgggcg

SEQ ID NO: 354 - DNA, 110 bp, Artificial Sequence
  synthetic construct
attgccagaaatggagaatcaggtacatggattataaatgtgaattacaatatatataatgtaaatatgtcgggcgggggcggcggggcgggcgcggggcgcggcgggcg

SEQ ID NO: 355 - DNA, 155 bp, Artificial Sequence
  synthetic construct
cgcccgccgcgccccgcgcccgccccgccgcccccgcccgtcattttgcatttgttttttaaaatctttagaactggatccagtggtatagaaatcttcgatttttaaattcttaattttaggttgcagtttcatcactgtctgcggtaatcaag

SEQ ID NO: 356 - DNA, 219 bp, Artificial Sequence
  synthetic construct
cgcccgccgcgccccgcgcccgccccgccgcccccgcccgcttcgatttttaaattcttaattttaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcaggt

SEQ ID NO: 357 - DNA, 142 bp, Artificial Sequence
  synthetic construct
cgcccgccgcgccccgcgcccgccccgccgcccccgcccggtttttcatggcgtagtaaggttttcactaatgagcttgccattctttctattttattttttgtttactagggttctgttgaagataccactggctctcagt

SEQ ID NO: 358 - DNA, 186 bp, Artificial Sequence
  synthetic construct
tttactagggttctgttgaagataccactggctctcagtctctggctgccttgctgaataagtgtaaaacccctcaaggacaaagacttgttaaccagtggattaagcagcctctcatggataagaacagaatagaggagaggtatcgggcgggggcggcggggcgggcgcggggcgcggcgggcg

SEQ ID NO: 359 - DNA, 180 bp, Artificial Sequence
  synthetic construct
tcaaggacaaagacttgttaaccagtggattaagcagcctctcatggataagaacagaatagaggagaggtatgttattagtttatactttcgttagttttatgtaacctgcagttacccacatgattataccacttattcgggcgggggcggcggggcgggcgcggggcgcggcgggcg

SEQ ID NO: 360 - DNA, 160 bp, Artificial Sequence
  synthetic construct
cgcccgccgcgccccgcgcccgccccgccgcccccgcccggttgagacttacgtgcttagttgataaattttaattttatactaaaatattttacattaattcaagttaatttatttcagattgaatttagtggaagcttttgtagaagatgcagaattg

SEQ ID NO: 361 - DNA, 176 bp, Artificial Sequence
  synthetic construct
cgcccgccgcgccccgcgcccgccccgccgcccccgcccgatttatttcagattgaatttagtggaagcttttgtagaagatgcagaattgaggcagactttacaagaagatttacttcgtcgattcccagatcttaaccgacttgccaagaagtttcaaagacaagcagcaaact

SEQ ID NO: 362 - DNA, 244 bp, Artificial Sequence
  synthetic construct
gacttgccaagaagtttcaaagacaagcagcaaacttacaagattgttaccgactctatcagggtataaatcaactacctaatgttatacaggctctggaaaaacatgaaggtaacaagtgattttgtttttttgttttccttcaactcatacaatatatacttggcaatgtgctgtcctcataaagttggtggtggtgactcacgggcgggggcggcggggcgggcgcggggcgcggcgggcg

SEQ ID NO: 363 - DNA, 190 bp, Artificial Sequence
  synthetic construct
cgcccgccgcgccccgcgcccgccccgccgcccccgcccgtttggatcaaatgatgcttgtttatctcagtcaaaattttatgatttgtattctgtaaaatgagatctttttatttgtttgttttactactttcttttaggaaaacaccagaaattattgttggcagtttttgtgactcctcttactgat

SEQ ID NO: 364 - DNA, 155 bp, Artificial Sequence
  synthetic construct
ttgtgactcctcttactgatcttcgttctgacttctccaagtttcaggaaatgatagaaacaactttagatatggatcaggtatgcaatatactttttaatttaagcagtagttacgggcgggggcggcggggcgggcgcggggcgcggcgggcg

SEQ ID NO: 365 - DNA, 196 bp, Artificial Sequence
  synthetic construct
ctgacttctccaagtttcaggaaatgatagaaacaactttagatatggatcaggtatgcaatatactttttaatttaagcagtagttatttttaaaaagcaaaggccactttaagaaagtttgtagatttttctttttagtatctaattgtagcaccgggcgggggcggcggggcgggcgcggggcgcggcgggcg

SEQ ID NO: 366 - DNA, 150 bp, Artificial Sequence
  synthetic construct
agaaattattgttggcagtttttgtgactcctcttactgatcttcgttctgacttctccaagtttcaggaaatgatagaaacaactttagatatggatcaggtatgcaatcgggcgggggcggcggggcgggcgcggggcgcggcgggcg

SEQ ID NO: 367 - DNA, 267 bp, Artificial Sequence
  synthetic construct
cgcccgccgcgccccgcgcccgccccgccgcccccgcccgaatatttgctttataatttctgtctttacccattatttataggattttgtcactttgttctgtttgcaggtggaaaaccatgaattccttgtaaaaccttcatttgatcctaatctcagtgaattaagagaaataatgaatgacttggaaaagaagatgcagtcaacattaataagtgcagccagagatcttggtaagaatgggtcattggaggttggaataattct

SEQ ID NO: 368 - DNA, 131 bp, Artificial Sequence
  synthetic construct
cgcccgccgcgccccgcgcccgccccgccgcccccgcccggaattacattgaaaaatggtagtaggtatttatggaatactttttcttttcttcttgattatcaaggcttggaccctggcaaacagattaa

SEQ ID NO: 369 - DNA, 195 bp, Artificial Sequence
  synthetic construct
tcttcttgattatcaaggcttggaccctggcaaacagattaaactggattccagtgcacagtttggatattactttcgtgtaacctgtaaggaagaaaaagtccttcgtaacaataaaaactttagtactgtagatatccagaagaatggtgttacgggcgggggcggcggggcgggcgcggggcgcggcgggcg

SEQ ID NO: 370 - DNA, 240 bp, Artificial Sequence
  synthetic construct
tgcacagtttggatattactttcgtgtaacctgtaaggaagaaaaagtccttcgtaacaataaaaactttagtactgtagatatccagaagaatggtgttaaatttaccaacaggtttgcaagtcgttattatatttttaaccctttattaattccctaaatgctctaacatgatgtgaatgttctatgataagttttaccgggcgggggcggcggggcgggcgcggggcgcggcgggcg

SEQ ID NO: 371 - DNA, 198 bp, Artificial Sequence
  synthetic construct
cgcccgccgcgccccgcgcccgccccgccgcccccgcccgtttggatatgtttcacgtagtacacattgcttctagtacacattttaatatttttaataaaactgttatttcgatttgcagcaaattgacttctttaaatgaagagtataccaaaaataaaacagaatatgaagaagcccaggatgccattgttaaag

SEQ ID NO: 372 - DNA, 242 bp, Artificial Sequence
  synthetic construct
cgcccgccgcgccccgcgcccgccccgccgcccccgcccggcaaattgacttctttaaatgaagagtataccaaaaataaaacagaatatgaagaagcccaggatgccattgttaaagaaattgtcaatatttcttcaggtaaacttaatagaactaataatgttctgaatgtcacctggcttttggtaacagaagaaaaatcatgatatttgaagtgtgttttgttattttcgcaagccat

SEQ ID NO: 373 - DNA, 193 bp, Artificial Sequence
  synthetic construct
cgcccgccgcgccccgcgcccgccccgccgcccccgcccgaggaaatgggttttgaattcccaaatggggggattaaatgtatttttacggcttatatctgtttattattcagtattcctgtgtacattttctgtttttatttttatacaggctatgtagaaccaatgcagacactcaatgatgtgttagctc

SEQ ID NO: 374 - DNA, 152 bp, Artificial Sequence
  synthetic construct
cgcccgccgcgccccgcgcccgccccgccgcccccgcccgatttttatacaggctatgtagaaccaatgcagacactcaatgatgtgttagctcagctagatgctgttgtcagctttgctcacgtgtcaaatggagcacctgttccatatgt

SEQ ID NO: 375 - DNA, 251 bp, Artificial Sequence
  synthetic construct
tggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggtaaaaaacctggtttttgggctttgtgggggtaacgttttgttcgggcgggggcggcggggcgggcgcggggcgcggcgggcg

SEQ ID NO: 376 - DNA, 162 bp, Artificial Sequence
  synthetic construct
cagctttgctcacgtgtcaaatggagcacctgttccatatgtacgaccagccattttggagaaaggacaaggaagaattatattaaaagcatccaggcatgcttgtgttgaagttcaagatgcgggcgggggcggcggggcgggcgcggggcgcggcgggcg

SEQ ID NO: 377 - DNA, 177 bp, Artificial Sequence
  synthetic construct
cgcccgccgcgccccgcgcccgccccgccgcccccgcccgaggactaacaatccatttattagtagcagaaagaagtttaaaatcttgctttctgatataatttgttttgtaggccccaatatgggaggtaaatcaacatatattcgacaaactggggtgatagtactcatggccca

SEQ ID NO: 378 - DNA, 193 bp, Artificial Sequence
  synthetic construct
cgcccgccgcgccccgcgcccgccccgccgcccccgcccgatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtc

SEQ ID NO: 379 - DNA, 194 bp, Artificial Sequence
  synthetic construct
ttgtggactgcatcttagcccgagtaggggctggtgacagtcaattgaaaggagtctccacgttcatggctgaaatgttggaaactgcttctatcctcaggtaagtgcatctcctagtcccttgaagatagaaatgtatgtctctgtcctgtgacgggcgggggcggcggggcgggcgcggggcgcggcgggcg

SEQ ID NO: 380 - DNA, 326 bp, Artificial Sequence
  synthetic construct
cgcccgccgcgccccgcgcccgccccgccgcccccgcccggtatgtgtatgttaccacattttatgtgatgggaaatttcatgtaattatgtgcttcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaacta

SEQ ID NO: 381 - DNA, 190 bp, Artificial Sequence
  synthetic construct
ataatctacatgtcacagcactcaccactgaagagaccttaactatgctttatcaggtgaagaaaggtatgtactattggagtactctaaattcagaacttggtaatgggaaacttactacccttgaaatcatcagtaattgccttattccgggcgggggcggcggggcgggcgcggggcgcggcgggcg

SEQ ID NO: 382 - DNA, 304 bp, Artificial Sequence
  synthetic construct
gtctcttctcatgctgtcccctcacgcttccccaaatttcttataggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagaggtttgtcagtttgttttcatagtttaacttagcttctctattcgggcgggggcggcggggcgggcgcggggcgcggcgggcg

SEQ ID NO: 383 - DNA, 285 bp, Artificial Sequence
  synthetic construct
ttactaatgggacattcacatgtgtttcagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtcgggcgggggcggcggggcgggcgcggggcgcggcgggcg

SEQ ID NO: 384 - DNA, 260 bp, Artificial Sequence
  synthetic construct
cgcccgccgcgccccgcgcccgccccgccgcccccgcccgcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcg

SEQ ID NO: 385 - DNA, 166 bp, Artificial Sequence
  synthetic construct
ggcgggggaagttatccagcggccagctaatgctatcaaagagatgattgagaactggtacggagggagtcgagccgggctcacttaagggctacgacttaacgggccgcgtcactcaatggcgcgcgggcgggggcggcggggcgggcgcggggcgcggcgggcg

SEQ ID NO: 386 - DNA, 160 bp, Artificial Sequence
  synthetic construct
cgcccgccgcgccccgcgcccgccccgccgcccccgcccgcaaagagatgattgagaactggtacggagggagtcgagccgggctcacttaagggctacgacttaacgggccgcgtcactcaatggcgcggacacgcctctttgcccgggcagaggcatg

SEQ ID NO: 387 - DNA, 221 bp, Artificial Sequence
  synthetic construct
cgcccgccgcgccccgcgcccgccccgccgcccccgcccgggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatg

SEQ ID NO: 388 - DNA, 196 bp, Artificial Sequence
  synthetic construct
cgcccgccgcgccccgcgcccgccccgccgcccccgcccgttatcattgcttggctcatattaaaatatgtacattagagtagttgcagactgataaattattttctgtttgatttgccagtttagatgcaaaatccacaagtattcaagtgattgttaaagagggaggcctgaagttgattcagatccaagacaa

SEQ ID NO: 389 - DNA, 178 bp, Artificial Sequence
  synthetic construct
cgcccgccgcgccccgcgcccgccccgccgcccccgcccggcaaaatccacaagtattcaagtgattgttaaagagggaggcctgaagttgattcagatccaagacaatggcaccgggatcagggtaagtaaaacctcaaagtagcaggatgtttgtgcgcttcatggaagagtcagg

SEQ ID NO: 390 - DNA, 226 bp, Artificial Sequence
  synthetic construct
cgcccgccgcgccccgcgcccgccccgccgcccccgcccggggaattcaaagagatttggaaaaatgagtaacatgattatttactcatctttttggtatctaacagaaagaagatctggatattgtatgtgaaaggttcactactagtaaactgcagtcctttgaggatttagccagtatttctacctatggctttcgaggtgaggtaagctaaagattcaagaa

SEQ ID NO: 391 - DNA, 229 bp, Artificial Sequence
  synthetic construct
atattgtatgtgaaaggttcactactagtaaactgcagtcctttgaggatttagccagtatttctacctatggctttcgaggtgaggtaagctaaagattcaagaaatgtgtaaaatatcctcctgtgatgacattgtctgtcatttgttagtatgtatttctcaacatagataaataaggtttggtaccgggcgggggcggcggggcgggcgcggggcgcggcgggcg

SEQ ID NO: 392 - DNA, 215 bp, Artificial Sequence
  synthetic construct
ggtgaggtgacagtgggtgacccagcagtgagtttttctttcagtctattttcttttcttccttaggctttggccagcataagccatgtggctcatgttactattacaacgaaaacagctgatggaaagtgtgcatacaggtatagtgctgacttcttttactcatatatattcacgggcgggggcggcggggcgggcgcggggcgcggcgggcg

SEQ ID NO: 393 - DNA, 183 bp, Artificial Sequence
  synthetic construct
tcatgttactattacaacgaaaacagctgatggaaagtgtgcatacaggtatagtgctgacttcttttactcatatatattcattctgaaatgtattttttgcctaggtctcagagtaatcctgtctcaacaccagtgttatccgggcgggggcggcggggcgggcgcggggcgcggcgggcg

SEQ ID NO: 394 - DNA, 123 bp, Artificial Sequence
  synthetic construct
cgcccgccgcgccccgcgcccgccccgccgcccccgcccggggattagtatctatctctctactggatattaatttgttatattttctcattagagcaagttactcagatggaaaactgaaag

SEQ ID NO: 395 - DNA, 128 bp, Artificial Sequence
  synthetic construct
ctgaaagcccctcctaaaccatgtgctggcaatcaagggacccagatcacggtaagaatggtacatgggagagtaaattgttgaagctcgggcgggggcggcggggcgggcgcggggcgcggcgggcg

SEQ ID NO: 396 - DNA, 114 bp, Artificial Sequence
  synthetic construct
gggacccagatcacggtaagaatggtacatgggagagtaaattgttgaagctttgtttgtataaatattggaatcgggcgggggcggcggggcgggcgcggggcgcggcgggcg

SEQ ID NO: 397 - DNA, 142 bp, Artificial Sequence
  synthetic construct
cgcccgccgcgccccgcgcccgccccgccgcccccgcccgtttgttatattttctcattagagcaagttactcagatggaaaactgaaagcccctcctaaaccatgtgctggcaatcaagggacccagatcacggtaagaat

SEQ ID NO: 398 - DNA, 288 bp, Artificial Sequence
  synthetic construct
cgcccgccgcgccccgcgcccgccccgccgcccccgcccgattcactatcttaagacctcgcttttgccaggacatcttgggttttattttcaagtacttctatgaatttacaagaaaaatcaatcttctgttcaggtggaggaccttttttacaacatagccacgaggagaaaagctttaaaaaatccaagtgaagaatatgggaaaattttggaagttgttggcaggtacagtccaaaatctgggagtgggtctctgagatttgtcatcaaagtaatgtgttctag

SEQ ID NO: 399 - DNA, 277 bp, Artificial Sequence
  synthetic construct
taactaaaagggggctctgacatctagtgtgtgtttttggcaactcttttcttactcttttgtttttcttttccaggtattcagtacacaatgcaggcattagtttctcagttaaaaaagtaagttcttggtttatgggggatggttttgttttatgaaaagaaaaaaggggatttttaatagtttgctggtggagataaggttatgatgtttcagtctcagccatgagacaataaacgggcgggggcggcggggcgggcgcggggcgcggcgggcg

SEQ ID NO: 400 - DNA, 176 bp, Artificial Sequence
  synthetic construct
cgcccgccgcgccccgcgcccgccccgccgcccccgcccggctggtggagataaggttatgatgtttcagtctcagccatgagacaataaatccttgtgtcttctgctgtttgtttatcagcaaggagagacagtagctgatgttaggacactacccaatgcctcaaccgtggaca

SEQ ID NO: 401 - DNA, 153 bp, Artificial Sequence
  synthetic construct
gggggcaaggagagacagtagctgatgttaggacactacccaatgcctcaaccgtggacaatattcgctccatctttggaaatgctgttagtcggtatgtcgataacctatatcgggcgggggcggcggggcgggcgcggggcgcggcgggcg

SEQ ID NO: 402 - DNA, 141 bp, Artificial Sequence
  synthetic construct
aaatgctgttagtcggtatgtcgataacctatataaaaaaatcttttacatttattatcttggtttatcattccatcacattattttggaacctttcaagacgggcgggggcggcggggcgggcgcggggcgcggcgggcg

SEQ ID NO: 403 - DNA, 289 bp, Artificial Sequence
  synthetic construct
cgcccgccgcgccccgcgcccgccccgccgcccccgcccggtaatgtttgagttttgagtattttcaaaagcttcagaatctcttttctaatagagaactgatagaaattggatgtgaggataaaaccctagccttcaaaatgaatggttacatatccaatgcaaactactcagtgaagaagtgcatcttcttactcttcatcaaccgtaagttaaaaagaaccacatgggaaatccactcacaggaaacacccacagggaattttatgggaccatggaaaaatttctg

SEQ ID NO: 404 - DNA, 251 bp, Artificial Sequence
  synthetic construct
cgcccgccgcgccccgcgcccgccccgccgcccccgcccgcaaagttagtttatgggaaggaaccttgtgtttttaaattctgattcttttgtaatgtttgagttttgagtattttcaaaagcttcagaatctcttttctaatagagaactgatagaaattggatgtgaggataaaaccctagccttcaaaatgaatggttacatatccaatgcaaactactcagtgaagaagtgcatcttcttactcttc

SEQ ID NO: 405 - DNA, 170 bp, Artificial Sequence
  synthetic construct
cgcccgccgcgccccgcgcccgccccgccgcccccgcccgcttcaaaatgaatggttacatatccaatgcaaactactcagtgaagaagtgcatcttcttactcttcatcaaccgtaagttaaaaagaaccacatgggaaatccactcacaggaaacacccacagggaat

SEQ ID NO: 406 - DNA, 254 bp, Artificial Sequence
  synthetic construct
cgcccgccgcgccccgcgcccgccccgccgcccccgcccgtgaatgtacacctgtgacctcacccctcaggacagttttgaactggttgctttctttttattgtttagatcgtctggtagaatcaacttccttgagaaaagccatagaaacagtgtatgcagcctatttgcccaaaaacacacacccattcctgtacctcaggtaatgtagcaccaaactcctcaaccaagactcacaaggaacagatgttcta

SEQ ID NO: 407 - DNA, 224 bp, Artificial Sequence
  synthetic construct
cgcccgccgcgccccgcgcccgccccgccgcccccgcccgttgaccactgtgtcatctggcctcaaatcttctggccaccacatacaccatatgtgggctttttctccccctcccactatctaaggtaattgttctctcttattttcctgacagtttagaaatcagtccccagaatgtggatgttaatgtgcaccccacaaagcatgaagttcacttcctgcac

SEQ ID NO: 408 - DNA, 160 bp, Artificial Sequence
  synthetic construct
cgcccgccgcgccccgcgcccgccccgccgcccccgcccgcagaatgtggatgttaatgtgcaccccacaaagcatgaagttcacttcctgcacgaggagagcatcctggagcgggtgcagcagcacatcgagagcaagctcctgggctccaattcctcc

SEQ ID NO: 409 - DNA, 173 bp, Artificial Sequence
  synthetic construct
cagcagcacatcgagagcaagctcctgggctccaattcctccaggatgtacttcacccaggtcagggcgcttctcatccagctacttctctggggcctttgaaatgtgcccggccagacgtgagagcccagatcgggcgggggcggcggggcgggcgcggggcgcggcgggcg

SEQ ID NO: 410 - DNA, 255 bp, Artificial Sequence
  synthetic construct
cgcccgccgcgccccgcgcccgccccgccgcccccgcccgttttttttaatacagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcac

SEQ ID NO: 411 - DNA, 188 bp, Artificial Sequence
  synthetic construct
catttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaacgggcgggggcggcggggcgggcgcggggcgcggcgggcg

SEQ ID NO: 412 - DNA, 205 bp, Artificial Sequence
  synthetic construct
agcccctgctgaagtggctgccaaaaatcagagcttggagggggatacaacaaaggggacttcagaaatgtcagagaagagaggacctacttccagcaaccccaggtatggccttttgggaaaagtacagcctacctcctttattctgtaataaaactgccttctcgggcgggggcggcggggcgggcgcggggcgcggcgggcg

SEQ ID NO: 413 - DNA, 167 bp, Artificial Sequence
  synthetic construct
cgcccgccgcgccccgcgcccgccccgccgcccccgcccgtgtccagtcagccccaggccattgtcacagaggataagacagatatttctagtggcagggctaggcagcaagatgaggagatgcttgaactcccagcccctgctgaagtggctgccaaaaatcagag

SEQ ID NO: 414 - DNA, 150 bp, Artificial Sequence
  synthetic construct
cgcccgccgcgccccgcgcccgccccgccgcccccgcccgaatttggctaagtttaaaaacaagaataataatgatctgcacttccttttcttcattgcagaaagagacatcgggaagattctgatgtggaaatggtggaagatgattcc

SEQ ID NO: 415 - DNA, 160 bp, Artificial Sequence
  synthetic construct
cgcccgccgcgccccgcgcccgccccgccgcccccgcccgcattgcagaaagagacatcgggaagattctgatgtggaaatggtggaagatgattcccgaaaggaaatgactgcagcttgtaccccccggagaaggatcattaacctcacgcggcgggcg

SEQ ID NO: 416 - DNA, 168 bp, Artificial Sequence
  synthetic construct
cgcccgccgcgccccgcgcccgccccgccgcccccgcccgtgattcccgaaaggaaatgactgcagcttgtaccccccggagaaggatcattaacctcactagtgttttgagtctccaggaagaaattaatgagcagggacatgagggtacgtaaacgctgtggcctg

SEQ ID NO: 417 - DNA, 140 bp, Artificial Sequence
  synthetic construct
cgcccgccgcgccccgcgcccgccccgccgcccccgcccgattaacctcactagtgttttgagtctccaggaagaaattaatgagcagggacatgagggtacgtaaacgctgtggcctgcctgggatgcatagggcctca

SEQ ID NO: 418 - DNA, 145 bp, Artificial Sequence
  synthetic construct
cgcccgccgcgccccgcgcccgccccgccgcccccgcccgggtcaatgaagtggggttggtaggattctattacttacctgttttttggttttattttttgttttgcagttctccgggagatgttgcataaccactccttcgtgg

SEQ ID NO: 419 - DNA, 242 bp, Artificial Sequence
  synthetic construct
agttctccgggagatgttgcataaccactccttcgtgggctgtgtgaatcctcagtgggccttggcacagcatcaaaccaagttataccttctcaacaccaccaagcttaggtaaatcagctgagtgtgtgaacaagcagagctactacaacaatggtccagggagcacaggcacaaaagctaaggagagcagcatgaggtacgggcgggggcggcggggcgggcgcggggcgcggcgggcg

SEQ ID NO: 420 - DNA, 294 bp, Artificial Sequence
  synthetic construct
ttcagggattacttctcccattttgtcccaactggttgtatctcaagcatgaattcagcttttccttaaagtcacttcatttttattttcagtgaagaactgttctaccagatactcatttatgattttgccaattttggtgttctcaggttatcggtaagtttagatccttttcacttctgaaatttcaactgatcgtttctgaaaatagtagctctccactaatatcttatttgtagtatgttaaatttttccgggcgggggcggcggggcgggcgcggggcgcggcgggcg

SEQ ID NO: 421 - DNA, 150 bp, Artificial Sequence
  synthetic construct
cgcccgccgcgccccgcgcccgccccgccgcccccgcccggccattctgatagtggattcttgggaattcaggcttcatttggatgctccgttaaagcttgctccttcatgttcttgcttcttcctaggagccagcaccgctctttgacc

SEQ ID NO: 422 - DNA, 220 bp, Artificial Sequence
  synthetic construct
gcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggtgtgacagccattcttataccgggcgggggcggcggggcgggcgcggggcgcggcgggcg

SEQ ID NO: 423 - DNA, 154 bp, Artificial Sequence
  synthetic construct
ggctgagatgcttgcagactatttctctttggaaattgatgaggtgtgacagccattcttatacttctgttgtattcttcaaataaaatttccagccgggtgcggtggctcatgcgggcgggggcggcggggcgggcgcggggcgcggcgggcg

SEQ ID NO: 424 - DNA, 261 bp, Artificial Sequence
  synthetic construct
cgcccgccgcgccccgcgcccgccccgccgcccccgcccgtgtttaaactatgacagcattatttcttgttcccttgtcctttttcctgcaagcaggaagggaacctgattggattaccccttctgattgacaactatgtgccccctttggagggactgcctatcttcattcttcgactagccactgaggtcagtgatcaagcagatactaagcatttcggtacatgcatgtgtgctggagggaaagggcaaatgaccacc

SEQ ID NO: 425 - DNA, 217 bp, Artificial Sequence
  synthetic construct
cgcccgccgcgccccgcgcccgccccgccgcccccgcccgtgtgatctccgtttagaatgagaatgtttaaattcgtacctattttgaggtattgaatttctttggaccaggtgaattgggacgaagaaaaggaatgttttgaaagcctcagtaaagaatgcgctatgttctattccatccggaagcagtacatatctgaggagtcgaccctctcag

SEQ ID NO: 426 - DNA, 156 bp, Artificial Sequence
  synthetic construct
cgcccgccgcgccccgcgcccgccccgccgcccccgcccgtgcgctatgttctattccatccggaagcagtacatatctgaggagtcgaccctctcaggccagcaggtacagtggtgatgcacactggcaccccaggactagcgggcgggggcggc

SEQ ID NO: 427 - DNA, 285 bp, Artificial Sequence
  synthetic construct
aagtctttccagacccagtgcacatcccatcagccaggacaccagtgtatgttgggatgcaaacagggaggcttatgacatctaatgtgttttccagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacacgggcgggggcggcggggcgggcgcggggcgcggcgggcg

SEQ ID NO: 428 - DNA, 160 bp, Artificial Sequence
  synthetic construct
aaggccttgcgctcacacattctgcctcctaaacatttcacagaagatggaaatatcctgcagcttgctaacctgcctgatctatacaaagtctttgagaggtggttaaatatggttattcgggcgggggcggcggggcgggcgcggggcgcggcgggcg

SEQ ID NO: 429 - DNA, 197 bp, Artificial Sequence
  synthetic construct
cgcccgccgcgccccgcgcccgccccgccgcccccgcccgcagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacacgggcgg

(19) United States
(12) Patent Application Publication
(10) Pub. No.: US 2010/0112551 A1
(43) Pub. Date: May 6, 2010

(54) Approaches to identifying mutations associated with hereditary nonpolyposis colorectal cancer

(76) Inventors: Charles Dunlop, Irvine, CA (US); Anja Kammesheidt, Laguna Beach, CA (US)

Correspondence Address: BIOTECH BEACH LAW GROUP , PC, 5677 OBERLIN DRIVE, SUITE 204, SAN DIEGO, CA, 92121, US

(21) Appl. No.: 11/596,782

(22) PCT Filed: Jun. 14, 2005
(86) PCT No.: PCT/US05/20721

§ 371 Date: Jun. 21, 2007

Related U.S. Application Data
(60) Provisional application No. 60/579,773, filed on Jun. 14, 2004.

(51) Int. Cl. C12Q 1/68

(52) U.S. Cl. 435/6

(57) ABSTRACT

The present invention relates to the field of genetic screening. More specifically, the described embodiments concern methods to screen multiple samples, in a single assay, for the presence or absence of mutations or polymorphisms in a plurality of genes. Approaches to screen for the presence or absence of mutations that are associated with Hereditary Nonpolyposis Colorectal Cancer (HNPCC) and approaches to design primers that generate extension products that facilitate the resolution of multiple extension products in a single lane of a gel or in a single run on a column are also provided.

### FIELD OF THE INVENTION

The present invention relates to the field of genetic screening and diagnostics. More specifically, the described embodiments concern methods to screen multiple samples, in a single assay, for the presence or absence of mutations or polymorphisms that relate to Hereditary Nonpolyposis Colorectal Cancer (HNPCC).

### BACKGROUND OF THE INVENTION

Hereditary Nonpolyposis Colorectal Cancer (HNPCC) is the most common hereditary form of colon cancer. It is a genetic syndrome caused by mutations in any one of five or more genes that code for proteins involved with repair of damaged or aberrant DNA, two of which are the human mismatch repair genes mutL homolog 1 (MLH1) and mutS homologue 2 (MSH2). Individuals that inherit mutations associated with HNPCC are at a much higher risk for colon cancer than the general population (80% chance of developing color cancer, vs. 4%) and at an earlier age (average age of onset of colon cancer: 44 years old, vs. 65 years of age for the general population). Individuals with HNPCC also have a higher risk of getting certain other forms of cancer (Lynch, H. et al. Cancer 78:1149 (1996)). There is a great need for approaches to identify mutations and polymorphisms that relate to this deadly disease.

Current DNA-based diagnostics allow for the identification of a single mutation or polymorphism or gene per analysis. Although high-throughput methods and gene chip technology have enabled the ability to screen multiple samples or multiple loci within the same sample, these approaches require several independent reactions, which increases the time required to process clinical samples and drastically increases the cost. Further, because of time and expense, conventional diagnostic approaches focus on the identification of the presence of DNA fragments that are associated with a high frequency of mutation, leaving out analysis of other loci that may be critical to diagnose a disease. The need for more approaches for the diagnosis of genetic disease is manifest.

With the advent of multiplex Polymerase Chain Reaction (PCR), the ability to use multiple primer sets to generate multiple extension products from a single gene is at hand. By hybridizing isolated DNA with multiple sets of primers that flank loci of interest on a single gene, it is possible to generate a plurality of extension products in a single PCR reaction corresponding to fragments of the gene. As the number of primers increases, however, the complexity of the reaction increases and the ability to resolve the extension products using conventional techniques fails. Further, since many diseases are caused by changes of a single nucleotide, the rapid detection of the presence or absence of these mutations or polymorphisms is frustrated by the fact that the PCR products that indicate both the diseased and non-diseased state are of the same size.

Developments in gel electrophoresis and high performance liquid chromatography (HPLC), however, have enabled the separation of double-stranded DNAs based upon differences in their melting behaviors, which has allowed investigators to resolve DNA fragments having a single mutation or single polymorphism. Techniques such as temporal temperature gradient gel electrophoresis (TTGE) and denaturing high performance liquid chromatography (DHPLC) have been used to screen for small changes or point mutations in DNA fragments.

The separation principle of TTGE, for example, is based on the melting behavior of DNA molecules. In a denaturing polyacrylamide gel, double-stranded DNA is subject to conditions that will cause it to melt in discrete segments called “melting domains.” The melting temperature Tm of these domains is sequence-specific. When the Tm of the lowest melting domain is reached, the DNA will become partially melted, creating branched molecules. Partial melting of the DNA reduces its mobility in a polyacrylamide gel. Since the Tm of a particular melting domain is sequence-specific, the presence of a mutation or polymorphism will alter the melting profile of that DNA in comparison to the wild-type or non-polymorphic DNA. That is, a heteroduplex DNA consisting of a wild-type or non-polymorphic strand annealed to mutant or poymorphic strand, will melt at a lower temperature than a homoduplex DNA strand consisting of two wild-type or non-polymorphic strands. Accordingly, the DNA containing the mutation or polymorphism will have a different mobility compared to the wild-type or non-polymorphic DNA.

Similarly, the separation principle of DHPLC is based on the melting or denaturing behavior of DNA molecules. As the use and understanding of HPLC developed, it became apparent that when HPLC analyses were carried out at a partially denaturing temperature, i.e., a temperature sufficient to denature a heteroduplex at the site of base pair mismatch, homoduplexes could be separated from heteroduplexes having the same base pair length. (See e.g., Hayward-Lester, et al., Genome Research 5:494 (1995); Underhill, et al., Proc. Natl. Acad. Sci. USA 93:193 (1996); Oefner, et al., DHPLC Workshop, Stanford University, Palo Alto, Calif., (Mar. 17, 1997); Underhill, et al., Genome Research 7:996 (1997); Liu, et al., Nucleic Acid Res., 26:1396 (1998), all of which and the references contained therein are hereby expressly incorporated by reference in their entireties).

Techniques such as Matched Ion Polynucleotide Chromatography (MIPC) and Denaturing Matched Ion Polynucleotide Chromatography (DMIPC) have also been employed to increase the sensitivity of detection. It was soon realized that DHPLC, which for the purposes of this disclosure includes but is not limited to, MIPC, DMIPC, and ion-pair reverse phase high-performance liquid chromatography, could be used to separate heteroduplexes from homoduplexes that differed by as little as one base pair. Various DHPLC techniques have been described in U.S. Pat. Nos. 5,795,976; 5,585,236; 6,024,878; 6,210,885; Huber, et al., Chromatographia 37:653 (1993); Huber, et al., Anal. Biochem. 212:351 (1993); Huber, et al., Anal. Chem. 67:578 (1995); ODonovan et al., Genomics 52:44 (1998), Am J Hum Genet. December; 67(6):1428-36 (2000); Ann Hum Genet. September:63 (Pt 5):383-91 (1999); Biotechniques, April; 28(4):740-5 (2000); Biotechniques. November; 29(5):1084-90, 1092 (2000); Clin Chem. August; 45(8 Pt 1):1133-40 (1999); Clin Chem. April; 47(4):635-44 (2001); Genomics. August 15; 52(1):44-9 (1998); Genomics. March 15; 56(3):247-53 (1999); Genet Test.; 1(4):237-42 (1997-98); Genet Test.:4(2):125-9 (2000); Hum Genet. June; 106(6):663-8 (2000); Hum Genet. November; 107(5):483-7 (2000); Hum Genet. November; 107(5):488-93 (2000); Hum Mutat. December; 16(6):518-26 (2000); Hum Mutat. 15(6):556-64 (2000); Hum Mutat. March; 17(3):210-9 (2001); J Biochem Biophys Methods. November 20; 46(1-2):83-93 (2000); J Biodhem Biophys Methods. January 30; 47(1-2):5-19 (2001); Mutat Res. November 29; 430(1):13-21 (1999); Nucleic Acids Res. March 1; 28(5):E13 (2000); and Nucleic Acids Res. October 15; 28(20):E89 (2000), all of which, including the references contained therein, are hereby expressly incorporated by reference in their entireties. Despite the efforts of many, there remains a need for more approaches to screen and identify mutations and/or polymorphisms in genes, in particular, genes that relate to Hereditary Nonpolyposis Colorectal Cancer.

### SUMMARY OF THE INVENTION

Aspects of the invention concern rapid and inexpensive but efficient approaches to determine the presence or absence of mutations and/or polymorphisms that relate to Hereditary Nonpolyposis Colorectal Cancer (HNPCC). Several oligonucleotide primers specific for the human mismatch repair genes, mutL homolog 1 (MLH1) and mutS homologue 2 (MSH2), have been developed (e.g., Tables A and 2). These primers and oligonucleotides that are any number between 1-75 nucleotides upstream or downstream of said primers are unique in sequence and in their ability to generate extension products that melt evenly over vast stretches of nucleotides, which greatly improves the sensitivity of detection (e.g., single base mutations). It was then realized that by grouping extension products with similar melting behaviors, one can rapidly and efficiently separate multiple extension products on the basis of melting behavior on the same lane of a TTGE gel or in the same run on a DHPLC. Accordingly, a rapid, inexpensive and efficient approach to diagnose a subject at risk for HNPCC was discovered, whereby extension products are generated from a subject's DNA using the primers described herein, the extension products are grouped or mixed according to their melting behavior, and the grouped or mixed extension products are separated on the basis of melting behavior (e.g., one group per lane of TTGE gel). Not only does the pooling of extension products reduce cost and the time to perform the analysis but, because the extension products are optimized for melting behavior, the sensitivity of detection remains very high.

By one approach, for example, a method of identifying the presence or absence of a genetic marker in the human mismatch repair genes MLH1 and MSH2 of a subject is conducted by providing a DNA sample from said subject; providing at least one primer set from Table A; contacting said DNA and said at least one primer set; generating an extension product from said at least one primer set that comprises a region of DNA that includes the location of said genetic marker; separating said extension product on the basis of melting behavior; and identifying the presence or absence of said genetic marker in said subject by analyzing the melting behavior of said extension product. In related embodiments, at least 2, 3, 4, 5, 6, 7, or 8 primer sets from Table A are contacted with said DNA. In more related embodiments, the extension products generated from said 2, 3, 4, 5, 6, 7, or 8 primer sets are grouped according to Table D and separated on the basis of melting behavior. Optionally, the extension products and/or the sample nucleic acid used in the approaches above can be sequenced so as to verify and/or identify the mutation or polymorphism.

In another set of embodiments, a method of identifying the presence or absence of a genetic marker in the human mismatch repair genes mutL homolog 1 (MLH1) and mutS homologue 2 (MSH2) of a subject is conducted by providing a DNA sample from said subject; providing at least one primer set that is any number between 1-75 nucleotides upstream or downstream of a primer set from Table A; contacting said DNA and said at least one primer set; generating an extension product from said at least one primer set that comprises a region of DNA that includes the location of said genetic marker, separating said extension product on the basis of melting behavior; and identifying the presence or absence of said genetic marker in said subject by analyzing the melting behavior of said extension product. In related embodiments, at least 2, 3, 4, 5, 6, 7, or 8 primer sets from Table A are contacted with said DNA. In more related embodiments, the extension products generated from said 2, 3, 4, 5, 6, 7, or 8 primer sets are grouped according to Table D and separated on the basis of melting behavior. As above, optionally, the extension products and/or the sample nucleic acid used in these approaches can be sequenced so as to verify and/or identify the mutation or polymorphism.

### BRIEF DESCRIPTION OF THE DRAWINGS

FIG. 1 shows a melting curve for the extension product MLH1 2A spanning the beginning of exon 2 and nucleotides ˜100-188 of the depicted fragment. The x axis shows the number of nucleotides and the y axis shows the temperature.

FIG. 2 shows a melting curve for the extension product MLH1 2B covering the end of exon 2 and nucleotides ˜100-171 of the depicted fragment. The x axis shows the number of nucleotides and the y axis shows the temperature.

FIG. 3 shows a melting curve for the extension product MSH2 9 covering exon 9 and nucleotides ˜100-260 of the depicted fragment. The x axis shows the number of nucleotides and the y axis shows the temperature.

FIG. 4 shows a melting curve for the extension product MSH2 15 covering exon 15 and nucleotides ˜48-230 of the depicted fragment. The x axis shows the number of nucleotides and the y axis shows the temperature.

FIG. 5 shows a melting curve for the extension product MLH1 3A spanning the beginning of exon 3 and nucleotides ˜100-218 of the depicted fragment. The x axis shows the number of nucleotides and the y axis shows the temperature.

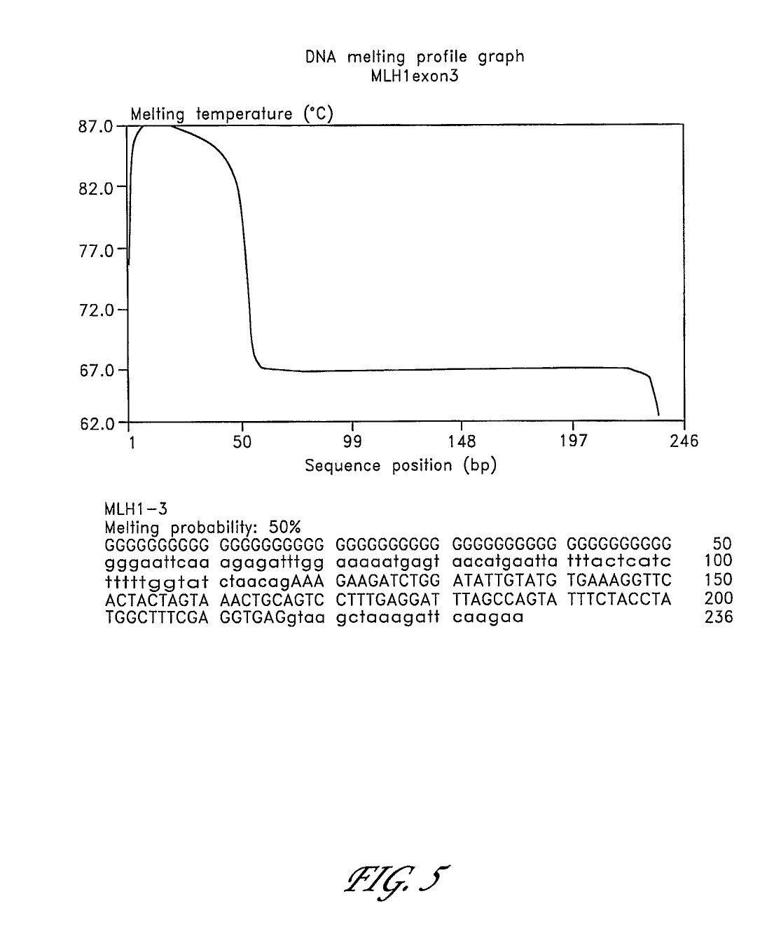

FIG. 6 shows a melting curve for the extension product MLH1 3B spanning the end of exon 3 and nucleotides ˜23-130 of the depicted fragment. The x axis shows the number of nucleotides and they axis shows the temperature.

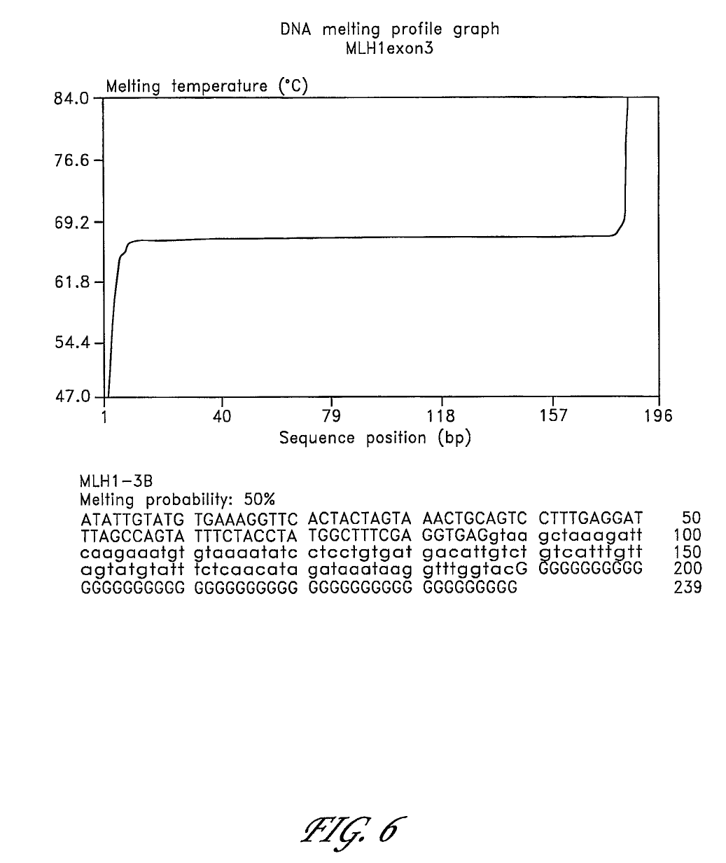

FIG. 7 shows results from experiments using primers with fluorescent tags to amplify portions of exon 10 of the Cystic Fibrosis Transmembrane Regulator (CTFR) gene. Two polymorphisms were amplified in this experiment: deltaF508 (DF508) and M470V. These results reveal the homozygous state of the clinical DNA samples used in the reactions when the products are mixed with wildtype DNA before analysis via TTGE. Texas Red (tr) and Oregon Green (og) tags are used. Banding patterns for wild type (WT), heterozygous (HET), homozygous (HOMO) and mixtures of these patterns (in the right hand side lanes, containing mixtures of tr and og products) are displayed.

### DETAILED DESCRIPTION OF THE PREFERRED EMBODIMENT

Embodiments described herein concern a novel approach to screen for the presence or absence of multiple mutations or polymorphisms in a plurality of genes, in particular, genes associated with Hereditary Nonpolyposis Colorectal Cancer (HNPCC). Particularly preferred embodiments concern approaches to screen multiple loci in the human mismatch repair genes mutL homolog 1 (MLH1) and mutS homologue 2 (MSH2) so as to determine the presence or absence of a mutation or polymorphism that may indicate a suseptibility to Hereditary Nonpolyposis Colorectal Cancer (HNPCC) and/or other cancers. Similar approaches have been used to identify the presence or absence or polymorphisms or mutations related to cystic fibrosis, which are described in U.S. patent application Ser. Nos. 10/300,683; 60/333,351; and 60/486,864, all of which are hereby expressly incorporated by reference in their entireties.

Several embodiments permit very sensitive detection of single base mutations, single base mismatches, and small nuclear polymorphisms (SNPs), as well as, larger alterations in DNA at multiple loci, in a plurality of genes, in multiple samples. Additionally, by employing a DNA standard or by screening a plurality of DNA samples in the same assay, improved sensitivity of detection can be obtained. A novel approach to designing primers and extension products generated therefrom is described in the context of an assay that was performed to detect the presence or absence of genetic markers, polymorphisms, or mutations on the human mismatch repair genes mutL homolog 1 (MLH1) and mutS homologue 2 (MSH2). By identifying the presence or absence of these polymorphisms or mutations, an understanding of susceptibility to Hereditary Nonpolyposis Colorectal Cancer (HNPCC) can be obtained.

Embodiments include methods of identifying the presence or absence of a plurality of genetic markers in a subject in the same gene or separate genes. One method is practiced, for example, by providing a DNA sample from said subject, providing a plurality of nucleic acid primer sets that hybridize to said DNA at regions that flank said plurality of genetic markers, wherein each primer set has a first and a second primer and, wherein said plurality of genetic markers exist on the same gene or a plurality of genes, contacting said DNA and said plurality of nucleic acid primer sets in a single reaction vessel or multiple reaction vessels, generating, in said reaction vessel(s), a plurality of extension products that comprise regions of DNA that include the location of said plurality of genetic markers, separating said plurality of extension products on the basis of melting behavior in a single lane or multiple lanes of a gel or a single run or multiple runs on a column, and identifying the presence or absence of said plurality of genetic markers in said subject by analyzing the melting behavior of said plurality of extension products. In some aspects of this method the separation on the basis of melting behavior is accomplished by TTGE and in other embodiments the separation on the basis of melting behavior is accomplished by DHPLC. In some embodiments, said extension products are first separated by size for a period sufficient to separate populations of extension products and then separated by melting behavior. The size separation can be accomplished on the TTGE gel or DHPLC column prior to separating on the basis of melting behavior.

Preferably, after generating the extension products by an amplification technique (e.g., Polymerase Chain Reaction or PCR), the extension products are grouped and pooled according to their predicted and/or actual melting behavior. In this way, multiple extension products, which correspond to different regions on the same gene or different' regions on a plurality of genes can be separated on the same lane of a TTGE gel or in the same run on a DHPLC column. By carefully designing the primers, such that the extension products generated therefrom melt over large stretches of DNA (approximately 25, 50, 75, 100, 125, or 150 nucleotides) at roughly the same temperature (within up to 1.5° C. of one another), it was unexpectedly discovered that multiple extension products (2, 3, 4, 5, 6 or more) can be separated on the same lane of a TTGE gel or in the same run on an DHPLC column, thereby substantially reducing the cost of conducting the analysis and increasing the speed of analysis.

In some embodiments, either the first or the second primer comprise a GC clamp. In other aspects of this embodiment, either the first or the second primer hybridize to a sequence within an intron. Preferably, at least one of the plurality of genetic markers is indicative of Hereditary Nonpolyposis Colorectal Cancer (HNPCC). In other embodiments, the plurality of primer sets consist of at least 3, 4, 5, 6, or 7 primer sets. Additionally, in some embodiments, the plurality of genes consist of at least 2, 3, 4, 5, 6, or 7 genes that are related to Hereditary Nonpolyposis Colorectal Cancer (HNPCC). The method above preferably generates the extension products using the Polymerase Chain Reaction (PCR) and the method can be supplemented by a step in which a control DNA is added.

Another embodiment concerns a method of identifying the presence or absence of a plurality of genetic markers in a plurality of subjects. This method is practiced by: providing a DNA sample from said plurality of subjects, providing a plurality of nucleic acid primer sets that hybridize to said DNA at regions that flank said plurality of genetic markers, wherein each primer set has a first and a second primer and, wherein said plurality of genetic markers exist on the same gene or on a plurality of genes, contacting said DNA and said plurality of nucleic acid primer sets in a single reaction vessel or multiple vessels, generating, in said reaction vessel(s), a plurality of extension products that comprise regions of DNA that include the location of said plurality of genetic markers, separating said plurality of extension products on the basis of melting behavior in a single lane or multiple lanes of a gel or a single run or multiple runs on a column, and identifying the presence or absence of said plurality of genetic markers in said plurality of subjects by analyzing the melting behavior of said plurality of extension products. In some aspects of this embodiment, the separation on the basis of melting behavior is accomplished by TTGE and in other embodiments the separation on the basis of melting behavior is accomplished by DHPLC. Again, preferred genetic markers for identification using the approaches above, concern genes that are associated with Hereditary Nonpolyposis Colorectal Cancer (HNPCC).

As above, preferably, after generating the extension products by the amplification technique (e.g., PCR) from the plurality of subjects, the extension products are grouped and pooled according to their predicted and/or actual melting behavior. By separating multiple extension products generated from a plurality of subjects in the same lane of a TTGE gel or in the same run on a DHPLC column, the cost of analysis is substantially reduced. Because the incidence of polymorphism or mutation in the population as a whole is small, the large scale screening, described above, can be performed. When a polymorphism and/or mutation is detected in this type of assay, single subject assays can be performed, as described above, to identify the subject(s) that have the polymorphism and/or mutation. Optionally, the extension products and/or the nucleic acid samples themselves can be sequenced so as to verify and/or identify the mutation or polymorphism.

In more embodiments, the plurality of subjects consist of at least 2, 3, 4, 5, 6, or 7 subjects. In more aspects of this embodiment, the plurality of primer sets consist of at least 3, 4, 5, 6, or 7 primer sets. Additionally, in some embodiments, the plurality of genes consist of at least 2, 3, 4, 5, 6, or 7 genes. The method above preferably generates the extension products using PCR and the method can be supplemented by a step in which a control DNA is added.

Still another embodiment involves a method of identifying the presence or absence of a mutation or polymorphism in a subject related to Hereditary Nonpolyposis Colorectal Cancer (HNPCC). This method is practiced by: providing a DNA sample from said subject, generating a population of extension products from said sample, wherein said extension products comprise a region of said DNA that corresponds to the location of said mutation or polymorphism, providing at least one control DNA, wherein said control DNA corresponds to the extension product but lacks said mutation or polymorphism, contacting said control DNA and said population of extension products in a single reaction vessel, thereby forming a mixed DNA sample, heating said mixed DNA sample to a temperature sufficient to denature said control DNA and said DNA sample, cooling said mixed DNA sample to a temperature sufficient to anneal said control DNA and said DNA sample, separating said mixed sample on the basis of melting behavior in a single lane or multiple lanes of a gel or a single run or multiple runs on a column, and identifying the presence or absence of said mutation or polymorphism by analyzing the melting behavior of said mixed DNA sample.

By this approach, the addition of the control DNA followed by the heating and cooling steps, forces heteroduplex formation, if a polymorphism or mutation is present, which facilitates identification. In some aspects of this embodiment, the control DNA is DNA obtained or amplified from a second subject and the presence or absence of a mutation or polymorphism is known. In other aspects of the invention, heteroduplex formation can be forced by pooling the extension products generated from a plurality of subjects and denaturing and annealing, as above. Because the predominant genotype in a plurality of subjects lacks polymorphisms or mutations in the gene(s) analyzed, the majority of the DNA will force heteroduplex formation with any polymorphic or mutant DNA in the pool. Accordingly, the identification of mutant and/or polymorphic DNA is facilitated and the cost of the analysis is reduced. In some aspects of this embodiment, the separation on the basis of melting behavior is accomplished by TTGE and in other embodiments the separation on the basis of melting behavior is accomplished by DHPLC.

Still more embodiments concern the primers or groups of primers disclosed herein (preferably MLH1 and MSH2 specific primers), extension products generated from said primers, kits containing said nucleic acids, and methods of using these primers, groups of primers, or extension products to diagnose a risk for a disease (e.g., HNPCC). These nucleic acid primers can be used to efficiently determine the presence or absence of a polymorphism or mutation in a multiplex PCR reaction that screens a plurality of genes and a plurality of subjects in a single reaction vessel or multiple reaction vessels. Additionally, reaction vessels comprising a DNA sample, and a plurality of nucleic acid primer sets that hybridize to said DNA sample at regions that flank a plurality of genetic markers, wherein said plurality of genetic markers exist on a single gene or a plurality of genes are embodiments. Further, a reaction vessel comprising a plurality of DNA samples obtained from a plurality of subjects and a plurality of nucleic acid primer sets that hybridize to said plurality of DNA samples at regions that flank a plurality of genetic markers, wherein said plurality of genetic markers exist on a plurality of genes or on a single gene are embodiments.

Still more aspects of the invention include a reaction vessel containing a plurality of extension products (2, 3, 4, 5, 6, 7, 8, 9, or 10 or more), which melt at approximately the same temperature (e.g., 0° C.-1.5° C. from one another). That is, in some approaches, the extension products are generated in separate vessels using individual primers sets but the extension products with similar melting behaviors are pooled prior to loading onto a TTGE gel or DHPLC. The pooled extension products are loaded onto a single lane of a gel and resolved by melting behavior. In some embodiments, differing fluorescent labels are employed in the individual PCR reactions so that the extension products generated therefrom fluoresce at different wavelengths (e.g., produce a different color under a detector) so as to facilitate identification after the pooled extension products are resolved on the gel or column.

Other embodiments concern a gel having lanes and adapted to separate different DNAs comprising a plurality of extension products, in a single lane of said gel, wherein said plurality of extension products melt at approximately the same temperature but are resolvable on said gel and, which correspond to regions of DNA located on a plurality of genes or on a single gene and, wherein said regions of DNA comprise loci that indicate a genetic trait and a gel having lanes and adapted to separate different DNAs comprising a plurality of extension products, in a single lane of said gel, wherein said plurality of extension products correspond to regions of DNA located on a plurality of genes or on a single gene in a single individual or a plurality of subjects and, wherein said regions of DNA comprise loci that indicate a genetic trait.

Additional embodiments include a DHPLC column adapted to separate different DNAs comprising a plurality of extension products, wherein said plurality of extension products melt at approximately the same temperature but are resolvable on said column and, which correspond to regions of DNA located on a plurality of genes or a single gene or and, wherein said regions of DNA comprise loci that indicate a genetic trait and a DHPLC column adapted to separate different DNAs comprising a plurality of extension products, wherein said plurality of extension products correspond to regions of DNA located on a plurality of genes or on a single gene in a single individual or a plurality of subjects and, wherein said regions of DNA comprise loci that indicate a genetic trait. More description of the compositions and methods described above is provided in the in the following sections.

Approaches to Facilitate and Reduce the Cost of Genetic Analysis

Aspects of the invention described herein concern approaches to analyze DNA, samples for the presence or absence of a plurality of genetic markers that reside on a plurality of genes in a single assay. Some embodiments allow one to rapidly distinguish a plurality of DNA fragments in a single sample that differ only slightly in size and/or composition (e.g., a single base change, mutation, or polymorphism). Other embodiments concern methods to screen multiple genes from a subject, in a single assay, for the presence or absence of a mutation or polymorphism. An approach to achieve greater sensitivity of detection of mutations or polymorphisms present in a DNA sample is also provided. Preferred embodiments, however, include methods to screen multiple genes, in a plurality of DNA samples, in a single assay, for the presence or absence of mutations or polymorphisms.

It was discovered that multiple extension products that have slight differences in length and/or composition can be resolved by separating the DNA on the basis of melting temperature. By one approach, a plurality of varying lengths of double-stranded DNA are applied to a denaturing gel and the double-stranded DNAs are separated by applying an electrical current while the temperature of the gel is raised gradually. By slowly increasing the temperature while the DNA is electrically separated on a polyacrylamide gel containing a denaturant (e.g., urea), the dsDNA eventually denatures to partially single stranded (branched molecules) DNA. Because branched or heteroduplex DNA migrates more rapidly or more slowly than dsDNA or homoduplex DNA, one can quickly determine the differences in melting behavior between DNA fragments, compare this melting temperature to a standard DNA (e.g., a wild-type DNA or non-polymorphic DNA), and identify the presence or absence of a mutation or polymorphism in the screened DNA. This technique efficiently separates multiple DNA fragments, generated by a single multiplex PCR reaction on a plurality of loci from different genes (e.g., in one experiment, 10 different loci were analyzed in the same reaction and each of the extension products, some that differed by only a single mutation, were efficiently resolved).

It was also discovered that multiple extension products that have slight differences in length and/or composition can be resolved by separating the DNA by DHPLC. By one approach, a plurality of varying lengths of double-stranded DNA are applied to a ion-pair reverse phase HPLC column (e.g., alkylated non-porous poly(styrene-divinylbenzene)) that has been equilibrated to an appropriate denaturing temperature, depending on the size and composition of the DNA to be separated (e.g., 53° C. to 63° C.) in an appropriate buffer (e.g., 0.1 mM triethylamine acetate (TEAA) pH 7.0). Once applied to the column, the double stranded DNA binds to the matrix. By slowly increasing the presence of a denaturant (e.g., acetonitrile in TEAA), the dsDNA eventually denatures to partially single stranded (branched molecules) DNA and elutes from the column. Preferably a linear gradient is used to slowly elute the bound DNA. Detection can be accomplished using a U.V. detector, radioactivity, dyes, or fluorescence. In some embodiments, the extension products are first separated on the basis of size using a shallow gradient of denaturant for a time sufficient to separate individual populations of extension products and then on the basis of melting behavior using a deeper gradient of denaturant. The techniques described in the following references can also be modified for use with aspects of the invention: U.S. Pat. Nos. 5,795,976; 5,585,236; 6,024,878; 6,210,885; Huber, et al., Chromatographia 37:653 (1993); Huber, et al., Anal. Biochem. 212:351 (1993); Huber, et al., Anal. Chem. 67:578 (1995); ODonovan et al., Genomics 52:44 (1998), Am J Hum Genet. December; 67(6):1428-36 (2000); Ann Hum Genet. September:63 (Pt 5):383-91 (1999); Biotechniques, April; 28(4):740-5 (2000); Biotechniques. November; 29(5):1084-90, 1092 (2000); Clin Chem. August; 45(8 Pt 1):1133-40 (1999); Clin Chem. April; 47(4):635-44 (2001); Genomics. August 15; 52(1):44-9 (1998); Genomics. March 15; 56(3):247-53 (1999); Genet Test.; 1(4):237-42 (1997-98); Genet Test:4(2):125-9 (2000); Hum Genet. June; 106(6):663-8 (2000); Hum Genet. November; 107(5):483-7 (2000); Hum Genet. November; 107(5):488-93 (2000); Hum Mutat. December; 16(6):518-26 (2000); Hum Mutat. 15(6):556-64 (2000); Hum Mutat. March; 17(3):210-9 (2001); J Biochem Biophys Methods. November 20; 46(1-2):83-93 (2000); J Biochem Biophys Methods. January 30; 47(1-2):5-19 (2001); Mutat Res. November 29; 430(1):13-21 (1999); Nucleic Acids Res. March 1; 28(5):E13 (2000); and Nucleic Acids Res. October 15; 28(20):E89 (2000), all of which are hereby expressly incorpo'rated by reference in their entireties including the references cited therein.

Because branched or heteroduplex DNA elutes either more rapidly or more slowly than homoduplex DNA, one can quickly determine the differences in melting behavior between DNA fragments, compare this melting temperature to a standard DNA (e.g., a wild-type or non-polymorphic homoduplex DNA), and identify the presence or absence of a mutation or polymorphism in the screened DNA. This technique efficiently separates multiple DNA fragments, generated by a single multiplex PCR reaction on a plurality of loci from different genes.

Some of the embodiments described herein have adapted the DNA separation techniques described above to allow for high-throughput genetic screening of organisms (e.g., plant, virus, bacteria, mold, yeast, and animals including humans). Typically, multiple primers that flank genetic markers (e.g., mutations or polymorphisms that indicate a congenital disease or a trait) on different genes are employed in a single amplification reaction or multiple amplification reactions and the multiple extension products are separated on a denaturing gel or by DHPLC according to their melting behavior. The presence or absence of mutations or polymorphisms, also referred to as “genetic markers”, in the subject's DNA are then detected by identifying an aberrant melting behavior in the extension products (e.g., migration on a gel that is too fast or too slow or elution from a DHPLC column that is too fast or too slow). Advantageously, some embodiments provide a greater understanding of a subject's health because more loci that are indicative of disease, for example, are analyzed in a single assay. Further, some embodiments drastically reduce the cost of performing such diagnostic assays because many different genes and markers for disease can be screened simultaneously in a single assay.

By one approach, for example, a biological sample from the subject (e.g., blood) is obtained by conventional means and the DNA is isolated. Next, the DNA is hybridized with a plurality of nucleic acid primers that flank regions of a plurality of genetic loci or markers that are associated with or linked to the plurality of traits to be analyzed. Although 10 different loci have been detected in a single assay (requiring 20 primers), more or less loci can be screened in a single assay depending on the needs of the user. Preferably, each assay has sufficient primers to screen at least three different loci, which may be located on three different genes. That is, the embodied assays can employ sufficient primers to screen at least 3, 4, 5, 6, 7, 8, 9, 10, 11, 12, 13, 14, 15, 16, 17, 18, 19, 20, 24 or more, independent loci or markers that are indicative of a disease in a single assay (e.g., in the same tube or multiple tubes) and these loci can be on different genes. Because more than one loci or marker can be detected by a single set of primers, the detection of 20 different markers, for example, can be accomplished with less than 40 primers. However, in many assays, a different set of primers is needed to detect each different loci. Thus, in several embodiments, at least 6, 8, 10, 12, 14, 16, 18, 20, 22, 24, 26, 28, 30, 32, 34, 36, 38, 40, or more primers are used.

Desirably, the primers hybridize to regions of human DNA that flank markers or loci associated with or linked to human diseases such as: familial hypercholesterolemia (FH), cystic fibrosis, Tay-sachs, thalassemia, sickle cell disease, phenylketonuria, galactosemia, fragile X syndrome, hemophilia A, myotonic dystrophy, medium-chain acyl CoA dehydrogenase, maturity onset diabetes, cystinuria, methylmolonic acidemia, urea cycle disorders, hereditary fructose intolerance, hereditary hemachromatosis, neonatal thrombocytopenia, Gaucher's disease, tyrosinemia, Wilson's disease, alcaptonuria, hypolactasia, Baker's disease, argininemia Adenomatous polyposis coli (APC), Adult Polycystic Kidney disease, a-1-antitrypsin deficiency, Duchenne Muscular Dystrophy, Hemophilia A, Hereditary Nonpolyposis colorectal cancer, Huntingtons disease, Marfan syndrome, Myotonic dystrophy, Neurofibromatosis, Osteogenesis imperfecta, Retinoblastoma, Sickle cell disease, Freidrichs ataxia, Hemoglobinopathies, Leber's hereditary optic neuropathy, MCAD, Canavan's disease, Retintitus Pigmentosa, Bloom Syndrome, Fanconi anemia, and Neimann Pick disease. It is particularly preferred that the primers hybridize to regions of DNA that flank markers associated with Hereditary Nonpolyposis Colorectal Cancer (HNPCC). It should be understood, however, that the list above is not intended to limit the invention in any way and the techniques described herein can be used to detect and identify any gene or mutation or polymorphism desired (e.g., polymorphisms or mutations associated with alcohol dependence, obesity, and cancer).

Once the primers are hybridized to the subject's DNA, a plurality of extension products having the marker or loci indicative of the trait are generated. Preferably, the extension products are generated through a polymerase-driven amplification reaction, such as multiplex PCR or multiplex Ligase Chain Reaction (LCR). In some embodiments, one or more fluorescent labels are employed. That is, by some methods, individual extension products are generated by PCR in the presence of different fluorescent labels so that the resulting extension products are fluoresce at different wavelengths (e.g., different colors are seen for each individual extension product on a detector). These embodiments facilitate the analysis of multiple patient samples in the same assay or multiple markers on the same or different genes. The extension products are then pooled according to similar melting behaviors and then the pooled samples are separated on the basis of melting behavior (e.g., TTGE or DHPLC).

In some approaches, for example, the extension products are isolated from the reactants in the amplification reaction, suspended in a non-denaturing loading buffer, and are loaded on a TTGE denaturing gel (e.g., an 8%, 7M urea polyacrylamide gel). The sample can be heated to a temperature sufficient to denature a DNA duplex and then cooled to a temperature that allows reannealing, prior to suspending the DNA in the non-denaturing loading buffer. The extension products are then loaded into a single lane or multiple lanes, as desired. Next, an electrical current is applied to the gel and extension products.

Subsequently, the temperature of the denaturing gel is gradually raised, while maintaining the electrical current, so as to separate the extension products on the basis of their melting behaviors. Once the fragments have been separated by size and melting behavior, one can identify the presence or absence of mutations or polymorphisms at the screened loci by analyzing the migration behavior of the extension products. By employing the fluorescent labels above, one can rapidly identify the differing extension products or patient samples, as well.

In other approaches, the extension products are isolated from the reactants and suspended in a DHPLC buffer (e.g., 0.1M TEAA pH 7.0). The extension products are then injected onto a DHPLC column (e.g., an ion-pair reverse phase HPLC column composed of alkylated non-porous poly(styrene-divinylbenzene)) that has been equilibrated to an appropriate denaturing temperature, depending on the size and composition of the DNA to be separated (e.g., 53° C. to 63° C.) in an appropriate buffer (e.g., 0.1 mM triethylamine acetate (TEAA) pH 7.0) and the extension products are allowed to bind. The presence of a denaturant (e.g., acetonitrile in TEAA) on the column is gradually raised over time so as to slowly elute the extension products from the column. Preferably a linear gradient is used. Presence of the extension products in the eluant is preferably accomplished using a UV detector (e.g., at 260 and/or 280 nm), however, greater sensitivity may be obtained using radioactivity, binding dyes, fluorescence or the techniques described in U.S. Pat. Nos. 5,795,976; 5,585,236; 6,024,878; 6,210,885; Huber, et al., Chromatographia 37:653 (1993); Huber, et al., Anal. Biochem. 212:351 (1993); Huber, et al., Anal. Chem. 67:578 (1995); and O'Donovan et al., Genomics 52:44 (1998), which are all hereby incorporated by reference in their entireties including the references cited therein.

The appearance of a slower or faster migrating band at a temperature below or above the predicted melting point for the particular extension product in the TTGE approach, for example, indicates the presence of a mutation or polymorphism in the subject's DNA. Similarly, the appearance of a slower or faster eluting peak at a concentration of denaturant predicted to elute a wild-type or non-polymorphic homoduplex extension product in the DHPLC approach indicates the presence of a mutation or polymorphism in the subject's DNA. A heterozygous sample will display both homoduplex bands (wild-type homoduplexes and mutant homoduplexes), as well as, two heteroduplex bands that are the product of mutant/wild-type annealing. Because of base pair mismatches in these fragments, they melt significantly sooner than the two homoduplex bands. Accordingly, a user can rapidly identify the presence or absence of a mutation or polymorphism at the screened loci by either the TTGE or DHPLC approach and determine whether the tested subject has a predilection for a disease.

In a related embodiment, greater sensitivity is obtained by adding a “standard” DNA or “control” DNA to the DNA to be screened prior to amplification or after amplification, prior to separation of the DNA on the TTGE gel or DHPLC column. This insures the presence of heteroduplexes in the case of either a homozygous mutant, which normally would not display heteroduplexes, or a heterozygous mutant. Desired DNA standards include, but are not limited to, DNA that is wild-type for at least one of the traits that are being screened. Preferred standards include, but are not limited to, DNA that is wild-type for all of the traits that are being screened. A DNA standard can also be a mutant or polymorphic DNA. In some embodiments, particularly when the control DNA is added after amplification, the DNA standard is an extension product generated from a wild-type genomic DNA or a mutant genomic DNA. By this approach, the amplification phase of the method is performed as described above. That is, DNA from the subject to be screened and the DNA standard are hybridized with nucleic acid primers that flank regions of the genetic loci or markers that are associated with or linked to the traits being tested. In some embodiments, the DNA standard extension products are fluorescently labeled differently than the extension products generated from the screened samples so as to facilitate identification.

Extension products are then generated. If the subject being tested has at least one trait that is detected by the assay (e.g., a congenital disorder), then two populations of extension products are generated, a first population that corresponds to the standard DNA and a second population that corresponds to the subject's DNA having at least one mutation or polymorphism. Next, preferably, the two populations of extension products are isolated from the amplification reactants and are denatured by heat (e.g., 95° C. for 5 minutes), then are allowed to anneal by cooling (e.g., ice for 5 minutes). This ensures the formation of the heteroduplex bands in the presence of any relatively small mutation (e.g., point mutation, small insertion, or small deletion). The isolation and denaturing/annealing steps are not practiced with some embodiments, however.

Subsequently, by the TTGE approach, the two populations of extension products are suspended in a non-denaturing loading buffer and loaded on a denaturing polyacrylamide gel and separated on the basis of melting behavior, as described above. By the DHPLC approach, the two populations of extension products are suspended in a suitable buffer (e.g., 0.1M TEAA pH 7.0), loaded onto a buffer and temperature equilibrated DHPLC column and a linear gradient of denaturant is applied, as described above. Because the two populations of extension products are not perfectly complementary, they form heteroduplexes. Heteroduplexes are less stable than homoduplexes, have a lower melting temperature, and are easily differentiated from homoduplexes using the DNA separation techniques described above. One can identify the presence or absence of mutations or polymorphisms at the screened loci, for example, by comparing the migration behavior or elution behavior of the extension products generated from the screened DNA with the migration behavior or elution behavior of the DNA standard. If heteroduplexes are present, generally, two additional bands that correspond to the single extension product will appear on the gel or the extension products will elute from the column more rapidly than the control or standard DNA alerting the user to the presence of a mutation or polymorphism. Accordingly, a significant increase in sensitivity is obtained and a user can rapidly identify the presence or absence of a mutation or polymorphism in the tested DNA sample and, thereby, determine whether the screened subject has a predilection for a particular trait (e.g., a congenital disease). As stated above, by employing different fluorescent labels during individual amplification reactions, different fluorescently labeled extension products can be generated and the identification of particular markers can be facilitated.

Similarly, an increase in sensitivity can be obtained by mixing DNA from a plurality of subjects prior to amplification. Because the frequency of mutations or polymorphisms for most disorders are very low in the population, most of the extension products generated are wild-type DNA. Thus, most of the pool of DNA behaves as a DNA standard. That is, the predominant structure formed upon annealing after denaturation is a homoduplex, which can be rapidly distinguished from any heteroduplex that would appear if a subject were to have a polymorphism or mutation. Of course, extension products previously generated from multiple subjects can be used as control DNA by mixing the previously generated extension products with the extension products generated from the DNA that is being screened prior to electrophoresis. In several embodiments, the DNA from at least 2 subjects is mixed. Desirably, the DNA from at least 3 subjects is mixed. Preferably, the DNA from at least 4 subjects is mixed. It should be understood, however, that the DNA from at least 2, 3, 4, 5, 6, 7, 8, 9, 10, 11, 12, 13, 14, 15, 16, 17, 18, 19, 20, or more subjects can be mixed prior to amplification or prior to separation on the basis of melting behavior, in accordance with some of the described embodiments. Again, by employing different fluorescent labels during individual amplification reactions, different fluorescently labeled extension products can be generated and the identification of genetic markers, in particular the same markers on different subjects (e.g., the amplification reactions for different subjects employ different fluorescent markers) can be facilitated.

In one embodiment, for example, DNA from a plurality of subjects to be tested is obtained by conventional methods, pooled, and hybridized with the desired nucleic acid primers. Extension products are then generated, as before. If at least one of the subjects being tested has at least one congenital disorder that is detected by the screen then two populations of extension products will be generated, a first population that corresponds to DNA from subjects that have the wild-type gene and a second population that corresponds to DNA from subjects having at least one mutant or polymorphic gene.

By one approach, the two populations of extension products are then isolated from the amplification reactants, suspended in a non-denaturing loading buffer, denatured by heat, annealed by cooling, and are separated by TTGE, as described above. By another approach, the two populations of extension products are isolated from the amplification reactants, suspended in a DHPLC loading buffer (0.1M TEAA pH 7.0), denatured by heat, annealed by cooling, and are separated on a DHPLC column, as described above. The presence of a subject in the DNA pool having at least one mutation or polymorphism is identified by analyzing the migration behavior of the DNA on the gel or the elution behavior from the column. The appearance of a slower or faster migrating band at a temperature below or above the predicted melting point for a particular extension product on the gel indicates the presence of a mutation or polymorphism in the DNA from one of the subjects. Similarly, the appearance of a slower or faster eluting extension product from the DHPLC column indicates the presence of a mutation or polymorphism in the DNA from one of the subjects. By repeating the analysis with smaller and smaller pools of samples, one can identify the individual(s) in the pool that has the mutation or polymorphism. Additionally, DNA standards can be used, as described above, to facilitate identification of the individual(s) having the mutation or polymorphism. Advantageously, some embodiments can be used to screen multiple samples at multiple loci that are on found on a plurality of genes in a single assay, thus, increasing sample throughput. The analysis of a plurality of DNA samples in the same assay also unexpectedly provides greater sensitivity. The section below describes a DNA separation technique that can be used with the embodiments described herein.

Multiple Extension Products of Similar Composition can be Separated on the Same Lane of a Denaturing Gel or in the Same Run on a DHPLC Column

It was discovered that multiple fragments of DNA, which vary slightly in length and/or composition, can be rapidly and efficiently resolved on the basis of melting behavior. Although the preferred methods for differentiating multiple fragments of DNA on the basis of melting behavior involve TTGE gel electrophoresis and DHPLC, it is contemplated that other conventional techniques that are amenable to DNA separation on the basis of melting behavior can be equivalently employed (e.g., size exclusion chromatography, ion exchange chromatography, and reverse phase chromatography on high pressure (e.g., HPLC), low pressure (e.g., FPLC), gravity-flow, or spin-columns, as well as, thin layer chromatography).

By one approach, a polyacrylamide gel having a porosity sufficient to resolve the DNA fragments on the basis of size (e.g., 4-20% acrylamide/bis acrylamide gel having a set concentration of denaturant) is used. The amount of denaturant in the gel (e.g., urea or formamide) can vary according to the length and composition of the DNA to be resolved. The concentration of urea in a polyacrylamide gel, for example, can be 3M, 3.5M, 4M, 4.5M, 5M, 5.5M, 6M, 6.5M, 7M, 7.5M, or 8M. In preferred embodiments, an 8% polyacrylamide gel with 7M urea is used. It should be emphasized, however, that other types of polyacrylamide gels, equivalents thereof, and agarose gels can be used.

The DNA samples to be resolved are placed in a non-denaturing buffer and can be loaded directly to the gel. In some embodiments, for example, when heteroduplex formation is desired to increase the sensitivity of the assay, it is desirable to heat the double stranded DNA to a temperature that permits denaturation (e.g., 95° C. for 5-10 minutes) and then slowly cool the DNA to a temperature that allows annealing (e.g., ice for 5-10 minutes) prior to mixing with the loading buffer. Preferably, the DNA is loaded onto the gel in a total volume of 10-20 μl. Preferably, a Temporal Temperature Gradient Gel Electrophoresis (TTGE) apparatus is used. A commercially available system that is suitable for this technique can be obtained from BioRad. The gel can be run at 120, 130, 140, 150, 175, 200, 220, 250, 275, or 300 V for 1.5-10 hours, for example.

Once the DNA has been loaded, an electrical current is applied to begin separating the fragments on the bass of size and the temperature of the gel is raised gradually. In one embodiment, for example, the melting behavior separation is performed by raising the temperature beyond 60° C., 61° C., 62° C., 63° C., 64° C., 65° C., 66° C., 67° C., 68° C., 69° C., 70° C., 71° C., 72° C., 73° C., 74° C., or 75° C. at approximately 5.0 C.°/hour-0.5° C./hour in 0.1° C. increments.

Once the extension products have been separated by melting behavior, the gel can be stained to reveal the separated DNA. Many conventional stains are suitable for this purpose including, but not limited to, ethidium bromide stain (e.g., 1% ethidium bromide in a 1.25×Tris Acetate EDTA pH 8.0 (TAE) solution), fluorescent stains, silver stains, and colloidal gold stains. In some embodiments, it is desirable to destain the gel (e.g., 20 minutes in a 1.25×TAE solution). After staining, the gel can be analyzed visually (e.g., under a U.V. lamp) and/or with a digital camera and computer software such as, the Eagle Eye System by Stratagene or the Gel Documentation System (BioRad). Additionally, when fluorescent markers are employed, conventional detectors that emit various wavelengths of light can be used so as to identify the presence and position of separated fluorescently labeled extension products.

Mutations or polymorphisms are easily identified by comparing the migration behavior of the DNA to be screened with the migration behavior of a control DNA and/or by monitoring the melting temperature of the extension products generated from the screened DNA. Desirable “control” DNA or “standard” DNA includes a DNA that is wild-type or non-polymorphic for at least one loci that is screened and preferred standard DNA is wild-type or non-polymorphic for all of the loci that are being screened. Because this DNA separation technique is sufficiently sensitive to identify a single base pair substitution in a DNA fragment up to 600 base pairs in length, small changes in the melting behaviors and migration of the extension products can be rapidly identified. The standard or control DNA can also be fluorescently labeled (preferably with a fluorescent label that is different than the one employed for the screened samples) to facilitate the analysis.

By another approach, DHPLC is used to resolve heteroduplex and homoduplex molecules of several PCR extension products in a single assay. Preferably, the heteroduplex and homoduplex extension products are separated from each other by ion-pair reverse phase high performance liquid chromatography. In one embodiment, a DHPLC column that contains alkylated non-porous poly(styrene-divinylbenzene) is used. Preferably, the DHPLC column is equilibrated in an appropriate degassed buffer, referred to as Buffer “A” (e.g., 0.1M TEAA pH 7.0) and is kept at a constant temperature somewhat below the predicted melting temperature of the extension products (e.g., 53° C.-60° C., preferably 50° C.). A plurality of extension products that may be generated from a plurality of different loci, as described herein, are suspended in Buffer A and are injected onto the DHPLC column. The Buffer A is then allowed to run through the column for a time sufficient to insure that the extension products have adequately bound to the column. Preferably, flow rate and the amount of gas (e.g., argon or helium) are adjusted and kept constant so that the pressure on the column does not exceed the recommended level. Gradually, degassed denaturing buffer, referred to as Buffer “B”, (e.g., 0.1M TEAA pH 7.0 and 25% acetonitrile) is applied to the column. Although an isocratic gradient can be used, a gradual linear gradient is preferred. By one approach, to separate fragments that range in size from 200-450 bp, for example, a gradient of 50%-65% Buffer B (0.1M TEAA pH 7.0 and 25% acetonitrile) is used. Of course, as the size of extension products to be separated on the DHPLC column decreases, the gradient and/or the amount of denaturant in Buffer B can be reduced, whereas, as the size of extension products to be separated on the DHPLC column increases, the gradient and/or the amount of denaturant in Buffer B can be increased.

The DHPLC column is designed such that double stranded DNA binds well but as the extension products become partially denatured the affinity to the column is reduced until a point is reached at which the particular extension product can no longer adhere to the column matrix. Typically, heteroduplexes denature before homoduplexes, thus, they would be expected to elute more rapidly from the column than homoduplexes.

In some embodiments, particularly embodiments concerning the separation of a plurality of different extension products (e.g., extension products generated from a plurality of loci), the choice of primers and, thus, the extension products generated therefrom, requires careful design. For example, a GC-clamp or other artificial sequence can be used to adjust the melting characteristics and increase the length of a particular DNA fragment, if needed, to facilitate separation on the DHPLC or improve resolution of the extension products. By one approach, each set of primers in a multiplex reaction are designed and selected to generate an extension product that has a unique homoduplex and heteroduplex elution behavior. In this manner, each species can be easily identified.

By another approach, each set of primers are designed to generate extension products that have homoduplexes with very similar melting characteristics. By this strategy, all of the homoduplexes will elute at the same or very similar concentration of denaturant, which is different than the concentration of denaturant required to elute the heteroduplexes. Accordingly, the elution of a species of extension product outside of the expected range for the homoduplexes indicates the presence of a mutation or polymorphism.

In the case that the extension products happen to have overlapping retention times/elution behaviors, the DHPLC conditions can be adjusted to include a primary separation on the basis of size prior to increasing the concentration of the denaturant on the column to improve resolution. The techniques described in Huber, et al., Anal. Chem. 67:578 (1995), hereby expressly incorporated by reference in its entirety, can be adapted for use with the novel DHPLC separation approach described herein. In one embodiment, for example, the alkylated non-porous poly(styrene-divinylbenzene) DHPLC column can be used to separate the extension products on the basis of size for a time sufficient to group the various populations of extension products (i.e., the homoduplexes and heteroduplexes generated from a single independent set of primers constitute a single population of extension products) prior to separating on the basis of melting behavior.

By one approach, the extension products are applied to the column, as above, in Buffer A and a shallow linear gradient of Buffer B (e.g., 30%-50% of a solution of 0.1M TEAA pH 7.0 and 25% acetonitrile for 200-450 by extension products) is applied so as to resolve the various populations of extension products. Then, a deeper linear gradient of Buffer B (e.g., 50%-65% of a solution of 0.1M TEAA pH 7.0 and 25% acetonitrile for 200-450 by extension products) is applied to resolve the homoduplexes from the heteroduplexes within each individual population of extension product. In this manner, the homoduplexes and heteroduplexes from each population of extension product can be resolved despite having overlapping elution behaviors.

It should be understood that the separation based on size can be performed at virtually any temperature as long as the extension products do not denature on the column, however, the amount of denaturant in Buffer B and the type of gradient may have to be adjusted. For example, the size separation can be accomplished at 4° C.-23° C., or 23° C.-40° C., or 40°-50° C., or 50° C.-60° C. Additionally, the size separation can be accomplished while the column is being gradually equilibrated to the temperature that is going to be used for the DHPLC. It should also be understood that the size separation can be performed on the same column with the appropriate gradient (shallow for a time sufficient to separate on the basis of size followed by a deeper gradient to separate on the basis of melting behavior). Additionally, columns in series can be used to separate extension products that have overlapping retention times/elution behaviors. For example, a first DHPLC column can be used to separate on the basis of size and a second DHPLC column can be used to separate on the basis melting behavior.

Mutations or polymorphisms are easily identified using the DHPLC techniques above by comparing the elution behavior of the DNA to be screened with the elution behavior of a control DNA. As above, desirable “control” DNA or “standard” DNA includes a DNA that is wild-type or non-polymorphic for at least one loci that is screened and preferred standard DNA is wild-type or non-polymorphic for all of the loci that are being screened. Control or standard DNA can also include extension products that are homoduplexes by virtue of a mutation or polymorphism or plurality of mutations or polymorphisms. Since the elution behavior of the wild type or non-polymorphic DNA or a homozygous mutant or polymorphism, represents the elution behavior of a homoduplex, one can use DHPLC values obtained from separating these controls, such as the retention time, elution time, or amount of denaturant required to elute the homoduplex as a basis for comparison to a screened sample to identify the presence of homoduplexes. Similarly, a control DNA can be a known heteroduplex and the elution behavior values described above can be used to identify the presence of a heteroduplex in a screened sample.

Additionally, the separated extension products can be collected after passing through the DHPLC column or TTGE gel or reamplified and sequenced to verify the existence of the mutation or polymorphism. Further, the identified products can be isolated from the gel and sequenced. Sequencing can be performed using the conventional dideoxy approach (e.g., Sequenase kit) or an automated sequencer. Preferably, all possible mutant fragments are sequenced using the CEQ 2000 automated sequencer from Beckman/Coulter and the accompanying analysis software. The mutations or polymorphisms identified by sequencing can be compiled along with the respective melting behaviors and the sizes of extension products. This data can be recorded in a database so as to generate a profile for each loci.

Additionally, this profile information can be recorded with other subject-specific information, for example family or medical history, so as to generate a subject profile. By creating such databases, individual mutations can be better characterized. Mutation analysis hardware and software can also be employed to aid in the identification of mutations or polymorphisms. For example, the “ALFexpress II DNA Analysis System”, available from Amersham Pharmacia Biotech and the “Mutation Analyser 1.01”, also available from Amersham Pharmacia Biotech, can be used. Mutation Analyser automatically detects mutations in sample sequence data, generated by the ALFexpress II DNA analysis instrument. The section below describes embodiments that allow for the identification of a mutation or polymorphism at multiple loci in a plurality of genes in a single assay.

Identification of the Presence or Absence of a Mutation or Polymorphism at Multiple Loci in a Plurality of Genes in a Single Assay

The DNA separation techniques described herein can be used to rapidly identify the presence or absence of a mutation or polymorphism at multiple loci in a plurality of genes in a single assay (e.g., in a single reaction vessel or multiple reaction vessels). Accordingly, a biological sample containing DNA is obtained from a subject and the DNA is isolated by conventional means. For some applications, it may be desired to screen the RNA of a subject for the presence of a genetic disorder (e.g., a congenital disease that arises through a splicing defect). In this case, a biological sample containing RNA is obtained, the RNA is isolated, and then is converted to cDNA by methods well known to those of skill in the art. DNA from a subject or cDNA synthesized from the mRNA obtained from a subject can be easily and efficiently isolated by various techniques known in the art. Also known in the art is the ability to amplify DNA fragments from whole cells, which can also be used with the embodiments described herein. Thus, the DNA sample for use with the embodiments described herein need only be isolated in the sense that the DNA is in a form that allows for PCR amplification.

In some embodiments, genomic DNA is isolated from a biological sample by using the Amersham Pharmacia Biotech “GenomicPrep Blood DNA Isolation Kit”. The isolation procedure involves four steps: (1) cell lysis (cells are lysed using an anionic detergent in the presence of a DNA preservative, which limits the activity of endogenous and exogenous Dnases); (2) RNAse treatment (contaminating RNA is removed by treatment with RNase A); (3) protein removal (cytoplasmic and nuclear proteins are removed by salt precipitation); and (4) DNA precipitation (genomic DNA is isolated by alcohol precipitation). EXAMPLE 1 also describes an approach that was used to isolate DNA from human blood.

Once the sample DNA has been obtained, primers that flank the desired loci to be screened are designed and manufactured. Preferably, optimal primers and optimal primer concentrations are used. Desirably, the concentrations of reagents, as well as, the parameters of the thermal cycling are optimized by performing routine amplifications using control templates. Primers can be made by any conventional DNA synthesizer or are commercially available. Optimal primers desirably reduce non-specific annealing during amplification and also generate extension products that resolve reproducibly on the basis of size or melting behavior and, preferably, both. Preferably, the primers are designed to hybridize to sample DNA at regions that flank loci that can be used to diagnose a trait, such as a congenital disease (e.g., loci that have mutations or polymorphisms that indicate a human disease).

Desirably, the primers are designed to detect loci that diagnose conditions selected from the group consisting of familial hypercholesterolemia (FH), cystic fibrosis, Tay-sachs, thalassemia, sickle cell disease, phenylketonuria, galactosemia, fragile X syndrome, hemophilia A, myotonic dystrophy, medium-chain acyl CoA dehydrogenase, maturity onset diabetes, cystinuria, methylmolonic acidemia, urea cycle disorders, hereditary fructose intolerance, hereditary hemachromatosis, neonatal thrombocytopenia, Gaucher's disease, tyrosinemia, Wilson's disease, alcaptonuria, hypolactasia, Baker's disease, argininemia Adenomatous polyposis coli (APC), Adult Polycystic Kidney disease, a-1-antitrypsin deficiency, Duchenne Muscular Dystrophy, Hemophilia A, Hereditary Nonpolyposis colorectal cancer, Huntingtons disease, Marfans syndrome, Myotonic dystrophy, Neurofibromatosis, Osteogenesis imperfecta, Retinoblastoma, Sickle cell disease, Freidrichs ataxia, Hemoglobinopathies, Leber's hereditary optic neuropathy, MCAD, Canavan's disease, Retintitus Pigmentosa, Bloom Syndrome, Fanconi anemia, and Neimann Pick disease. Preferably, the primers are designed to detect the presence or absence of polymorphisms or mutation associated with Hereditary Nonpolyposis Colorectal Cancer (HNPCC). Primers can be designed to amplify any region of DNA, however, including those regions known to be associated with diseases such as alcohol dependence, obesity, and cancer. It should be understood that the embodiments described herein can be used to detect any gene, mutation, or polymorphism found in plants, virus, molds, yeast, bacteria, and animals.

Preferred primers are designed and manufactured to have a GC rich “clamp” at one end of a primer, which allows the dsDNA to denature in a “zipper-like” fashion. As one of skill will appreciate, PCR requires a “primer set”, which includes a first and a second primer, only one of which has the GC clamp so as to allow for separation of the double stranded molecule from one end only. Since the GC clamp is significantly stable, the rest of the fragment melts but does not completely separate until a point after the inflection point of the DNA, which contains the mutation or polymorphism of interest. The denaturant in the gel or on the column allows the temperature of melting to be lower and allows the inflection point of the melt to be longer in terms of temperature and, thus, the sensitivity to temperature at the inflection point is less (i.e., increment temperature=less increment melting), which increases the resolution.

Additionally, desirable primers are designed with a properly placed GC-clamp so that extension products that contain a single melting domain are produced. Preferably, the primers are selected to complement regions of introns that flank exons containing the genetic markers of interest so that polymorphisms or mutations that reside within the early portions of exons are not masked by the GC clamp. For example, it was discovered that GC clamps significantly perturb melting behavior and can prevent the detection of a polymorphism or mutation by melting behavior if the mutation or polymorphism resides too close to the GC clamp (e.g., within 40 nucleotides). By performing amplification reactions with control templates, optimal primer design and optimal concentration can be determined. The use of computer software, including, but not limited to, WinMelt or MacMelt (Bio-Rad) and Primer Premire 5.0 can aid in the creation and optimization of primers and proper positioning of the GC-clamp. Accordingly, many of the primers and groupings of primers described herein, as used in a particular assay (e.g., to screen for HNPCC) are embodiments of the invention. EXAMPLE 2 further describes the design and optimization of primers that allowed for the high-throughput multiplex PCR technique described herein.

Once optimal primers are designed and selected, the DNA sample is screened using the inventive multiplex PCR technique. In some embodiments, for example, approximately 25 ng-500 ng of template DNA (preferably, 200 ng for human genomic DNA) is suspended in a buffer comprising: 10 mM Tris (pH 8.4), 50 mM KCl, 1.5 mM MgCl2, 200 μM dNTPs, 50 pmol of each primer, and 1 unit Taq polymerase per primer set in a total volume of 50 μl. Preferably, amplification is performed under the same conditions that were used to design the primers. In some embodiments, for example, amplification is performed on a conventional thermal cycler for 30 cycles, wherein each cycle is: 1 minute @ 95° C., 58° C. for 1 minute, 72° C. for 1 minute. Final extension is performed at 72° C. for 5 minutes. When the primers have a GC clamp, it was found that conditions often favor an amplification reaction having over 40 cycles, wherein each cycle is: 35 seconds @ 95° C., 120 seconds @ 50-57° C., and 60 seconds+3 seconds/cycle @ 72° C. Thermal cyclers are available from a number of scientific suppliers and most are suitable for the embodiments described herein.

Once the PCR reaction is complete, the extension products are desirably isolated by centrifugal microfiltration using a standard PCR cleanup cartridge, for example, Qiagen's QIAquick 96 PCR Purification Kit, according to manufacture's instructions. Isolation or purification of the extension products is not necessary to practice the invention, however. The isolated extension products can then be suspended in a non-denaturing loading buffer and either loaded directly on a DHPLC column or TTGE denaturing gel. The sample can also be denatured by heating (e.g., 95° C. for 5-10 minutes) and annealed by cooling (e.g., ice for 5-10 minutes) prior to loading onto the DHPLC column or TTGE denaturing gel. The various extension products are then separated on a TTGE denaturing gel or DHPLC column on the basis of melting behavior, as described above and, after separation, the extension products can be analyzed for the presence or absence of polymorphisms or mutations. EXAMPLES 3 and 4 describe experiments that verified that multiple loci on a plurality of genes can be screened in a single assay. The section below describes a method of genetic analysis, wherein improved sensitivity of detection was obtained by adding a DNA standard to the screened DNA.

Improved Sensitivity was Obtained Wizen a DNA Standard was Mixed with the Screened DNA

It was also discovered that greater sensitivity in the inventive multiplex PCR reactions described herein can be obtained by mixing a DNA standard with the DNA to be tested prior to conducting amplification or after amplification but prior to separation on the basis of melting behavior. Desired DNA standards include, but are not limited to, DNA that is wild-type for at least one of the traits that are being screened and preferred DNA standards include, but are not limited to, DNA that is wild-type for all of the traits that are being screened. DNA standards can also be mutant or polymorphic DNA. In some embodiments, particularly when the control DNA is added after amplification, the DNA standard is an extension product generated from a wild-type genomic DNA or a mutant genomic DNA. Optionally, the control DNA can be labeled with a fluorescent label, which can be a label that is different than the fluorescent label used to label the extension products generated from the screened sample DNA. In this manner, the standard or control DNA is easily differentiated from the DNA that is being screened.

By one approach, the DNA from the subject to be screened and the DNA standard are pooled and then the amplification reaction, as described above, is performed. Accordingly, optimal primers are designed and selected and approximately 25 ng-500 ng of template DNA (preferably, 200 ng for human genomic DNA) is suspended in a buffer comprising: 10 mM Tris (pH 8.4), 50 mM KCl, 1.5 mM MgCl2, 200 μM dNTPs, 50 pmol of each primer, and 1 unit Taq polymerase per primer set in a total volume of 50 μl. Preferably, amplification is performed under the same conditions that were used to design the primers. In some embodiments, amplification is performed on a conventional thermal cycler for 30 cycles, wherein each cycle is: 1 minute @ 95° C., 58° C. for 1 minute, 72° C. for 1 minute. Final extension is performed at 72° C. for 5 minutes. When the primers have a GC clamp, however, conditions often favor an amplification reaction having over 40 cycles, wherein each cycle is: 35 seconds @ 95° C., 120 seconds @ 50-57° C., and 60 seconds+3 seconds/cycle @ 72° C.

If the subject being tested has at least one disorder that is detected by the assay then two populations of extension products are generated, a first population that corresponds to the standard DNA and a second population that corresponds to the subject's DNA having at least one mutation or polymorphism. The pool of extension products are desirably isolated from the amplification reactants, as above, and are suspended in a non-denaturing loading buffer. Preferably, the extension products are then denatured by heat (e.g., 95° C. for 5 minutes), and are allowed to anneal by cooling (e.g., ice for 5 minutes) prior to loading on the TTGE denaturing gel or DHPLC column. In this manner, the formation of heteroduplexes will be favored if the subject has a mutation or polymorphism because the two populations of extension products are not perfectly complementary. However, the isolation and denaturing/annealing steps are not necessary for some embodiments.

By another approach, the DNA standard is added to the extension products generated from the tested subject's DNA after the amplification reaction. As above, the pooled DNA sample is preferably denatured by heat (e.g., 95° C. for 5 minutes), and allowed to anneal by cooling (e.g., ice for 5 minutes). This second approach also produces heteroduplexes if the extension product and the DNA standard are not perfectly complementary.

Next, the TTGE denaturing gel or DHPLC column is loaded and the extension products are separated on the basis of melting behavior, as described above. Since heteroduplexes are less stable than homoduplexes and have a lower melting temperature, the presence or absence of a mutation or polymorphism in the tested DNA sample is easily determined. By comparing the migration behavior or elution behavior of the extension products generated from the screened DNA with the migration behavior of the DNA standard, a user can rapidly determine the presence or absence of a mutation or polymorphism (e.g., two additional bands that correspond to the single extension product will appear on the gel when a mutation or polymorphism is present in the tested DNA or a population of extension products will elute from the DHPLC column earlier than homoduplex controls or the majority of homoduplexes present in the sample). The section below describes a method of genetic analysis, wherein improved efficiency and sensitivity of detection was obtained by screening multiple DNA samples in the same assay.

Improved Sensitivity was Obtained when Multiple DNA Samples were Screened in the Same Assay

It was also discovered that an improved sensitivity of detection and increased throughput could be obtained by mixing DNA from a plurality of subjects prior to amplification. Because the frequency of mutations or polymorphisms for most disorders are very low in the population, most of the extension products generated correspond to wild-type or non-polymorphic DNA. Accordingly, most of the DNA in a reaction comprising DNA from a plurality of subjects behave similar to a DNA standard. That is, the predominant structure formed upon annealing after denaturation is a homoduplex, which can be rapidly distinguished from any heteroduplex that would appear if a subject were to have a mutation or polymorphism. Although the reaction is “dirty” from the perspective that the identity of each subject's DNA is not known initially, the identity of any polymorphic or mutant DNA can be determined through a process of elimination. For example, by repeating the analysis with smaller and smaller pools of samples, one can identify the individual(s) in the pool that have the mutation or polymorphism. Additionally, DNA standards can be used, as described above, to facilitate identification of the individual(s) having the mutation or polymorphism. Optionally, the each DNA can be labeled with a different fluorescent label so that identification of the variant is easily determined.

By one approach, DNA from a plurality of subjects to be tested is obtained by conventional methods, pooled, and hybridized with the desired nucleic acid primers. Accordingly, optimal primers are designed and selected and approximately 25 ng-500 ng of template DNA (preferably, 200 ng for human genomic DNA) is suspended in a buffer comprising: 10 mM Tris (pH 8.4), 50 mM KCl, 1.5 mM MgCl2, 200 μM dNTPs, 50 pmol of each primer, and 1 unit Taq polymerase per primer set in a total volume of 50 μl. Preferably, amplification is performed under the same conditions that were used to design the primers. In some embodiments, amplification is performed on a conventional thermal cycler for 30 cycles, wherein each cycle is: 1 minute @ 95° C., 58° C. for 1 minute, 72° C. for 1 minute. Final extension is performed at 72° C. for 5 minutes. When the primers have a GC clamp, however, conditions often favor an amplification reaction having over 40 cycles, wherein each cycle is: 35 seconds @ 95° C., 120 seconds @ 50-57° C., and 60 seconds+3 seconds/cycle @ 72° C.

The pool of extension products are preferably isolated from the amplification reactants, as above, and are suspended in a non-denaturing loading buffer. Preferably, the extension products are then denatured by heat (e.g., 95° C. for 5 minutes), and are allowed to anneal by cooling (e.g., ice for 5 minutes). In this manner, the formation of heteroduplexes will be favored if the subject has a mutation or polymorphism because the two types of extension products are not perfectly complementary. Again, the isolation and denaturing/annealing steps are not performed in some embodiments and fluorescent labels can be employed.

Next, the TTGE denaturing gel or DHPLC column is loaded and the extension products are separated on the basis of melting behavior, as described above. When one of the subjects being tested has at least one trait that is detected by the screen, heteroduplexes are detected on the gel or eluting from the DHPLC column. The assay can be then repeated with smaller pools of samples and assays with a DNA standard can be conducted with individual samples to confirm the identity of the subject having the mutation or polymorphism. EXAMPLE 5 describes an experiment that verified that an improved sensitivity can be obtained by mixing a plurality of DNA samples. EXAMPLE 6 describes an experiment that verified that multiple genes and multiple loci therein can be screened in a plurality of subjects, in a single assay. EXAMPLE 7 describes the screening of multiple genes and multiple loci therein, in a plurality of subjects, in a single assay using a DHPLC approach. The section below describes the optimization of primer design in the context of an approach that was used to detect mutations and/or polymorphisms in the CFTR gene.

Optimization of Primer Design and Extension Product Design Facilitates Identification of Genetic Markers Associated with HNPCC

Using the approaches detailed in the previous sections, a preferred embodiment concerns the identification of the presence or absence of genetic markers, mutations, or polymorphisms that are associated with HNPCC. The sequences of genes associated with HNPCC can be found in U.S. Pat. Nos. 5,922,855; 6,165,713; 6,191,268; 6,538,108 and U.S. patent application Ser. Nos. 08/209,521 and 08/154,792, all of which are hereby expressly incorporated by reference in their entireties.

By one approach, almost the entire coding sequences for the mismatch repair genes mutL homolog 1 (MLH1) and mutS homologue 2 (MSH2) are scanned for the presence or absence of genetic markers, mutations, or polymorphisms that contribute to HNPCC. (See EXAMPLE 8). TABLE A provides the sequences of exons of the MLH1 and MSH2 genes and several oligonucleotide primers that have been used to screen regions of these genes for the presence or absence of genetic markers, polymorphisms, and mutations that are associated with HNPCC. Where indicated, the notation (*) refers to a GC clamp, an additional non-genetic GC rich sequence that is added to one of the two primers in a pair to add stability to the PCR product, as explained above and in Example 2 below. TABLE B also lists many oligonucleotide primers that have been used to screen regions of the MLH1 and MSH2 genes for the presence or absence of genetic markers, polymorphisms, and mutations that are associated with HNPCC. TABLE B also shows the starting and ending point for each primer as it relates to the publicly available gene sequence for the MLH1 and MSH2 genes (GenBank Accession NoS. AY217549 and NM000251, the contents of which are expressly incorporated by reference in its entirety). It is contemplated that primers that are any number between 5, 10, 15, 20, 25, 30, 35, 40, 45, 50, 55, 60, 65, 70, 75, 80, 85, 90, 95, 100 or more nucleotides upstream or downstream of the primers identified in TABLE A or B can be used with embodiments of the invention so long as these primers produce extension products that melt over long stretches of DNA (approximately 25, 50, 75, 100, 125, or 150 nucleotides) at approximately the same temperature (within 0° C.-1.5° C.) and are resolvable on a TTGE gel or DHPLC column.

As detailed above, the sequences of the MLH1 and MSH2 genes are readily available. Accordingly, embodiments include methods of diagnosing HNPCC with primers that are any number from 1-75 nucleotides upstream or down stream from the beginning or ending of the primers listed in TABLE A or B, preferably using the approaches described herein. It is also preferred that said methods use primers that produce extension products that melt over long stretches of DNA (approximately 25, 50, 75, 100, 125, or 150 nucleotides) at approximately the same temperature (within 0° C.-1.5° C.) and are resolvable on a TTGE gel or DHPLC column. Preferably, these extension products are obtained, grouped, and separated as described below.

By one approach, samples of DNA were obtained from several subjects to be screened using the approaches described herein and were disposed in a plurality of 96-well micro-titer plates such that a single row of each plate corresponded to a single tested subject. In some cases, 7 total plates were used per assay, wherein each plate has 7 sample lanes (i.e., 7 subjects analyzed) and an eighth lane was used for positive control sample DNA. Amplification buffer, amplification enzyme (e.g., Taq polymerase), and DNTPs were added to the sample DNA in each well, as described above, and a plurality of primer sets that encompass most of the gene (e.g., 84 primer sets) were to yield a final volume of 10 μl. The primer sets that were employed in a first set of tests are identified in TABLE A. TABLE C describes the plate setup for these amplification reactions as well as a protocol for PCR reactions, whereas TABLE D describes the conditions for the TTGE separation for these tests and describes the groupings for the various fragments for TTGE separation. Preferred methods of diagnosing HNPCC employ the primers of TABLE A to generate extension products that are grouped according to TABLE D and separated by melting behavior (e.g., TTGE). By using this approach, a rapid, inexpensive, and efficient diagnosis of the presence or absence of a marker associated with HNPCC can be ascertained. The names of the extension products, “fragments” in TABLE C and TABLE D correspond to the names of the primer sets used throughout. The top line numbering on the master plate chart of TABLE C refers to the location of the well on the 96 well plate, the “MLH stack” or “MSH stack” of TABLE D refers to the grouping pool of the extension products prior to TTGE and the alternating shaded and unshaded sections of TABLE D show grouping pools of extension products that can be run under the same TTGE conditions (which are shown under “Run group”).

Although multiplex PCR reactions can be employed, preferably, each primer set is run in an individual reaction. Conditions for PCR were, in one case for example: 5 minutes at 96° C. for initial denaturing followed by 35 total cycles of: 30 seconds at 94° C. and 30 seconds at the annealing temperature or at a gradient of 49° C. to 63° C. and a final 10 minutes at 72° C. to complete synthesis of any partial products. Most preferred are primers that have an annealing temperature between 49° C. and 63° C., though many of the primer sets have annealing temperatures that are at 49° C., 52° C., 59° C., and 62.4° C. An approximately 3° C. window is allowed for each plate (e.g., primers having annealing temperatures that are within 3° C. of one another are grouped on a single plate). Programs such as WINMELT were used to determine whether the primers could be grouped into various primer sets that have similar annealing temperatures so that individual groups of primers can be amplified by Polymerase Chain Reaction (PCR) on the same plate.

Once the extension products had been generated they were grouped, pooled, and mixed with loading dye. Eight Multi G groups (Multi-Grouping pools of extension products) were used for the extension products “fragments” generated by the various primer sets, which belong to one of the eight groups are identified in TABLE C and TABLE D (some of the run groups on the separate MLH1 and MSH2 Table have identical conditions). After grouping and pooling, the samples were loaded onto a TTGE gel. TABLE D also lists the start and stop temperatures for the TTGE, for each Multi G group, under ‘run conditions’. Preferably, the TTGE is run with a very shallow temperature gradient, e.g., about 1.0° C./hour for a total of three hours, at high voltage, e.g., 150 volts. Once the separation was complete, the gels were grouped, stained with ethidum bromide, and analyzed by the Decode system. The analysis above was rapid, inexpensive, and very effective at detecting mutations and/or polymorphisms, many of which go undetected or are not analyzed by others in the field.

Whereas many in the field seek to design primers that optimally anneal with a template DNA, it has been discovered that primers can also be designed to produce an optimal extension product (e.g., a fragment of short length with a reliable and rapid melting point). Preferably, primers are designed to generate extension products that are approximately 100-300 nucleotides in length and that have long stretches of DNA that melt at approximately the same temperature (e.g., DNA stretches that are 25, 35, 45, 55, 65, 75, 85, 95, 100, 125, 15, 175, or 200 nucleotides that melt at the same temperature or within about a 0° C. to about a 1.5° C. temperature difference).

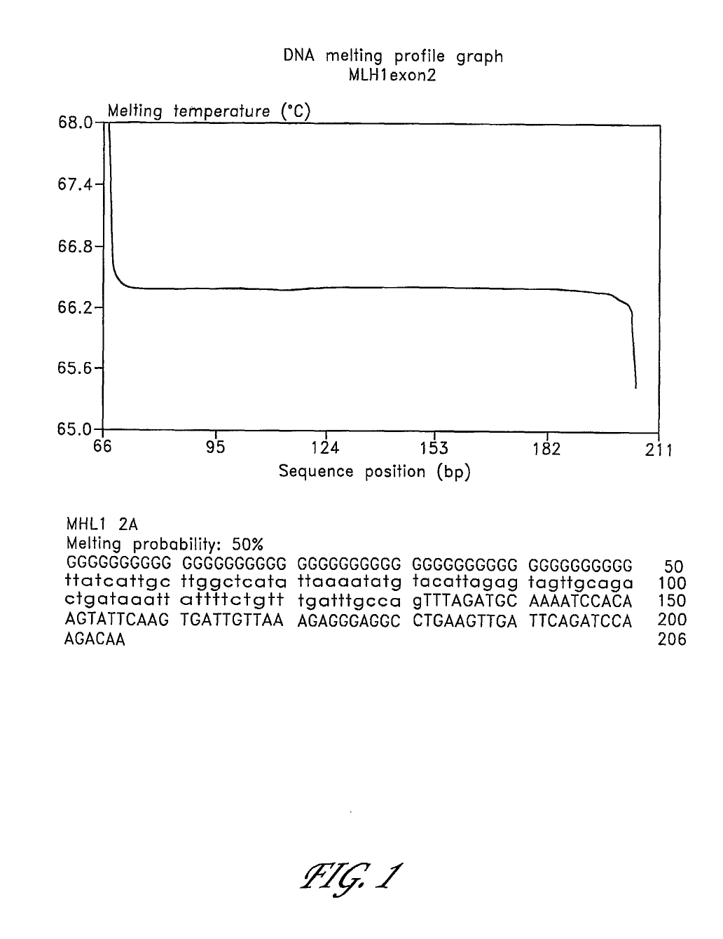

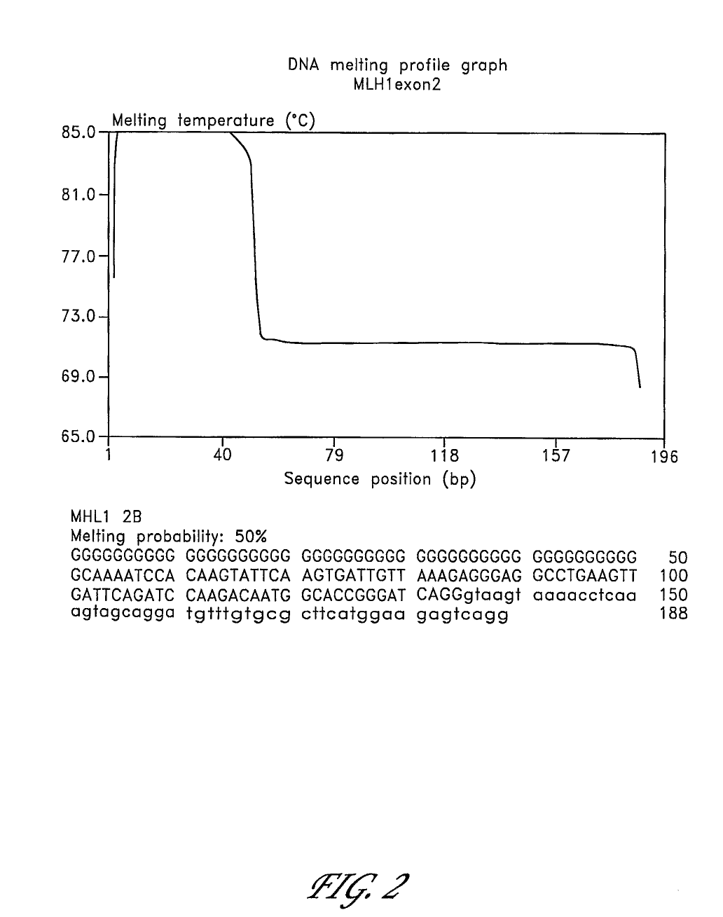

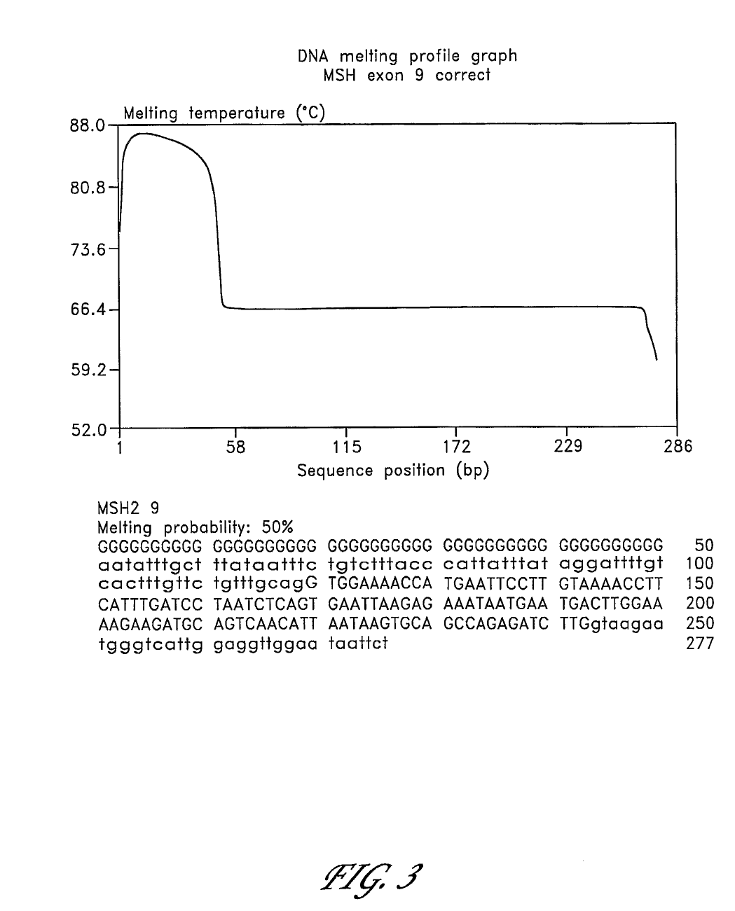

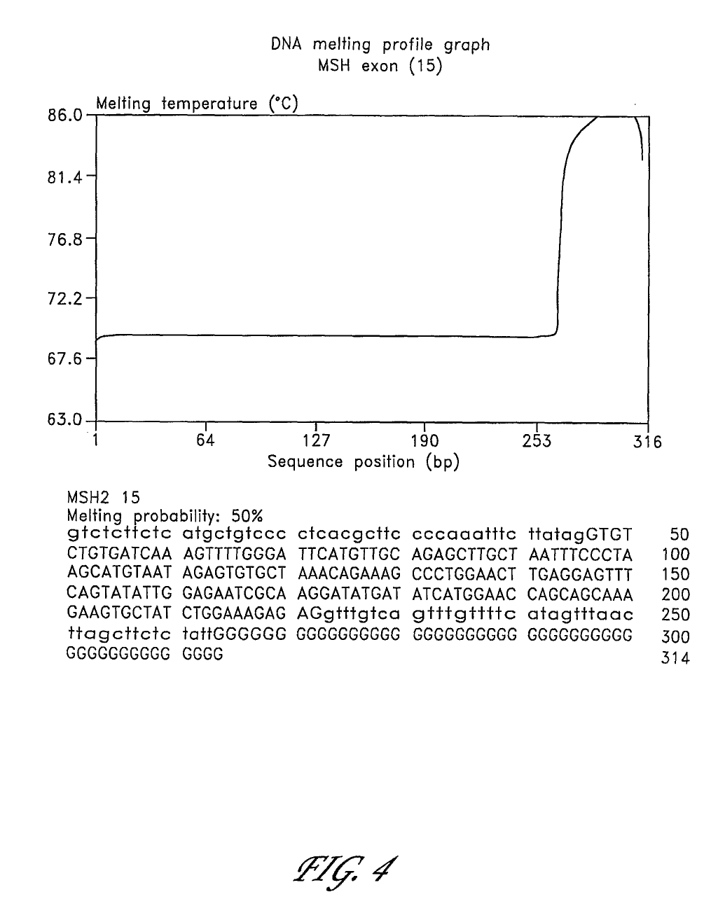

Programs such as WINMELT were used to evaluate the melting behavior of extension products generated from the various primer sets and test TTGE separation of the extension products generated by the various primer sets were also performed to ensure that the predicted melting behavior was represented on the gel. For example, FIGS. 1-4 show graphs of four extension products produced by two primer sets that amplify portions of the cystic fibrosis gene (CTFR). The flat melting curve shown in these figures is preferred for the applications described herein because the extension products melt rapidly and are quickly retarded in the gel, which improves resolution and allows multiple different extension products to be separated in the same lane on a TTGE gel. That is, by grouping extension products that have flat melting profiles, which are within, approximately 1.5° C. of one another, it allows a shallow TTGE temperature ramp (e.g., 1° C. change per hour for 3 hours) or shallow DHPLC temperature ramp, which increases the sensitivity, allowing multiple extension products to be separated in the same lane, which increases throughput and reduces the cost of the analysis.

By analogy, TABLE D shows several of the characteristics of the extension products generated by the primers described herein. In particular, the PCR annealing temperature for the primer set used to generate the extension product (“PCR temp.”) is provided. Further, the Multi

G/stack group is also listed. The following examples describe the foregoing methodologies in greater detail. The first example describes an approach that was used to isolate DNA from human blood.

### Example 1

A sample of blood was obtained from a subject to be tested by phlebotomy. A portion of the sample (e.g., approximately 1.0 ml) was added to approximately three times the sample volume or 3.0 ml of a lysis solution (10 mM KHCO₃, 155 mM NH₄Cl, 0.1 mM EDTA) and was mixed gently. The lysis solution and blood were allowed to react for approximately five minutes. Next, the sample was' centrifuged (×500 g) for approximately 2 minutes and the supernatant was removed. Some of the supernatant was left (e.g., on the walls of the vessel) to facilitate suspension. The pellet was then vortexed for approximately 5-10 seconds. An extraction solution, which contains chaotrope and detergent (Qiagen), was then added (e.g., 500 μl), the sample was vortexed again for approximately 5-10 seconds, and the solution was allowed to react for five minutes at room temperature.

Next, a GFX column, which are pre-packed columns containing a glass fiber matrix, was placed under vacuum (e.g., a Microplex 24 vacuum system) and the extracted solution containing the DNA was transferred to the column (e.g., in 500 μl aliquots). Once all of the sample has been passed through the column, the vacuum was allowed to continue for approximately 5 minutes. Subsequently, a wash solution (Tris-EDTA buffer in 80% ethanol) was added (e.g., approximately 500 μl) under vacuum. Once the wash solution had been drained from the column, the vacuum was allowed to continue for approximately 15 minutes. The GFX columns containing the DNA were then placed into sterile microfuge tubes but the lids were kept open.

Elution buffer (10 mM Tris-HCl, 1 mM EDTA, pH 8.0) was then added to the column (e.g., approximately 100 μl of buffer that was heated to approximately 70° C.) and the buffer was allowed to react with the column for approximately 2 minutes. Then, the tubes containing the columns were centrifuged at ×5000 g for approximately 1.5 minutes. After centrifugation, the column was discarded and the microfuge tube containing the isolated DNA was stored at −20° C. The example below describes the design and optimization of primers that allowed for the inventive high-throughput multiplex PCR technique, described herein.

### Example 2

Sets of primers for PCR amplification were designed for every exon of the following genes: Cystic Fibrosis Transmembrane Reductase (CFTR), Beta-hexosaminidase alpha chain (HEXA), PAH, Alpha globin-2 (HBA2), Beta globin (BBB), Glucocerebrosidase (GBA), Galactose-1-phosphae uridyl transferase (GALT), Medium chain acyl-CoA dehydrogenase (MCAD), Protease inhibitor 1 (PI), Factor VIII, FMR1, and Aspartoacylase (ASPA). The primers were designed from sequence information that was available from GenBank or from sequence information obtained from Ambry Genetics Corporation. Information regarding mutations or polymorphisms was obtained from The Human Gene Mutation Database.

One of the primers in each primer set contained a GC-clamp. It was discovered that the addition of a GC-clamp significantly altered the melting profile of the DNA extension product. Further, proper positioning of the GC-clamp served to level the melting profile. It was desired to position the GC-clamp so that a single melting domain across the fragment was created. Proper positioning of the GC-clamp was oftentimes needed to prevent the GC-clamp from masking the presence of a mutation or polymorphism (e.g., if the mutation or polymorphism is too close to the GC-clamp). Software was also used to optimize primer design. For example, many primers were designed with the aid of Primer Premiere 4.0 and 5.0 and appropriate positioning of the GC-clamps was determined using WinMelt software from BioRad. To maintain sensitivity of the test, the primers were designed to anneal at a minimum of 40 base pairs either upstream or downstream of the nearest known mutation in the intronic region of the genes.

Although multiplex PCR can be technically difficult when using the quantity of primers described herein, it was discovered that almost all of the PCR artifacts disappeared when salt concentration, temperature, primer selection, and primer concentration were carefully optimized. Optimization was determined for each primer set alone and in combination with other primer sets. Optimization experiments were conducted using Master Mix from Qiagen and a Thermocyler from MJ Research. The conditions for thermal cycling were 5 minutes @ 95° C. for the initial denaturation, then 30 cycles of: 30 seconds @ 94° C., 45 seconds @ 48-68° C., and 1 minute @ 72° C. A final extension was performed at 72° C. for 10 minutes.

In addition to primer compatibility, primers were selected to facilitate identification of extension products by electrophoresis. To optimize primer design in this regard, separate PCR reactions were conducted for each individual set of primers and the extension products were separated by the inventive DNA separation technique, described above. Identical parameters were maintained for each assay and the migration behavior for each extension product was analyzed (e.g., compared to a standard to determine a R_{f} value for each fragment). An R_{f} value is a unit less value that characterizes a fragment's mobility relative to a standard under set conditions. In many primer optimization experiments, for example, the generated extension products were compared to a standard extension product obtained from amplification of the first exon of the PAH (phenylalanine hydroxylase) gene. A measurement of the distance of migration of each band in comparison to the distance of migration of the first exon of PAH was recorded and the R_{f} value was calculated according to the following:

$${R}_{f} = \frac{( migration distance of fragment ) cm}{( migration distance of PAH exon 1 ) cm}$$

By conducting these experiments, it was verified that the selected primers did not produce extension products that overlapped on the gel. Optimal primer selection was obtained when optimal PCR parameters were maintained and the extension products produced dissimilar R_{f} values. Finally, the multiplex PCR was tested with all sets of primers and it was verified that few artifacts were created during amplification. Embodiments of the invention include the primers provided in the Tables and sequence listing provided herein and methods of using said primers and/or groups of primers. The example below describes an experiment that verified that the embodiments described herein effectively screen multiple loci present on a plurality of genes in a single assay.

### Example 3

Two independent PCR reactions were conducted to demonstrate that multiple loci on a plurality of genes can be screened in a single assay using an embodiment of the invention. In a first reaction, seven different loci from four different genes were screened and, in the second reaction, eight different loci from four different genes were screened. The primers used in each multiplex reaction are provided in Table 1.

TABLE 1*

| Multiplex #1 | Multiplex #2 |
|---|---|
| Factor VIII 4 (SEQ. ID. Nos. 300 and 318) | CFTR 23 (SEQ. ID. Nos. 296 and 314) |
| Factor VIII 11 (SEQ. ID. Nos. 302 and 320) | CFTR 18 (SEQ. ID. Nos. 295 and 313) |
| Factor VIII 24 (SEQ. ID. Nos. 303 and 321) | Factor VIII 11 (SEQ. ID. Nos. 302 and 320) |
| PAH 9 (SEQ. ID. Nos. 311 and 329) | Factor VIII 3 (SEQ. ID. Nos. 299 and 317) |
| GBA 6 (SEQ. ID. Nos. 308 and 326) | CFTR 24 (SEQ. ID. Nos. 330 and 331) |
| Factor VIII 1 (SEQ. ID. Nos. 297 and 315) | GBA 4 (SEQ. ID. Nos. 307 and 325) |
| GALT 9 (SEQ. ID. Nos. 310 and 328) | GALT 9 (SEQ. ID. Nos. 310 and 328) |
| | GBA 3 (SEQ. ID. Nos. 306 and 324) |

*Primers are stored in a 50 μM storage stock and a 12.5 μM working stock.
Abbreviations are: Phenyl alanine hydroxylase (PAH), Glucocerebrosidase (GBA), Galactose-1- phosphate uridyl transferase (GALT), and cystic fibrosis transmembrane reductase (CFTR). The numbers following the abbreviations represent the exons probed.

The amplification was carried out in 25 μl reactions using a 2× Hot Start Master Mix, which contains Hot Start Taq DNA Polymerase, and a final concentration of 1.5 mM MgCl₂ and 200 μM of each dNTP (commercially available from Qiagen). In each reaction, 12.50 of Hot Start Master Mix was mixed with 1 of μlgenomic DNA (approximately 200 ng genomic DNA), which was purified from blood using a commercially available blood purification kit (Pharmacia or Amersham). Primers were then added to the mixture (0.5 μM final concentration of each primer). Then, ddH₂O was added to bring the final volume to 25 μl.

Thermal cycling for the Multiplex #1 reaction was performed using the following parameters: 15 minutes @ 95° C. for 1 cycle; 30 seconds @ 94° C., 1 minute (4) 53° C., 1 minute and 30 seconds (4) 72° C. for 35 cycles; and 10 minutes @ 72° C. for 1 cycle. Thermal cycling for the Multiplex #2 reaction was performed using the following parameters: 15 minutes (4) 95° C. for 1 cycle; 30 seconds @ 94° C., 1 minute @49° C., 1 minute and 30 seconds @ 72° C. for 35 cycles; and 10 minutes @ 72° C. for 1 cycle.

After the amplification was finished, approximately 5 μl of each PCR product was mixed with 5 μlof non-denaturing gel loading dye (70% glycerol, 0.05% bromophenol blue, 0.05% xylene cyanol, 2 mM EDTA). The DNA in the two reactions was then separated on the basis of melting behavior on separate denaturing gels. Each gel was a 16×16 cm, 1 mm thick, 7M urea, 8% acrylamide/bis(37.5:1) gel composed in 1.25×TAE (50 mM Tris, 25 mM acetic acid, 1.25 mM EDTA). Separation was conducted for 4 hours at 150 V on the Dcode system (BioRad) and the temperature ranged from 51° C. to 63° C. with a temperature ramp rate of 3° C./hour. Subsequently, the gels were stained in 1 μg/ml ethidium bromide in 1.25×TAE for 3 minutes and destained in 1.25×TAE buffer for 20 minutes. The gels were then photographed using the Gel Doc 1000 system from BioRad.

The primers in Table 1 were selected and manufactured because they produced extension products with very different R_{f} values and the extension products were clearly resolved by separation on the basis of melting behavior. Although some bands were more visible than others on the gel, seven distinct bands were observed on the gel loaded with extension products generated from the Multiplex 1 reaction and eight distinct bands were observed on the gel loaded with extension products generated from the Multiplex 2 reaction. These results verified that the described method effectively screened multiple loci on a plurality of genes in a single assay. The example below describes another experiment that verified that the embodiments described herein can be used to effectively screen multiple loci present on a plurality of genes in a single assay.

### Example 4

Experiments were conducted to differentiate extension products generated from wild-type DNA and extension products generated from mutant DNA. Samples of genomic DNA that had been previously identified to contain mutations or polymorphisms were purchased from Coriell Cell Repositories. The mutation or polymorphism that was analyzed in this experiment was the delta-F508 mutation of the CFTR gene. This mutation is a 3 by deletion in exon 10 of the CFTR gene. Other loci analyzed in these experiments included the Fragile X gene, exon 17; Fragile X gene, exon 3; Factor VIII gene exon 2; and the Factor VIII gene, exon 7. Both the known mutant and a control wild-type for CFTR exon 10 were amplified within a multiplex reaction and individually. PCR amplification was conducted as described in EXAMPLE 3; however, 0.25 μM (final concentration) of each primer was used. The primers used in these experiments were CFTR 10 (SEQ. ID. Nos. 294 and 312), FragX 17 (SEQ. ID. Nos. 305 and 323), FragX 3 (SEQ. ID. Nos. 304 and 322), Factor VIII 7 (SEQ. ID. Nos. 301 and 319) and Factor VIII 2 (SEQ. ID. Nos. 298 and 316). The numbers following the abbreviations represent the exons probed.

The DNA templates that were analyzed included known wild-type genomic DNA, known mutant genomic DNA, mixed wild-type genomic DNA from various subjects, and mixed wild-type and mutant genomic DNA. Approximately 200 ng of genomic DNA was added to each reaction. The mixed wild-type and mutant DNA sample had approximately 100 ng of each DNA type. Thermal cycling was carried out with a 15-minute. step at 95° C. to activate the Hot Start Polymerase, followed by 30 cycles of 30 seconds at @ 94 C, 1 minute at @ 53° C., 1 minute and 30 seconds at @ 72° C.; and 72° C. for 10 minutes.

After amplification, approximately 5 μl of the PCR product was mixed with 5 μl of non-denaturing gel loading dye (70% glycerol, 0.05% bromophenol blue, 0.05% xylene cyanol, 2 mM EDTA). The samples were then separated on a 16×16 cm, 1 mm thick, 6M urea, 6% acrylamide/bis (37.5:1) gel in 1.25×TAE (50 mM Tris, 25 mM acetic acid, 1.25 mM EDTA) for 5 hours at 130 V using the Dcode system (BioRad). The temperature ranged from 40° C. to 50° C. at a temperature ramp rate of 2° C./hour. The gels were then stained in 1 μg/ml ethidium bromide in 1.25×TAE for 3 minutes and destained in 1.25×TAE buffer for 20 minutes. The gels were then photographed using the Gel Doc 1000 system from BioRad.

The resulting gel revealed that the lane containing the extension products generated from the wild-type DNA using the CFTR10 primers had a mobility commensurate to the wild-type DNA standard, as did the extension products generated from the other primers and the wild-type DNA. That is, a single band appeared on the gel in these lanes. The lane containing the extension products generated from the template having the F508 mutant, on the other hand, showed 2 bands. One of the bands had the same mobility as the extension products generated from the wild-type or DNA standard and the other band migrated slightly faster. These two populations of bands represent the two populations of homoduplexes (i.e., wild-type/wild-type and mutant/mutant). The top band is the wild-type homoduplex and the lower band is the mutant F508 homoduplex. Similarly, the lane that contained the wild-type/mutant DNA mix exhibited two populations of extension products, one representing the wild-type homoduplex population and the other representing the mutant homoduplex. Since F508 is a 3 by deletion it failed to form heteroduplex bands in sufficient quantity to be visible on the gel. Thus, this experiment demonstrated that the described method effectively screened multiple genes, in a single assay, and detected the presence of a polymorphism in one of the screened genes. The example below describes an experiment that demonstrated that an improved sensitivity can be obtained by mixing a plurality of DNA samples.

### Example 5

This example describes two experiments that verified that an improved sensitivity of detection can be obtained by (1) mixing the DNA samples from a plurality of subjects prior to amplification or by (2) mixing amplification products before separation on the basis of melting behavior. In these experiments, PCR amplifications of exon 9 of the GBA gene (Glucocerebrosidase gene) were used. DNA samples known to contain a mutation in exon 9 of the GBA gene were purchased from Coriell Cell Repositories. These DNA samples contain a homozygous mutation in exon 9 of the GBA gene (the N370S mutation).

In a first experiment, single amplification of exon 9 was performed in a 25 μl reaction. A Taq PCR Master Mix (containing Taq DNA Polymerase and a final concentration of 1.5 mM MgCl₂ and 200 μM dNTPs)(Qiagen) was mixed with 0.5 μM (final concentration) of primers (SEQ. ID. Nos. 309 and 327). The template genomic DNAs analyzed in this experiment included wild-type DNA, mutant DNA, and various mixtures of wild-type and mutant DNA. For the single non-mixed reactions, approximately 200 ng of genomic DNA was used for amplification. In the mixed samples, approximately 200 ng of DNA was again used, however, the percentage of wild-type to mutant genomic DNA varied. Thermal cycling was performed according to the following parameters: 10 minutes @ 94° C.; 30 cycles of 30 seconds @ 94° C., 1 minute@ 44.5° C., and 1 minute and 30 seconds @ 72° C.; and 10 minutes @ 72° C.

In the second experiment, the amplification products were mixed prior to separation on the basis of melting behavior. Amplification of both wild-type and mutant (N370S) exon 9 of the GBA gene was performed using 25 μl reactions, as before. The Taq Master Mix obtained from Qiagen was mixed with 200 ng of genomic DNA and 0.5 μM final concentration of both primers (SEQ. ID. Nos. 309 and 327). PCR was carried out for 30 cycles with an annealing temperature of 56° C. for 1 minute. The denaturation and elongation steps were 94° C. for 30 seconds and 72° C. for 1 minute and 30 seconds. Final elongation was carried out at 72° C. for 10 minutes. The extension products obtained from the single amplification of wild-type GBA exon 9 was then mixed with the extension products obtained from the single amplification of the mutant GBA exon 9. Next, the pooled DNA was subjected to denaturation at 95° C. for 10 minutes and cooled on ice for 5 minutes, then heated to 65° C. for 5 minutes and cooled to 4° C. This denaturation and annealing procedure was performed to facilitate the formation of heteroduplex DNA.

Once the extension products from both experiments were in hand, approximately 5 μl of both the prior to PCR mixture and post PCR mixture were loaded on 16×16 cm, 1 mm thick gels (7M Urea/8% acrylamide (37.5:1) gel in 1.25×TAE) using the gel loading dye and the Dcode system (BioRad), described above. The DNA on the gel was then separated at 150 V for 5 hours and the temperature was uniformly raised 2° C./hour throughout the run starting at 50° C. and ending at 60° C.

The gel was stained in 1 μg/ml ethidium bromide in 1.25×TAE buffer for 3 minutes and destained in buffer for 20 minutes.

It should be noted that the GBA gene has a pseudo gene, which was co-amplified by the procedure above. An extension product generated from this psuedo gene migrated slightly faster than the extension product generated from the true expressed gene on the gel. In all lanes, the band representing the extension product generated from the psuedo gene was present. Then next fastest band on the gel was the extension product generated from the GBA exon 9 wild-type allele. The extension product generated from the mutant GBA exon 9 allele comigrated with the wild-type allele and was virtually indistinguishable on the basis of melting behavior due to the single base difference.

The heteroduplexes formed in the mixed samples were easily differentiated from the homoduplexes. The samples mixed prior to PCR showed both homoduplexes (wild-type and mutant) along with heteroduplexes, which appeared higher on the gel. Thus, by mixing samples, either prior to amplification or prior to separation on the basis of melting behavior an improved sensitivity of detection was obtained. Since homoduplex bands no longer need to be resolved to identify a mutation or polymorphism, only the heteroduplex bands need to be resolved, the throughput of diagnostic analysis was greatly improved. The example below describes experiments that verified that the embodiments taught herein can be used to effectively screen multiple genes in a plurality of subjects, in a single assay, for the presence or absence of a polymorphism or mutation.

### Example 6

Two experiments were conducted to verify that multiple genes from a plurality of subjects can be screened in a single assay for the presence or absence of a genetic marker (e.g. a polymorphism or mutation) that is indicative of disease. These experiments also demonstrated that an improved sensitivity of detection could be obtained by mixing DNA samples either prior to generation of extension products or prior to separation on the basis of melting behavior.

In both experiments, five different extension products were generated from three different genes in a single reaction vessel. The five different extension products were generated using the following primers: Factor VIII 1 (SEQ. ID. Nos. 297 and 315); GBA 9 (SEQ. ID. Nos. 309 and 327); GBA 11 (SEQ. ID. Nos. 332 and 333); GALT 5 (SEQ. ID. Nos. 334 and 335), and GALT 8 (SEQ. ID. Nos. 336 and 337). Abbreviations are: Glucocerebrosidase (GBA) and Galactose-1-phosphate uridyl transferase (GALT). The numbers following the abbreviations represent the exons probed.

Extension products were generated for each experiment in 25:1 amplification reactions using Qiagen's 2× Hot Start Master Mix (Contains Hot Start Taq DNA Polymerase, and a final concentration of 1.5 mM MgCl₂ and 200 :M of each dNTP). To each reaction, 12.5 μl of Hot Start Master Mix was added to 1 μl of genomic DNA (approximately 200 ng genomic DNA for the mutant DNA sample and the wild-type DNA sample), which was purified from human blood using Pharmacia Amersham Blood purification kits. For the experiment in which the DNA samples from a plurality of subjects were mixed prior to generation of the extension products, approximately 100 ng of wild-type genomic DNA was mixed with approximately 100 ng of mutant N370S genomic DNA. In both experiments, primers were added to achieve a final concentration of 0.5 :M for each primer and a final volume of 25 μl was obtained by adjusting the volume with ddH₂O.

Thermal cycling for both experiments was performed using the following parameters: 15 minutes @ 95° C. for 1 cycle; 30 seconds @ 94° C., one minute @ 57° C., and one minute 30 seconds @ 72° C. for 35 cycles; and 10 minutes @ 72° C. for 1 cycle. After amplification, the extension products generated from the wild-type and mutant templates (the un-mixed samples) were separated from the PCR reactants using a PCR Clean Up kit (Qiagen). Then, approximately 10 μL of the wild-type and mutant DNA were removed from each tube and gently mixed in a single reaction vessel. This preparation was then denatured at 95° C. for 1 minute and rapidly cooled to 4° C. for 5 minutes. Finally, the preparation was brought to 65° C. for an additional 1.5 minutes. The extension products generated from the mixed sample (wild-type DNA and mutant DNA mixed prior to amplification) were stored until loaded onto a denaturing gel.

Next, approximately 10 μl of the unmixed sample was combined with 10 μl of loading dye and approximately 5:1 of the mixed sample was combined with 5:1 of loading dye. The loading dye was composed of 70% glycerol, 0.05% bromophenol blue, 0.05% xylene cyanol, and 2 mM EDTA). The samples in loading dye were then loaded on separate 16×16 cm, 1 min thick, 7M urea, 8% acrylamide/bis(37.5:1) gels in 1.25×TAE (50 mM Tris, 25 mM acetic acid, 1.25 mM EDTA). The DNA was separated on the basis of melting behavior for 5 hours at 150 V on the Dcode system (BioRad). The temperature ranged from 56° C. to 68° C. at a temperature ramp rate of 2° C./hr. The gels were then stained in 1 μg/ml ethidium bromide in 1.25×TAE for 3 minutes and destained in 1.25×TAE buffer for 20 minutes. The gels were photographed using the Gel Doc 1000 system (BioRad).

In all lanes of the gel, 5 extension products generated from three different genes were visible in the following order from top to bottom: Factor VIII 1, GBA 9, GBA 11, GALT 8, and GALT 5. Two different extension products were generated from the GBA 9 primers, as described above. The less intense band below the homoduplex bands corresponded to an extension product generated from the pseudogene. In the lanes loaded with extension products generated from only the wild-type or mutant DNA template, it was difficult to distinguish the wild type homoduplex from the N370S mutant homoduplex. In the lane loaded with the extension products generated from the mixed DNA templates (wild-type and mutant DNA mixed prior to amplification) and the lane loaded with extension products (generated from wild type and mutant DNA separately) that were mixed after amplification, heteroduplex bands were easily visualized. These experiments verified that multiple genes can be screened in a plurality of individuals in a single assay and that a single nucleotide mutation or polymorphism can be detected. Further, these experiments demonstrate that screening a plurality of DNA samples in a single reaction vessel or adding a control DNA before or after amplification greatly improves the sensitivity of detection. By practicing the methods taught in this example, the throughput of diagnostic screening can be drastically improved and the cost of identifying genetic traits can be significantly reduced. The example below describes approaches to screen multiple genes in a plurality of subjects, in a single assay, for the presence or absence of a polymorphism or mutation using DHPLC.

### Example 7

Multiple genes in a plurality of subjects, in a single assay, can be screened for the presence or absence of a polymorphism or mutation using a DHPLC separation approach. For example, five different extension products can be generated using the following primers: Factor VIII 1 (SEQ. ID. Nos. 297 and 315); GBA 9 (SEQ. ID. Nos. 309 and 327); GBA 11 (SEQ. ID. Nos. 332 and 333); GALT 5 (SEQ. ID. Nos. 334 and 335), and GALT 8 (SEQ. ID. Nos. 336 and 337). Abbreviations are: Glucocerebrosidase (GBA) and Galactose-1-phosphate uridyl transferase (GALT). The numbers following the abbreviations represent the exons probed. The extension products can be generated in 25:1 amplification reactions using Qiagen's 2× Hot Start Master Mix (Contains Hot Start Taq DNA Polymerase, and a final concentration of 1.5 mM MgCl₂ and 200 μM of each dNTP).

To each reaction, 12.5 μl of Hot Start Master Mix is added to 1 μl of genomic DNA (approximately 200 ng genomic DNA for the mutant DNA sample and the wild-type DNA sample), which is purified from human blood using Pharmacia Amersham Blood purification kits. By another approach, the DNA samples from a plurality of subjects can be mixed prior to generation of the extension products. In this case, approximately 100 ng of wild-type genomic DNA is mixed with approximately 100 ng of mutant N370S genomic DNA. Primers are added to achieve a final concentration of 0.5 μM for each primer and a final volume of 25 μl is obtained by adjusting the volume with ddH₂O.

Thermal cycling is performed using the following parameters: 15 minutes @ 95° C. for 1 cycle; 30 seconds @ 94° C., one minute @ 57° C., and one minute 30 seconds @ 72° C. for 35 cycles; and 10 minutes @ 72° C. for 1 cycle. After amplification, the extension products generated from the wild-type and mutant templates (if un-mixed samples) are separated from the PCR reactants using a PCR Clean Up kit (Qiagen). Then, approximately 10 :L of the wild-type and mutant DNA are removed from each tube and gently mixed in a single reaction vessel. This preparation is then denatured at 95° C. for 1 minute and rapidly cooled to 4° C. for 5 minutes. Finally, the preparation is brought to 65° C. for an additional 1.5 minutes. The extension products generated from the mixed sample (wild-type DNA and mutant DNA mixed prior to amplification) can be stored until loaded onto a DHPLC column.

Next, the extension products are loaded on to a 50×4.6 mm ion pair reverse phase HPLC column that is equilibrated in degassed Buffer A (0.1 M triethylamine acetate (TEAA) pH 7.0) at 56° C. A linear gradient of 40%-50% of degassed Buffer B (0.1 M triethylamine acetate (TEAA) pH 7.0 and 25% acetonitrile) is then performed over 2.5 minutes at a flow rate of 0.9 ml/min at 56° C., followed by a linear gradient of 50%-55.3% Buffer B over 0.5 minutes, and finally a linear gradient of 55.3%-61% Buffer B over 4 minutes. U.V. absorption is monitored at 260 nm, recorded and plotted against retention time.

When the loaded sample is un-mixed extension products, the extension products generated from only the wild-type or mutant DNA template, it is difficult to distinguish the wild type homoduplex from the N370S mutant homoduplex. When the loaded sample is the mixed extension products, the extension products generated from the mixed DNA templates (wild-type and mutant DNA mixed prior to amplification), or the extension products (generated from wild type and mutant DNA separately) that were mixed after amplification, heteroduplex elution behavior is detected. By practicing the methods taught in this example, the throughput of diagnostic screening can be drastically improved and the cost of identifying genetic traits can be significantly reduced. The example below describes an approach that was used to diagnostically screen patient samples for the presence or absence of polymorphisms or mutations on genes associated with HNPCC.

### Example 8

Sets of primers for PCR amplification were designed for every exon of the MLH1 and MSH2 genes. The primers were designed from sequence information that was available from GenBank or from sequence information obtained from Ambry Genetics Corporation. Information regarding mutations or polymorphisms was obtained from The Human Gene Mutation Database.

Primer sets and PCR stacking groups were designed for optimal sensitivity for TTGE, as described above. DNA from one individual was amplified with each primer set in a separate reaction, then stacked in average groups of three fragments/gel for gel analysis. Preferably, one of the primers in each primer set contained a GC-clamp. It was discovered that the addition of a GC-clamp significantly altered the melting profile of the DNA extension product. Further, proper positioning of the GC-clamp served to level the melting profile. It was desired to position the GC-clamp so that a tight single melting domain across the fragment was created. Proper positioning of the GC-clamp was often times needed to prevent the GC-clamp from masking the presence of a mutation or polymorphism (e.g., if the mutation or polymorphism is too close to the GC-clamp). Software was also used to optimize primer design. For example, many primers were designed with the aid of Pruner Premiere 4.0 and 5.0 and appropriate positioning of the GC-clamps was determined using WinMelt software from BioRad. To maintain sensitivity of the test, the primers were designed to anneal at a minimum of 40 base pairs either upstream or downstream of the nearest known mutation in the intronic region of the genes.

Optimization was determined for each primer set. Optimization experiments were conducted using Hotstart Master Mix from Qiagen and a Thermocyler from MJ Research. Resulting PCR conditions for all fragments were 15 minutes @ 95° C. for the initial denaturation, then 35 cycles of 30 seconds @ 94° C., 30 seconds @ 46-62° C., and 30 seconds @ 72° C. A final extension was performed at 72° C. for 10 minutes. Approximately 15 ul PCR reactions contained 7.5 ul Qiagen 2× Hotstart Master Mix, 50-200 ng genomic DNA, sense and antisense primer for each fragment at a final concentration of 0.5-1 μM. Prior to gel loading and stacking of gel groups PCR samples were heated and re-annealed to provide best heteroduplex formation. PCR product was heated to 95° C. for 5 min, 50° C. for 10 min, then brought to 4° C.

PCR products (approximately 4-8 μl each depending on signal strength) were then assembled for groups of equal melting characteristics and mixed with loading dye consisting of 70% glycerol, 0.05% bromophenol blue, 0.05% xylene cyanol, 2 mM EDTA). DNA was separated on denaturing gels (7 M urea, 8% acrylamide/bis(37.5:1) in 50 mM Tris, 25 mM acetic acid, 1.25 mM EDTA) for 3-5 hours at 125 V or 150 V on the Dcode system. (Biorad). Temperature ranged from 45.5° C. to 64° C. with ramp rates of 1.0-1.5° C./hr depending on gel groups. The gels were stained in 1 :g/ml ethidium bromide in 1.25×TAE for 3 minutes and destained in 1.25×TAE buffer for 20 minutes. The gels were photographed using the Gel Doc 1000 system (BioRad). Table 2 below lists the primers used in this assay. TABLE D shows the TTGE gel grouping (MLH or MSH stacking group) and temperatures used for TTGE separation (under “Run group”). TABLE D also names the extension products generated from the various primer sets employed and the positions of each fragment on the gel after separation (listed in order). Previous experiments, described above, have demonstrated that extension products generated from primers that are any number between 1-75 nucleotides upstream or downstream from the primers listed in TABLE A (e.g., the primer sets listed in Table 2) can be grouped and efficiently separated in accordance with rules set forth herein. Preferably, the primers listed in Table 2 are used to generate extension products that are grouped according to TABLE D and are separated on the basis of melting behavior (e.g., TTGE). In Table 2, the notation “(*)-” indicates the presence of a GC-rich clamp sequence, the sequence of which is given at the bottom of the Table.

TABLE 2

| | | |
|---|---|---|
| Primer name | SEQ ID | Primer sequence |

| | | |
|---|---|---|
| MLH1-1A-s: | 3 | 5′ (*)-CAATAGCTGCCGCTGA 3′ |
| MLH1-1A-as: | 4 | 5′ CGCTGGATAACTTCCC 3′ |
| MLH1-1B-s: | 5 | 5′ GGCGGGGGAAGTTAT 3′ |
| MLH1-1B-as: | 6 | 5′ (*)-CGCGCCATTGAGTGAC 3′ |
| MLH1-1C-s: | 7 | 5′ (*)-CAAAGAGATGATTGAGAAC 3′ |
| MLH1-1C-as: | 8 | 5′ CATGCCTCTGCCCGG 3′ |
| MLH1-1D-s: | 9 | 5′ (*)-GGAAGAACGTGAGCACGA 3′ |
| MLH1-1D-as: | 10 | 5′ CATTAGCTGGCCGCTG 3′ |
| MLH1-2A-s: | 16 | 5′ (*)-TTATCATTGCTTGGCT 3′ |
| MLH1-2A-as: | 17 | 5′ TTGTCTTGGATCTGAATC 3′ |
| MLH1-2B-s: | 18 | 5′ (*)-GCAAAATCCACAAGTATT 3′ |
| MLH1-2B-as: | 19 | 5′ CCTGACTCTTCCATGAA 3′ |
| MLH1-3A-s: | 23 | 5′ (*)-GGGAATTCAAAGAGAT 3′ |
| MLH1-3A-as: | 24 | 5′ TTCTTGAATCTTTAGCTT 3′ |
| MLH1-3B-s: | 25 | 5′ ATATTGTATGTGAAAGGTTCAC 3′ |
| MLH1-3B-as: | 26 | 5′ (*)-ACCAAACCTTATTTATCTATGT 3′ |
| MLH1-4A-s4 | 32 | 5′ GGTGAGGTGACAGTGGGT 3′ |
| MLH1-4A-as4 | 33 | 5′ (*)-TGAATATATATGAGTAAAAGAAGTCAG 3′ |
| MLH1-4B-s2 | 34 | 5′ TCATGTTACTATTACAACGAAAA 3′ |
| MLH1-4B-as2 | 35 | 5′ (*)-GATAACACTGGTGTTGAGACA 3′ |
| MLH1-5a-s: | 39 | 5′ (*)-GGGATTAGTATCTATCTCT 3′ |
| MLH1-5A-as: | 40 | 5′ GGCTTTCAGTTTTCC 3′ |
| MLH1-5B-s2: | 41 | 5′ CTGAAAGCCCCTCCTA 3′ |
| MLH1-5B-as2: | 42 | 5′ (*)-AGCTTCAACAATTTACTCTC 3′ |
| MLH1-5C-s2: | 43 | 5′ CAAGGGACCCAGATCAC 3′ |
| MLH1-5C-as2: | 44 | 5′ (*)-CCAATATTTATACAAACAAAGC 3′ |
| MLH1-5D-s | 45 | 5′ (*)-TTTGTTATATTTTCTCATTAGAG 3′ |
| MLH1-5D-s | 46 | 5′ ATTCTTACCGTGATCTGG 3′ |
| MLH1-6-5-s | 50 | 5′ (*)-ATTCACTATCTTAAGACCTCGCT 3′ |
| MLH1-6-5-as | 51 | 5′ CTAGAACACATTACTTTGATGACAA 3′ |
| MLH1-7-s: | 55 | 5′ TAACTAAAAGGGGGCT 3′ |
| MLH1-7-as: | 56 | 5′ (*)-TTTATTGTCTCATGGCT 3′ |
| MLH1-8A-s: | 60 | 5′ (*)-GCTGGTGGAGATAAGG 3′ |
| MLH1-8A-as: | 61 | 5′ TGTCCACGGTTGAGG 3′ |
| MLH1-8B-s: | 62 | 5′ GGGGGCAAGGAGAGACAGTAG 3′ |
| MLH1-8B-as2: | 63 | 5′ (*)-ATATAGGTTATCGACATACC 3′ |
| MLH1-8C-s2: | 64 | 5′ AAATGCTGTTAGTC 3′ |
| MLH1-8C-as: | 65 | 5′ (*)-TCTTGAAAGGTTCCAA 3′ |
| MLH1-9A-3-s | 69 | 5′ (*)-GTAATGTTTGAGTTTTGAGTATTTTC 3′ |
| MLH1-9A-3-as | 70 | 5′ CAGAAATTTTTCCATGGTCC 3′ |
| MLH1-9B-s | 71 | 5′ (*)-CAAAGTTAGTTTATGGGAAGGA 3′ |
| MLH1-9B-as | 72 | 5′ GAAGAGTAAGAAGATGCACTTCTT 3′ |
| MLH1-9C-s | 73 | 5′ (*)-CTTCAAAATGAATGGTTACATAT 3′ |
| MLH1-9C-as | 74 | 5′ ATTCCCTGTGGGTGTTTC 3′ |
| MLH1-10-s: | 78 | 5′ (*)-TGAATGTACACCTGTGAC 3′ |
| MLH1-10-as: | 79 | 5′ TAGAACATCTGTTCCTTG 3′ |
| MLH1-11A-s: | 83 | 5′ (*)-TTGACCACTGTGTCATC 3′ |
| MLH1-11A-as: | 84 | 5′ GTGCAGGAAGTGAACT 3′ |
| MLH1-11B-s: | 85 | 5′ (*)-CAGAATGTGGATGTTAATG 3′ |
| MLH1-11B-as: | 86 | 5′ GGAGGAATTGGAGCC 3′ |
| MLH1-11C-s4: | 87 | 5′ CAGCAGCACATCGAGAG 3′ |
| MLH1-11C-as4: | 88 | 5′ (*)-ATCTGGGCTCTCACGTCT 3′ |
| MLH1-12B-s: | 92 | 5′ (*)-TTTTTTTTAATACAGACTTTG 3′ |
| MLH1-12B-as: | 93 | 5′ GTGACAATGGCCTGG 3′ |
| MLH1-12C-s: | 94 | 5′ CATTTCTGCAGCCTCT 3′ |
| MLH1-12C-as: | 95 | 5′ (*)-TTTTTGGCAGCCACT 3′ |
| MLH1-12D-s3: | 96 | 5′ AGCCCCTGCTGAAGTG 3′ |
| MLH1-12D-as3: | 97 | 5′ (*)-AGAAGGCAGTTTTATTACAGA 3′ |
| MLH1-12E-s: | 98 | 5′ (*)-TGTCCAGTCAGCCCCA 3′ |
| MLH1-12E-as: | 99 | 5′ CTCTGATTTTTGGCAGC 3′ |
| MLH1-13A-s: | 106 | 5′ (*)-AATTTGGCTAAGTTTAA 3′ |
| MLH1-13A-as: | 107 | 5′ GGAATCATCTTCCACC 3′ |
| MLH1-13B-s2: | 108 | 5′ (*)-CATTGCAGAAAGAGACATC 3′ |
| MLH1-13B-as3: | 109 | 5′ CGCCCGCCGCGGTGAGGTTAATGATCCTTCT 3′ |
| MLH1-13C-s1: | 110 | 5′ (*)-TGATTCCCGAAAGGAAATGAC 3′ |
| MLH1-13C-as1: | 111 | 5′ CAGGCCACAGCGTTTACGTACCCTCATG 3′ |
| MLH1-13D-s: | 112 | 5′ (*)-ATTAACCTCACTAGTGTTTTG 3′ |
| MLH1-13D-as: | 113 | 5′ TGAGGCCCTATGCATC 3′ |
| MLH1-14A-s: | 117 | 5′ (*)-GGTCAATGAAGTGGGG 3′ |
| MLH1-14A-as: | 118 | 5′ CCACGAAGGAGTGGTTA 3′ |
| MLH1-14B-s: | 119 | 5′ AGTTCTCCGGGAGATG 3′ |
| MLH1-14B-as: | 120 | 5′ (*)-TACCTCATGCTGCTCTC 3′ |
| MLH1-15-s: | 124 | 5′ TTCAGGGATTACTTCTC 3′ |
| MLH1-15-as: | 125 | 5′ (*)-GAAAAATTTAACATACTACA 3′ |
| MLH1-16A-s: | 129 | 5′ (*)-GCCATTCTGATAGTGGA 3′ |
| MLH1-16A-as2: | 130 | 5′ TCTAAGGCAAGCATGGCAA |
| MLH1-16B-s: | 131 | 5′ GCACCGCTCTTTGA 3′ |
| MLH1-16B-as: | 132 | 5′ (*)-GTATAAGAATGGCTGTCA 3′ |
| MLH1-16C-s2: | 133 | 5′ GGCTGAGATGCTTGCAG 3′ |
| MLH1-16C-as2: | 134 | 5′ (*)-CATGAGCCACCGCAC 3′ |
| MLH1-17-s: | 138 | 5′ (*)-TGTTTAAACTATGACAGCA 3′ |
| MLH1-17-as: | 139 | 5′ TGGTCATTTGCCCTT 3′ |
| MLH1-18A-s: | 143 | 5′ (*)-TGTGATCTCCGTTTAGAA 3′ |
| MLH1-18A-as2: | 144 | 5′ CTGAGAGGGTCGACTCC 3′ |
| MLH1-18B-s3: | 145 | 5′ (*) TGCGCTATGTTCTATTCCA 3′ |
| MLH1-18B-as3: | 146 | 5′ GCCGCCCCCGCCCGCTAGTCCTGGGGTGCCA 3′ |
| MLH1-19A-s: | 150 | 5′ CAAGTCTTTCCAGACCC 3′ |
| MLH1-19A-as: | 151 | 5′ (*)-TGTATAGATCAGGCAGGT 3′ |
| MLH1-19B-s4 | 153 | 5′ AAGCCTTGCGCTCACAC 3′ |
| MLH1-19B-as4 | 155 | 5′ (*)-AATAACCATATTTAACACCTCTCAA 3′ |
| MLH1-19C-s: | 152 | 5′ (*)-CAGAAGATGGAAATATCCTGC 3′ |
| MLH1-19C-as: | 153 | 5′ CCGCCCGTGTATATCACACTTTGATACAACACT3′ |
| (*) clamp is | 344 | CGCCCGCCGCGCCCCGCGCCCGCCCCGCCGCCCCCGCCCG |
| MSH2-2B-s3 | 167 | 5′ (*)-GGAGCAAAGAATCTGCAGAG 3′ |
| MSH2-2B-as3 | 168 | 5′ TAATTACCTTATATGCCAAATACCA 3′ |
| MSH2-2C-s: | 165 | 5′ ATAAGGCATCCAAGGAGAA 3′ |
| MSH2-2C-as: | 166 | 5′ (*)-ATCTACTTAAAATACTAAAACACAAT 3′ |
| MSH2-3A-s: | 174 | 5′ (*)-AACATTTTATTAATAAGGTTC 3′ |
| MSH2-3A-as: | 175 | 5′ ATTGCCAGGAGAAGC 3′ |
| MSH2-3B-s2: | 176 | 5′ (*)-ATTTTTACTTAGGCTTCTCCTG 3′ |
| MSH2-3B-as2: | 177 | 5′ CAGTTTCCCCATGTCTCC 3′ |
| MSH2-3C-s: | 178 | 5′ AATGTGTTTTACCCGGAG 3′ |
| MSH2-3C-as: | 179 | 5′ (*)-CTTAAATGAAACAGTATCATGTC 3′ |
| MSH2-4A-s: | 183 | 5′ (*)-TCCTTTTCTCATAGTAGTTTA 3′ |
| MSH2-4A-as: | 184 | 5′ TTGAGGTCCTGATAAATG 3′ |
| MSH2-4A-s2: | 185 | 5 (*)-TTTCTTTCAAAATAGATAATTC 3′ |
| MSH2-4A-as2: | 186 | 5′ TTTTTGCCTTTCAACA 3′ |
| MSH2-4B-2s: | 187 | 5′ ATTTATCAGGACCTCAA 3′ |
| MSH2-4B-2as: | 188 | 5′ (*)-TGTAATTCACATTTATAATC 3′ |
| MSH2-4C-s: | 189 | 5′ ATTGCCAGAAATGGAG 3′ |
| MSH2-4C-as: | 190 | 5′ (*)-ACATATTTACATTATATATATTGT 3 |
| MSH2-5A-s: | 194 | 5′ (*)-TTCATTTTGCATTTGTT 3′ |
| MSH2-5A-as: | 195 | 5′ CTTGATTACCGCAGAC 3′ |
| MSH2-5B-s: | 196 | 5′ (*)-ATCTTCGATTTTTAAATTC 3′ |
| MSH2-5B-as: | 197 | 5′ AAAGGTTAAGGGCTCTG 3′ |
| MSH2-6A-s: | 203 | 5′ (*)-GTTTTTCATGGCGTAG 3′ |
| MSH2-6A-as: | 204 | 5′ ACTGAGAGCCAGTGGTA 3′ |
| MSH2-6B-s2: | 205 | 5′ TTTACTAGGGTTCTGTTGAAGA 3′ |
| MSH2-6B-as: | 206 | 5′ (*)-ATACCTCTCCTCTATTCTG 3′ |
| MSH2-6C-s: | 207 | 5′ TCAAGGACAAAGACTTGT 3′ |
| MSH2-6C-as: | 208 | 5′ (*)-CATATTACAATAAGTGGTATAAT 3′ |
| MSH2-7A-s: | 212 | 5′ (*)-GTTGAGACTTACGTGCTT 3′ |
| MSH2-7A-as2: | 213 | 5′ CAATTCTGCATCTTCTACAAA 3′ |
| MSH2-7B-s2: | 214 | 5′ (*)-ATTTCAGATTGAATTTAGTGG 3′ |
| MSH2-7B-as2: | 215 | 5′ AGTTTGCTGCTTGTCTTTG 3′ |
| MSH2-7C-s3: | 216 | 5′ GACTTGCCAAGAAGTTT 3′ |
| MSH2-7C-as2: | 217 | 5′ (*)-TGAGTCACCACCACCAAC 3′ |
| MSH2-8A-s: | 221 | 5′ (*)-TTTGGATCAAATGATGC 3′ |
| MSH2-8A-as: | 222 | 5′ ATCAGTAAGAGGAGTCACA 3′ |
| MSH2-8B-s: | 223 | 5′ TTGTGACTCCTCTTACTG 3′ |
| MSH2-8B-as: | 224 | 5′ (*)-AATAACTACTGCTTAAATTAA 3′ |
| MSH2-8C-s: | 225 | 5′ CTGACTTCTCCAAGTTTC 3′ |
| MSH2-8C-as: | 226 | 5′ (*)-GTGCTACAATTAGATACTAAA 3′ |
| MSH2-8D-s: | 227 | 5′ AGAAATTATTGTTGGCAGTT 3′ |
| MSH2-8D-as: | 228 | 5′ (*)-ATTGCATACCTGATCCATATC 3′ |
| MSH2-9A-s2: | 232 | 5′ (*)-AATATTTGCTTTATAATTTC 3′ |
| MSH2-9A-as2: | 233 | 5′ AGAATTATTCCAACCTC 3′ |
| MSH2-10A-s: | 237 | 5′ (*)-GAATTACATTGAAAAATGG 3′ |
| MSH2-10A-as: | 238 | 5′ TTAATCTGTTTGCCAGG 3′ |
| MSH2-10B-s2: | 239 | 5′ TCTTCTTGATTATCAAGGC 3′ |
| MSH2-10B-as2: | 240 | 5′ (*)-ACACCATTCTTCTGGATA 3′ |
| MSH2-10C-s3: | 241 | 5′ TGCACAGTTTGGATATTACTT 3′ |
| MSH2-10C-as3: | 242 | 5′ (*)-GTAAAACTTATCATAGAACATTCAC 3′ |
| MSH2-11A-s2: | 246 | 5′ (*)-TTTGGATATGTTTCACGTA 3′ |
| MSH2-11A-as2: | 247 | 5′ CTTTAACAATGGCATCCT 3′ |
| MSH2-11B-s2: | 248 | 5′ (*)-GCAAATTGACTTCTTTAAATG 3′ |
| MSH2-11B-as2: | 249 | 5′ ATGGCTTGCGAAAATAAC 3′ |
| MSH2-12A-s | 253 | 5′ (*)-AGGAAATGGGTTTTGAA 3′ |
| MSH2-12A-as: | 254 | 5′ GAGCTAACACATCATTGAGT 3′ |
| MSH2-12B-s: | 255 | 5′ (*)-ATTTTTATACAGGCTATGTAG 3′ |
| MSH2-12B-as: | 256 | 5′ ACATATGGAACAGGTGCT 3′ |
| MSH2-12C-s: | 257 | 5′ TGGAGCACCTGTTCCAT 3′ |
| MSH2-12C-as: | 258 | 5′ (*)-AACAAAACGTTACCCCC 3′ |
| MSH2-12E-s: | 259 | 5′ CAGCTTTGCTCACGTGTCA 3′ |
| MSH2-12E-as: | 260 | 5′ (*)-CATCTTGAACTTCAACACAAGC 3′ |
| MSH2-13A-s: | 264 | 5′ (*)-TAGGACTAACAATCCATT 3′ |
| MSH2-13A-as: | 265 | 5′ TGGGCCATGAGTACTA 3′ |
| MSH2-13B-s: | 266 | 5′ (*)-ATGGGAGGTAAATCAAC 3′ |
| MSH2-13B-as: | 267 | 5′ GACTCCTTTCAATTGACT 3′ |
| MSH2-13C-s4: | 268 | 5′ TTGTGGACTGCATCTTAGCC 3′ |
| MSH2-13C-5as: | 269 | 5′ (*)-TCACAGGACAGAGACATACATTTC 3′ |
| MSH2-14A-s3 | 273 | 5′ (*)-GTATGTGTATGTTACCACATT 3′ |
| MSH2-14A-as3 | 274 | 5′ TAGTTAAGGTCTCTTCAGTG 3′ |
| MSH2-14B-s | 275 | 5′ ATAATCTACATGTCACAGCA 3′ |
| MSH2-14B-as | 276 | 5′ (*)-GAATAAGGCAATTACTGAT 3′ |
| MSH2-15A-s | 280 | 5′ GTCTCTTCTCATGCTGTC 3′ |
| MSH2-15A-as | 281 | 5′ (*)-AATAGAGAAGCTAAGTTAAAC 3′ |
| MSH2-16A-s | 285 | 5′ TTACTAATGGGACATTCACATG 3′ |
| MSH2-16A-as | 286 | 5′ (*)-ACAATAGCTTATCAATATTACCTTC 3′ |
| * clamp is | 344 | CGCCCGCCGCGCCCCGCGCCCGCCCCGCCGCCCCCGCCCG |

In particular embodiments of the invention, primers used to amplify DNA regions from patient samples are labeled with fluorescent tags. Fluorescently tagged primers are used to detect the presence of PCR products without chemical staining as well as the origins of a product when two or more reaction products are mixed and analyzed in the same gel lane.

### Example 9

In this example, fluorescently labeled primers that detect the presence of absence of polymorphisms in the CTFR gene were employed. Exon 10 of the CFTR gene was amplified with a primer set that detects the entire exon using a PCR protocol similar to that of Example 8. PCR was performed as described in Example 8 with a primer set that was modified with Texas Red (primers were obtained from MWG Biotech), and a second primer set that was modified with Oregon Green (also from MWG). Extension products were analyzed on TTGE side by side after being forced into a heteroduplex against themselves or by mixing with a control DNA. The extension products were analyzed on TTGE and the common mutation for deltaF508 and polymorphism M470V was observed.

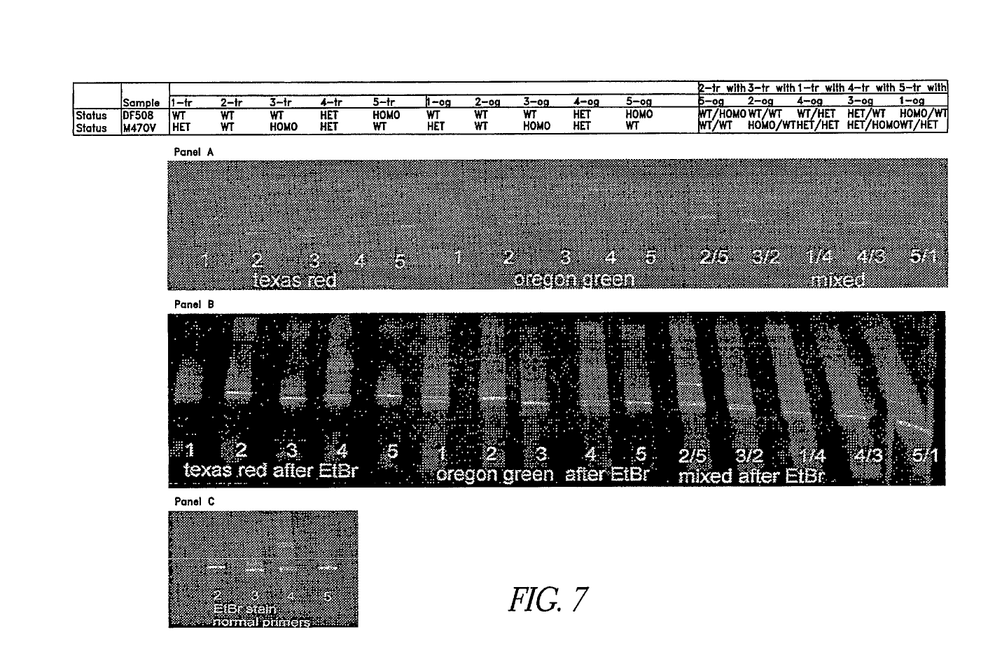

Results revealed the same banding pattern on TTGE for each individual fragment regardless of the modification state of the primer. Results also indicate the homozygous state of the DNA samples if the samples were mixed with wildtype DNA, which appears as a visually apparent heterozygous banding pattern (FIG. 7, Panel A). Poststaining of TTGE gels in EtBr also showed the same banding pattern for those products amplified with Texas Red modified or Oregon Green modified primers and unmodified primers. (FIG. 7, Panels B and C).

This example demonstrates that the use of fluorescently labeled primers allows one to rapidly identify the presence or absence of polymorphisms in an analyzed gene without staining or autoradiography and to rapidly differentiate the identity of individual extension products that are mixed and segregated on the same lane of a TTGE gel.

### Example 10

In one embodiment of the invention, the techniques described above in Example 8 can be used to screen DNA samples isolated from patient blood samples for mutations associated with HNPCC. In some embodiments of the invention, if a DNA sample generates a positive result in the assay, the existence of one or more mutations associated with HNPCC is confirmed with DNA sequencing of the relevant exons. Table E provides primer pairs to be used for the sequencing of each exon of the MSH2 and MLH1 genes, including first and second choices in some instances. A protocol for PCR-based sequencing reactions using these primers, as well as the primer sequences themselves, are also provided. Using the primers, the primer pairings and the protocol provided, a person with skill in the art is able to sequence any or all of the exons of the MSH2 and MLH1 genes and confirm the existence of HNPCC-related or other mutations in the coding sequences of these genes.

### Example 11

Using a protocol similar to that of Example 8, the HNPCC assay is performed with primers that have been modified with a fluorescent label for visualization on a fluorescent imager. In this Example, the short primer (without the GC clamp sequence) of each primer pair listed in Table 2 is modified by the addition of a fluorescent label such as Texas Red (absorption peak 595 nm, emission peak 615 nm) or Oregon Green (absorption peak 496 nm, emission peak 524 nm) (primers are obtained from MWG Biotech). The GC clamp primer is used in the unmodified form.

Primer sets and PCR stacking groups are designed for optimal sensitivity for TTGE, as described in Example 8. In particular embodiments, DNA from one individual is amplified with each primer set in a separate reaction, then stacked in average groups of three fragments/gel for gel analysis. PCR conditions for all fragments are as follows: 15 minutes @ 95° C. for the initial denaturation, then 35 cycles of: 30 seconds @ 94° C., 30 seconds @ 47-58.5° C., and 30 seconds @ 72° C. A final extension is performed at 72° C. for 10 minutes. The approximately 15 ul PCR reactions contain 7.5 ul Qiagen 2× Hotstart Master Mix, 50-200 ng genomic DNA, sense and antisense primers for each fragment at a final concentration of 0.5-1 uM. Prior to gel loading and stacking of gel groups, PCR samples are heated and re-annealed to provide best heteroduplex formation. Each PCR product is heated to 95° C. for 5 min, 50° C. for 10 min, then brought to 4° C. PCR products (approximately 4-8 μl each depending on signal strength) are then assembled into groups of products with equal melting characteristics and mixed with loading dye consisting of 70% glycerol, 0.05% bromophenol blue, 0.05% xylene cyanol, 2 mM EDTA). DNA is separated on denaturing gels (7 M urea, 8% acrylamide/bis(37.5:1) in 50 mM Tris, 25 mM acetic acid, 1.25 mM EDTA) for 3-5 hours at 125 V or 150 V on the Dcode system. (Biorad). Temperature ranges from 45° C. to 67° C. are used with ramp rates of 1.0-1.5° C./hr, depending on gel groups. The gels are imaged on a fluorescent image, and images are captured in the respective channel. Gels can also be photographed using the Versadoc 1000 system (BioRad).

Resulting images show extension products in the respective channel, e.g. presenting as a red pattern for Texas Red modified primers, and as a green pattern for Oregon Green modified primers.

Moreover, since the labeled extension products fluoresce in different spectra, this method allows for the simultaneous visualization of multiple DNA samples at once. For example, if one sample of primer has been previously amplified with Texas Red modified primers and the another with Oregon Green modified primers. one can multiplex the same extension product from 2 or more different DNA samples at the gel stage of the process.

In a specific embodiment, DNA from one individual is amplified with each primer set in separate reactions, using short primers labeled with the Texas Red fluorescent tag. DNA from another individual is amplified with primer sets labeled with the Oregon Green fluorescent tag. Prior to gel loading and stacking of gel groups, Texas Red tagged extension product and Oregon Green tagged extension product are mixed at equal ratios, and re-annealed to provide heteroduplex formation. Mixed PCR products are heated to 95° C. for 5 min, 50° C. for 10 min, then brought to 4° C.

The PCR products (approximately 4-8 μl of each depending on signal strength) are then assembled into groups of products with equal melting characteristics and mixed with loading dye. DNA is separated on denaturing gels, and gels are imaged on a fluorescent imager. Images for each gel are captured in both channels, after which they are overlayed for viewing of both colors. Whenever the extension products have identical sequence, the banding pattern appears as yellow on the overlay image. If one extension product is missing, the other extension product will be visible (red or green). Moreover, since all products are forced into a heteroduplex, any one homozygous mutation appear as a heterozygous pattern after having been mixed with wildtype sequence. The heterozygous pattern may present as a distinct pattern of 2 yellow, 1 red and 1 green band, or as a compressed yellow pattern of all 4 bands, depending on the specific melting temperature shift of each duplex. Most importantly, this mandatory heteroduplex formation of every fragment in the assay facilitates homozygous detection. This provides an advantage over conventional TTGE, since the homozygous mutations can be the most difficult to resolve on gel. In addition, the cost for analyzing samples is reduced because each gel is loaded with a multiple number of DNA samples.

As noted above, heteroduplexes have one or more mismatched base pairs between the two strands comprising the duplex. Creating heteroduplexes in the TTGE samples permits a greater difference in melting temperatures between PCR products with different sequences than would be seen between homoduplexes differing in sequence by only one or a few bases. Heteroduplex formation assists with the melting temperature (Tₘ) calculations in various Tm calculating software programs, such as the Bio-Rad Winmelt software. In order to get efficient and sensitive TTGE PCR fragments, it is helpful to have the regions of sensitivity be linear within 0.1° C. Consistent predictions of Tₘ ranges within that level of specificity are difficult to obtain. By increasing the difference in melting temperature of double stranded PCR products in a sample through the formation of heteroduplexes, the need for precise melting temperature predictions is reduced.

Another aspect of the invention involves the importance of analysis consistencies in the laboratory. In TTGE, SSCP, DGGE, or any other denaturing assay, the primary determinant for the detection of an abnormality is the mobility shift of the fragment. Even if the assay works technically, the shift may be so slight that it is only apparent if it is known that there is a mutation on the input DNA. Mobility shifts should be visually significant in order to be detected every single time. By creating multicolor heteroduplex under denaturing conditions, color change is added to the visual criteria whereby the mutation can be detected. This additional visual criteria increases the sensitivity of the assay.

Although the invention has been described with reference to embodiments and examples, it should be understood that various modifications can be made without departing from the spirit of the invention. Accordingly, the invention is limited only by the following claims.

TABLE A

hMLH1
genomic seq. and primers

5′ upstream seq.
Aggtagcgggcagtagccgcttcagggagggacgaagagacccagcaacccacagagttgagaaat
(SEQ ID NO.: 1)
Exon 1
ttgactggca ttcaagctgt
ccaatcaata gctgccgctg aagggtgggg ctggatggcg taagctacag ctgaaggaag
aacgtgagca cgaggcactg aggtgattgg ctgaaggcac ttccgttgag catctagacg
tttccttggctctt ctggcgccaa aATGTCGTTC GTGGCAGGGG TTATTCGGCG GCTGGACGAG

| | |
|---|---|
| 61 | ACAGTGGTGA ACCGCATCGC GGCGGGGGAA GTTATCCAGC GGCCAGCTAA TGCTATCAAA |
| 121 | GAGATGATTG AGAACTGgta cggagggagt cgagccgggc tcacttaagg gctacgactt |
| 181 | aacgggccgc gtcactcaat ggcgcggaca cgcctctttg cccgggcaga ggcatgtaca |
| 241 | gcgcatgccc acaacggcgg aggccgccgg gttccctgac gtgccagtca ggccttctcc |
| 301 | ttttccgcag accgtgtgtt tctttaccgc tctcccccga gaccttttaa gggttgtttg |
| 361 | gagtgtaagt ggaggaatat acgtagtgtt gtcttaatgg taccgttaac taagtaagga |
| 421 | agccacttaa tttaaaatta tgtatgcaga acatgcgaag ttaaaagatg tataaaagct |
| 481 | taagatgggg agaaaaacct tttttcagag ggtactgtgt tactgttttc ttgcttttca |

(SEQ ID NO.: 2)

| | | |
|---|---|---|
| MLH1-1A-s: | 5′ (*)-CAATAGCTGCCGCTGA 3′ | (SEQ ID NO.: 3) |
| MLH1-1A-as: | 5′ CGCTGGATAACTTCCC 3′ | (SEQ ID NO.: 4) |
| MLH1-1B-s: | 5′ GGCGGGGGAAGTTATC 3′ | (SEQ ID NO.: 5) |
| MLH1-1B-as: | 5′ (*)-CGCGCCATTGAGTGAC 3′ | (SEQ ID NO.: 6) |
| MLH1-1C-s: | 5′ (*)-CAAAGAGATGATTGAGAAC | (SEQ ID NO.: 7) |
| MLH1-1C-AS: | 5′ CATGCCTCTGCCCGG | (SEQ ID NO.: 8) |
| MLH1-1D-S: | 5′ (*)-GGAAGAACGTGAGCACGA | (SEQ ID NO.: 9) |
| MLH1-1D-AS: | 5′ CATTAGCTGGCCGCTG | (SEQ ID NO.: 10) |
| Sense tag: | TCTGCCTTTTTCTTCCATCGGG | (SEQ ID NO.: 11) |
| Antisensense tag: | TCCCCAACCCCCTAAAGCGA | (SEQ ID NO.: 12) |
| MLH1-1seq-s: | TCTGCCTTTTTCTTCCATCGGGGCTTCAGGGAGGGACGAAGA | (SEQ ID NO.: 13) |
| MLH1-1seq-as: | TCCCCAACCCCCTAAAGCGA TGCGCTGTACATGCCTCTGC | (SEQ ID NO.: 14) |

Exon 2

| | |
|---|---|
| 2401 | gattctcctg ccttagcctc ctgagtagct gggattacag gcatgcgtca ccatgcctgg |
| 2461 | ctaattttgt atttttagta caaatggggt ttctccatgt tggtcaggct ggtctcaaac |
| 2521 | tcctgacctc aggtgatcca cccgccttgg cctcccaaag tgctgggatt atgggtgtga |
| 2581 | gccattgcgc ctggccagaa aattcattga cttcctaaag atttattaac tttctgcatt |
| 2641 | actttttttt ttcccctcca tcgtaaatat aaaagggaat agtagagaaa atcattcaga |
| 2701 | attttatttt ttagtgacat tatttagtga cattttatta gagtcactta ggaacctgag |
| 2761 | gctgaataaa gttcaggtaa aagtaaaatt agttgagaag agacatctgc caaaagaaat |
| 2821 | ctatttttaa cttcacttgc tgtctttcct agaggaacag aaatagtgct gaatgtccta |
| 2881 | ttagaaatga tggttgctct gcccgtctct tccctctctc tcacacaata tgtaaactca |
| 2941 | tacagtgtat gagcctgtaa gacaaaggaa aaacacgtta atgaggcact attgtttgta |
| 3001 | tttggagttt gttatcattg cttggctcat attaaaatat gtacattaga gtagttgcag |
| 3061 | actgataaat tattttctgt ttgatttgcc agTTTAGATG CAAAATCCAC AAGTATTCAA |
| 3121 | GTGATTGTTA AAGAGGGAGG CCTGAAGTTG ATTCAGATCC AAGACAATGG CACCGGGATC |
| 3181 | AGGgtaagta aaacctcaaa gtagcaggat gtttgtgcgc ttcatggaag agtcaggacc |
| 3241 | tttctctgtt ctggaaacta ggcttttgca gatgggattt tttcactgaa aaattcaaca |
| 3301 | ccaacaataa atatttattg agtacctatt atttgctggg cactgttcag gggatgtgtc |
| 3361 | agtgaataaa atagattaaa atctattctc ttctgatgct tacattatag tggtgggaga |
| 3421 | caaaatgggt ataataaata ttatattaga tagcattaag tgctgtggag aaaactaaag |
| 3481 | cagggaggaa gataggagtg tgcaagccag aaaggttgca attaaattga gtagttcagg |
| 3541 | aaggcttcaa tatggatgtg atatttgaga gaccggtgga agtcaaggag caagttgtga |

(SEQ ID NO.: 15)
Gels:

| | | |
|---|---|---|
| MLH1-2A-s: | 5′ (*)-TTATCATTGCTTGGCT 3′ | (SEQ ID NO.: 16) |
| MLH1-2A-as: | 5′ TTGTCTTGGATCTGAATC 3′ | (SEQ ID NO.: 17) |
| MLH1-2B-s: | 5′ (*)-GCAAAATCCACAAGTATT 3′ | (SEQ ID NO.: 18) |
| MLH1-2B-as: | 5′ CCTGACTCTTCCATGAA 3′ | (SEQ ID NO.: 19) |
| MLH1-2seq-s: | TCTGCCTTTTTCTTCCATCGGGTGCCCGTCTCTTCCCTCTCT | (SEQ ID NO.: 20) |
| MLH1-2seq-as: | TCCCCAACCCCCTAAAGCGACCTGAACAGTGCCCAGCAAA | (SEQ ID NO.: 21) |

Exon 3

| | |
|---|---|
| 7081 | acctgtaatc ccagccactc tggaggctga gacatgaaaa ttgcttgaac ccgggaggcg |
| 7141 | gaggttgcag tgagctgaga tctcgccact gcacttcagc ctgggtgaca gagcaagact |
| 7201 | ctgtctcaaa ggaggttgca gtgagctgag atctcgccac tgcacttcag cctgggtgac |
| 7261 | agagcaagac tctgtctcaa aaaaaaaaaa aacaaaaacc aagaaaagaa aaaaaaactc |
| 7321 | ttctaagagg attttttttt cctggattaa atcaagaaaa tgggaattca aagagatttg |
| 7381 | gaaaaatgag taacatgatt atttactcat ctttttggta tctaacagAA AGAAGATCTG |
| 7441 | GATATTGTAT GTGAAAGGTT CACTACTAGT AAACTGCAGT CCTTTGAGGA TTTAGCCAGT |
| 7501 | ATTTCTACCT ATGGCTTTCG AGGTGAGgta agctaaagat tcaagaaatg tgtaaaatat |
| 7561 | cctcctgtga tgacattgtc tgtcatttgt tagtatgtat ttctcaacat agataaataa |
| 7621 | ggtttggtac cttttacttg ttaaatgtat gcaaatctga gcaaacttaa tgaactttaa |
| 7681 | ctttcaaaga ctgagaattg ttcataaata aactatttta cctgcagaga cctctgatat |
| 7741 | atgtttcttg atggaagtac ccagtaccac ctatgaagtt ttcttgtcaa aaaatcaaat |
| 7801 | gtgaatctga tcattactta gatctaagta ccaatatatg aaaaatatag gagacaagga |
| 7861 | agcatggtaa atgatactga gattgggaga ctacatggaa aaagacttgt tcccttcaac |
| 7921 | agatagacag cagggaaaaa agaatagaga aaggagtaaa gaacctgtag attaaaagac |
| 7981 | atttaaggga catatgaacc aggtccagtg tatagatctt acctaaatcc tgatggagca |
| 8041 | aactataaaa aaattttttt gagacaaatg tttgaataca ggttgactat ttgatggcat |

(SEQ ID NO.: 22)

| | | |
|---|---|---|
| MLH1-3-s: | 5′ (*)-GGGAATTCAAAGAGAT 3′ | (SEQ ID NO.: 23) |
| MLH1-3-as: | 5′ TTCTTGAATCTTTAGCTT 3′ | (SEQ ID NO.: 24) |
| MLH1-3B-s: | 5′ ATATTGTATGTGAAAGGTTCAC 3′ | (SEQ ID NO.: 25) |
| MLH1-3B-as: | 5′ (*)-ACCAAACCTTATTTATCTATGT | (SEQ ID NO.: 26) |
| MLH1-3seq-s: | TCTGCCTTTTTCTTCCATCGGGCAAGACTCTGTCTCAAAGGAGGTT | (SEQ ID NO.: 27) |
| MLH1-3seq-as: | TCCCCAACCCCCTAAAGCGAGACAATGTCATCACAGGAGGAT | (SEQ ID NO.: 28) |
| MLH1-3seq-s2- | cctggattaaatcaagaaaatggg | (SEQ ID NO.: 29) |
| internal | | |
| MLH1-3seq-as2 | TCCCCAACCCCCTAAAGCGACATTAAGTTTGCTCAGATTTGCATA | (SEQ ID NO.: 30) |

to be used with MLH1-3seq-s for PCR and tagged seq
Exon 4

| | |
|---|---|
| 10261 | gagatgctgt cacacagacc ccgtcatagc acagttcctg agttacatct ttacatactg |
| 10321 | tagtatcctt cttgtgaaaa aagatacaga ttccaaaggt ctgagaaacc aatcttggtt |
| 10381 | ataaagggga aaaatggtca tgggttttta aaatttgttt tgtcttaatt gcatttcaaa |
| 10441 | tttacatttc taaatgaata attgcttata taaagcagtt ttgattaaca atataaaaca |
| 10501 | ctatctattt ggagtgattc ctttacccat ttctgaaggc aagttttaaa aattactaga |
| 10561 | agacacttca ttgagaatat tattaaacat gcctatagtt ctaccacctc aacacaattg |
| 10621 | cttattaaca cattaatgtt ttggtgtgtt ttggactttt taatatgtat ttttcacttg |
| 10681 | ttctagtaat tatgctacag attgatcatt tctttttcaa catgtcatca aagcaagtga |
| 10741 | gcaaagtgct catcgttgcc acatattaat acaaaatgga agcagcagtt cagataacct |
| 10801 | ttccctttgg tgaggtgaca gtgggtgacc cagcagtgag tttttctttc agtctatttt |
| 10861 | cttttcttcc ttagGCTTTG GCCAGCATAA GCCATGTGGC TCATGTTACT ATTACAACGA |
| 10921 | AAACAGCTGA TGGAAAGTGT GCATACAGgt atagtgctga cttcttttac tcatatatat |
| 10981 | tcattctgaa atgtattttt tgcctaggtc tcagagtaat cctgtctcaa caccagtgtt |
| 11041 | atcttttttg gcagagatct tgagtacgtt ttcttttctc cttattgata aattgataat |
| 11101 | cctcaaggat gattattagg tgatactctt acttcatgga ttcttaaaag atatgattta |
| 11161 | acatattaca agtgcctagc aaggtgtctg ttacacgtag gtattttaag taaatggtag |
| 11221 | ctgctgatgt aatttctgcc cctttgccct tcagttgggg tattgctttg gaccgattag |
| 11281 | agggctgtgg ctgggatgct aaaggttcat gtttccttag ctggctcctg agccaccagc |
| 11341 | tcccaccacc tgtgtatacc tgtgctagtt tgccttccca caagtagctg ctggctatct |
| 11401 | gttatgctgg tacagttttc agaaactgat gaatggcctt tgaacagaac aaaaatgaga |
| 11461 | ttcagaataa caaaattgca cctttgtttt tataagcact ggccattcac tagttgaaga |
| 11521 | ctggtaggaa tacctaattc atgccaaaag aaagataatt tttaaaaatc acacaggttg |

(SEQ ID NO.: 31)

| | | |
|---|---|---|
| MLH1-4A-s4 | GGTGAGGTGACAGTGGGT | (SEQ ID NO.: 32) |
| MLH1-4A-as4 | (*)-TGAATATATATGAGTAAAAGAAGTCAG | (SEQ ID NO.: 33) |
| MLH1-4B-s2 | TCATGTTACTATTACAACGAAAA | (SEQ ID NO.: 34) |
| MLH1-4B-as2 | (*)-GATAACACTGGTGTTGAGACA | (SEQ ID NO.: 35) |
| MLH1-4-seq-s: | TCTGCCTTTTTCTTCCATCGGGCATGTCATCAAAGCAAGTGAGC | (SEQ ID NO.: 36) |
| MLH1-4-seq-as: | TCCCCAACCCCCTAAAGCGATGAGACAGGATTACTCTGAGACCT | (SEQ ID NO.: 37) |

Exon 5

| | |
|---|---|
| 12961 | catttgctgg aagaacagat agtttttcaa atccaattca aggactgggt atggtggctc |
| 13021 | atgcctgtaa tcccagcact ttgggaggcc gaggcaggcg tatccaggag ttcgagacta |
| 13081 | gcctgaccaa catggtgaaa ctccgtctct actaaaaata caaaattagc caggtgtggt |
| 13141 | ggtgggcacc tgtaatctca gctacttggg aggctgaggc aggagaatcg cttgaacctg |
| 13201 | gtaggcggag gttgtagtga gctgagattg tgccattgct ctccagcctg ggaaacaaga |
| 13261 | gcaaaactcc gtctcaaaaa aaaaaaaaat ccaattcaaa tgattatgga agtagtggag |
| 13321 | aaataaacag gaaaatgata aataattaag ataatatata atatggctat attttaatct |
| 13381 | attgttgata tgattttctc ttttcccctt gggattagta tctatctctc tactggatat |
| 13441 | taatttgtta tattttctca ttagAGCAAG TTACTCAGAT GGAAAACTGA AAGCCCCTCC |
| 13501 | TAAACCATGT GCTGGCAATC AAGGGACCCA GATCACGgta agaatggtac atgggagagt |
| 13561 | aaattgttga agctttgttt gtataaatat tggaataaaa aataaaattg cttctaagtt |
| 13621 | ttcagggtaa taataaaatg aatttgcact agttaatgga ggtcccaaga tatcctctaa |
| 13681 | gcaagataaa tgactattgg cttttgtggc atggcagcct gccacgtcct tgtctttttt |
| 13741 | aagggctagg agattcttta ttgggatggc aaaagtcaat ggcagggtag ttgtcattga |
| 13801 | aagaagatta agcttgaccc cagaaggcat gggttagagc ccagccttgt cactcaatgg |
| 13861 | ttgtatgtcc agaggcaagt cacttaacat cccttaaccc cagttttctc atctgtcaaa |
| 13921 | tgaagcaaag aatacttgcc ctcttgactt aaagggtgtc tgatgagaca tatgactgta |
| 13981 | tcattagctg ggagaaagtc catcgtgctg cctatgtata gtgcctcaag ttggtctctt |
| 14041 | tcccttctat gattacacaa agcactccgc tgtcatgtta tccatcccgc ccctccattc |

(SEQ ID NO.: 38)

| | | |
|---|---|---|
| MLH1-5A-s: | 5′ (*)-GGGATTAGTATCTATCTCT 3′ | (SEQ ID NO.: 39) |
| MLH1-5A-as: | 5′ GGCTTTCAGTTTTCC 3′ | (SEQ ID NO.: 40) |
| MLH1-5B-s2: | 5′ CTGAAAGCCCCTCCTA 3′ | (SEQ ID NO.: 41) |
| MLH1-5B-as2: | 5′ (*)-AGCTTCAACAATTTACTCTC 3′ | (SEQ ID NO.: 42) |
| MLH1-5C-s2: | 5′ CAAGGGACCCAGATCAC 3′ | (SEQ ID NO.: 43) |
| MLH1-5C-as2: | 5′ (*)-CCAATATTTATACAAACAAAGC 3′ | (SEQ ID NO.: 44) |
| MLH1-5D-s | 5′ (*)-TTTGTTATATTTTCTCATTAGAG | (SEQ ID NO.: 45) |
| MLH1-5D-s | 5′ ATTCTTACCGTGATCTGG | (SEQ ID NO.: 46) |
| MLH1-5seq-s2: | TCTGCCTTTTTCTTCCATCGGGCCCTTGGGATTAGTATCTATCTCT | (SEQ ID NO.: 47) |
| MLH1-5seq-as: | TCCCCAACCCCCTAAAGCGAGGACCTCCATTAACTAGTGCAA | (SEQ ID NO.: 48) |

Exon 6

| | |
|---|---|
| 14761 | atgcgtcacc atgcccggct aatttttgta tttttagtag agacagggtt tcaccatgtt |
| 14821 | ggccaggctg gtctcgaact cctgacctca ggtgacccac ccaccttggc ctcccaaagt |
| 14881 | tctgggatta cagacgtgag ccactgcacc cagcctgaaa aatatctttg aatgccatgt |
| 14941 | gatactatac ttgtcagttt acatgtgtgt cccactaaat catgtactct cctgagcagg |
| 15001 | atcatgcttt gtcttcatat tttctgtaca aagcaaagac tctgacacaa agctagcccc |
| 15061 | cagtgcatag ttgagaaatc agtgaatgaa tgtgggaggc aggaaaaatg tcctttaatt |
| 15121 | cttctgttaa tgctgtctta tccctggccc cagtcagtgc ttagaactgt gctgttggta |
| 15181 | aatataattg gattcactat cttaagacct cgcttttgcc aggacatctt gggttttatt |
| 15241 | ttcaagtact tctatgaatt tacaagaaaa atcaatcttc tgttcagGTG GAGGACCTTT |
| 15301 | TTTACAACAT AGCCACGAGG AGAAAAGCTT TAAAAAATCC AAGTGAAGAA TATGGGAAAA |
| 15361 | TTTTGGAAGT TGTTGGCAGg tacagtccaa aatctgggag tgggtctctg agatttgtca |
| 15421 | tcaaagtaat gtgttctagt gctcatacat tgaacagttg ctgagctaga tggtgaaaag |
| 15481 | taaaactagc ttacagatag tttctggtca aggtttagcc accaattttg cagtttctct |
| 15541 | catctcccca ggaaagagca gttggtcttt agatcaatga gagctctttt atggcagaca |
| 15601 | aaacaaagtg actctagcca acttgagcta aaaagaaatt tagtggaagg ctaggagtta |
| 15661 | ccacatgaag tgtgtgcagc tgccccttgg agagaataag aaccagggtg cctctgggac |
| 15721 | ttaacatcat tactgtactc cagttgtttt cattcttttc ctgactttgc tctagagtca |

(SEQ ID NO.: 49)

| | | |
|---|---|---|
| MLH1-6-5-s | (*)-ATTCACTATCTTAAGACCTCGCT | (SEQ ID NO.: 50) |
| MLH1-6-5-as | CTAGAACACATTACTTTGATGACAA | (SEQ ID NO.: 51) |
| MLH1-6seq-s: | TCTGCCTTTTTCTTCCATCGGGCTGTTAATGCTGTCTTATCCCTGG | (SEQ ID NO.: 52) |
| MLH1-6seq-as: | TCCCCAACCCCCTAAAGCGACCATCTAGCTCAGCAACTGTTCA | (SEQ ID NO.: 53) |

Exon 7

| | |
|---|---|
| 17461 | aatccttcgg ttcacgagct ctgtagagaa aagagaaata accgccaacc aagaaaagat |
| 17521 | tgggagatac tagaataaga cccaggggca ggaagaagcc agtgagaagg agggcatgtt |
| 17581 | gagagctctg agagagaata aaagcagggg ttgttggagc tagcttctca agatgtcctt |
| 17641 | gaggcaaacc agacctttgg gacactctga aaataaaact gaaagtgaag agattgtggg |
| 17701 | ccgaatgtgg tggctcacgc ctgtaatccc agcactttgg gaggtcgagg cgggtggatc |
| 17761 | acctgagatc aggagttcga taccagcctg gccaacatgg cgaaacgcca tctctactaa |
| 17821 | aaatacaaaa aaaattagct gggcctggtg gcaggcgcct ataatcccag ctactcggga |
| 17881 | ggctgaggcg ggagaatcgc ttgagtccag gaggcggagg ttgcagtgag ctgagatcgt |
| 17941 | gccattgcac tccagcctgg gcaacaagag caaaactctg tctcaaaaat aaataaaaat |
| 18001 | aaataaaaaa gagatagtgg cgtgatatcc ttgattctat cagcaaccta taaaagtaga |
| 18061 | gaggagtctg tgttttgatt cagtcacctt tagcattttt atttccatga agtttctgct |
| 18121 | ggtttatttt tctgtgggta aaatattaat aggctgtatg gagatatttt tctttatatg |
| 18181 | tacctttgtt tagattactc aactccacta atttatttaa ctaaaagggg gctctgacat |
| 18241 | ctagtgtgtg tttttggcaa ctcttttctt actcttttgt ttttcttttc cagGTATTCA |
| 18301 | GTACACAATG CAGGCATTAG TTTCTCAGTT AAAAAAgtaa gttcttggtt tatgggggat |
| 18361 | ggttttgttt tatgaaaaga aaaaagggga tttttaatag tttgctggtg gagataaggt |
| 18421 | tatgatgttt cagtctcagc catgagacaa taaatccttg tgtcttctgc tgtttgttta |
| 18481 | tcagcaagga gagacagtag ctgatgttag gacactaccc aatgcctcaa ccgtggacaa |
| 18541 | tattcgctcc atctttggaa atgctgttag tcggtatgtc gataacctat ataaaaaaat |
| 18601 | cttttacatt tattatcttg gtttatcatt ccatcacatt attttggaac ctttcaagat |
| 18661 | attatgtgtg ttaagagttt gctttagtca aatacacagg cttgttttat gcttcagatt |
| 18721 | tgttaatgga gttcttattt cacgtaatca acactttcta ggtgtatgta atctcctaga |
| 18781 | ttctgtggcg tgaatcatgt gttctttcaa ggtcttagtc ttgaaaatat ttatagtgta |
| 18841 | gtagaactat tttatcctcc aatgctcctt cttttccttg tatttccatt atcatcactt |
| 18901 | taggatttca cttatttatc attcaacatt tattaattgc ctctcatatt ccaggctttg |
| 18961 | tgctagaagt tagggatata aagacaaata agatatttcc tgcccttaaa gactagattc |
| 19021 | gtgttgctaa gtcttcatta tcaagaaaag cataagtggg gaaaagtgct tgcattatgg |

(SEQ ID NO.: 54)

| | | |
|---|---|---|
| MLH1-7-s: | 5′ TAACTAAAAGGGGGCT 3′ | (SEQ ID NO.: 55) |
| MLH1-7-as: | 5′ (*)-TTTATTGTCTCATGGCT 3′ | (SEQ ID NO.: 56) |
| MLH1-7seq-s: | TCTGCCTTTTTCTTCCATCGGGTTCCATGAAGTTTCTGCTGG | (SEQ ID NO.: 57) |
| MLH1-7seq-as: | TCCCCAACCCCCTAAAGCGACCTTATCTCCACCAGCAAACTA | (SEQ ID NO.: 58) |

Exon 8

| | |
|---|---|
| 18001 | aaataaaaaa gagatagtgg cgtgatatcc ttgattctat cagcaaccta taaaagtaga |
| 18061 | gaggagtctg tgttttgatt cagtcacctt tagcattttt atttccatga agtttctgct |
| 18121 | ggtttatttt tctgtgggta aaatattaat aggctgtatg gagatatttt tctttatatg |
| 18181 | tacctttgtt tagattactc aactccacta atttatttaa ctaaaagggg gctctgacat |
| 18241 | ctagtgtgtg tttttggcaa ctcttttctt actcttttgt ttttcttttc caggtattca |
| 18301 | gtacacaatg caggcattag tttctcagtt aaaaaagtaa gttcttggtt tatgggggat |
| 18361 | ggttttgttt tatgaaaaga aaaaagggga tttttaatag tttgctggtg gagataaggt |
| 18421 | tatgatgttt cagtctcagc catgagacaa taaatccttg tgtcttctgc tgtttgttta |
| 18481 | tcagCAAGGA GAGACAGTAG CTGATGTTAG GACACTACCC AATGCCTCAA CCGTGGACAA |
| 18541 | TATTCGCTCC ATCTTTGGAA ATGCTGTTAG TCGgtatgtc gataacctat ataaaaaaat |
| 18601 | cttttacatt tattatcttg gtttatcatt ccatcacatt attttggaac ctttcaagat |
| 18661 | attatgtgtg ttaagagttt gctttagtca aatacacagg cttgttttat gcttcagatt |
| 18721 | tgttaatgga gttcttattt cacgtaatca acactttcta ggtgtatgta atctcctaga |
| 18781 | ttctgtggcg tgaatcatgt gttctttcaa ggtcttagtc ttgaaaatat ttatagtgta |
| 18841 | gtagaactat tttatcctcc aatgctcctt cttttccttg tatttccatt atcatcactt |
| 18901 | taggatttca cttatttatc attcaacatt tattaattgc ctctcatatt ccaggctttg |
| 18961 | tgctagaagt tagggatata aagacaaata agatatttcc tgcccttaaa gactagattc |

(SEQ ID NO.: 59)

| | | |
|---|---|---|
| MLH1-8A-s: | 5′ (*)-GCTGGTGGAGATAAGG 3′ | (SEQ ID NO.: 60) |
| MLH1-8A-as: | 5′ TGTCCACGGTTGAGG 3′ | (SEQ ID NO.: 61) |
| MLH1-8B-s: | 5′ GGGGGCAAGGAGAGACAGTAG 3′ | (SEQ ID NO.: 62) |
| MLH1-8B-as2: | 5′ (*)-ATATAGGTTATCGACATACC 3′ | (SEQ ID NO.: 63) |
| MLH1-8C-s2: | 5′ AAATGCTGTTAGTC 3′ | (SEQ ID NO.: 64) |
| MLH1-8C-as: | 5′ (*)-TCTTGAAAGGTTCCAA 3′ | (SEQ ID NO.: 65) |
| MLH1-8seq-s: | TCTGCCTTTTTCTTCCATCGGGGGTTTATGGGGGATGGTTTTG | (SEQ ID NO.: 66) |
| MLH1-8seq-as: | TCCCCAACCCCCTAAAGCGACGCCACAGAATCTAGGAGATTACA | (SEQ ID NO.: 67) |

Exon 9

| | |
|---|---|
| 20401 | tattaacctt ccctccccag taaacactcc tgggaacaac acacattgta gaaccacgtt |
| 20461 | gtggtgctgt tcagtatagc aagtaattca gcagagataa gttcttggaa tctcatcttt |
| 20521 | gggatttagt tactaagata cattcaagtt tgagcaaaat aaggtctcag agcttggatt |
| 20581 | cattgttctg ttccagcaat tagagcagta cctggcacat agcacaagtg cttgaaaaca |
| 20641 | ctgactgagt agggtaggtg ggtgagtggg tgggtgggtg ggtgggtgga tggatggatg |
| 20701 | ggaggatggg tgggtgaatg ggtgaacaga caaatggatg gatgaatgga caggcacagg |
| 20761 | aggacctcaa atggaccaag tcttcggggc cctcatttca caaagttagt ttatgggaag |
| 20821 | gaaccttgtg tttttaaatt ctgattcttt tgtaatgttt gagttttgag tattttcaaa |
| 20881 | agcttcagaa tctcttttct aatagAGAAC TGATAGAAAT TGGATGTGAG GATAAAACCC |
| 20941 | TAGCCTTCAA AATGAATGGT TACATATCCA ATGCAAACTA CTCAGTGAAG AAGTGCATCT |
| 21001 | TCTTACTCTT CATCAACCgt aagttaaaaa gaaccacatg ggaaatccac tcacaggaaa |
| 21061 | cacccacagg gaattttatg ggaccatgga aaaatttctg atccataggt ttgattaaac |
| 21121 | atggagaaac ctcatggcaa agtttggttt tattgggaag catgtataat ttttgtccta |
| 21181 | agtctgtgct cagccctccc acatgtgctc attgctggtt gactgttgga gtctggttct |
| 21241 | tacctctaag aggaagccca ggagagggca taaagccagc acactgtcct cacctgatgg |
| 21301 | tgtcagagtc cttacgagta agccctagcc agaacattgc tggaagagat caagggccac |
| 21361 | tgtttgaaat tgcacagcag gatacggaaa aggggtacct taggtatagg cattgtcatt |
| 21421 | aaagaaattg ctaagatact tgagattttc ctgtttaagg aatgagcttt atgatacaaa |
| 21481 | gagcagttct aaaaattagg gagggaatta actaaattaa ttaggatatt tctcaaattc |
| 21541 | ctttacagtt tttgtctctc tgctgatata gtgtttacat gattgttatt tactaaacaa |
| 21601 | atgctatttt gtattgtgct ccttataact taattgttta ttacaaggtt ttgatggtga |

(SEQ ID NO.: 68)

| | | |
|---|---|---|
| MLH1-9A-3-s | (*)-GTAATGTTTGAGTTTTGAGTATTTTC | (SEQ ID NO.: 69) |
| MLH1-9A-3-as | CAGAAATTTTTCCATGGTCC | (SEQ ID NO.: 70) |
| MLH1-9B-s | (*)-CAAAGTTAGTTTATGGGAAGGA | (SEQ ID NO.: 71) |
| MLH1-9B-as | GAAGAGTAAGAAGATGCACTTCTT | (SEQ ID NO.: 72) |
| MLH1-9C-s | (*)-CTTCAAAATGAATGGTTACATAT | (SEQ ID NO.: 73) |
| MLH1-9C-as | ATTCCCTGTGGGTGTTTC | (SEQ ID NO.: 74) |
| MLH1-9seq-s: | TCTGCCTTTTTCTTCCATCGGGGGTGGGTGAATGGGTGAACA | (SEQ ID NO.: 75) |
| MLH1-9seq-as: | TCCCCAACCCCCTAAAGCGATTTGCCATGAGGTTTCTCCA | (SEQ ID NO.: 76) |

Exon 10

| | |
|---|---|
| 23461 | tgtctacacc ttaagccgcg gctcccgaag cacctagaac cggaagagtt ggctcactat |
| 23521 | ttagcacaca cacacgtcta taatagtgct ggccacttgg ggttggaatt agtttattta |
| 23581 | tcagcatgtt gtctcccagc acttggtgtg tgtgatatgc agtatgtatt tgcagaatga |
| 23641 | aaagtctgag ggctgacatc atatttccca ctgtgcccag aaagagcaca gttagtccac |
| 23701 | atgagctaat gggggcaaag ggaagtgagg agggagaatg tactgcctta tcatgttttc |
| 23761 | tattacttgg ctgaagtaaa acagtcccaa gccgatagta agatagtggg ctggaaagtg |
| 23821 | gcgacaggta aaggtgcacc tttcttcctg gggatgtgat gtgcatatca ctacagaaat |
| 23881 | gtctttcctg aggtgatttc atgactttgt gtgaatgtac acctgtgacc tcacccctca |
| 23941 | ggacagtttt gaactggttg ctttcttttt attgtttagA TCGTCTGGTA GAATCAACTT |
| 24001 | CCTTGAGAAA AGCCATAGAA ACAGTGTATG CAGCCTATTT GCCCAAAAAC ACACACCCAT |
| 24061 | TCCTGTACCT CAGgtaatgt agcaccaaac tcctcaacca agactcacaa ggaacagatg |
| 24121 | ttctatcagg ctctcctctt tgaaagagat gagcatgcta atagtacaat cagagtgaat |
| 24181 | cccatacacc actggcaaaa ggatgttctg tcccttctta caggtacaag gcacagtttt |
| 24241 | ccttcattta ttcactaatt tagcagaacc tcactaagag cctcctatat gccaggctct |
| 24301 | gcgttagcaa taaaaggaat gccatgcctc accccatcag gaggtgctga tagcttgtag |
| 24361 | gcggagtgga aacagatgtg ctctagaggc tctaaatatt acttctgctg gggtcagttg |
| 24421 | ggaagccaca acagctactg ttcatcttcc ataaaagaca atcagccggg cacagtggct |
| 24481 | cacacctgta aatcccagca ctttgggagg ctgaggtggg tggatcacaa ggtcaggtgt |

(SEQ ID NO.: 77)

| | | |
|---|---|---|
| MLH1-10-s: | 5′ (*)-TGAATGTACACCTGTGAC 3′ | (SEQ ID NO.: 78) |
| MLH1-10-as: | 5′ TAGAACATCTGTTCCTTG 3′ | (SEQ ID NO.: 79) |
| MLH1-10seq-s: | TCTGCCTTTTTCTTCCATCGGGGCTGGAAAGTGGCGACAGG | (SEQ ID NO.: 80) |
| MLH1-10seq-s | TCCCCAACCCCCTAAAGCGAGCCAGTGGTGTATGGGATTCA | (SEQ ID NO.: 81) |

Exon 11

| | |
|---|---|
| 26221 | gatggagtct tgctctgtcg ccaagctgga gtgcagtggc acgatctcgg cttactgcaa |
| 26281 | cctctgactc cctggttgaa gggattctcc tccctcagcc tcccgagtac ctgggattac |
| 26341 | aggcatgcgc caccacgccc agctaatttt tgtattttta gtagagacgt ggtttcatca |
| 26401 | tgttggccag gatggtctcg atctcctgac cttgtgatcc acccgcctcg gcctccccaa |
| 26461 | atgctgggat tacaggcgtg agccaccacg cccggccact tggcatgaat ttaattcccg |
| 26521 | ccataaacct gtgagatagg taattctgtt atatccactt tacaaatgaa gagactgagg |
| 26581 | caaagaaaga tgatgtaact tacgcaaagc tacacagctc ttaagtagca gtgccaatat |
| 26641 | ttgaacacac tcagactcga tcctgaggtt ttgaccactg tgtcatctgg cctcaaatct |
| 26701 | tctggccacc acatacacca tatgtgggct ttttctcccc ctcccactat ctaaggtaat |
| 26761 | tgttctctct tattttcctg acagTTTAGA AATCAGTCCC CAGAATGTGG ATGTTAATGT |
| 26821 | GCACCCCACA AAGCATGAAG TTCACTTCCT GCACGAGGAG AGCATCCTGG AGCGGGTGCA |
| 26881 | GCAGCACATC GAGAGCAAGC TCCTGGGCTC CAATTCCTCC AGGATGTACT TCACCCAGgt |
| 26941 | cagggcgctt ctcatccagc tacttctctg gggcctttga aatgtgcccg gccagacgtg |
| 27001 | agagcccaga tttttgcctg ttatttagga actttctttg caagtattac ctggatagtt |
| 27061 | ttaacatttt cttctttgaa cctagttata aaggtattgt gctgttgttc ctaggcttag |
| 27121 | agtcataagg cctgagctca cttcctcact ttgcctccat ctggaacctt agaccaactt |
| 27181 | cctaggaaaa cgagctgtct gaaaacagaa tagggtgcct cttcaatgtg ctcttcactg |
| 27241 | gagatgttca ggaggaggct actcccacct acacagggtg cagtggaggg tctgggcccc |
| 27301 | agggaggcag caggaagagt ggaaagagcg gaggctctac tgttggacag acctgggtta |

(SEQ ID NO.: 82)

| | | |
|---|---|---|
| MLH1-11A-s: | 5′ (*)-TTGACCACTGTGTCATC 3′ | (SEQ ID NO.: 83) |
| MLH1-11A-as: | 5′ GTGCAGGAAGTGAACT 3′ | (SEQ ID NO.: 84) |
| MLH1-11B-s: | 5′ (*)-CAGAATGTGGATGTTAATG 3′ | (SEQ ID NO.: 85) |
| MLH1-11B-as: | 5′ GGAGGAATTGGAGCC 3′ | (SEQ ID NO.: 86) |
| MLH1-11C-s4: | 5′ CAGCAGCACATCGAGAG 3′ | (SEQ ID NO.: 87) |
| MLH1-11C-as4: | 5′ CGCCCGCCGCGCCCCGCGCCCGCCCCGCCGCCCCCGCCCGATCTGG | (SEQ ID NO.: 88) |
| | GCTCTCACGTCT 3′ | |
| MLH1-11seq-s: | TCTGCCTTTTTCTTCCATCGGGAGACTGAGGCAAAGAAAGATG | (SEQ ID NO.: 89) |
| MLH1-11seq-as: | TCCCCAACCCCCTAAAGCGAAGGCAAAAATCTGGGCTCT | (SEQ ID NO.: 90) |

Exon 12

| | |
|---|---|
| 31681 | aagatgaaaa agttctagag atagctggtg gtgatggttg cgcaacaatg taaatgccac |
| 31741 | tgagctctca tttaaaaatg gttaaaatgg taaattttat atatatttta ccacaataaa |
| 31801 | aaaaagtctt cttctgggag caccccccca agacaaaaat atgaaaattt tacactgata |
| 31861 | cttccatttc aagataattt taagattata aggattttgc ttaattcttg aattttatac |
| 31921 | ctgtaaacct tttatacttc aaatttcggg cagaattgct tctataacaa tgataattat |
| 31981 | acctcatact agcttctttc ttagtactgc tccatttggg gacctgtata tctatacttc |
| 32041 | ttattctgag tctctccact atatatatat atatatatat atattttttt tttttttttt |
| 32101 | ttttaataca gACTTTGCTA CCAGGACTTG CTGGCCCCTC TGGGGAGATG GTTAAATCCA |
| 32161 | CAACAAGTCT GACCTCGTCT TCTACTTCTG GAAGTAGTGA TAAGGTCTAT GCCCACCAGA |
| 32221 | TGGTTCGTAC AGATTCCCGG GAACAGAAGC TTGATGCATT TCTGCAGCCT CTGAGCAAAC |
| 32281 | CCCTGTCCAG TCAGCCCCAG GCCATTGTCA CAGAGGATAA GACAGATATT TCTAGTGGCA |
| 32341 | GGGCTAGGCA GCAAGATGAG GAGATGCTTG AACTCCCAGC CCCTGCTGAA GTGGCTGCCA |
| 32401 | AAAATCAGAG CTTGGAGGGG GATACAACAA AGGGGACTTC AGAAATGTCA GAGAAGAGAG |
| 32461 | GACCTACTTC CAGCAACCCC AGgtatggcc ttttgggaaa agtacagcct acctccttta |
| 32521 | ttctgtaata aaactgcctt ctaactttgg cttttcatga atcacttgca tcttctctct |
| 32581 | gcctgacttg ccctctggaa tggtgctgga atggtcctgt ggccttgtcc actgtctgcc |
| 32641 | tttgaccata acttgaaagt cacccaccat agtgtccttt gaaataactt aaatgtccac |
| 32701 | agttccaagc atgagttaaa aacacttcag aatgtagagt agttgttcaa ttgaataaac |
| 32761 | acacacacca gaaaaaaaag caagtttatc ttttattttt agtaaagaat tttgatagag |
| 32821 | cctcaacacc agaaatggct agagagagaa gcctaacata tctggaggat tatttttcat |
| 32881 | cctacttaaa gctgctttca cttttttcag gaaaaaacac acgttctgaa tctaatttat |
| 32941 | aaaactccct ggccgggtgc tgtggctcac acctataatc ccagcacttt gggaggctga |

(SEQ ID NO.: 91)

| | | |
|---|---|---|
| MLH1-12B-s: | 5′ (*)-TTTTTTTTAATACAGACTTTG 3′ | (SEQ ID NO.: 92) |
| MLH1-12B-as: | 5′ GTGACAATGGCCTGG 3′ | (SEQ ID NO.: 93) |
| MLH1-12C-s: | 5′ CATTTCTGCAGCCTCT 3′ | (SEQ ID NO.: 94) |
| MLH1-12C-as: | 5′ (*)-TTTTTGGCAGCCACT 3′ | (SEQ ID NO.: 95) |
| MLH1-12D-s3: | 5′ AGCCCCTGCTGAAGTG 3′ | (SEQ ID NO.: 96) |
| MLH1-12D-as3: | 5′ (*)-AGAAGGCAGTTTTATTACAGA 3′ | (SEQ ID NO.: 97) |
| MLH1-12E-s: | 5′ (*)-TGTCCAGTCAGCCCCA | (SEQ ID NO.: 98) |
| MLH1-12E-as: | 5′ CTCTGATTTTTGGCAGC | (SEQ ID NO.: 99) |
| MLH1-12seq-s: | TCTGCCTTTTTCTTCCATCGGGTTTCGGGCAGAATTGCTTC | (SEQ ID NO.: 100) |
| MLH1-12seq-as: | TCCCCAACCCCCTAAAGCGAGCAGAGAGAAGATGCAAGTGATT | (SEQ ID NO.: 101) |
| 631 bp | | |
| MLH1-12seq-s2 | CAGACTTTGCTACCAGGACTTGCT | (SEQ ID NO.: 102) |
| internal | | |

to be used after amplification with first primer set, but use this for
seq instead of MLH1-12seq-s
ALTERNATIVE FCTL SEQ PRIMER SET:

| | | |
|---|---|---|
| MLH1-12seq-s2 | TCTGCCTTTTTCTTCCATCGGGATAGCTGGTGGTGATGGTTGCG | (SEQ ID NO.: 103) |
| MLH1-12seq-as2 | TCCCCAACCCCCTAAAGCGACCATTCCAGCACCATTCCAGAG | (SEQ ID NO.: 104) |

Exon 13

| | |
|---|---|
| 34801 | gcctggaaga catagtgaga ctctctctca aaaaaaaaaa aaaaaaaaaa ggaagtaagc |
| 34861 | attgtgaggg caggtacctt ctctgttttg ttcattgctg gatgtagtta gtatacagca |
| 34921 | gtatctgatg gatggataga tggaggaatg aatgaatgag acttcacaaa ttcagctcac |
| 34981 | ttgctcaagg ccctgcagct ctacgggatg aagctatact ccagagtcct gctacattgg |
| 35041 | ctgtgtggcc agctgctggg atctgagggt tgtcagataa gcagtctacc agagaacaga |
| 35101 | ctgatcttgt tggccttctg ccagcacagg ggttcattca cagctctgta gaaccagcac |
| 35161 | agagaagttg cttgctcctc caaaatgcaa cccacaaaat ttggctaagt ttaaaaacaa |
| 35221 | gaataataat gatctgcact tccttttctt cattgcagAA AGAGACATCG GGAAGATTCT |
| 35281 | GATGTGGAAA TGGTGGAAGA TGATTCCCGA AAGGAAATGA CTGCAGCTTG TACCCCCCGG |
| 35341 | AGAAGGATCA TTAACCTCAC TAGTGTTTTG AGTCTCCAGG AAGAAATTAA TGAGCAGGGA |
| 35401 | CATGAGGgta cgtaaacgct gtggcctgcc tgggatgcat agggcctcaa ctgccaaggt |
| 35461 | tttggaaatg gagaaagcag tcatgttgtc agagtggcca ctacagtttt gctgggcaag |
| 35521 | ctcctcttcc tttactaacc cacaatagca tcagcttaaa gacaattttt gattgggaga |
| 35581 | aaagggagaa aaataatctc tgtttatttt aattagcatt aattggtatt cttgttaaac |
| 35641 | cataggagtc agagtaaatc agccatttca ccaattttca gtttgtttct gtcttagcta |
| 35701 | acagcagtgt aatggtcagc aaaattctta tcttgtgtac tgaatggcat gtcctgttgc |
| 35761 | tgaaagtgca caggcttggg aggtagccat gagctcaaat cctggcacta ccacctctct |
| 35821 | tgtgtgacct tagactcctg acctttctat gcctcagttc tttcttacct ataaaatgaa |

(SEQ ID NO.: 105)

| | | |
|---|---|---|
| MLH1-13A-s: | 5′ (*)-AATTTGGCTAAGTTTAA 3′ | (SEQ ID NO.: 106) |
| MLH1-13A-as: | 5′ GGAATCATCTTCCACC 3′ | (SEQ ID NO.: 107) |
| MLH1-13B-s2: | 5′ (*)-CATTGCAGAAAGAGACATC 3′ | (SEQ ID NO.: 108) |
| MLH1-13B-as3: | 5′ GTGAGGTTAATGATCCTTCT 3′ | (SEQ ID NO.: 109) |
| MLH1-13C-s1: | 5′ (*)-TGATTCCCGAAAGGAAATGAC 3′ | (SEQ ID NO.: 110) |
| MLH1-13C-as1: | 5′ CAGGCCACAGCGTTTACGTACCCTCATG 3′ | (SEQ ID NO.: 111) |
| MLH1-13D-s: | 5′ (*)-ATTAACCTCACTAGTGTTTTG | (SEQ ID NO.: 112) |
| MLH1-13D-as: | 5′ TGAGGCCCTATGCATC | (SEQ ID NO.: 113) |
| MLH1-13seq-s: | TCTGCCTTTTTCTTCCATCGGGACTGATCTTGTTGGCCTTCTG | (SEQ ID NO.: 114) |
| MLH1-13seq-as: | TCCCCAACCCCCTAAAGCGATGGCCACTCTGACAACATGA | (SEQ ID NO.: 115) |

Exon 14

| | |
|---|---|
| 46261 | tggtctccta ttagactctc catttcaaac cattccatga ttttgtcctc cttttgccac |
| 46321 | cttccgagcc tgtaaaaact aatgtttgtg attcctgagg tttctctaat gtcttttaat |
| 46381 | aaagttgacc tcagagatct cgttacctct ctgagttcct gctttgtctt agattttgat |
| 46441 | ccttgagtgt tctttaatct tttagcaatt ccttgttgca tgttaaaaga ttagttatat |
| 46501 | tttattcctc atttgtgttc gttttcacca ggaggctcaa ttcaggcttc tttgcttact |
| 46561 | tggtgtctct agttctggtg cctggtgctt tggtcaatga agtggggttg gtaggattct |
| 46621 | attacttacc tgttttttgg ttttattttt tgttttgcag TTCTCCGGGA GATGTTGCAT |
| 46681 | AACCACTCCT TCGTGGGCTG TGTGAATCCT CAGTGGGCCT TGGCACAGCA TCAAACCAAG |
| 46741 | TTATACCTTC TCAACACCAC CAAGCTTAGg taaatcagct gagtgtgtga acaagcagag |
| 46801 | ctactacaac aatggtccag ggagcacagg cacaaaagct aaggagagca gcatgaggta |
| 46861 | gttgggaggg cacaggcttt ggagtcagac acatgtggtt tcaaatccaa gttcgaccat |
| 46921 | ttcccattta tttgactgta gacaagttac attcctaaac tatgtctcag atttctcatc |
| 46981 | tgtaagttgt ggtattacta gttaacatgc aggggttttg tttgtttgtt tgtttgtttg |
| 47041 | tttgtgaggg taagaaataa cccaagaagc ctagtccttg gtagttgctc agtgccctat |
| 47101 | aaatgttgtg aaccaggtgg tgagggtttg gtgctgctag agaattctgg tatctgctct |
| 47161 | gtgcaacaga gtactgtagg tgatgcaaga gaaagaagac ctgatgcctt ctttcctccc |

(SEQ ID NO.: 116)

| | | |
|---|---|---|
| MLH1-14A-s: | 5′ (*)-GGTCAATGAAGTGGGG 3′ | (SEQ ID NO.: 117) |
| MLH1-14A-as: | 5′ CCACGAAGGAGTGGTTA 3′ | (SEQ ID NO.: 118) |
| MLH1-14B-s: | 5′ AGTTCTCCGGGAGATG 3′ | (SEQ ID NO.: 119) |
| MLH1-14B-as: | 5′ (*)-TACCTCATGCTGCTCTC 3′ | (SEQ ID NO.: 120) |
| MLH1-14seq-s: | TCTGCCTTTTTCTTCCATCGGGTGTTCGTTTTCACCAGGAGG | (SEQ ID NO.: 121) |
| MLH1-14seq-as: | TCCCCAACCCCCTAAAGCGATCGAACTTGGATTTGAAACCAC | (SEQ ID NO.: 122) |

Exon 15

| | |
|---|---|
| 48301 | tttaggaaga ctccctgccc ttcctataca tttcacataa tttttaataa gttgtaaaaa |
| 48361 | agtgatttat aggattcttt gtaagtgggg gaagttaagc agacaaaaag tttttaaatc |
| 48421 | ttactgcaga gtgtcaggaa ccttttatag caccagacag gtagggacag aacatgagtg |
| 48481 | gcagcaagcc agacttggtc ttagtgctct aacctgtctg ttagaggctg gccagtcaga |
| 48541 | cccctggttg aagacgttgg gaatcccagc tctttggagg ggtaagagat tttgttagac |
| 48601 | tgttaaccag attccacagc caggcagaac tatttctgtc tcatccatgt ttcagggatt |
| 48661 | acttctccca ttttgtccca actggttgta tctcaagcat gaattcagct tttccttaaa |
| 48721 | gtcacttcat ttttattttc agTGAAGAAC TGTTCTACCA GATACTCATT TATGATTTTG |
| 48781 | CCAATTTTGG TGTTCTCAGG TTATCGgtaa gtttagatcc ttttcacttc tgaaatttca |
| 48841 | actgatcgtt tctgaaaata gtagctctcc actaatatct tatttgtagt atgttaaatt |
| 48901 | tttctaaaac ttctaaggat agttgctgta ttgtatgatt tgcatatgga ggtatctata |
| 48961 | agaagtttta tactttttag caaaatagtc atttggtagc caacttaaac aaatgtttat |
| 49021 | taatatagaa gttaataata tctactgata ctcggccggg tgcggtggct catgcctgta |
| 49081 | atcccaccac tttgggaggc tgaggcgggc agatcatttg aggtcaggag ttcaagacca |
| 49141 | gcctgaccaa tatgatgaaa ccctgtctct actaaattac aaatattagc agggtatggt |
| 49201 | ggtgggcgcc tgtaatccca gctactcagg aggctaaggc aggagaatca tttgaaccca |
| 49261 | ggaggcagag gttgcaatga gctgagatca cgccactgca ctccagcctg ggcaacagag |

(SEQ ID NO.: 123)

| | | |
|---|---|---|
| MLH1-15-s: | 5′ TTCAGGGATTACTTCTC 3′ | (SEQ ID NO.: 124) |
| MLH1-15-as: | 5′ (*)-GAAAAATTTAACATACTACA 3′ | (SEQ ID NO.: 125) |
| MLH1-15seq-s2: | TCTGCCTTTTTCTTCCATCGGGAGATTCCACAGCCAGGCAG | (SEQ ID NO.: 126) |
| MLH1-15seq-as2: | TCCCCAACCCCCTAAAGCGATACCTCCATATGCAAATCATACAA | (SEQ ID NO.: 127) |

Exon 16

| | |
|---|---|
| 53581 | gcattagatg atttacctga aatgtcattc aatttaactt actctccatc ctcacccgcc |
| 53641 | cagctttggt tatgaggcag tagaaagaaa tgatctgcct gtggttttct agaaatacga |
| 53701 | aagttgagtc cttaaggcta cacagaaaga aagtacctcc ccagggcttc acccttccca |
| 53761 | tcctttcagc aggctttttg tctgtcgtat cttctctgtt gaaatggcca ttgacaagag |
| 53821 | gaggaaaggg gttttgttgt ggattgttca ggcacttcct ttggggtata tgggggatga |
| 53881 | gtgttacatt tatggtttct cacctgccat tctgatagtg gattcttggg aattcaggct |
| 53941 | tcatttggat gctccgttaa agcttgctcc ttcatgttct tgcttcttcc tagGAGCCAG |
| 54001 | CACCGCTCTT TGACCTTGCC ATGCTTGCCT TAGATAGTCC AGAGAGTGGC TGGACAGAGG |
| 54061 | AAGATGGTCC CAAAGAAGGA CTTGCTGAAT ACATTGTTGA GTTTCTGAAG AAGAAGGCTG |
| 54121 | AGATGCTTGC AGACTATTTC TCTTTGGAAA TTGATGAGgt gtgacagcca ttcttatact |
| 54181 | tctgttgtat tcttcaaata aaatttccag ccgggtgcgg tggctcatgg ctgtaatccc |
| 54241 | agcactttgg gaggctgagg tgggcagata acttggggtc aggagttcaa aaccagctgg |
| 54301 | ccaacatgat gaaaccccgt ctctactaaa aaaatagaaa aattagccag gcgtggtggc |
| 54361 | gggtacctgt aatccaagct gctcaggagg ctgaggcaga agaatcactt aaacccaaga |
| 54421 | ggtagaagtt gcagtgagcc gagattgcac cactgcactc tagcctaggc gacagcgaga |
| 54481 | ctgcgtctca aaaaaaaaaa aaaagaacgt tccaaggtca ggactaggcc tcccctcaga |

(SEQ ID NO.: 128)

| | | |
|---|---|---|
| MLH1-16A-s: | 5′ (*)-GCCATTCTGATAGTGGA 3′ | (SEQ ID NO.: 129) |
| MLH1-16A-as2: | 5′ TCTAAGGCAAGCATGGCAA | (SEQ ID NO.: 130) |
| MLH1-16B-s: | 5′ GCACCGCTCTTTGA 3′ | (SEQ ID NO.: 131) |
| MLH1-16B-as: | 5′ (*)-GTATAAGAATGGCTGTCA 3′ | (SEQ ID NO.: 132) |
| MLH1-16C-s2: | 5′ GGCTGAGATGCTTGCAG 3′ | (SEQ ID NO.: 133) |
| MLH1-16C-as2: | 5′ (*)-CATGAGCCACCGCAC 3′ | (SEQ ID NO.: 134) |
| MLH1-16seq-s: | TCTGCCTTTTTCTTCCATCGGGGGTTTTGTTGTGGATTGTTCAGG | (SEQ ID NO.: 135) |
| MLH1-16seq-as: | TCCCCAACCCCCTAAAGCGATGGGATTACAGCCATGAGCC | (SEQ ID NO.: 136) |

Exon 17

| | |
|---|---|
| 54661 | gagccgaatc cctgcaggcc attataaatg agattatgcc atttgctccc atttcttctt |
| 54721 | attctttcat ttttggggct ctccatcttg atgtgttctt tggatcgtga acagatccaa |
| 54781 | agaaaaggtt gttctgccgt gctgtttgtc aggatgaaaa actctttttt aagtgtttag |
| 54841 | gtctgccccc agtgcccagc ccaatcaagt aacgtggtca cccagagtgg cagataggag |
| 54901 | cacaaggcct gggaaagcac tggagaaatg ggatttgttt aaactatgac agcattattt |
| 54961 | cttgttccct tgtccttttt cctgcaagca gGAAGGGAAC CTGATTGGAT TACCCCTTCT |
| 55021 | GATTGACAAC TATGTGCCCC CTTTGGAGGG ACTGCCTATC TTCATTCTTC GACTAGCCAC |
| 55081 | TGAGgtcagt gatcaagcag atactaagca tttcggtaca tgcatgtgtg ctggagggaa |
| 55141 | agggcaaatg accacccttt gatctggaat gataaagatg ataagggtgg gatagctgaa |
| 55201 | ggcctgctct catccccact aatattcatt cccagcaata ttcagcagtc ccatttacag |
| 55261 | ttttaacgcc taaagtatca catttcgttt tttagcttta agtagtctgt gatctccgtt |
| 55321 | tagaatgaga atgtttaaat tcgtacctat tttgaggtat tgaatttctt tggaccaggt |
| 55381 | gaattgggac gaagaaaagg aatgttttga aagcctcagt aaagaatgcg ctatgttcta |
| 55441 | ttccatccgg aagcagtaca tatctgagga gtcgaccctc tcaggccagc aggtacagtg |
| 55501 | gtgatgcaca ctggcacccc aggactagga caggacctca tacaatcttt aggagatgaa |

(SEQ ID NO.: 137)

| | | |
|---|---|---|
| MLH1-17-s: | 5′ (*)-TGTTTAAACTATGACAGCA 3′ | (SEQ ID NO.: 138) |
| MLH1-17-as: | 5′ TGGTCATTTGCCCTT 3′ | (SEQ ID NO.: 139) |
| MLH1-17seq-s: | TCTGCCTTTTTCTTCCATCGGGTTTAAGTGTTTAGGTCTGCCCC | (SEQ ID NO.: 140) |
| MLH1-17seq-as: | TCCCCAACCCCCTAAAGCGAGCTATCCCACCCTTATCATCTTT | (SEQ ID NO.: 141) |

Exon 18

| | |
|---|---|
| 54661 | gagccgaatc cctgcaggcc attataaatg agattatgcc atttgctccc atttcttctt |
| 54721 | attctttcat ttttggggct ctccatcttg atgtgttctt tggatcgtga acagatccaa |
| 54781 | agaaaaggtt gttctgccgt gctgtttgtc aggatgaaaa actctttttt aagtgtttag |
| 54841 | gtctgccccc agtgcccagc ccaatcaagt aacgtggtca cccagagtgg cagataggag |
| 54901 | cacaaggcct gggaaagcac tggagaaatg ggatttgttt aaactatgac agcattattt |
| 54961 | cttgttccct tgtccttttt cctgcaagca ggaagggaac ctgattggat taccccttct |
| 55021 | gattgacaac tatgtgcccc ctttggaggg actgcctatc ttcattcttc gactagccac |
| 55081 | tgaggtcagt gatcaagcag atactaagca tttcggtaca tgcatgtgtg ctggagggaa |
| 55141 | agggcaaatg accacccttt gatctggaat gataaagatg ataagggtgg gatagctgaa |
| 55201 | ggcctgctct catccccact aatattcatt cccagcaata ttcagcagtc ccatttacag |
| 55261 | ttttaacgcc taaagtatca catttcgttt tttagcttta agtagtctgt gatctccgtt |
| 55321 | tagaatgaga atgtttaaat tcgtacctat tttgaggtat tgaatttctt tggaccagGT |
| 55381 | GAATTGGGAC GAAGAAAAGG AATGTTTTGA AAGCCTCAGT AAAGAATGCG CTATGTTCTA |
| 55441 | TTCCATCCGG AAGCAGTACA TATCTGAGGA GTCGACCCTC TCAGGCCAGC AGgtacagtg |
| 55501 | gtgatgcaca ctggcacccc aggactagga caggacctca tacaatcttt aggagatgaa |
| 55561 | acttgcccat ctctaaaatt tcgggatttc tttgtaccca acaaggttca aacacaacag |
| 55621 | tcagctttta ttcatgattt ttacttccat ctgctgatgt agaacatacc tccagagtga |
| 55681 | cctcagaaat tgtcaaatgt gaaaacacaa gccatcacag tgagaaatgg gaggttgagt |
| 55741 | tagattgtct aaggctggag agtccatata ctcccactgt tagctctgaa gtgtgtagcc |
| 55801 | agtcttcaga ttctgggtca gttgcctcag tctctcttag cttttgcctt actctttatc |
| 55861 | cgaccactgc cctgccagga aaacaaggct ctataactcc tcttacaggt cagcttgaca |

(SEQ ID NO.: 142)

| | | |
|---|---|---|
| MLH1-18A-s: | 5′ (*)-TGTGATCTCCGTTTAGAA 3′ | (SEQ ID NO.: 143) |
| MLH1-18A-as2: | 5′ CTGAGAGGGTCGACTCC | (SEQ ID NO.: 144) |
| MLH1-18B-s3: | (*)-TGCGCTATGTTCTATTCCA 3′ | (SEQ ID NO.: 145) |
| MLH1-18B-as3: | 5′ GCCGCCCCCGCCCGCTAGTCCTGGGGTGCCA 3′ | (SEQ ID NO.: 146) |
| MLH1-18seq-s: | TCTGCCTTTTTCTTCCATCGGGAAGATGATAAGGGTGGGATAGC | (SEQ ID NO.: 147) |
| MLH1-18seq-as: | TCCCCAACCCCCTAAAGCGACCGAAATTTTAGAGATGGGC | (SEQ ID NO.: 148) |

Exon 19

| | |
|---|---|
| 56461 | tacttcctac agttgccatc caaatatcag tcaggatcag acatgatgtt agctcctgct |
| 56521 | acaataaaac cattttctcc ctgaatgaaa acaaaggttc cacaggagac agtcccacag |
| 56581 | agcagtggct tcttttcctc cctttaaaac ctcatgttgg ctggacacag tggctcacac |
| 56641 | ctgtaatccc agcattttag gaggctgagg tgggaagatg gcttaagccc aggagtttga |
| 56701 | ggctgtagag ctatgatcac accactgccc ttcagcctgg gtgacagagc aagaccttgt |
| 56761 | ctctaaataa acaaacaaac aaaaaatcct cttgtgttca ggcctgtggg atcccctgag |
| 56821 | aggctagccc acaagatcca cttcaaaagc cctagataac accaagtctt tccagaccca |
| 56881 | gtgcacatcc catcagccag gacaccagtg tatgttggga tgcaaacagg gaggcttatg |
| 56941 | acatctaatg tgttttccag AGTGAAGTGC CTGGCTCCAT TCCAAACTCC TGGAAGTGGA |
| 57001 | CTGTGGAACA CATTGTCTAT AAAGCCTTGC GCTCACACAT TCTGCCTCCT AAACATTTCA |
| 57061 | CAGAAGATGG AAATATCCTG CAGCTTGCTA ACCTGCCTGA TCTATACAAA GTCTTTGAGA |
| 57121 | GGTGTTAAat atggttattt atgcactgtg ggatgtgttc ttctttctct gtattccgat |
| 57181 | acaaagtgtt gtatcaaagt gtgatataca aagtgtacca acataagtgt tggtagcact |
| 57241 | taagacttat acttgccttc tgatagtatt cctttataca cagtggattg attataaata |
| 57301 | aatagatgtg tcttaacata ATTTCTTATTTAATTTTATTATGTATATATTGTGTCAGTTCAG |
| | ATGCCAAAAAGAGGTCTTGAACATGTCACAGGCTCTGATGGCACTGACCATGGAGAAAGCT |

(SEQ ID NO.: 149)

| | | |
|---|---|---|
| MLH1-19A-s: | 5′ CAAGTCTTTCCAGACCC 3′ | (SEQ ID NO.: 150) |
| MLH1-19A-as: | 5′ (*)-TGTATAGATCAGGCAGGT 3′ | (SEQ ID NO.: 151) |
| MLH1-19C-s: | 5′ (*)-CAGAAGATGGAAATATCCTGC 3′ | (SEQ ID NO.: 152) |
| MLH1-19C-as: | 5′ (need 8 GC's)-TGTATATCACACTTTGATACAACACT3′ | (SEQ ID NO.: 153) |
| MLH1-19B-s4 | AAGCCTTGCGCTCACAC | (SEQ ID NO.: 154) |
| MLH1-19B-as4 | (*)-AATAACCATATTTAACACCTCTCAA | (SEQ ID NO.: 155) |
| MLH1-19seq-s: | TCTGCCTTTTTCTTCCATCGGGGCTATGATCACACCACTGCCC | (SEQ ID NO.: 156) |
| MLH1-19seq-as: | TCCCCAACCCCCTAAAGCGACCTCTTTTTGGCATCTGAACTG | (SEQ ID NO.: 157) |

hMSH2
genomic seq. and primers

5′upstream region
tgttttcgaatgagtgaatcatcaacgagtggatgaaacgataatgtggctaacaggcagcagtaaggagg
ctgtgtagaataaacccgtaatcccgatgttggcagtttgcttagaaagaaaaagggaggcagtcggagag
gggcacacgttttaacaaaatactgggaggaggaggaaggctagttttttttttgttttcaagtttccttc
tgatgttactcccatgcttccgggcacattacgagctcagtgcctgccggaaatctcccacctggtggcaa
cctacccttgcatacaccccacccaggggcttcaagccttgcagctgagtaaacacagaaaggagctctac
taaggatgcgcgtctgcgggtttccgcgcgacctaggcgcaggcatgcgcagtagctaaagtcaccagcgt
gcgcgggaagctgggccgcgtctgcttatgattggttgccgcggcagactcccacccaccgaaacgcagcc
ctggaagctgattgggtgtggtcgccgtggccggacgccgctcgggggacgtgggaggggaggcgggaaac
(SEQ ID NO.: 158)
Exon 1

| | |
|---|---|
| 1 | ggcgggaaac agcttagtgg gtgtggggtc gcgcattttc ttcaaccagg aggtgaggag |
| 61 | gtttcgacAT GGCGGTGCAG CCGAAGGAGA CGCTGCAGTT GGAGAGCGCG GCCGAGGTCG |
| 121 | GCTTCGTGCG CTTCTTTCAG GGCATGCCGG AGAAGCCGAC CACCACAGTG CGCCTTTTCG |
| 181 | ACCGGGGCGA CTTCTATACG GCGCACGGCG AGGACGCGCT GCTGGCCGCC CGGGAGGTGT |
| 241 | TCAAGACCCA GGGGGTGATC AAGTACATGG GGCCGGCAGg tgagggccgg gacggcgcgt |
| 301 | gctggggagg gacccggggc cttgtggcgc ggctcctttc ccgcctcaga gagtgggcgg |
| 361 | tgagcagcct ctccagtgcg gaggcacggg ggcggaacgt tggtgcttgt gcggattccg |
| 421 | ccgtccccag gttctgcttg gctccggagg gacgcccccc tcagccctga aacccgtgcc |
| 481 | tctccagccg ccccggatct gaacttgtga tcacggagtg tttacgtcgt gccaggcatt |
| 541 | ttaatgcatt gttctagttc attttccagc agtcgcattc ctcgccttgg ccctacatgt |
| 601 | agcgctcatt acaaacacgg ccagaatctc ttattaacaa acagcagcca ggagtgagat |
| 661 | ttaaaataga ctgggggttt aggagaccct tttatgacac gtaattctgc tcccacgacg |
| 721 | ctcccattta taccgccggt ccagctaagg gtctggtaat ggagcgccgt tgaagagcag |
| 781 | tatgatgaag tggtcaggac caacggactc tggagctggg ctgcttggga tcaagtcgct |
| 841 | gcccctctgc ttattaacgt gtgaccttgg gccagtcatg gacgctatct gcttcagctc |
| 901 | agcattcagt gctctccgtc acccgacccc atctatccag gattatctct ccctggaaag |
| 961 | ctacaaacgt ctcaccctat gtgggccaaa tgttctggat aggcctagtt aacctcttct |

| | | |
|---|---|---|
| (SEQ ID NO.: 159) | | |
| MSH2-1seq-s | TCTGCCTTTTTCTTCCATCGGGGGCGGGAAACAGCTTAGTGG | (SEQ ID NO.: 160) |
| MSH2-1seq-as | TCCCCAACCCCCTAAAGCGACGCACTGGAGAGGCTGCTCA | (SEQ ID NO.: 161) |

ALTERNATIVE FCTL SEQ PRIMER SET:

| | | |
|---|---|---|
| MSH2-1seq-s2 | TCTGCCTTTTTCTTCCATCGGGGCGCAGTAGCTAAAGTCACCAG | (SEQ ID NO.: 162) |
| MSH2-1seq-as2 | TCCCCAACCCCCTAAAGCGAGAATCCGCACAAGCACCAAC | (SEQ ID NO.: 163) |
| Exon 2 | | |

| | |
|---|---|
| 4921 | gaattcccat gtattgtggg agggacctgg tgggagatag ttgaatcatg gggatggatc |
| 4981 | tttcccatgc tgttgtgata gtgaataagc ctcatgagat ctgatggttt taaaaacgga |
| 5041 | agtctacctg cacaagctct ttctttgcct gctgccatcc atgtaagaca tgacttgttc |
| 5101 | ctccttgcct tctgccatga ttgtgagacc tccccagcca tgtggaacta taagtccagt |
| 5161 | aagcctcttt ttcttcccag tctcgggtat gtctttatca gcagcatgaa gtccagctaa |
| 5221 | tacagtgctt gaacatgtaa tatctcaaat ctgtaatgta cttttttttt ttttaagGAG |
| 5281 | CAAAGAATCT GCAGAGTGTT GTGCTTAGTA AAATGAATTT TGAATCTTTT GTAAAAGATC |
| 5341 | TTCTTCTGGT TCGTCAGTAT AGAGTTGAAG TTTATAAGAA TAGAGCTGGA AATAAGGCAT |
| 5401 | CCAAGGAGAA TGATTGGTAT TTGGCATATA AGgtaattat cttccttttt aatttactta |
| 5461 | tttttttaag agtagaaaaa taaaaatgtg aagaatttaa ttgtgtttta gtattttaag |
| 5521 | tagattgtga tagtagaatg gtttgagaca ctttaatagc aattagcatg tggtttttaa |
| 5581 | aaagttgcag tttggctggt cgcagtggct catgcttgta atcccagtat tttgggaggc |
| 5641 | tgaggcaggt aggttgcctg agcccaggag ttcaagacca gcctgcccaa cgtggtaaag |
| 5701 | ccccatctct actgaagata aaaaaattta aaaaaattag ctggggctat tggcacacac |
| 5761 | ctgtggtccc agctaatcaa gaggatgagg ttagaggatc acttgagccc aggaggttga |
| 5821 | ggttacagtt taactttcag aggccaaggc aggaggattg cttgagtcca ggagtttgag |
| 5881 | accaccctgg ggaatgtagg gagatcccat ctctatagag ggatagatta gatagataat |
| 5941 | ttctgagggg aggggagggg gagggccagg gaaggggagg gaaaggggag gggagggcag |

| | | |
|---|---|---|
| (SEQ ID NO.: 164) | | |
| MSH2-2C-s: | 5′ ATAAGGCATCCAAGGAGAA 3′ | (SEQ ID NO.: 165) |
| MSH2-2C-as: | 5′ (*)-ATCTACTTAAAATACTAAAACACAAT 3′ | (SEQ ID NO.: 166) |
| MSH2-2B-s3 | (*)-GGAGCAAAGAATCTGCAGAG | (SEQ ID NO.: 167) |
| MSH2-2B-as3 | TAATTACCTTATATGCCAAATACCA | (SEQ ID NO.: 168) |
| MSH2-2seq-s2 | TCTGCCTTTTTCTTCCATCGGGTGCTGCCATCCATGTAAGAC | (SEQ ID NO.: 169) |
| MSH2-2seq-as2 | TCCCCAACCCCCTAAAGCGACCAGCCAAACTGCAACTTTT | (SEQ ID NO.: 170) |

ALTERNATIVE FCTL SEQ PRIMER SET:

| | | |
|---|---|---|
| MSH2-2seq-s3 | TCTGCCTTTTTCTTCCATCGGGTTCCTCCTTGCCTTCTGCCAT | (SEQ ID NO.: 171) |
| MSH2-2seq-as3 | TCCCCAACCCCCTAAAGCGAGGGATTACAAGCATGAGCCACTG | (SEQ ID NO.: 172) |

Exon 3
ccctggttcaagcttttctcccgcctcagcctcccgagtagctgggattacaggtgcatgctgcaacaccc
ggctaatttttgtatttttagtagagatggggtttcaccatgttggccaggacggtctcgatctcctgacc
tcgtgatccgcctgccttggcctcccaaagtgttgggattacaggcgtgagccacagcactcagccagtta
tttttttataagaaaacattttactggccaggcctggtggctcacacctgtaatcccagcactttgggagg
ccgaggcaggcggatcacgaggtcaggagttcgagaccagcctggccaacatggtgaaaccccatctctac
taaaaatacaaaaattagccaggcgtggtggtgtgcgcctgtattcccagctactggggaggctgaagcag
gagaatcgattgaacccttgaggcagaggttgcagtgagttgagatcgcaccattgcactctagcctgggt
gacagagcaagacttcatctcaaaaaaaagagaaaacattttattaataaggttcatagagtttggatttt
tcctttttgcttataaaattttaaagtatgttcaagagtttgttaaatttttaaaattttatttttactta
gGCTTCTCCTGGCAATCTCTCTCAGTTTGAAGACATTCTCTTTGGTAACAATGATATGTCAGCTTCCATTG
GTGTTGTGGGTGTTAAAATGTCCGCAGTTGATGGCCAGAGACAGGTTGGAGTTGGGTATGTGGATTCCATA
CAGAGGAAACTAGGACTGTGTGAATTCCCTGATAATGATCAGTTCTCCAATCTTGAGGCTCTCCTCATCCA
GATTGGACCAAAGGAATGTGTTTTACCCGGAGGAGAGACTGCTGGAGACATGGGGAAACTGAGACAGgtaa
gcaaattgagtctagtgatagaggagattccaggcctaggaaaggctctttaattgacatgatactgtttc
atttaaggaaaaataataaaaaaactcttttttttgtatctaattaaaataatgttctgatgtttacagaa
actttgtatatttaattggacattagaacaagctgtttgttgtgtaagatttattttacctcagatctttt
ctcccccctttcctttctgtcttgtgttccaaaagagtaattattacggtaaatattactgtaattatgga
tttatcaaataagatgcagttctttagcattttttgataaatcgagtggaactttagcctgttattttact
atttgttttattttaa (SEQ ID NO.: 173)

| | | |
|---|---|---|
| MSH2-3A-s: | 5′ (*)-AACATTTTATTAATAAGGTTC 3′ | (SEQ ID NO.: 174) |
| MSH2-3A-as: | 5′ ATTGCCAGGAGAAGC 3′ | (SEQ ID NO.: 175) |
| MSH2-3B-s2: | 5′ (*)-ATTTTTACTTAGGCTTCTCCTG 3′ | (SEQ ID NO.: 176) |
| MSH2-3B-as2: | 5′ CAGTTTCCCCATGTCTCC 3′ | (SEQ ID NO.: 177) |
| MSH2-3C-s: | 5′ AATGTGTTTTACCCGGAG 3′ | (SEQ ID NO.: 178) |
| MSH2-3C-as: | 5′ (*)-CTTAAATGAAACAGTATCATGTC 3′ | (SEQ ID NO.: 179) |
| MSH2-3seq-s4 | TCTGCCTTTTTCTTCCATCGGGGGTTCATAGAGTTTGGATTTTTCC | (SEQ ID NO.: 180) |
| MSH2-3seq-as4 | TCCCCAACCCCCTAAAGCGACCTTAAATGAAACAGTATCATGTCAA | (SEQ ID NO.: 181) |
| Exon 4 | | |

| | |
|---|---|
| 7501 | gtggcttgct cctgtaatcc tagctacttg ggaggctgag gcaggagaat tgcttgaacc |
| 7561 | tgggaggcag aggtagcagt gagccaagat cgtgtcaccg cattccatcc tgggcgacag |
| 7621 | tgagactctg tctcaaaaca aaaaaagagt tgttaccgtt gggactattt tttgaaagct |
| 7681 | ttatgtgaac gtaattttat attttgatga aaatttagtt tattgatgta aaaagtgtat |
| 7741 | cagtacatca tatcagtgtc ttgcacattg tataaacatt taatgtaggt gaatctgtta |
| 7801 | tcactatagt tatcaatgtt ataattttca tttttgcttt tcttattcct tttctcatag |
| 7861 | tagtttaaac tatttctttc aaaatagATA ATTCAAAGAG GAGGAATTCT GATCACAGAA |
| 7921 | AGAAAAAAAG CTGACTTTTC CACAAAAGAC ATTTATCAGG ACCTCAACCG GTTGTTGAAA |
| 7981 | GGCAAAAAGG GAGAGCAGAT GAATAGTGCT GTATTGCCAG AAATGGAGAA TCAGgtacat |
| 8041 | ggattataaa tgtgaattac aatatatata atgtaaatat gtaatatata ataaataata |
| 8101 | tgtaaactat agtgactttt tagaaggata tttctgtcat atttatctca aaacctaaac |
| 8161 | tgtgtatcaa tgatattaag cttttttttt tttttgagac agagtttcac ttttgttgcc |
| 8221 | caggctggag tacaatggcg cgatcttggc tcaccacatc ctctgcctcc caggttcaag |
| 8281 | tgatcctcct gccttggcct cctgagtagc tgggattaca ggcatgtgcc accacgcctg |
| 8341 | gctcatcttt tttgtatttt tagtagagat ggggtttctc tatgttggtc aggctggtct |
| 8401 | caaactcctg aacctcaggt gatccgcccg cctcgggctt ccaaagcgct gagattgcag |
| 8461 | gcatgagcca ctgtgtctgg cctattttta tagtttatgt acttggaatt atataatata |

| | | |
|---|---|---|
| (SEQ ID NO.: 182) | | |
| MSH2-4A-s: | 5′ (*)-TCCTTTTCTCATAGTAGTTTA 3′ | (SEQ ID NO.: 183) |
| MSH2-4A-as: | 5′ TTGAGGTCCTGATAAATG 3′ | (SEQ ID NO.: 184) |
| MSH2-4A-s2: | 5′ (*)-TTTCTTTCAAAATAGATAATTC 3′ | (SEQ ID NO.: 185) |
| MSH2-4A-as2: | 5′ TTTTTGCCTTTCAACA 3′ | (SEQ ID NO.: 186) |
| MSH2-4B-2s: | 5′ ATTTATCAGGACCTCAA 3′ | (SEQ ID NO.: 187) |
| MSH2-4B-2as: | 5′ (*)-TGTAATTCACATTTATAATC 3′ | (SEQ ID NO.: 188) |
| MSH2-4C-s: | 5′ ATTGCCAGAAATGGAG 3′ | (SEQ ID NO.: 189) |
| MSH2-4C-as: | 5′ (*)-ACATATTTACATTATATATATTGT 3′ | (SEQ ID NO.: 190) |
| MSH2-4seq-s2: | TCTGCCTTTTTCTTCCATCGGGgcattccatcctgggcga | (SEQ ID NO.: 191) |
| MSH2-4seq-as2: | TCCCCAACCCCCTAAAGCGACAGCCTGGGCAACAAAAGTG | (SEQ ID NO.: 192) |
| Exon 5 | | |

| | |
|---|---|
| 9361 | agagacgggg tttcactatg ttggctaggc tggtctcaaa ctcctagcct cgagtcatcc |
| 9421 | acccgcctcg tcctcccgga gtgcttggat tacagcatga gccactgcgc ccggccccca |
| 9481 | ttttagtttt gatggacatt tgggtaattt tcttttttgg ctattctaaa taatgctgca |
| 9541 | attactgtta attttcacct tgtaaaaacc attttcaaat ctcaagagat taacctttag |
| 9601 | ttttcttggt ttggattggg aaggaacacc aaggaaaatg agggacttca gaatttattt |
| 9661 | tcattttgca tttgtttttt aaaatcttta gaactggatc cagtggtata gaaatcttcg |
| 9721 | atttttaaat tcttaatttt agGTTGCAGT TTCATCACTG TCTGCGGTAA TCAAGTTTTT |
| 9781 | AGAACTCTTA TCAGATGATT CCAACTTTGG ACAGTTTGAA CTGACTACTT TTGACTTCAG |
| 9841 | CCAGTATATG AAATTGGATA TTGCAGCAGT CAGAGCCCTT AACCTTTTTC AGgtaaaaaa |
| 9901 | aaaaaaaaaa aaaaaaaaaa agggttaaaa atgttgaatg gttaaaaaat gttttcattg |
| 9961 | acatatactg aagaagctta taaaggagct aaaatatttt gaaatattat tatacttgga |
| 10021 | ttagataact agctttaaat ggctgtattt ttctctcccc tcctccactc cactttttaa |
| 10081 | cttttttttt tttaagtcag agtctcactt gttccctagg ccagagtgca gtggcacaat |
| 10141 | ctcagcccac tctaacctcc acctcccaag tagttgggat tacagttgcc tgccaccatg |
| 10201 | cctggttaat ttttatattt ttagtagggt tgcggggaca gggtttcacc atgttggcca |
| 10261 | ggttggtctc aaacttctga ccttaggtga tcctcccacc tcggcttccc aaagtgctgg |
| 10321 | gattacaggc ttgagccatc gtgcccagcc tactttttac ttttttagag actgggcttg |

| | | |
|---|---|---|
| (SEQ ID NO.: 193) | | |
| MSH2-5A-s: | 5′ (*)-TTCATTTTGCATTTGTT 3′ | (SEQ ID NO.: 194) |
| MSH2-5A-as: | 5′ CTTGATTACCGCAGAC 3′ | (SEQ ID NO.: 195) |
| MSH2-5B-s: | 5′ (*)-ATCTTCGATTTTTAAATTC 3′ | (SEQ ID NO.: 196) |
| MSH2-5B-as: | 5′ AAAGGTTAAGGGCTCTG 3′ | (SEQ ID NO.: 197) |
| MSH2-5seq-s2: | TCTGCCTTTTTCTTCCATCGGGTTCTTGGTTTGGATTGGGAAGG | (SEQ ID NO.: 198) |
| MSH2-5seq-as2: | TCCCCAACCCCCTAAAGCGAGGGGAGAGAAAAATACAGCCAT | (SEQ ID NO.: 199) |

ALTERNATIVE FCTL SEQ PRIMER SET:

| | | |
|---|---|---|
| MSH2-5seq-s3: | TCTGCCTTTTTCTTCCATCGGGAGTTTTGATGGACATTTGGGTAA | (SEQ ID NO.: 200) |
| MSH2-5seq-as3: | TCCCCAACCCCCTAAAGCGAGTTAAAAAGTGGAGTGGAGGAGG | (SEQ ID NO.: 201) |
| Exon 6 | | |

| | |
|---|---|
| 11101 | atggggtttc atcttgttgg ctaggctgga ctctaactcc aggtgatctg cctgcctcgg |
| 11161 | cctcccaaat tgatgggatt acaggtgtaa accactgggc ctggcctagc aatttaaaat |
| 11221 | gacattctaa gaagttttat gtctaaatct gcagtaagtg gctgggtgac gtggctcatg |
| 11281 | cctgtaatcc caacgctttg ggagtccagg gtgggaggat gacttgaggc caggagttga |
| 11341 | gaccagcctg ggcaacatag tgagactctg tctctacaaa agaaaaaatt agcggggctt |
| 11401 | agtggcgtgc gcctgtagtc tcagctactc gaaaggctga agtgggagga ttctttgagc |
| 11461 | cccaagggtt ctggcttgcc gtgagccagg atggcaccac tgcactccag tctgggcaat |
| 11521 | agagtcagac cctgtctcaa caaataaaat aaaactgtag taattataaa gtggttttgg |
| 11581 | ctgggggaga aatgtacagt tgaacatacg gattaagagg ttgaaagttg gtcttaggaa |
| 11641 | gaggaacttt ttgtggaaat ttcttaatat ttgaagaata ttatgttatt gttcctctgt |
| 11701 | ttttcatggc gtagtaaggt tttcactaat gagcttgcca ttctttctat tttatttttt |
| 11761 | gtttactagG GTTCTGTTGA AGATACCACT GGCTCTCAGT CTCTGGCTGC CTTGCTGAAT |
| 11821 | AAGTGTAAAA CCCCTCAAGG ACAAAGACTT GTTAACCAGT GGATTAAGCA GCCTCTCATG |
| 11881 | GATAAGAACA GAATAGAGGA GAGgtatgtt attagtttat actttcgtta gttttatgta |
| 11941 | acctgcagtt acccacatga ttataccact tattgtaata tgcagttttg gaagtatatg |
| 12001 | ttaccattta actgtacaga gtacatagta atagagtggt aattatttag attgattaaa |
| 12061 | gaactcattt ttttaaataa gttttttttt tttcactata aaagtttatt ttatttgaga |
| 12121 | tggtatggta tcgaacatgt tcatattgtg tgtaatcgtg ggtaaattac tcaaccttta |
| 12181 | tgtcatagtt tcttcacctt taaaatgaca ttaataaaag agctacttaa taggattata |
| 12241 | agcatgagat gatttaatat acataaaata cttacagtct gatatatagg aagcacttaa |
| 12301 | ctctttatcc tagaaaagat ttaaggtgac cttaacatat atgtcagaaa atctttaaaa |
| 12361 | ttgtggaaat aaaaggttgt ataattctgc tatcctaaaa ttactagtat ttcaatatat |

| | | |
|---|---|---|
| (SEQ ID NO.: 202) | | |
| MSH2-6A-s: | 5′ (*)-GTTTTTCATGGCGTAG 3′ | (SEQ ID NO.: 203) |
| MSH2-6A-as: | 5′ ACTGAGAGCCAGTGGTA 3′ | (SEQ ID NO.: 204) |
| MSH2-6B-s2: | 5′ TTTACTAGGGTTCTGTTGAAGA | (SEQ ID NO.: 205) |
| MSH2-6B-as: | 5′ (*)-ATACCTCTCCTCTATTCTG 3′ | (SEQ ID NO.: 206) |
| MSH2-6C-s: | 5′ TCAAGGACAAAGACTTGT 3′ | (SEQ ID NO.: 207) |
| MSH2-6C-as: | 5′ (*)-CATATTACAATAAGTGGTATAAT 3′ | (SEQ ID NO.: 208) |
| MSH2-6seq-s: | TCTGCCTTTTTCTTCCATCGGGTGAACATACGGATTAAGAGG | (SEQ ID NO.: 209) |
| MSH2-6seq-as: | TCCCCAACCCCCTAAAGCGACATATACTTCCAAAACTGCA | (SEQ ID NO.: 210) |
| Exon 7 | | |

| | |
|---|---|
| 24181 | ttttttttga gacagagtct tgctcttgtt gcccaggctg gagtgccatg gcatgatctc |
| 24241 | agtgcaccac aatctctgct tcccaggttt aagcgattct cctgcctcag cctcccaagt |
| 24301 | agatgggatc acaggcatga gccaccatgc ctggctaatt ttgtattttt tgtacagacg |
| 24361 | gggtttctcc atgttggtca ggccagtctc gaactcccta cctcaggtga tctgcctgcc |
| 24421 | tcggcctctc aaagtgctgg gattacaggt gtgagccact gcgcccagca gattcaagct |
| 24481 | ttttaaatgg aattttgagc tgatttagtt gagacttacg tgcttagttg ataaatttta |
| 24541 | attttatact aaaatatttt acattaattc aagttaattt atttcagATT GAATTTAGTG |
| 24601 | GAAGCTTTTG TAGAAGATGC AGAATTGAGG CAGACTTTAC AAGAAGATTT ACTTCGTCGA |
| 24661 | TTCCCAGATC TTAACCGACT TGCCAAGAAG TTTCAAAGAC AAGCAGCAAA CTTACAAGAT |
| 24721 | TGTTACCGAC TCTATCAGGG TATAAATCAA CTACCTAATG TTATACAGGC TCTGGAAAAA |
| 24781 | CATGAAGgta acaagtgatt ttgttttttt gttttccttc aactcataca atatatactt |
| 24841 | ggcaatgtgc tgtcctcata aagttggtgg tggtgactca ctcttaggac acattcagat |
| 24901 | ttcttttttt tttttttttg agaaggagtc ttgctccgtt gccaaggcta gagtgcagtg |
| 24961 | gcacaatctc agctcactgc aacctctgcc tcctgggttc aagcgattct cctgcctcag |
| 25021 | cttcctgagt ggctgggatt acaggcatgt gccaccatgc ccggctaatt tttgtacttt |
| 25081 | tagttttacc atgttggcca ggttcgtctg gaactcccaa tctcaggtga cccacctgcc |

| | | |
|---|---|---|
| (SEQ ID NO.: 211) | | |
| MSH2-7A-s: | 5′ (*)-GTTGAGACTTACGTGCTT 3′ | (SEQ ID NO.: 212) |
| MSH2-7A-as2: | 5′ CAATTCTGCATCTTCTACAAA | (SEQ ID NO.: 213) |
| MSH2-7B-s2: | 5′ (*)-ATTTCAGATTGAATTTAGTGG 3′ | (SEQ ID NO.: 214) |
| MSH2-7B-as2: | 5′ AGTTTGCTGCTTGTCTTTG 3′ | (SEQ ID NO.: 215) |
| MSH2-7C-s3: | 5′ GACTTGCCAAGAAGTTT 3′ | (SEQ ID NO.: 216) |
| MSH2-7C-as2: | 5′ (*)-TGAGTCACCACCACCAAC 3′ | (SEQ ID NO.: 217) |
| MSH2-7seq-s3: | TCTGCCTTTTTCTTCCATCGGGGCTGATTTAGTTGAGACTTACGTGC | (SEQ ID NO.: 218) |
| MSH2-7seq-as2: | TCCCCAACCCCCTAAAGCGAGAGGACAGCACATTGCCAAG | (SEQ ID NO.: 219) |

Exon 8

| | |
|---|---|
| 40081 | tataagaaat gaaattcatt tagtcataat taatgtcatg tttctgcatc tatattactt |
| 40141 | gttgggttta cagacgaggt agtgtattat tagtgggaag ctttgagtgc tacatcatct |
| 40201 | ccctttctat aaaataaatt gagtacgaaa caatttgaat taaaacacct gagtaaatag |
| 40261 | taactttgga gacctgctgt actatttgta ccttttggat caaatgatgc ttgtttatct |
| 40321 | cagtcaaaat tttatgattt gtattctgta aaatgagatc tttttatttg tttgttttac |
| 40381 | tactttcttt tagGAAAACA CCAGAAATTA TTGTTGGCAG TTTTTGTGAC TCCTCTTACT |
| 40441 | GATCTTCGTT CTGACTTCTC CAAGTTTCAG GAAATGATAG AAACAACTTT AGATATGGAT |
| 40501 | CAGgtatgca atatactttt taatttaagc agtagttatt tttaaaaagc aaaggccact |
| 40561 | ttaagaaagt ttgtagattt ttctttttag tatctaattg tagcaccttt gtggacagtg |
| 40621 | gatgtaatat taagtgacag atgggaaaag gatttttaaa aaaatagcaa ctgtttcagt |
| 40681 | ggatgaaata aagattatta gcagagaaaa tgaatattgg gcataactgt cctggtgaaa |
| 40741 | gacaatctca taaatgaaca atttcataat ttcgtaaatg caactgcatt ttattttcaa |
| 40801 | agagaaggaa aattatagtc actggaaacg gaaagagaag ttagaggtaa acataggaca |
| 40861 | cacaagaaaa ctttcatttt gtttattttc ttgtttttct tttgagacag ggtttccctc |

| | | |
|---|---|---|
| (SEQ ID NO.: 220) | | |
| MSH2-8A-s: | 5′ (*)-TTTGGATCAAATGATGC 3′ | (SEQ ID NO.: 221) |
| MSH2-8A-as: | 5′ ATCAGTAAGAGGAGTCACA 3′ | (SEQ ID NO.: 222) |
| MSH2-8B-s: | 5′ TTGTGACTCCTCTTACTG 3′ | (SEQ ID NO.: 223) |
| MSH2-8B-as: | 5′ (*)-AATAACTACTGCTTAAATTAA 3′ | (SEQ ID NO.: 224) |
| MSH2-8C-s: | 5′ CTGACTTCTCCAAGTTTC 3′ | (SEQ ID NO.: 225) |
| MSH2-8C-as: | 5′ GTGCTACAATTAGATACTAAA 3′ | (SEQ ID NO.: 226) |
| MSH2-8D-s: | 5′ AGAAATTATTGTTGGCAGTT | (SEQ ID NO.: 227) |
| MSH2-8D-as: | 5′ (*)-ATTGCATACCTGATCCATATC | (SEQ ID NO.: 228) |
| MSH2-8seq-s: | TCTGCCTTTTTCTTCCATCGGGAATAGTAACTTTGGAGACCTGC | (SEQ ID NO.: 229) |
| MSH2-8seq-as: | TCCCCAACCCCCTAAAGCGACAGGACAGTTATGCCCAATA | (SEQ ID NO.: 230) |
| Exon 9 | | |

| | |
|---|---|
| 57541 | cacattgaac gttatttggt aatttttaga gaggacattt taaatgttta ggaaaaatat |
| 57601 | aaataaaatg tagaatacta ttgggggcat atacatcatc agcactgtaa ctgtttcata |
| 57661 | tgaatcattt ttgtacatat agaactctaa agtcctaatg aacagaattt tacatttcta |
| 57721 | taaatagaaa gtccttaata gttgtgactg aataacttat ggatagcaaa ttatttaact |
| 57781 | gaaaacagta aaatttaagt gggaggaaat atttgcttta taatttctgt ctttacccat |
| 57841 | tatttatagg attttgtcac tttgttctgt ttgcagGTGG AAAACCATGA ATTCCTTGTA |
| 57901 | AAACCTTCAT TTGATCCTAA TCTCAGTGAA TTAAGAGAAA TAATGAATGA CTTGGAAAAG |
| 57961 | AAGATGCAGT CAACATTAAT AAGTGCAGCC AGAGATCTTG gtaagaatgg gtcattggag |
| 58021 | gttggaataa ttcttttgtc tatacactgt atagacaaaa tattgatgcc agaattattt |
| 58081 | tataagttcc ctgtccccaa gatgatgact tcacatctct gtcaaacaga aatcgcccaa |
| 58141 | caggcccttg tatgatgtca tttaaacaag ccctatttta aatgtcacct ccactggtaa |
| 58201 | caggatactc ctaggaggat caccaagccc aattcttcta ggagtagtgc attgattagg |
| 58261 | ctttggggtt tccaagcagt tcattaatgt cacttttgga aaaagtctgt ctttcatacc |

| | | |
|---|---|---|
| (SEQ ID NO.: 231) | | |
| MSH2-9-s2: | 5′ (*)-AATATTTGCTTTATAATTTC 3′ | (SEQ ID NO.: 232) |
| MSH2-9-as2: | 5′ AGAATTATTCCAACCTC 3′ | (SEQ ID NO.: 233) |
| MSH2-9seq-s: | TCTGCCTTTTTCTTCCATCGGGGAAAGTCCTTAATAGTTGTGACTG | (SEQ ID NO.: 234) |
| MSH2-9seq-as: | TCCCCAACCCCCTAAAGCGAGGGAACTTATAAAATAATTCTGGC | (SEQ ID NO.: 235) |
| Exon 10 | | |

| | |
|---|---|
| 61141 | tcatgcataa ctcctcgagg gtggggttac accttaatcc atcctcaggt gctcatggta |
| 61201 | attggggcaa atatgttgcc cagtgctggt gctctgcagc cttggatggg tttacccaga |
| 61261 | aagcagcttt caagtcagaa actaacattc ataagggagt taaggatttt ataaatagat |
| 61321 | atccataatt catgtagttt tcaagtaagt agtatttgaa tcttttctgg ttagataata |
| 61381 | attgtgagta tgttgtcata taataacagt atgtttttca ctatttaaat aattttagaa |
| 61441 | ttacattgaa aaatggtagt aggtatttat ggaatacttt ttcttttctt cttgattatc |
| 61501 | aagGCTTGGA CCCTGGCAAA CAGATTAAAC TGGATTCCAG TGCACAGTTT GGATATTACT |
| 61561 | TTCGTGTAAC CTGTAAGGAA GAAAAAGTCC TTCGTAACAA TAAAAACTTT AGTACTGTAG |
| 61621 | ATATCCAGAA GAATGGTGTT AAATTTACCA ACAGgtttgc aagtcgttat tatattttta |
| 61681 | accctttatt aattccctaa atgctctaac atgatgtgaa tgttctatga taagttttac |
| 61741 | taatgtagtc atcaggtaag agtcaagctt tcttccatag agcagtcagc tgtcgcaaca |
| 61801 | ccatttgtta aatagtccgt ctgttctcca ttgactgaag tggtactttg ggtctatttt |
| 61861 | aaagactcta cttttacctc gtctcaccat tcttttgtct acacaaaata tattttatcg |

| | | |
|---|---|---|
| (SEQ ID NO.: 236) | | |
| MSH2-10A-s: | 5′ (*)-GAATTACATTGAAAAATGG 3′ | (SEQ ID NO.: 237) |
| MSH2-10A-as: | 5′ TTAATCTGTTTGCCAGG 3′ | (SEQ ID NO.: 238) |
| MSH2-10B-s2: | 5′ TCTTCTTGATTATCAAGGC 3′ | (SEQ ID NO.: 239) |
| MSH2-10B-as2: | 5′ (*)-ACACCATTCTTCTGGATA 3′ | (SEQ ID NO.: 240) |
| MSH2-10C-s3: | 5′ TGCACAGTTTGGATATTACTT 3′ | (SEQ ID NO.: 241) |
| MSH2-10C-as3: | 5′ (*)-GTAAAACTTATCATAGAACATTCAC 3′ | (SEQ ID NO.: 242) |
| MSH2-10seq-s: | TCTGCCTTTTTCTTCCATCGGGTCATAAGGGAGTTAAGGATTT | (SEQ ID NO.: 243) 494/536 |
| MSH2-10seq-as: | TCCCCAACCCCCTAAAGCGACTGCTCTATGGAAGAAAGCT | (SEQ ID NO.: 244) |
| Exon 11 | | |

| | |
|---|---|
| 65461 | gttctggggt tacaggcgtg agccaccacg cccggctgtc ttcaatctta aataaggatt |
| 65521 | ccatttaaat attttgtaaa aggacacaga tcacagtttt actcagggga atataattgt |
| 65581 | tatagcagga attgtgccat tgcgctattc caaacagtgt aaaagaacat taataaattg |
| 65641 | aattctaact acatttgtcc ctaaggagtt gttcgttttc cacttgtatt tccattttaa |
| 65701 | ttatcattat ttggatgttt cataggatac tttggatatg tttcacgtag tacacattgc |
| 65761 | ttctagtaca cattttaata tttttaataa aactgttatt tcgatttgca gCAAATTGAC |
| 65821 | TTCTTTAAAT GAAGAGTATA CCAAAAATAA AACAGAATAT GAAGAAGCCC AGGATGCCAT |
| 65881 | TGTTAAAGAA ATTGTCAATA TTTCTTCAGg taaacttaat agaactaata atgttctgaa |
| 65941 | tgtcacctgg cttttggtaa cagaagaaaa atcatgatat ttgaagtgtg ttttgttatt |
| 66001 | ttcgcaagcc attacattct gactatttaa tatgttaggt ttcctatata aaataaggca |
| 66061 | tggtatgtta cagtaggaca cataactgga agttactctt gcacatagaa acaaaaaatg |
| 66121 | gcagaaaagc acaaaactta ctatagttgt aacagggaaa ggaaacacta gggcctacaa |
| 66181 | cgtactaatg tcttgggtca tctatgggct catgaggctc taggttatgg aagtaaatac |

| | | |
|---|---|---|
| (SEQ ID NO.: 245) | | |
| MSH2-11A-s2: | 5′ TTTGGATATGTTTCACGTA 3′ | (SEQ ID NO.: 246) |
| MSH2-11A-as2: | 5′ CTTTAACAATGGCATCCT 3′ | (SEQ ID NO.: 247) |
| MSH2-11B-s2: | 5′ GCAAATTGACTTCTTTAAATG 3′ | (SEQ ID NO.: 248) |
| MSH2-11B-as2: | 5′ ATGGCTTGCGAAAATAAC 3′ | (SEQ ID NO.: 249) |
| MSH2-11seq-s: | TCTGCCTTTTTCTTCCATCGGGCATTTGTCCCTAAGGAGTTGTTC | (SEQ ID NO.: 250) |
| MSH2-11seq-as: | TCCCCAACCCCCTAAAGCGACAGAATGTAATGGCTTGCGA | (SEQ ID NO.: 251) |
| Exon 12 | | |

| | |
|---|---|
| 69361 | tgtggcgcaa tctcagctta ctgcaacttc caccttctgg gttcatgcaa ttctggtgcc |
| 69421 | tcagcctccc aagtatctgg gtttacagac atgcaccacc atacctggct aatttttgta |
| 69481 | tttttggtag agatggggtt tcgccgtgtt accaggctgg tcttgaattc ctggccccat |
| 69541 | gtgatccccc ggcctcatgc gatctgcccg cctcagcctc cctaagtgct gggattatag |
| 69601 | gcgtgagcca cccaacccag ccagtactct gtttttgata gctattcaca atgggaaagg |
| 69661 | atgtagcaac acattttaac cctatgttga gttttaggtg ggttcctttg aaattttgtt |
| 69721 | aaggctaact tttgttaatt tttttaaaaa agtgtaaatt aggaaatggg ttttgaattc |
| 69781 | ccaaatgggg ggattaaatg tatttttacg gcttatatct gtttattatt cagtattcct |
| 69841 | gtgtacattt tctgttttta tttttataca gGCTATGTAG AACCAATGCA GACACTCAAT |
| 69901 | GATGTGTTAG CTCAGCTAGA TGCTGTTGTC AGCTTTGCTC ACGTGTCAAA TGGAGCACCT |
| 69961 | GTTCCATATG TACGACCAGC CATTTTGGAG AAAGGACAAG GAAGAATTAT ATTAAAAGCA |
| 70021 | TCCAGGCATG CTTGTGTTGA AGTTCAAGAT GAAATTGCAT TTATTCCTAA TGACGTATAC |
| 70081 | TTTGAAAAAG ATAAACAGAT GTTCCACATC ATTACTGgta aaaaacctgg tttttgggct |
| 70141 | ttgtgggggt aacgttttgt tttttttttt ttttttttaa tcttggagta gaaatatatt |
| 70201 | taaaattgat ggagaaaatt cccagttctt aacattagaa agggaatata ttattcttac |
| 70261 | cagttagtaa tctattcaca tttggtttag agggaagatt tagaaggtga gataaaagct |
| 70321 | tgtgagagaa tagtgtattc atgtgaaact tcttccatgg gttcagagca tttagaaaca |
| 70381 | aacatccctt cacactcaaa gcttaccttt gagccagtcc tccaatagtg aggtctttga |
| 70441 | aggtcaggcc aaattggctg tgggaggacc tcaggttagg ataggaatta ttttaagaca |
| 70501 | tggcactata ttcatgtgaa actcgcaaaa actagccttg catataggct catgtatcat |
| 70561 | gtctcagctg agatgtttga gagatcttaa ctagattcta gaaaacaaaa aaggaagtag |

| | | |
|---|---|---|
| (SEQ ID NO.: 252) | | |
| MSH2-12A-s: | 5′ (*)-AGGAAATGGGTTTTGAA 3′ | (SEQ ID NO.: 253) |
| MSH2-12A-as: | 5′ GAGCTAACACATCATTGAGT 3′ | (SEQ ID NO.: 254) |
| MSH2-12B-s: | 5′ (*)-ATTTTTATACAGGCTATGTAG 3′ | (SEQ ID NO.: 255) |
| MSH2-12B-as: | 5′ ACATATGGAACAGGTGCT 3′ | (SEQ ID NO.: 256) |
| MSH2-12C-s: | 5′ TGGAGCACCTGTTCCAT 3′ | (SEQ ID NO.: 257) |
| MSH2-12C-as: | 5′ (*)-AACAAAACGTTACCCCC 3′ | (SEQ ID NO.: 258) |
| MSH2-12E-s: | 5′ CAGCTTTGCTCACGTGTCA | (SEQ ID NO.: 259) |
| MSH2-12E-as: | 5′ (*)-CATCTTGAACTTCAACACAAGC | (SEQ ID NO.: 260) |
| MSH2-12seq-s: | TCTGCCTTTTTCTTCCATCGGGTGTTGAGTTTTAGGTGGGTTCC | (SEQ ID NO.: 261) |
| MSH2-12seq-as: | TCCCCAACCCCCTAAAGCGATACCCCCACAAAGCCCAAA | (SEQ ID NO.: 262) |
| Exon 13 | | |

| | |
|---|---|
| 71041 | atgggcagta actctgtcca catctttggg caggctgtgg ttctgccttt atatgctatg |
| 71101 | tcagtgtaaa cctacgcgat taatcatcag tgtacagttt aggactaaca atccatttat |
| 71161 | tagtagcaga aagaagttta aaatcttgct ttctgatata atttgttttg tagGCCCCAA |
| 71221 | TATGGGAGGT AAATCAACAT ATATTCGACA AACTGGGGTG ATAGTACTCA TGGCCCAAAT |
| 71281 | TGGGTGTTTT GTGCCATGTG AGTCAGCAGA AGTGTCCATT GTGGACTGCA TCTTAGCCCG |
| 71341 | AGTAGGGGCT GGTGACAGTC AATTGAAAGG AGTCTCCACG TTCATGGCTG AAATGTTGGA |
| 71401 | AACTGCTTCT ATCCTCAGgt aagtgcatct cctagtccct tgaagataga aatgtatgtc |
| 71461 | tctgtcctgt gagaaggaaa agtatatttg cagattctca tgtaaaaaca tctgagaatg |
| 71521 | tttgtcttag tttaatagtt gttttcctgt ggactttata tactttgtat tgtcttaaaa |
| 71581 | gagtgattga tggtagctac ggaaaacttt gatttttaaa attgtctctt taagtagaca |
| 71641 | atttataagc tactggtacg agttcacctt ataaatctcc actaccatgt ttttgcttgg |
| 71701 | actgttcaca cttcctggaa tggtccttct tgccgtttat ccaacttctt tctaattttt |
| 71761 | aagtccctaa tgatgggaat tctatttctg tagtgatttt tctggtcata cgaccgtaag |

| | | |
|---|---|---|
| (SEQ ID NO.: 263) | | |
| MSH2-13A-s: | 5′ (*)-TAGGACTAACAATCCATT 3′ | (SEQ ID NO.: 264) |
| MSH2-13A-as: | 5′ TGGGCCATGAGTACTA 3′ | (SEQ ID NO.: 265) |
| MSH2-13B-s: | 5′ (*)-ATGGGAGGTAAATCAAC 3′ | (SEQ ID NO.: 266) |
| MSH2-13B-as: | 5′ GACTCCTTTCAATTGACT 3′ | (SEQ ID NO.: 267) |
| MSH2-13C-s4: | 5′ TTGTGGACTGCATCTTAGCC | (SEQ ID NO.: 268) |
| MSH2-13C-5as: | TCACAGGACAGAGACATACATTTC | (SEQ ID NO.: 269) |
| MSH2-13seq-s: | TCTGCCTTTTTCTTCCATCGGGGCTATGTCAGTGTAAACCTACGC | (SEQ ID NO.: 270) |
| MSH2-13seq-as: | TCCCCAACCCCCTAAAGCGACTTCTCACAGGACAGAGACATACA | (SEQ ID NO.: 271) |
| Exon 14 | | |

| | |
|---|---|
| 72661 | ccgttgtttg ttcatgttca tgaccttttt ttttttttcc tattctcctc ccttcctccc |
| 72721 | tccctccctc ccttccttcc ttccctcctt ccctccttcc ctccctccct cccacacaaa |
| 72781 | ggtgtgtgct accatacctg gctagttttt aatttttttt tttttttttt tttttagagg |
| 72841 | caaggtctca ctatgttgct caggctggtc tgggctcaag tgatcctccc acctccgcct |
| 72901 | tccaaagtgc tgggattaca gacgtgagcc atcatgcctg gcccttgccc atttttctat |
| 72961 | tgaagtttta gtgcttttta ttgactttgt ttatatatta agataatcca ttatgtttgt |
| 73021 | ggcatatcct tcccaatgta ttgtcttaat tttgtttttg tatgtgtatg ttaccacatt |
| 73081 | ttatgtgatg ggaaatttca tgtaattatg tgcttcagGT CTGCAACCAA AGATTCATTA |
| 73141 | ATAATCATAG ATGAATTGGG AAGAGGAACT TCTACCTACG ATGGATTTGG GTTAGCATGG |
| 73201 | GCTATATCAG AATACATTGC AACAAAGATT GGTGCTTTTT GCATGTTTGC AACCCATTTT |
| 73261 | CATGAACTTA CTGCCTTGGC CAATCAGATA CCAACTGTTA ATAATCTACA TGTCACAGCA |
| 73321 | CTCACCACTG AAGAGACCTT AACTATGCTT TATCAGGTGA AGAAAGgtat gtactattgg |
| 73381 | agtactctaa attcagaact tggtaatggg aaacttacta cccttgaaat catcagtaat |
| 73441 | tgccttattc taagttagta taaattattg atgttgttat agaacccatt taccccttaa |
| 73501 | ttcacagtct gggggtagga acatgtacat catatttctg tatctcatag taggaccact |
| 73561 | cattctaaag cattcacaga aagaattatc tgtactcttt ttgggacaga atctcgttct |
| 73621 | gttgcccagg ctggagtgcg atctcggctc actgcaacct ccgcctcccg ggttcaagcg |
| 73681 | attctcctgc ctcagcttcc cgagtagctg ggattacagg cgcctgccac cacacctggc |
| 73741 | taatttttat atttttagta gagacggggt ttcaccatgc tggccaggct ggtctcgaat |
| 73801 | tcctgacctc aggcaatcca cccgtctcgg cctcccaaag tgctgggatt acaggtgtga |

| | | |
|---|---|---|
| (SEQ ID NO.: 272) | | |
| MSH2-14A-s3 | 5′ (*)-GTATGTGTATGTTACCACATT 3′ | (SEQ ID NO.: 273) |
| MSH2-14A-as3: | 5′ TAGTTAAGGTCTCTTCAGTG 3′ | (SEQ ID NO.: 274) |
| MSH2-14B-s: | 5′ ATAATCTACATGTCACAGCA 3′ | (SEQ ID NO.: 275) |
| MSH2-14B-as: | 5′ (*)-GAATAAGGCAATTACTGAT 3′ | (SEQ ID NO.: 276) |
| MSH2-14seq-s: | TCTGCCTTTTTCTTCCATCGGGATGTTTGTGGCATATCCTTCC | (SEQ ID NO.: 277) |
| MSH2-14seq-as: | TCCCCAACCCCCTAAAGCGATAGTAAGTTTCCCATTACCAAGTTC | (SEQ ID NO.: 278) |
| Exon 15 | | |

| | |
|---|---|
| 75181 | ccctccctta ccttcccatg aaatgagaaa gcctcagaga tagtggcttg attaattttt |
| 75241 | ctttagatta agatatttgt ctaagccttt aaggtttatc tattgagctt ttttgtctcc |
| 75301 | tatttttatt tttcctacta tgtttgtcga ggataaaata cagcactgtg tgccaagtca |
| 75361 | taatcacttt tcatttgaga cttaattaaa atgcctttat tttaatgata tatttggcta |
| 75421 | atgtatttga agtaatccga aattaagttt tctaatgaca aggtgagaag gataaattcc |
| 75481 | atttacataa attgctgtct cttctcatgc tgtcccctca cgcttcccca aatttcttat |
| 75541 | agGTGTCTGT GATCAAAGTT TTGGGATTCA TGTTGCAGAG CTTGCTAATT TCCCTAAGCA |
| 75601 | TGTAATAGAG TGTGCTAAAC AGAAAGCCCT GGAACTTGAG GAGTTTCAGT ATATTGGAGA |
| 75661 | ATCGCAAGGA TATGATATCA TGGAACCAGC AGCAAAGAAG TGCTATCTGG AAAGAGAGgt |
| 75721 | ttgtcagttt gttttcatag tttaacttag cttctctatt attacataaa caggacacta |
| 75781 | agatgaaggt tttttgttgt tgtttgtttt cctctgtgtt tctagtgctt attttttaat |
| 75841 | cagttttttt gatggcaaag aatctatctc tgtgttattt tgatttctgc agtatataca |
| 75901 | tctgcatgat caatattcga tttcaagtac caaagtagga gtaaaggaat attaacctag |
| 75961 | gtttaaaatt agtcatttca ctaaaattag ttattatgga cgatagatgt ctaggtatat |
| 76021 | ctttgttcat aaacgaatat atcaagttca gttattaaat tacacattag gtaagaaaag |
| 76081 | gacaaagaaa taaaaaagca tgattcataa ttcctgccct ctatttgtct agaatttagt |

| | | |
|---|---|---|
| (SEQ ID NO.: 279) | | |
| MSH2-15A-s | 5′ GTCTCTTCTCATGCTGTC 3′ | (SEQ ID NO.: 280) |
| MSH2-15A-as | 5′ (*)-AATAGAGAAGCTAAGTTAAAC 3′ | (SEQ ID NO.: 281) |
| MSH2-15seq-s: | TCTGCCTTTTTCTTCCATCGGGTTGGCTAATGTATTTGAAGTAATCC | (SEQ ID NO.: 282) |
| MSH2-15seq-as: | TCCCCAACCCCCTAAAGCGAACACAGAGGAAAACAAACAACAA | (SEQ ID NO.: 283) |
| Exon 16 | | |

| | |
|---|---|
| 77041 | gactctttta tgcaatctct tgtttccagt tagaatagaa gtcgtgtact tttgataaca |
| 77101 | ttaattataa tatattttga gccctgtgag gttggtaaca ttattcccat tttatgaatg |
| 77161 | aggaatgtgt gttaaggagt ttgcccaaga gtcacatagc aagtcatagt catgctctct |
| 77221 | gaagcagcaa taacttggca ataaaataaa aatgaagcat cttctgtatg tgttaacttt |
| 77281 | tcagtgactg tttatgcctt ccagtattct ttgtaaacct tgaattcttt ttttcacaga |
| 77341 | tgattaaagt ttatcaattg taaaggtgga ggaatttggg aactagacag tgcacacata |
| 77401 | aataataaat atgttcttca aatattgggt gggctaatgt gggaggagtt tgagaccagc |
| 77461 | ctgggcaaca tagtgagacc ctcgtctcta aaaatatgaa aaataaaaaa aaaatttttt |
| 77521 | aaatgtgtga tatgtttaga tggaaatgaa acaatttgtc actgtctaac atgactttta |
| 77581 | gaaaagatat tttaattact aatgggacat tcacatgtgt ttcagCAAGG TGAAAAAATT |
| 77641 | ATTCAGGAGT TCCTGTCCAA GGTGAAACAA ATGCCCTTTA CTGAAATGTC AGAAGAAAAC |
| 77701 | ATCACAATAA AGTTAAAACA GCTAAAAGCT GAAGTAATAG CAAAGAATAA TAGCTTTGTA |
| 77761 | AATGAAATCA TTTCACGAAT AAAAGTTACT ACGTGAaaaa tcccagtaat ggaatgaagg |
| 77821 | taatattgat aagctattgt ctgtaatagt tttatattgt tttatattaa ccctttttcc |
| 77881 | atagtgttaa ctgtcagtgc ccatgggcta tcaacttaat aagatattta gtaatatttt |
| 77941 | actttgagga cattttcaaa gatttttatt ttgaaaaatg agagctgtaa ctgaggactg |
| 78001 | tttgcaattg acataggcaa taataagtga tgtgctgaat tttataaata aaatcatgta |
| 78061 | gtttgtgg |

| | | |
|---|---|---|
| (SEQ ID NO.: 284) | | |
| MSH2-16A-s: | 5′ TTACTAATGGGACATTCACATG 3′ | (SEQ ID NO.: 285) |
| MSH2-16A-as: | 5′ (*)-ACAATAGCTTATCAATATTACCTTC 3′ | (SEQ ID NO.: 286) |
| MSH2-16seq-s: | TCTGCCTTTTTCTTCCATCGGGGTAAAGGTGGAGGAATTTGGG | (SEQ ID NO.: 287) |
| MSH2-16seq-as: | TCCCCAACCCCCTAAAGCGAGGCACTGACAGTTAACACTATGGA | (SEQ ID NO.: 288) |

(*) = CGCCCGCCGCGCCCCGCGCCCGCCCCGCCGCCCCCGCCCG (SEQ ID NO.: 344)

TABLE B

MLH1 AND MSH2 TTGE PRIMERS
MLH1 TTGE Primers 3/3/04 AK
OMIM 120436
gene map 3p21.3, Locus ID 4292
mRNA NM_000249
earlier gonomic contig used AY_217549
Reference numbering below refers to NC-000003, human chromosome 3.
NC-000003 human chromosome 3. Region encompassing MLH1 gene from 36992890 . . . 37053065.

| | | | | | | |
|---|---|---|---|---|---|---|
| | | | start | | | |
| primer name | SEQ ID | start | acc. | end | end acc. | primer sequence |

| | | | | | | |
|---|---|---|---|---|---|---|
| | | | | | | this is 5′-3′ sequence for each sense and |
| exon 1 | | | | | | antisense primer. (*) = GC clamp |
| MLH1-1A-s: | 3 | 2635 | 36995524 | 2650 | 36995539 | 5′ (*)-CAATAGCTGCCGCTGA 3′ |
| MLH1-1A-as: | 4 | 2839 | 36995728 | 2854 | 36995743 | 5′ CGCTGGATAACTTCCC 3′ |
| MLH1-1B-s: | 5 | 2834 | 36995723 | 2848 | 36995737 | 5′ GGCGGGGGAAGTTAT 3′ |
| MLH1-1B-as: | 6 | 2944 | 36995833 | 2959 | 36995848 | 5′ (*)-CGCGCCATTGAGTGAC 3′ |
| MLH1-1C-s: | 7 | 2870 | 36995759 | 2888 | 36995777 | 5′ (*)-CAAAGAGATGATTGAGAAC 3′ |
| MLH1-1C-as: | 8 | 2975 | 36995864 | 2989 | 36995878 | 5′ CATGCCTCTGCCCGG 3′ |
| MLH1-1D-s: | 9 | 2685 | 36995574 | 2702 | 36995591 | 5′ (*)-GGAAGAACGTGAGCACGA 3′ |
| MLH1-1D-as: | 10 | 2850 | 36995739 | 2865 | 36995754 | 5′ CATTAGCTGGCCGCTG 3′ |
| exon 2 | | | | | | |
| MLH1-2A-s: | 16 | 5765 | 36998654 | 5780 | 36998669 | 5′ (*)-TTATCATTGCTTGGCT 3′ |
| MLH1-2A-as: | 17 | 5903 | 36998792 | 5920 | 36998809 | 5′ TTGTCTTGGATCTGAATC 3′ |
| MLH1-2B-s: | 18 | 5853 | 36998742 | 5870 | 36998759 | 5′ (*)-GCAAAATCCACAAGTATT 3′ |
| MLH1-2B-as: | 19 | 5974 | 36998863 | 5990 | 36998879 | 5′ CCTGACTCTTCCATGAA 3′ |
| exon 3 | | | | | | |
| MLH1-3A-s: | 23 | 10115 | 37003004 | 10130 | 37003019 | 5′ (*)-GGGAATTCAAAGAGAT 3′ |
| MLH1-3A-as: | 24 | 10283 | 37003172 | 10300 | 37003189 | 5′ TTCTTGAATCTTTAGCTT 3′ |
| MLH1-3B-s: | 25 | 10195 | 37003084 | 10216 | 37003105 | 5′ ATATTGTATGTGAAAGGTTCAC 3′ |
| MLH1-3B-as: | 26 | 10360 | 37003249 | 10381 | 37003270 | 5′ (*)-ACCAAACCTTATTTATCTATGT 3′ |
| exon 4 | | | | | | |
| MLH1-4A-s4 | 32 | 13562 | 37006451 | 13579 | 37006468 | 5′ GGTGAGGTGACAGTGGGT 3′ |
| MLH1-4A-as4 | 33 | 13710 | 37006599 | 13736 | 37006625 | 5′ (*)-TGAATATATATGAGTAAAAGAAGTCAG 3′ |
| MLH1-4B-s2 | 34 | 13654 | 37006543 | 13676 | 37006565 | 5′ TCATGTTACTATTACAACGAAAA 3′ |
| MLH1-4B-as2 | 35 | 13776 | 37006665 | 13796 | 37006685 | 5′ (*)-GATAACACTGGTGTTGAGACA 3′ |
| exon 5 | | | | | | |
| MLH1-5a-s: | 39 | 16164 | 37009053 | 16182 | 37009071 | 5′ (*)-GGGATTAGTATCTATCTCT 3′ |
| MLH1-5A-as: | 40 | 16234 | 37009123 | 16248 | 37009137 | 5′ GGCTTTCAGTTTTCC 3′ |
| MLH1-5B-s2: | 41 | 16240 | 37009129 | 16255 | 37009144 | 5′ CTGAAAGCCCCTCCTA 3′ |
| MLH1-5B-as2: | 42 | 16308 | 37009197 | 16327 | 37009216 | 5′ (*)-AGCTTCAACAATTTACTCTC 3′ |
| MLH1-5C-s2: | 43 | 16273 | 37009162 | 16289 | 37009178 | 5′ CAAGGGACCCAGATCAC 3′ |
| MLH1-5C-as2: | 44 | 16325 | 37009214 | 16346 | 37009235 | 5′ (*)-CCAATATTTATACAAACAAAGC 3′ |
| MLH1-5D-s | 45 | 16197 | 37009086 | 16219 | 37009108 | 5′ (*)-TTTGTTATATTTTCTCATTAGAG 3′ |
| MLH1-5D-s | 46 | 16281 | 37009170 | 16298 | 37009187 | 5′ ATTCTTACCGTGATCTGG 3′ |
| exon 6 | | | | | | |
| MLH1-6-5-s | 50 | 17945 | 37010834 | 17967 | 37010856 | 5′ (*)-ATTCACTATCTTAAGACCTCGCT 3′ |
| MLH1-6-5-as | 51 | 18168 | 37011057 | 18192 | 37011081 | 5′ CTAGAACACATTACTTTGATGACAA 3′ |
| exon 7 | | | | | | |
| MLH1-7-s: | 55 | 20971 | 37013860 | 20986 | 37013875 | 5′ TAACTAAAAGGGGGCT 3′ |
| MLH1-7-as: | 56 | 21191 | 37014080 | 21207 | 37014096 | 5′ (*)-TTTATTGTCTCATGGCT 3′ |
| exon 8 | | | | | | |
| MLH1-8A-s: | 60 | 21157 | 37014046 | 21172 | 37014061 | 5′ (*)-GCTGGTGGAGATAAGG 3′ |
| MLH1-8A-as: | 61 | 21278 | 37014167 | 21292 | 37014181 | 5′ TGTCCACGGTTGAGG 3′ |
| MLH1-8B-s: | 62 | 21238 | 37014127 | 21258 | 37014147 | 5′ GGGGGCAAGGAGAGACAGTAG 3′ |
| MLH1-8B-as2: | 63 | 21326 | 37014215 | 21345 | 37014234 | 5′ (*)-ATATAGGTTATCGACATACC 3′ |
| MLH1-8C-s2: | 64 | 21312 | 37014201 | 21325 | 37014214 | 5′ AAATGCTGTTAGTC 3′ |
| MLH1-8C-as: | 65 | 21397 | 37014286 | 21412 | 37014301 | 5′ (*)-TCTTGAAAGGTTCCAA 3′ |
| exon 9 | | | | | | |
| MLH1-9A-3-s | 69 | 23605 | 37016494 | 23630 | 31016519 | 5′ (*)-GTAATGTTTGAGTTTTGAGTATTTTC 3′ |
| MLH1-9A-3-as | 70 | 23834 | 37016723 | 23853 | 37016742 | 5′ CAGAAATTTTTCCATGGTCC 3′ |
| MLH1-9B-s | 71 | 23554 | 37016443 | 23575 | 37016464 | 5′ (*)-CAAAGTTAGTTTATGGGAAGGA 3′ |
| MLH1-9B-as | 72 | 23741 | 37016630 | 23764 | 31016653 | 5′ GAAGAGTAAGAAGATGCACTTCTT 3′ |
| MLH1-9C-s | 73 | 23698 | 37016587 | 23720 | 37016609 | 5′ (*)-CTTCAAAATGAATGGTTACATAT 3′ |
| MLH1-9C-as | 74 | 23810 | 37016699 | 23827 | 37016716 | 5′ ATTCCCTGTGGGTGTTTC 3′ |
| exon 10 | | | | | | |
| MLH1-10-s: | 78 | 26665 | 37019554 | 26682 | 37019571 | 5′ (*)-TGAATGTACACCTGTGAC 3′ |
| MLH1-10-as: | 79 | 26861 | 37019750 | 26878 | 37019767 | 5′ TAGAACATCTGTTCCTTG 3′ |
| exon 11 | | | | | | |
| MLH1-11A-s: | 83 | 29423 | 37022312 | 29439 | 37022328 | 5′ (*)-TTGACCACTGTGTCATC 3′ |
| MLH1-11A-as: | 84 | 25951 | 37018840 | 29606 | 37022495 | 5′ GTGCAGGAAGTGAACT 3′ |
| MLH1-11B-s: | 85 | 29553 | 37022442 | 29571 | 37022460 | 5′ (*)-CAGAATGTGGATGTTAATG 3′ |
| MLH1-11B-as: | 86 | 29658 | 37022547 | 29672 | 37022561 | 5′ GGAGGAATTGGAGCC 3′ |
| MLH1-11C-s4: | 87 | 29631 | 37022520 | 29647 | 37022536 | 5′ CAGCAGCACATCGAGAG 3′ |
| MLH1-11C-as4: | 88 | 29746 | 37022635 | 29763 | 37022652 | 5′ (*)-ATCTGGGCTCTCACGTCT 3′ |
| exon 12 | | | | | | |
| MLH1-12B-s: | 92 | 34849 | 37027738 | 34869 | 37027758 | 5′ (*)-TTTTTTTTAATACAGACTTTG 3′ |
| MLH1-12B-as: | 93 | 35049 | 37027938 | 35063 | 37027952 | 5′ GTGACAATGGCCTGG 3′ |
| MLH1-12C-s: | 94 | 35009 | 37027898 | 35024 | 37027913 | 5′ CATTTCTGCAGCCTCT 3′ |
| MLH1-12C-as: | 95 | 35142 | 37028031 | 35156 | 37028045 | 5′ (*)-TTTTTGGCAGCCACT 3′ |
| MLH1-12D-s3: | 96 | 35130 | 37028019 | 35145 | 37028034 | 5′ AGCCCCTGCTGAAGTG 3′ |
| MLH1-12D-as3: | 97 | 35274 | 37028163 | 35294 | 37028183 | 5′ (*)-AGAAGGCAGTTTTATTACAGA 3′ |
| MLH1-12E-s: | 98 | 35036 | 37027925 | 35051 | 37027940 | 5′ (*)-TGTCCAGTCAGCCCCA 3′ |
| MLH1-12E-as: | 99 | 35146 | 37028035 | 35162 | 37028051 | 5′ CTCTGATTTTTGGCAGC 3′ |
| exon 13 | | | | | | |
| MLH1-13A-s: | 106 | 37950 | 37030839 | 37966 | 37030855 | 5′ (*)-AATTTGGCTAAGTTTAA 3′ |
| MLH1-13A-as: | 107 | 37950 | 37030839 | 37966 | 37030855 | 5′ GGAATCATCTTCCACC 3′ |
| MLH1-13B-s2: | 108 | 38003 | 37030892 | 38021 | 37030910 | 5′ (*)-CATTGCAGAAAGAGACATC 3′ |
| MLH1-13B-as3: | 109 | 38093 | 37030982 | 38112 | 37031001 | 5′ CGCCCGCCGCGGTGAGGTTAATGATCCTTCT 3′ |
| MLH1-13C-s1: | 110 | 38053 | 37030942 | 38073 | 37030962 | 5′ (*)-TGATTCCCGAAAGGAAATGAC 3′ |
| MLH1-13C-as1: | 111 | 38153 | 37031042 | 38180 | 37031069 | 5′ CAGGCCACAGCGTTTACGTACCCTCATG 3′ |
| MLH1-13D-s: | 112 | 38102 | 37030991 | 38122 | 37031011 | 5′ (*)-ATTAACCTCACTAGTGTTTTG 3′ |
| MLH1-13D-as: | 113 | 38186 | 37031075 | 38201 | 37031090 | 5′ TGAGGCCCTATGCATC 3′ |
| exon 14 | | | | | | |
| MLH1-14A-s: | 117 | 49344 | 37042233 | 49359 | 37042248 | 5′ (*)-GGTCAATGAAGTGGGG 3′ |
| MLH1-14A-as: | 118 | 49432 | 37042321 | 49448 | 37042337 | 5′ CCACGAAGGAGTGGTTA 3′ |
| MLH1-14B-s: | 119 | 49411 | 37042300 | 49426 | 37042315 | 5′ AGTTCTCCGGGAGATG 3′ |
| MLH1-14B-as: | 120 | 49596 | 37042485 | 49612 | 37042501 | 5′ (*)-TACCTCATGCTGCTCTC 3′ |
| exon 15 | | | | | | |
| MLH1-15-s: | 124 | 51403 | 37044292 | 51419 | 37044308 | 5′ TTCAGGGATTACTTCTC 3′ |
| MLH1-15-as: | 125 | 51637 | 37044526 | 51656 | 37044545 | 5′ (*)-GAAAAATTTAACATACTACA 3′ |
| exon 16 | | | | | | |
| MLH1-16A-s: | 129 | 56658 | 37049547 | 56674 | 37049563 | 5′ (*)-GCCATTCTGATAGTGGA 3′ |
| MLH1-16A-as2: | 130 | 56768 | 37049657 | 56786 | 37049675 | 5′ TCTAAGGCAAGCATGGCAA 3′ |
| MLH1-16B-s: | 131 | 56752 | 37049641 | 56765 | 37049654 | 5′ GCACCGCTCTTTGA 3′ |
| MLH1-16B-as: | 132 | 56914 | 37049803 | 56930 | 37049819 | 5′ (*)-GTATAAGAATGGCTGTCA 3′ |
| MLH1-16C-s2: | 133 | 56868 | 37049757 | 56884 | 37049773 | 5′ GGCTGAGATGCTTGCAG 3′ |
| MLH1-16C-as2: | 134 | 56967 | 37049856 | 56981 | 37049870 | 5′ (*)-CATGAGCCACCGCAC 3′ |
| exon 17 | | | | | | |
| MLH1-17-s: | 138 | 57689 | 37050578 | 57706 | 37050595 | 5′ (*)-TGTTTAAACTATGACAGCA 3′ |
| MLH1-17-as: | 139 | 57892 | 37050781 | 57906 | 37050795 | 5′ TGGTCATTTGCCCTT 3′ |
| exon 18 | | | | | | |
| MLH1-18A-s: | 143 | 58060 | 37050949 | 58077 | 37050966 | 5′ (*)-TGTGATCTCCGTTTAGAA 3′ |
| MLH1-18A-as2: | 144 | 58220 | 37051109 | 58236 | 37051125 | 5′ CTGAGAGGGTCGACTCC 3′ |
| MLH1-18B-s3: | 145 | 58179 | 37051068 | 58197 | 37051086 | 5′ (*)-TGCGCTATGTTCTATTCCA 3′ |
| MLH1-18B-as3: | 146 | 58264 | 37051153 | 58280 | 37051169 | 5′ GCCGCCCCCGCCCGCTAGTCCTGGGGTGCCA 3′ |
| exon 19 | | | | | | |
| MLH1-19A-s: | 150 | 59615 | 37052504 | 59631 | 37052520 | 5′ CAAGTCTTTCCAGACCC 3′ |
| MLH1-19A-as: | 151 | 59843 | 37052732 | 59860 | 37052749 | 5′ (*)-TGTATAGATCAGGCAGGT 3′ |
| MLH1-19B-s4 | 153 | 59774 | 37052663 | 59790 | 37052679 | 5′ AAGCCTTGCGCTCACAC 3 |
| MLH1-19B-as4 | 155 | 59867 | 37052756 | 59891 | 37052780 | 5′ (*)-AATAACCATATTTAACACCTCTCAA 3′ |
| MLH1-19C-s: | 152 | 59813 | 37052702 | 59833 | 37052722 | 5′ (*)-CAGAAGATGGAAATATCCTGC 3′ |
| MLH1-19C-as: | 153 | 59937 | 37052826 | 59962 | 37052851 | 5′ CCGCCCGTGTATATCACACTTTGATACAACACT3′ |
| * clamp is | 344 | | | | | CGCCCGCCGCGCCCCGCGCCCGCCCCGCCGCCCCCGCCCG |

MLH1 Sequencing Primers

| | | | | | | |
|---|---|---|---|---|---|---|
| | | | | | | this is 5′-3′ sequence for each sense and |
| | | | | | | antisense primer. Primers have tags** |
| MLH1-1seq-s | 13 | 2562 | 36995451 | 2581 | 36995470 | TCTGCCTTTTTCTTCCATCGGGGCTTCAGGGAGGGACGAAGA |
| MLH1-1seq-as | 14 | 2979 | 36995868 | 2998 | 36995887 | TCCCCAACCCCCTAAAGCGATGCGCTGTACATGCCTCTGC |
| MLH1-2seq-s | 20 | 5653 | 36998542 | 5672 | 36998561 | TCTGCCTTTTTCTTCCATCGGGTGCCCGTCTCTTCCCTCTCT |
| MLH1-2seq-as | 21 | 6085 | 36998974 | 6104 | 36998993 | TCCCCAACCCCCTAAAGCGACCTGAACAGTGCCCAGCAAA |
| MLH1-3seq-s | 27 | 9947 | 37002836 | 9970 | 37002859 | TCTGCCTTTTTCTTCCATCGGGCAAGACTCTGTCTCAAAGGAGGTT |
| MLH1-3seq-as2 | 30 | 10401 | 37003290 | 10425 | 37003314 | TCCCCAACCCCCTAAAGCGACATTAAGTTTGCTCAGATTTGCATA |
| MLH1-4seq-s | 36 | 13474 | 37006363 | 13495 | 37006384 | TCTGCCTTTTTCTTCCATCGGGCATGTCATCAAAGCAAGTGAGC |
| MLH1-4seq-as | 37 | 13759 | 37006648 | 13782 | 37006671 | TCCCCAACCCCCTAAAGCGATGAGACAGGATTACTCTGAGACCT |
| MLH1-5seq-s2 | 47 | 16159 | 37009048 | 16182 | 37009071 | TCTGCCTTTTTCTTCCATCGGGCCCTTGGGATTAGTATCTATCTCT |
| MLH1-5seq-as | 48 | 16397 | 37009286 | 16418 | 37009307 | TCCCCAACCCCCTAAAGCGAGGACCTCCATTAACTAGTGCAA |
| MLH1-6seq-s | 52 | 17877 | 37010766 | 17900 | 37010789 | TCTGCCTTTTTCTTCCATCGGGCTGTTAATGCTGTCTTATCCCTGG |
| MLH1-6seq-as | 53 | 18204 | 37011093 | 18226 | 37011115 | TCCCCAACCCCCTAAAGCGACCATCTAGCTCAGCAACTGTTCA |
| MLH1-7seq-s | 59 | 20856 | 37013745 | 20875 | 37013764 | TCTGCCTTTTTCTTCCATCGGGTTCCATGAAGTTTCTGCTGG |
| MLH1-7seq-as | 58 | 21151 | 37014040 | 21172 | 37014061 | TCCCCAACCCCCTAAAGCGACCTTATCTCCACCAGCAAACTA |
| MLH1-8seq-s | 66 | 21100 | 37013989 | 21120 | 37014009 | TCTGCCTTTTTCTTCCATCGGGGGTTTATGGGGGATGGTTTTG |
| MLH1-8seq-as | 67 | 21520 | 37014409 | 21543 | 37014432 | TCCCCAACCCCCTAAAGCGACGCCACAGAATCTAGGAGATTACA |
| MLH1-9seq-s | 75 | 23462 | 37016351 | 23481 | 37016370 | TCTGCCTTTTTCTTCCATCGGGGGTGGGTGAATGGGTGAACA |
| MLH1-9seq-as | 76 | 23875 | 37016764 | 23894 | 37016783 | TCCCCAACCCCCTAAAGCGATTTGCCATGAGGTTTCTCCA |
| MLH1-10seq-s | 80 | 26563 | 37019452 | 26581 | 37019470 | TCTGCCTTTTTCTTCCATCGGGGCTGGAAAGTGGCGACAGG |
| MLH1-10seq-as | 81 | 26929 | 37019818 | 26949 | 37019838 | TCCCCAACCCCCTAAAGCGAGCCAGTGGTGTATGGGATTCA |
| MLH1-11seq-s | 89 | 29324 | 37022213 | 29344 | 37022233 | TCTGCCTTTTTCTTCCATCGGGAGACTGAGGCAAAGAAAGATG |
| MLH1-11seq-as | 90 | 29753 | 37022642 | 29771 | 37022660 | TCCCCAACCCCCTAAAGCGAAGGCAAAAATCTGGGCTCT |
| MLH1-12seq-s | 100 | 34696 | 37027585 | 34714 | 37027603 | TCTGCCTTTTTCTTCCATCGGGTTTCGGGCAGAATTGCTTC |
| MLH1-12seq-as | 101 | 35312 | 37028201 | 35334 | 37028223 | TCCCCAACCCCCTAAAGCGAGCAGAGAGAAGATGCAAGTGATT |
| alternate | | | | | | |
| MLH1-12seq-s2 | 103 | 34453 | 37027342 | 34474 | 37027363 | TCTGCCTTTTTCTTCCATCGGGATAGCTGGTGGTGATGGTTGCG |
| MLH1-12seq-as2 | 104 | 35345 | 37028234 | 35366 | 37028255 | TCCCCAACCCCCTAAAGCGACCATTCCAGCACCATTCCAGAG |
| MLH1-13seq-s | 114 | 37852 | 37030741 | 37872 | 37030761 | TCTGCCTTTTTCTTCCATCGGGACTGATCTTGTTGGCCTTCTG |
| MLH1-13seq-as | 115 | 38233 | 37031122 | 38252 | 37031141 | TCCCCAACCCCCTAAAGCGATGGCCACTCTGACAACATGA |
| MLH1-14seq-s | 121 | 49268 | 37042157 | 49287 | 37042176 | TCTGCCTTTTTCTTCCATCGGGTGTTCGTTTTCACCAGGAGG |
| MLH1-14seq-as | 122 | 49647 | 37042536 | 49668 | 37042557 | TCCCCAACCCCCTAAAGCGATCGAACTTGGATTTGAAACCAC |
| MLH1-15seq-s2 | 126 | 51361 | 37044250 | 51379 | 37044268 | TCTGCCTTTTTCTTCCATCGGGAGATTCCACAGCCAGGCAG |
| MLH1-15seq-as2 | 127 | 51683 | 37044572 | 51706 | 37044595 | TCCCCAACCCCCTAAAGCGATACCTCCATATGCAAATCATACAA |
| MLH1-16seq-s | 135 | 56582 | 37049471 | 56604 | 37049493 | TCTGCCTTTTTCTTCCATCGGGGGTTTTGTTGTGGATTGTTCAGG |
| MLH1-16seq-as | 136 | 56974 | 37049863 | 56993 | 37049882 | TCCCCAACCCCCTAAAGCGATGGGATTACAGCCATGAGCC |
| MLH1-17seq-s | 140 | 57580 | 37050469 | 57601 | 37050490 | TCTGCCTTTTTCTTCCATCGGGTTTAAGTGTTTAGGTCTGCCCC |
| MLH1-17seq-as | 141 | 59926 | 37052815 | 57948 | 37050837 | TCCCCAACCCCCTAAAGCGAGCTATCCCACCCTTATCATCTTT |
| MLH1-18seq-s | 147 | 57927 | 37050816 | 57948 | 37050837 | TCTGCCTTTTTCTTCCATCGGGAAGATGATAAGGGTGGGATAGC |
| MLH1-18seq-as | 148 | 58317 | 37051206 | 58336 | 37051225 | TCCCCAACCCCCTAAAGCGACCGAAATTTTAGAGATGGGC |
| MLH1-19seq-s | 156 | 59462 | 37052351 | 59482 | 37052371 | TCTGCCTTTTTCTTCCATCGGGGCTATGATCACACCACTGCCC |
| MLH1-19seq-as | 157 | 60108 | 37052997 | 60129 | 37053018 | TCCCCAACCCCCTAAAGCGACCTCTTTTTGGCATCTGAACTG |
| MSH2/MLH1 | | | | | | tagged primer in DTCS reaction |
| ** sense tag is | 289 | | | | | TCTGCCTTTTTCTTCCATCGGG |
| ** antisense tag is | 290 | | | | | TCCCCAACCCCCTAAAGCGA |

| | |
|---|---|
| MLH1 | Sequencing Primers internal instead of tagged primer in sense direction |

| | | | | | | |
|---|---|---|---|---|---|---|
| MLH1-3seq-s2-int | 29 | 10094 | 37002983 | 10117 | 37003006 | CCTGGATTAAATCAAGAAAATGGG |
| MLH1-12seq-s2-int | 102 | 34861 | 37027750 | 34884 | 37027773 | CAGACTTTGCTACCAGGACTTGCT |

TABLE C
Primer master set up for MLH1 and MSH2

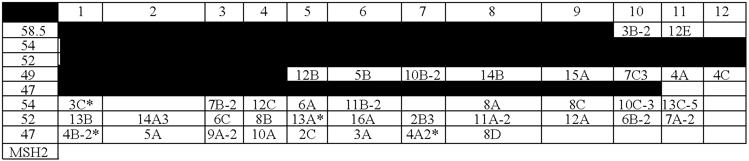

rev. 040404

HNPCC ASSAY PCR SET UP AND STACKING AK 040404

PART 1: PCR
Primer plate
Primer plate has 13.5 ul of primer mix at 5 uM or 10 uM as shown below. Heat sealed.
Take from freezer, thaw at room temp for a few min, spin down 1 min 1500 g, open carefully. Keep cool on cooler block.
Log date of primer plate made:
Log date of primer plate used:
Log number of primer plate used:

| | |
|---|---|
| Visually Inspect volume | ok? |

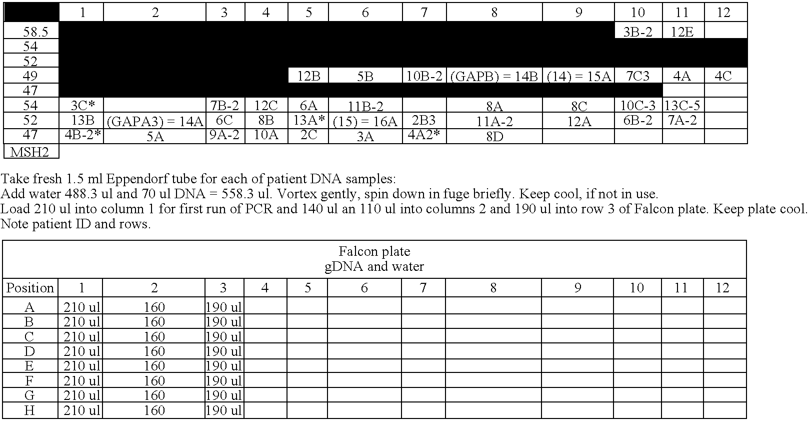

Run HNPCC PCR program on Biomek. Biomek set up as below. Use fresh box of P20 and P250 in each block.
7 plate set-up run:
Pipette manually 67.5 ul of hotstart master mix to each primer well. Pipette up and down three times. Avoid bubbles.
Pause after transfer for visual inspection and quick spin 1500 g, 1 min if necessary. Make sure all bubbles are gone.
Place primer plate on robot with other labeled plates.
Run program, transfer 9 ul of primer/MM from one well to each well of column A-H of corresponding primer plate.
Multi eject, no tip touch if have P250 (takes up 9 × 9 ul). Asp. height and rate 5/3, eject 10/4.
Program pauses after all primers have been dispensed. Inspect and quick spin if necessary-otherwise continue.
Replace primer master with Falcon plate with gDNA in B3
Add gDNA/water from Falcon rows 1 and 2 with multi20, 6 ul per well, tip touch.
Asp. Heights and rates are 10/6, 60/3, last 5/6, eject 60/3. Tip change after plate.
Remove PCR plates from Biomek.
Carefully shake DNA down from edge of PCR plates, heat seal. Vortex gently 30 sec., spin 1500 g 1 min.
Run PCR

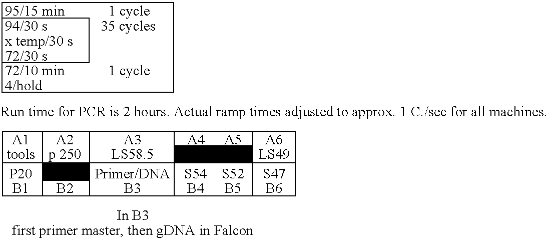

Store plates at −20 unless proceeding to force het and stacking programs. Quick spin prior to storage.
PART 2:
Force heteromers:
Before stacking, force het for 5 min at 95 C., 10 min at 50 C., 4 min/hold. Keep plates at 4.
PART 3:
Stacking program
Take PCR plates from 4 C., fresh Falcon plates and set up Biomek as below.
Spin PCR plates briefly 1500 g, 1 min. to collect volume.
Load 200 ul 2x loading dye into rows 1 and 2 of master Falcon plate at A2.

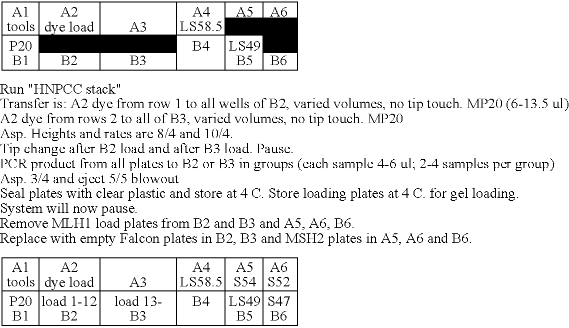

continue stacking program
Transfer is: A2 dye from row 1 to all wells of B2, varied volumes, no tip touch. MP20 (6-13.5 ul)
A2 dye from rows 2 and 3 to all of B3, varied volumes, no tip touch. MP20
Asp. Heights and rates are 8/4 and 10/4.
Tip change after B2 load and after B3 load. Pause.
PCR product from all plates to B2 or B3 in groups (each sample 4-6 ul; 2-4 samples per group)
Asp. 3/4 and eject 5/5 blowout
Seal plates with clear plastic and store at 4 C. Store loading plates at 4 C. for gel loading.

TABLE D

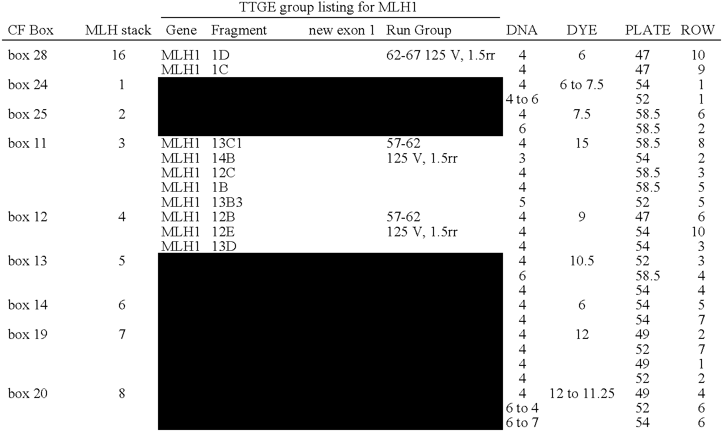

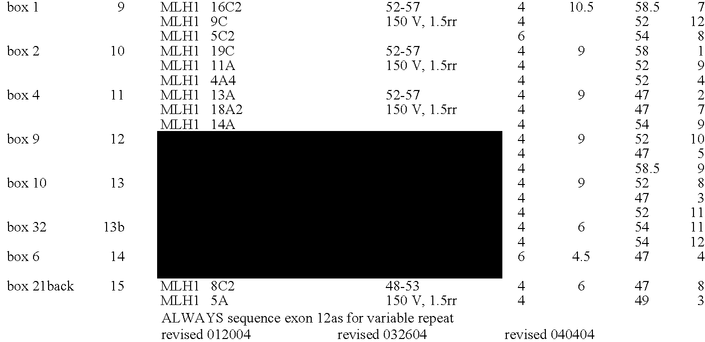

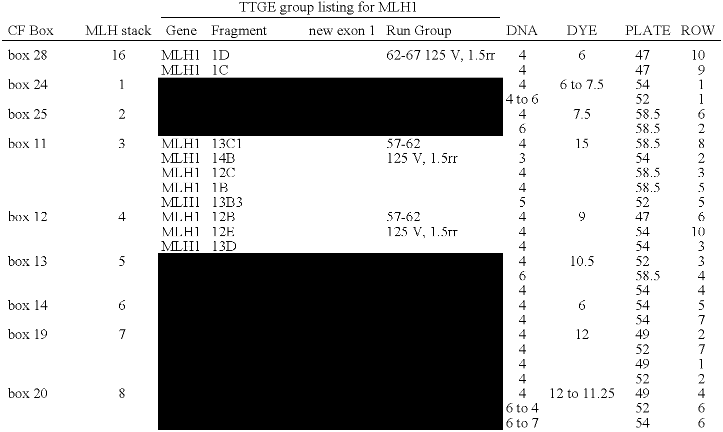

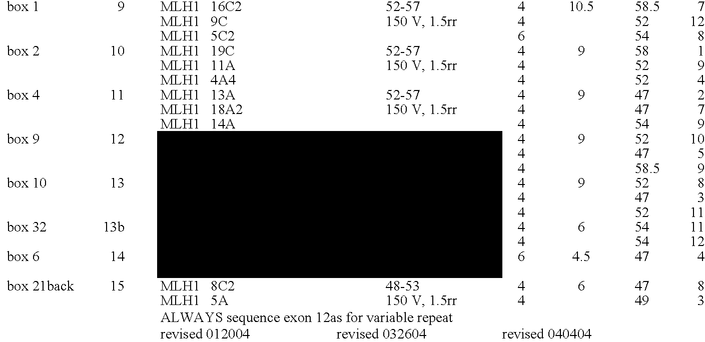

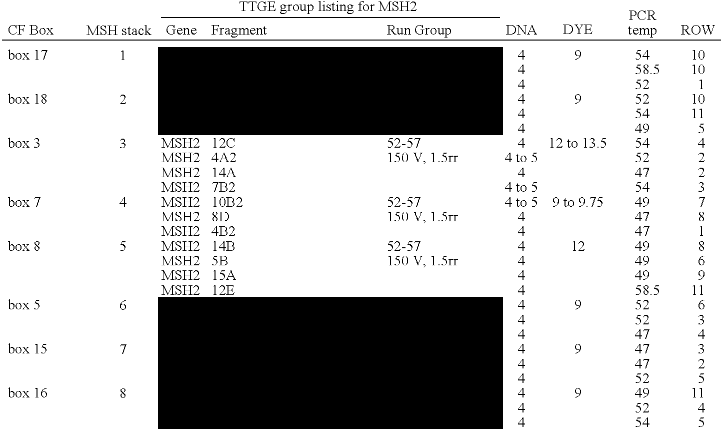

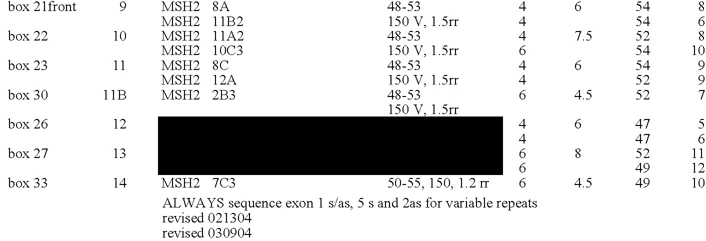

TABLE E
MSH2 and MLH1 SEQ Primers

| Exon | MSH2 Primer set for PCR | PCR anneal |
|---|---|---|
| 1 | first choice: MSH2-1seq-s2/as2 | 61.8 |
| 1 | second choice: MSH2-1seq-s/as | 61.8 |
| 2 | first choice MSH2-2seq-s2/as2 | 61.8 |
| 2 | second choice: MSH2-2seq-s3/as3 | 69 |
| 3 | first choice: MSH2-3seq-s/as4 | 61.8 |
| 3 | second choice: MSH2-3seq-s4/as4 | 56.7 |
| 4 | MSH2-4seq-s2/as2 | 61.8 |
| 5 | first choice: MSH2-5seq-s3/as3 | 61.8 |
| 5 | second choice: MSH2-5seq-s2/as2 | 61.8 |
| 6 | MSH2-6seq-s/as | 56.7 |
| 7 | MSH2-7seq-s3/as2 | 61.8 |
| 8 | MSH2-8seq-s/as | 56.7 |
| 9 | MSH2-9seq-s/as | 56.7 |
| 10 | MSH2-10seq-s/as | 56.7 |
| 11 | MSH2-11seq-s/as | 56.7 |
| 12 | MSH2-12seq-s/as | 61.8 |
| 13 | MSH2-13seq-s/as | 56.7 |
| (GAP) = 14 | (MSH2-GAPseq-s/as) = (MSH2-14seq-s/as) | 61.8 |
| (14) = 15 | (MSH2-14seq-s/as) = (MSH2-15seq-s/as) | 56.7 |
| (15) = 16 | (MSH2-15seq-s/as) = (MSH2-16seq-s/as) | 56.7 |
| Exons | MSH2 Sequencing Primers | |
| all exons | MSH2 s tag | |
| all exons | MSH2 as tag | |
| 2 | MSH2-2seq-s2-int added 081303 | |
| 5 | MSH2-5seq-as2-int added 081303 | |
| Exon | MLH1 Primer set for PCR. | PCR anneal |
| 1 | MLH1-1seq-s/as | 63.4 |
| 2 | MLH1-2seq-s/as | 63.4 |
| 3 | MLH1-3seq-s/as2 | 63.4 |
| 4 | MLH1-4seq-s/as | 63.4 |
| 5 | MLH1-5seq-s2/as1 | 59.6 |
| 6 | MLH1-6seq-s/as | 63.4 |
| 7 | MLH1-7seq-s/as | 59.6 |
| 8 | MLH1-8seq-s/as | 59.6 |
| 9 | MLH1-9seq-s/as | 63.4 |
| 10 | MLH1-10seq-s/as | 63.4 |
| 11 | MLH1-11seq-s/as | 63.4 |
| 12 | first choice: MLH1-12seq-s/as | 59.6 |
| 12 | second choice: MLH1-12seq-s2/as2 | 59.6 |
| 13 | MLH1-13seq-s/as | 63.4 |
| 14 | MLH1-14seq-s/as | 63.4 |
| 15 | MLH1-15seq-s2/as2 | 63.4 |
| 16 | MLH1-16seq-s/as | 63.4 |
| 17 | MLH1-17seq-s/as | 63.4 |
| 18 | MLH1-18seq-s/as | 63.4 |
| 19 | MLH1-19seq-s/as | 63.4 |
| Exons | MLH1 Sequencing Primers | |
| all but 3, 12 | MSH2 s tag | |
| all | MSH2 as tag | |
| 3 | MLH1-3seq-s2-int | |
| 12 | MLH1-12seq-s2-int | |

PCR Volumes

| | | | |
|---|---|---|---|
| Add | 5 | ul | TaqMM or Hotstar TaqMM |
| | 0.5 | ul | gDNA |
| | 1.0 | ul | primer mix at 5 uM S and AS primer |
| | 3.5 | ul | water |
| | 10 | ul | total |

PCR Conditions

| | | | |
|---|---|---|---|
| 1 | 95 C. | minutes | or 15 min with hotstar |
| 2 | 94 C. | 30 seconds | TAQMM |
| 3 | annealing temp as indicated above | 30 seconds | |
| 4 | 72 C. | 45-60 seconds | 4 links to 2 30-35x |
| 5 | 72 C. | 10 minutes | |
| 6 | 4 C. | forever | |

EXO SAP IT Volumes

| | | |
|---|---|---|
| | Exo (uL) | PCR Prod (uL) |
| Add | 1 | to 2.5 |
| | 2 | 5 |

EXO SAP IT Conditions

| | | |
|---|---|---|
| 1 | 37 C. | 60 minutes |
| 2 | 72 C. | 15 minutes |

DTCS Volumes

| | | | |
|---|---|---|---|
| Add | 4.0-4.5 | uL | of dH2O |
| | 0.5-1.0 | uL | of Exo Sap it Product |
| | 1.0 | uL | of 1.6 uM Primer (sense or anti-sense) |
| | 4.0 | uL | DTCS solution |
| | 10 | uL | Total |

DTCS Conditions

| | | | |
|---|---|---|---|
| 1 | 96 C. | 20 seconds | |
| 2 | 50 C. | 20 seconds | |
| 3 | 60 C. | 4 minutes | 3 links to 1 35x |
| 4 | 4 C. | forever | |

Primer stock 5 uM mixed:

| | | |
|---|---|---|
| 10 | ul | 50 uM sense primer |
| 10 | ul | 50 uM antisense primer |
| 80 | ul | water |
| 100 | ul | total |

CEQ 2000 Run Conditions
Injection Time: 20 seconds
Run Time: 65-85 minutes
these times are exceptions to the default parameters
Rev 002 MSH2 and MLH1 Sequencing Primers
AK
4/25/2003
8/15/2003
9/17/2003
2/13/2004
3/6/2004
3/26/2004

| | | | |
|---|---|---|---|
| Exon | Primer | Seq. ID No. | Sequence |

MSH2 and MLH1 Sequencing Primers
2/13/2004
AK
MSH2

| | | | |
|---|---|---|---|
| 1 | MSH2-1seq-s2 | 162 | TCTGCCTTTTTCTTCCATCGGGGCGCAGTAGCTAAAGTCACCAG |
| | MSH2-1seq-as2 | 163 | TCCCCAACCCCCTAAAGCGAGAATCCGCACAAGCACCAAC |
| | alternate | | |
| 1 | MSH2-1seq-s | 160 | TCTGCCTTTTTCTTCCATCGGGGGCGGGAAACAGCTTAGTGG |
| | MSH2-1seq-as | 161 | TCCCCAACCCCCTAAAGCGACGCACTGGAGAGGCTGCTCA |
| 2 | MSH2-2seq-s2 | 169 | TCTGCCTTTTTCTTCCATCGGGTGCTGCCATCCATGTAAGAC |
| | MSH2-2seq-as2 | 170 | TCCCCAACCCCCTAAAGCGACCAGCCAAACTGCAACTTTT |
| | alternate | | |
| 2 | MSH2-2seq-s3 | 171 | TCTGCCTTTTTCTTCCATCGGGTTCCTCCTTGCCTTCTGCCAT |
| | MSH2-2seq-as3 | 172 | TCCCCAACCCCCTAAAGCGAGGGATTACAAGCATGAGCCACTG |
| 3 | MSH2-3seq-s | 291 | TCTGCCTTTTTCTTCCATCGGGCAGAGCAAGACTTCATCTCA |
| | MSH2-3seq-as4 | 181 | TCCCCAACCCCCTAAAGCGACCTTAAATGAAACAGTATCATGTCAA |
| | alternate | | |
| | MSH2-3seq-s4 | 180 | TCTGCCTTTTTCTTCCATCGGGGGTTCATAGAGTTTGGAATTTTTCC |
| | MSH2-3seq-as4 | 181 | TCCCCAACCCCCTAAAGCGACCTTAAATGAAACAGTATCATGTCAA |
| 4 | MSH2-4seq-s2 | 191 | TCTGCCTTTTTCTTCCATCGGGGCATTCCATCCTGGGCGA |
| | MSH2-4seq-as2 | 192 | TCCCCAACCCCCTAAAGCGACAGCCTGGGCAACAAAAGTG |
| 5 | MSH2-5seq-s3 | 200 | TCTGCCTTTTTCTTCCATCGGGAGTTTTGATGGACATTTGGGTAA |
| | MSH2-5seq-as3 | 201 | TCCCCAACCCCCTAAAGCGAGTTAAAAAGTGGAGTGGAGGAGG |
| | alternate | | |
| 5 | MSH2-5seq-s2 | 198 | TCTGCCTTTTTCTTCCATCGGGTTCTTGGTTTGGATTGGGAAGG |
| | MSH2-5seq-as2 | 199 | TCCCCAACCCCCTAAAGCGAGGGGAGAGAAAAATACAGCCAT |
| 6 | MSH2-6seq-s | 209 | TCTGCCTTTTTCTTCCATCGGGTGAACATACGGATTAAGAGG |
| | MSH2-6seq-as | 210 | TCCCCAACCCCCTAAAGCGACATATACTTCCAAAACTGCA |
| 7 | MSH2-7seq-s3 | 218 | TCTGCCTTTTTCTTCCATCGGGGCTGATTTAGTTGAGACTTACGTGC |
| | MSH2-7seq-as2 | 219 | TCCCCAACCCCCTAAAGCGAGAGGACAGCACATTGCCAAG |
| 8 | MSH2-8seq-s | 229 | TCTGCCTTTTTCTTCCATCGGGAATAGTAACTTTGGAGACCTGC |
| | MSH2-8seq-as | 230 | TCCCCAACCCCCTAAAGCGACAGGACAGTTATGCCCAATA |
| 9 | MSH2-9seq-s | 234 | TCTGCCTTTTTCTTCCATCGGGGAAAGTCCTTAATAGTTGTGACTG |
| | MSH2-9seq-as | 235 | TCCCCAACCCCCTAAAGCGAGGGAACTTATAAAATAATTCTGGC |
| 10 | MSH2-10seq-s | 243 | TCTGCCTTTTTCTTCCATCGGGTCATAAGGGAGTTAAGGATTT |
| | MSH2-10seq-as | 244 | TCCCCAACCCCCTAAAGCGACTGCTCTATGGAAGAAAGCT |
| 11 | MSH2-11seq-s | 250 | TCTGCCTTTTTCTTCCATCGGGCATTTGTCCCTAAGGAGTTGTTC |
| | MSH2-11seq-as | 251 | TCCCCAACCCCCTAAAGCGACAGAATGTAATGGCTTGCGA |
| 12 | MSH2-12seq-s | 261 | TCTGCCTTTTTCTTCCATCGGGTGTTGAGTTTTAGGTGGGTTCC |
| | MSH2-12seq-as | 262 | TCCCCAACCCCCTAAAGCGATACCCCCACAAAGCCCAAA |
| 13 | MSH2-13seq-s | 270 | TCTGCCTTTTTCTTCCATCGGGGCTATGTCAGTGTAAACCTACGC |
| | MSH2-13seq-as | 271 | TCCCCAACCCCCTAAAGCGACTTCTCACAGGACAGAGACATACA |
| (GAP) = ex14 | (MSH2-GAPseq-s) = MSH2-14seq-s | 277 | TCTGCCTTTTTCTTCCATCGGGATGTTTGTGGCATATCCTTCC |
| | (MSH2-GAPseq-as) = MSH2-14seq- | 278 | TCCCCAACCCCCTAAAGCGATAGTAAGTTTCCCATTACCAAGTTC |
| | as | | |
| (14) = ex15 | (MSH2-14seq-s) = MSH2-15seq-s | 282 | TCTGCCTTTTTCTTCCATCGGGTTGGCTAATGTATTTGAAGTAATCC |
| | (MSH2-14seq-as) = MSH2-15seq-as | 283 | TCCCCAACCCCCTAAAGCGAACACAGAGGAAAACAAACAACAA |
| (15) = ex16 | (MSH2-15seq-s) = MSH2-16seq-s | 287 | TCTGCCTTTTTCTTCCATCGGGGTAAAGGTGGAGGAATTTGGG |
| | (MSH2-15seq-as) = MSH2-16seq-as | 288 | TCCCCAACCCCCTAAAGCGAGGCACTGACAGTTAACACTATGGA |

MLH1

| | | | |
|---|---|---|---|
| 1 | MLH1-1seq-s | 13 | TCTGCCTTTTTCTTCCATCGGGGCTTCAGGGAGGGACGAAGA |
| | MLH1-1seq-as | 14 | TCCCCAACCCCCTAAAGCGATGCGCTGTACATGCCTCTGC |
| 2 | MLH1-2seq-s | 20 | TCTGCCTTTTTCTTCCATCGGGTGCCCGTCTCTTCCCTCTCT |
| | MLH1-2seq-as | 21 | TCCCCAACCCCCTAAAGCGACCTGAACAGTGCCCAGCAAA |
| 3 | MLH1-3seq-s | 27 | TCTGCCTTTTTCTTCCATCGGGCAAGACTCTGTCTCAAAGGAGGTT |
| | MLH1-3seq-as2 | 30 | TCCCCAACCCCCTAAAGCGACATTAAGTTTGCTCAGATTTGCATA |
| 4 | MLH1-4seq-s | 36 | TCTGCCTTTTTCTTCCATCGGGCATGTCATCAAAGCAAGTGAGC |
| | MLH1-4seq-as | 37 | TCCCCAACCCCCTAAAGCGATGAGACAGGATTACTCTGAGACCT |
| 5 | MLH1-5seq-s2 | 47 | TCTGCCTTTTTCTTCCATCGGGCCCTTGGGATTAGTATCTATCTCT |
| | MLH1-5seq-as | 48 | TCCCCAACCCCCTAAAGCGAGGACCTCCATTAACTAGTGCAA |
| 6 | MLH1-6seq-s | 52 | TCTGCCTTTTTCTTCCATCGGGCTGTTAATGCTGTCTTATCCCTGG |
| | MLH1-6seq-as | 53 | TCCCCAACCCCCTAAAGCGACCATCTAGCTCAGCAACTGTTCA |
| 7 | MLH1-7seq-s | 57 | TCTGCCTTTTTCTTCCATCGGGTTCCATGAAAGTTTCTGCTGG |
| | MLH1-7seq-as | 58 | TCCCCAACCCCCTAAAGCGACCTTATCTCCACCAGCAAACTA |
| 8 | MLH1-8seq-s | 66 | TCTGCCTTTTTCTTCCATCGGGGGTTTATGGGGGATGGTTTTG |
| | MLH1-8seq-as | 67 | TCCCCAACCCCCTAAAGCGACGCCACAGAATCTAGGAGATTACA |
| 9 | MLH1-9seq-s | 75 | TCTGCCTTTTTCTTCCATCGGGGGTGGGTGAATGGGTGAACA |
| | MLH1-9seq-as | 76 | TCCCCAACCCCCTAAAGCGATTTGCCATGAGGTTTCTCCA |
| 10 | MLH1-10seq-s | 80 | TCTGCCTTTTTCTTCCATCGGGGCTGGAAAGTGGCGACAGG |
| | MLH1-10seq-as | 81 | TCCCCAACCCCCTAAAGCGAGCCAGTGGTGTATGGGATTCA |
| 11 | MLH1-11seq-s | 89 | TCTGCCTTTTTCTTCCATCGGGAGACTGAGGCAAAGAAAGATG |
| | MLH1-11seq-as | 90 | TCCCCAACCCCCTAAAGCGAAGGCAAAAATCTGGGCTCT |
| 12 | MLH1-12seq-s | 100 | TCTGCCTTTTTCTTCCATCGGGTTTCGGGCAGAATTGCTTC |
| | MLH1-12seq-as | 101 | TCCCCAACCCCCTAAAGCGAGCAGAGAGAAGATGCAAGTGATT |
| | alternate | | |
| 12 | MLH1-12seq-s2 | 103 | TCTGCCTTTTTCTTCCATCGGGATAGCTGGTGGTGATGGTTGCG |
| | MLH1-12seq-as2 | 104 | TCCCCAACCCCCTAAAGCGACCATTCCAGCACCATTCCAGAG |
| 13 | MLH1-13seq-s | 114 | TCTGCCTTTTTCTTCCATCGGGACTGATCTTGTTGGCCTTCTG |
| | MLH1-13seq-as | 115 | TCCCCAACCCCCTAAAGCGATGGCCACTCTGACAACATGA |
| 14 | MLH1-14seq-s | 121 | TCTGCCTTTTTCTTCCATCGGGTGTTCGTTTTCACCAGGAGG |
| | MLH1-14seq-as | 122 | TCCCCAACCCCCTAAAGCGATCGAACTTGGATTTGAAACCAC |
| 15 | MLH1-15seq-s2 | 126 | TCTGCCTTTTTCTTCCATCGGGAGATTCCACAGCCAGGCAG |
| | MLH1-15seq-as2 | 127 | TCCCCAACCCCCTAAAGCGATACCTCCATATGCAAATCATACAA |
| 16 | MLH1-16seq-s | 135 | TCTGCCTTTTTCTTCCATCGGGGGTTTTGTTGTGGATTGTTCAGG |
| | MLH1-16seq-as | 136 | TCCCCAACCCCCTAAAGCGATGGGATTACAGCCATGAGCC |
| 17 | MLH1-17seq-s | 140 | TCTGCCTTTTTCTTCCATCGGGTTTAAGTGTTTAGGTCTGCCCC |
| | MLH1-17seq-as | 141 | TCCCCAACCCCCTAAAGCGAGCTATCCCACCCTTATCATCTTT |
| 18 | MLH1-18seq-s | 147 | TCTGCCTTTTTCTTCCATCGGGAAGATGATAAGGGTGGGATAGC |
| | MLH1-18seq-as | 148 | TCCCCAACCCCCTAAAGCGACCGAAATTTTAGAGATGGGC |
| 19 | MLH1-19seq-s | 156 | TCTGCCTTTTTCTTCCATCGGGGCTATGATCACACCACTGCCC |
| | MLH1-19seq-as | 157 | TCCCCAACCCCCTAAAGCGACCTCTTTTTGGCATCTGAACTG |

| | |
|---|---|
| MSH2/MLH1 | Sequencing Primers |

| | | | |
|---|---|---|---|
| all exons | MSH2 s tag | 289 | TCTGCCTTTTTCTTCCATCGGG |
| all exons | MSH2 as tag | 290 | TCCCCAACCCCCTAAAGCGA |

| | |
|---|---|
| MLH1 | Sequencing Primers internal instead of tagged primer in sense direction |

| | | | |
|---|---|---|---|
| 3 | MLH1-3seq-s2-int | 29 | ctggattaaatcaagaaaatggg |
| 12 | MLH1-12seq-s2-int | 102 | CAGACTTTGCTACCAGGACTTGCT |

| | |
|---|---|
| MSH2 | Sequencing Primers internal instead of tagged primer in that direction |

| | | | |
|---|---|---|---|
| 2 | MSH2-2seq-s2-int | 292 | GGAGCAAAGAATCTGCAGAGTGTT |
| 5 | MSH2-5seq-as2-int | 293 | CTGAAAAAGGTTAAGGGCTCTGACT |

rev. 091703 AK
rev. 112003 AK
rev. 021304 AK
rev. 030604 AK
rev. 032604 AK

note old name for exon 14-16 in brackets

TABLE F
EXTENSION PRODUCTS GENERATED FOR TTGE ASSAY

Clamp region sense corresponds to:
5′ CGCCCGCCGCGCCCCGCGCCCGCCCCGCCGCCCCCGCCCG (SEQ ID NO.: 344)
Clamp region rev. complement
5′ CGGGCGGGGGCGGCGGGGCGGGCGCGGGGCGCGGCGGGCG (SEQ ID NO.: 345)
MSH2 2B-3
CGCCCGCCGCGCCCCGCGCCCGCCCCGCCGCCCCCGCCCGgGAGCAAAG
AATCTGCAGAGTGTTGTGCTTAGTAAAATGAATTTTGAATCTTTTGTAAAAGA
TCTTCTTCTGGTTCGTCAGTATAGAGTTGAAGTTTATAAGAATAGAGCTGGA
AATAAGGCATCCAAGGAGAATgattggtatttggcatataaggtaatta
(SEQ ID NO.: 346)
MSH2 2C
ATAAGGCATCCAAGGAGAATGATTGGTATTTGGCATATAAGgtaattatcttccttttta
atttacttatttttttaagagtagaaaaataaaaatgtgaagaatttaattgtgttttagtattttaagtagatCGGGC
GGGGGCGGCGGGGCGGGCGCGGGGCGCGGCGGGCG (SEQ ID NO.: 347)
MSH2 3A
CGCCCGCCGCGCCCCGCGCCCGCCCCGCCGCCCCCGCCCGaacattttattaata
aggttcatagagtttggatttttcctttttgcttataaaattttaaagtatgttcaagagtttgttaaatttttaaaattttattttt
acttagGCTTCTCCTGGCAAT (SEQ ID NO.: 348)
MSH2 3B2
CGCCCGCCGCGCCCCGCGCCCGCCCCGCCGCCCCCGCCCGatttttacttagGC
TTCTCCTGGCAATCTCTCTCAGTTTGAAGACATTCTCTTTGGTAACAATGATA
TGTCAGCTTCCATTGGTGTTGTGGGTGTTAAAATGTCCGCAGTTGATGGCCA
GAGACAGGTTGGAGTTGGGTATGTGGATTCCATACAGAGGAAACTAGGACT
GTGTGAATTCCCTGATAATGATCAGTTCTCCAATCTTGAGGCTCTCCTCATC
CAGATTGGACCAAAGGAATGTGTTTTACCCGGAGGAGAGACTGCTGGAGAC
ATGGGGAAACTG (SEQ ID NO.: 349)
MSH2 3C
AATGTGTTTTACCCGGAGGAGAGACTGCTGGAGACATGGGGAAACTGAGAC
AGgtaagcaaattgagtctagtgatagaggagattccaggcctaggaaaggctctttaattgacatgatactgttt
catttaagCGGGCGGGGGCGGCGGGGCGGGCGCGGGGCGCGGCGGGCG
(SEQ ID NO.: 350)
MSH2 4A
CGCCCGCCGCGCCCCGCGCCCGCCCCGCCGCCCCCGCCCGtccttttctcatagta
gtttaaactatttctttcaaaatagATAATTCAAAGAGGAGGAATTCTGATCACAGAAAGA
AAAAAAGCTGACTTTTCCACAAAAGACATTTATCAGGACCTCAA
(SEQ ID NO.: 351)
MSH2 4A2
CGCCCGCCGCGCCCCGCGCCCGCCCCGCCGCCCCCGCCCGtttctttcaaaatag
ATAATTCAAAGAGGAGGAATTCTGATCACAGAAAGAAAAAAAGCTGACTTTT
CCACAAAAGACATTTATCAGGACCTCAACCGGTTGTTGAAAGGCAAAA
(SEQ ID NO.: 352)
MSH2 4B2
ATTTATCAGGACCTCAACCGGTTGTTGAAAGGCAAAAAGGGAGAGCAGATG
AATAGTGCTGTATTGCCAGAAATGGAGAATCAGgtacatggattataaatgtgaattacaC
GGGCGGGGGCGGCGGGGCGGGCGCGGGGCGCGGCGGGCG
(SEQ ID NO.: 353)
MSH2 4C
ATTGCCAGAAATGGAGAATCAGgtacatggattataaatgtgaattacaatatatataatgtaaata
tgtCGGGCGGGGGCGGCGGGGCGGGCGCGGGGCGCGGCGGGCG
(SEQ ID NO.: 354)
MSH2 5A
CGCCCGCCGCGCCCCGCGCCCGCCCCGCCGCCCCCGCCCGtcattttgcatttgttt
tttaaaatctttagaactggatccagtggtatagaaatcttcgatttttaaattcttaattttagGTTGCAGTTTC
ATCACTGTCTGCGGTAATCAAG
(SEQ ID NO.: 355)
MSH2 5B
CGCCCGCCGCGCCCCGCGCCCGCCCCGCCGCCCCCGCCCGcttcgatttttaaatt
cttaattttagGTTGCAGTTTCATCACTGTCTGCGGTAATCAAGTTTTTAGAACTCT
TATCAGATGATTCCAACTTTGGACAGTTTGAACTGACTACTTTTGACTTCAGC
CAGTATATGAAATTGGATATTGCAGCAGTCAGAGCCCTTAACCTTTTTCAGgt
(SEQ ID NO.: 356)
MSH2 6A
CGCCCGCCGCGCCCCGCGCCCGCCCCGCCGCCCCCGCCCGgtttttcatggcgta
gtaaggttttcactaatgagcttgccattctttctattttattttttgtttactagGGTTCTGTTGAAGATACCA
CTGGCTCTCAGT (SEQ ID NO.: 357)
MSH2 6B2
tttactagGGTTCTGTTGAAGATACCACTGGCTCTCAGTCTCTGGCTGCCTTGCT
GAATAAGTGTAAAACCCCTCAAGGACAAAGACTTGTTAACCAGTGGATTAAG
CAGCCTCTCATGGATAAGAACAGAATAGAGGAGAGgtatCGGGCGGGGGCG
GCGGGGCGGGCGCGGGGCGCGGCGGGCG (SEQ ID NO.: 358)
MSH2 6C
TCAAGGACAAAGACTTGTTAACCAGTGGATTAAGCAGCCTCTCATGGATAAG
AACAGAATAGAGGAGAGgtatgttattagtttatactttcgttagttttatgtaacctgcagttacccacatg
attataccacttattCGGGCGGGGGCGGCGGGGCGGGCGCGGGGCGCGGCGGGCG
(SEQ ID NO.: 359)
MSH2 7A2
CGCCCGCCGCGCCCCGCGCCCGCCCCGCCGCCCCCGCCCGgttgagacttacgt
gcttagttgataaattttaattttatactaaaatattttacattaattcaagttaatttatttcagATTGAATTTAGT
GGAAGCTTTTGTAGAAGATGCAGAATTG (SEQ ID NO.: 360)
MSH2 7B2
CGCCCGCCGCGCCCCGCGCCCGCCCCGCCGCCCCCGCCCGatttatttcagATT
GAATTTAGTGGAAGCTTTTGTAGAAGATGCAGAATTGAGGCAGACTTTACAA
GAAGATTTACTTCGTCGATTCCCAGATCTTAACCGACTTGCCAAGAAGTTTC
AAAGACAAGCAGCAAACT (SEQ ID NO.: 361)
MSH2 7C3
GACTTGCCAAGAAGTTTCAAAGACAAGCAGCAAACTTACAAGATTGTTACCG
ACTCTATCAGGGTATAAATCAACTACCTAATGTTATACAGGCTCTGGAAAAA
CATGAAGgtaacaagtgattttgtttttttgttttccttcaactcatacaatatatacttggcaatgtgctgtcctcata
aagttggtggtggtgactcaCGGGCGGGGGCGGCGGGGCGGGCGCGGGGCGCGG
CGGGCG (SEQ ID NO.: 362)
MSH2 8A
CGCCCGCCGCGCCCCGCGCCCGCCCCGCCGCCCCCGCCCGtttggatcaaatga
tgcttgtttatctcagtcaaaattttatgatttgtattctgtaaaatgagatctttttatttgtttgttttactactttcttttagGA
AAACACCAGAAATTATTGTTGGCAGTTTTTGTGACTCCTCTTACTGAT
(SEQ ID NO.: 363)
MSH2 8B
TTGTGACTCCTCTTACTGATCTTCGTTCTGACTTCTCCAAGTTTCAGGAAATG
ATAGAAACAACTTTAGATATGGATCAGgtatgcaatatactttttaatttaagcagtagttaCGG
GCGGGGGCGGCGGGGCGGGCGCGGGGCGCGGCGGGCG
(SEQ ID NO.: 364)
MSH2 8C
CTGACTTCTCCAAGTTTCAGGAAATGATAGAAACAACTTTAGATATGGATCA
Ggtatgcaatatactttttaatttaagcagtagttatttttaaaaagcaaaggccactttaagaaagtttgtagatttttc
tttttagtatctaattgtagcacCGGGCGGGGGCGGCGGGGCGGGCGCGGGGCGCGG
CGGGCG (SEQ ID NO.: 365)
MSH2 8D
AGAAATTATTGTTGGCAGTTTTTGTGACTCCTCTTACTGATCTTCGTTCTGAC
TTCTCCAAGTTTCAGGAAATGATAGAAACAACTTTAGATATGGATCAGgtatgca
atCGGGCGGGGGCGGCGGGGCGGGCGCGGGGCGCGGCGGGCG
(SEQ ID NO.: 366)
MSH2 9A2
CGCCCGCCGCGCCCCGCGCCCGCCCCGCCGCCCCCGCCCGaatatttgctttata
atttctgtctttacccattatttataggattttgtcactttgttctgtttgcagGTGGAAAACCATGAATTCCT
TGTAAAACCTTCATTTGATCCTAATCTCAGTGAATTAAGAGAAATAATGAATG
ACTTGGAAAAGAAGATGCAGTCAACATTAATAAGTGCAGCCAGAGATCTTGg
taagaatgggtcattggaggttggaataattct (SEQ ID NO.: 367)
MSH2 10A
CGCCCGCCGCGCCCCGCGCCCGCCCCGCCGCCCCCGCCCGgaattacattgaa
aaatggtagtaggtatttatggaatactttttcttttcttcttgattatcaagGCTTGGACCCTGGCAAACA
GATTAA (SEQ ID NO.: 368)
MSH2 10B2
tcttcttgattatcaagGCTTGGACCCTGGCAAACAGATTAAACTGGATTCCAGTGCA
CAGTTTGGATATTACTTTCGTGTAACCTGTAAGGAAGAAAAAGTCCTTCGTA
ACAATAAAAACTTTAGTACTGTAGATATCCAGAAGAATGGTGTTACGGGCGG
GGGCGGCGGGGCGGGCGCGGGGCGCGGCGGGCG
(SEQ ID NO.: 369)
MSH2 10C3
TGCACAGTTTGGATATTACTTTCGTGTAACCTGTAAGGAAGAAAAAGTCCTT
CGTAACAATAAAAACTTTAGTACTGTAGATATCCAGAAGAATGGTGTTAAATT
TACCAACAGgtttgcaagtcgttattatatttttaaccctttattaattccctaaatgctctaacatgatgtgaatgtt
ctatgataagttttacCGGGCGGGGGCGGCGGGGCGGGCGCGGGGCGCGGCGGG
CG (SEQ ID NO.: 370)
MSH2 11A2
CGCCCGCCGCGCCCCGCGCCCGCCCCGCCGCCCCCGCCCGtttggatatgtttca
cgtagtacacattgcttctagtacacattttaatatttttaataaaactgttatttcgatttgcagCAAATTGACTT
CTTTAAATGAAGAGTATACCAAAAATAAAACAGAATATGAAGAAGCCCAGGA
TGCCATTGTTAAAG (SEQ ID NO.: 371)
MSH2 11B2
CGCCCGCCGCGCCCCGCGCCCGCCCCGCCGCCCCCGCCCGgCAAATTGA
CTTCTTTAAATGAAGAGTATACCAAAAATAAAACAGAATATGAAGAAGCCCA
GGATGCCATTGTTAAAGAAATTGTCAATATTTCTTCAGgtaaacttaatagaactaata
atgttctgaatgtcacctggcttttggtaacagaagaaaaatcatgatatttgaagtgtgttttgttattttcgcaagcc
at (SEQ ID NO.: 372)
MSH2 12A
CGCCCGCCGCGCCCCGCGCCCGCCCCGCCGCCCCCGCCCGaggaaatgggtttt
gaattcccaaatggggggattaaatgtatttttacggcttatatctgtttattattcagtattcctgtgtacattttctgttttt
atttttatacagGCTATGTAGAACCAATGCAGACACTCAATGATGTGTTAGCTC
(SEQ ID NO.: 373)
MSH2 12B2
CGCCCGCCGCGCCCCGCGCCCGCCCCGCCGCCCCCGCCCGatttttatacagGC
TATGTAGAACCAATGCAGACACTCAATGATGTGTTAGCTCAGCTAGATGCTG
TTGTCAGCTTTGCTCACGTGTCAAATGGAGCACCTGTTCCATATGT
(SEQ ID NO.: 374)
MSH2 12C
TGGAGCACCTGTTCCATATGTACGACCAGCCATTTTGGAGAAAAGGACAAGG
AAGAATTATATTAAAAGCATCCAGGCATGCTTGTGTTGAAGTTCAAGATGAA
ATTGCATTTATTCCTAATGACGTATACTTTGAAAAAGATAAACAGATGTTCCA
CATCATTACTGgtaaaaaacctggtttttgggctttgtgggggtaacgttttgttCGGGCGGGGGCG
GCGGGGCGGGCGCGGGGCGCGGCGGGCG (SEQ ID NO.: 375)
MSH2 12E
cagctttgctcacgtgtcaaaTGGAGCACCTGTTCCATATGTACGACCAGCCATTTTGG
AGAAAGGACAAGGAAGAATTATATTAAAAGCATCCAGGCATGCTTGTGTTGA
AGTTCAAGATGCGGGCGGGGGCGGCGGGGCGGGCGCGGGGCGCGGCGG
GCG (SEQ ID NO.: 376)
MSH2 13A
CGCCCGCCGCGCCCCGCGCCCGCCCCGCCGCCCCCGCCCGaggactaacaat
ccatttattagtagcagaaagaagtttaaaatcttgctttctgatataatttgttttgtagGCCCCAATATGGG
AGGTAAATCAACATATATTCGACAAACTGGGGTGATAGTACTCATGGCCCA
(SEQ ID NO.: 377)
MSH2 13B
CGCCCGCCGCGCCCCGCGCCCGCCCCGCCGCCCCCGCCCGATGGGAGGT
AAATCAACATATATTCGACAAACTGGGGTGATAGTACTCATGGCCCAAATTG
GGTGTTTTGTGCCATGTGAGTCAGCAGAAGTGTCCATTGTGGACTGCATCTT
AGCCCGAGTAGGGGCTGGTGACAGTCAATTGAAAGGAGTC
(SEQ ID NO.: 378)
MSH2 13C5
TTGTGGACTGCATCTTAGCCCGAGTAGGGGCTGGTGACAGTCAATTGAAAG
GAGTCTCCACGTTCATGGCTGAAATGTTGGAAACTGCTTCTATCCTCAGgtaa
gtgcatctcctagtcccttgaagatagaaatgtatgtctctgtcctgtgaCGGGCGGGGGCGGCGGG
GCGGGCGCGGGGCGCGGCGGGCG (SEQ ID NO.: 379)
MSH2 14A3
CGCCCGCCGCGCCCCGCGCCCGCCCCGCCGCCCCCGCCCGgtatgtgtatgttac
cacattttatgtgatgggaaatttcatgtaattatgtgcttcagGTCTGCAACCAAAGATTCATTAAT
AATCATAGATGAATTGGGAAGAGGAACTTCTACCTACGATGGATTTGGGTTA
GCATGGGCTATATCAGAATACATTGCAACAAAGATTGGTGCTTTTTGCATGT
TTGCAACCCATTTTCATGAACTTACTGCCTTGGCCAATCAGATACCAACTGTT
AATAATCTACATGTCACAGCACTCACCACTGAAGAGACCTTAACTA
(SEQ ID NO.: 380)
MSH2 14B
ATAATCTACATGTCACAGCACTCACCACTGAAGAGACCTTAACTATGCTTTAT
CAGGTGAAGAAAGgtatgtactattggagtactctaaattcagaacttggtaatgggaaacttactaccctt
gaaatcatcagtaattgccttattcCGGGCGGGGGCGGCGGGGCGGGCGCGGGGCGC
GGCGGGCG (SEQ ID NO.: 381)
MSH2 15A
gtctcttctcatgctgtcccctcacgcttccccaaatttcttatagGTGTCTGTGATCAAAGTTTTGGG
ATTCATGTTGCAGAGCTTGCTAATTTCCCTAAGCATGTAATAGAGTGTGCTA
AACAGAAAGCCCTGGAACTTGAGGAGTTTCAGTATATTGGAGAATCGCAAG
GATATGATATCATGGAACCAGCAGCAAAGAAGTGCTATCTGGAAAGAGAGgtt
tgtcagtttgttttcatagtttaacttagcttctctattCGGGCGGGGGCGGCGGGGCGGGCGCG
GGGCGCGGCGGGCG (SEQ ID NO.: 382)
MSH2 16A
ttactaatgggacattcacatgtgtttcagCAAGGTGAAAAAATTATTCAGGAGTTCCTGTCC
AAGGTGAAACAAATGCCCTTTACTGAAATGTCAGAAGAAAACATCACAATAA
AGTTAAAACAGCTAAAAGCTGAAGTAATAGCAAAGAATAATAGCTTTGTAAAT
GAAATCATTTCACGAATAAAAGTTACTACGTGAaaaatcccagtaatggaatgaaggtaa
tattgataagctattgtCGGGCGGGGGCGGCGGGGCGGGCGCGGGGCGCGGCGG
GCG (SEQ ID NO.: 383)
MLH1
Clamp region sense corresponds to:
5′ CGCCCGCCGCGCCCCGCGCCCGCCCCGCCGCCCCCGCCCG
(SEQ ID NO.: 344)
Clamp region rev. complement
5′ CGGGCGGGGGCGGCGGGGCGGGCGCGGGGCGCGGCGGGCG
(SEQ ID NO.: 345)
MLH1 1A
CGCCCGCCGCGCCCCGCGCCCGCCCCGCCGCCCCCGCCCGcaatagctgccgc
tgaagggtggggctggatggcgtaagctacagctgaaggaagaacgtgagcacgaggcactgaggtgattg
gctgaaggcacttccgttgagcatctagacgtttccttggctcttctggcgccaaaATGTCGTTCGTGGC
AGGGGTTATTCGGCGGCTGGACGAGACAGTGGTGAACCGCATCGCGGCGG
GGGAAGTTATCCAGCG (SEQ ID NO.: 384)
MLH1 1B
GGCGGGGGAAGTTATCCAGCGGCCAGCTAATGCTATCAAAGAGATGATTGA
GAACTGgtacggagggagtcgagccgggctcacttaagggctacgacttaacgggccgcgtcactcaatg
gcgcg CGGGCGGGGGCGGCGGGGCGGGCGCGGGGCGCGGCGGGCG
(SEQ ID NO.: 385)
MLH1 1C
CGCCCGCCGCGCCCCGCGCCCGCCCCGCCGCCCCCGCCCGCAAAGAGAT
GATTGAGAACTGgtacggagggagtcgagccgggctcacttaagggctacgacttaacgggccgcgt
cactcaatggcgcggacacgcctctttgcccgggcagaggcatg
(SEQ ID NO.: 386)
MLH1 1D
CGCCCGCCGCGCCCCGCGCCCGCCCCGCCGCCCCCGCCCGggaagaacgtga
gcacgaggcactgaggtgattggctgaaggcacttccgttgagcatctagacgtttccttggctcttctggcgcca
aaATGTCGTTCGTGGCAGGGGTTATTCGGCGGCTGGACGAGACAGTGGTGA
ACCGCATCGCGGCGGGGGAAGTTATCCAGCGgccagctaatg
(SEQ ID NO.: 387)
MLH1 2A
CGCCCGCCGCGCCCCGCGCCCGCCCCGCCGCCCCCGCCCGttatcattgcttggc
tcatattaaaatatgtacattagagtagttgcagactgataaattattttctgtttgatttgccagTTTAGATGCA
AAATCCACAAGTATTCAAGTGATTGTTAAAGAGGGAGGCCTGAAGTTGATTC
AGATCCAAGACAA (SEQ ID NO.: 388)
MLH1 2B
CGCCCGCCGCGCCCCGCGCCCGCCCCGCCGCCCCCGCCCGGCAAAATCC
ACAAGTATTCAAGTGATTGTTAAAGAGGGAGGCCTGAAGTTGATTCAGATCC
AAGACAATGGCACCGGGATCAGGgtaagtaaaacctcaaagtagcaggatgtttgtgcgcttca
tggaagagtcagg (SEQ ID NO.: 389)
MLH1 3A
CGCCCGCCGCGCCCCGCGCCCGCCCCGCCGCCCCCGCCCGgggaattcaaag
agatttggaaaaatgagtaacatgattatttactcatctttttggtatctaacagAAAGAAGATCTGGATA
TTGTATGTGAAAGGTTCACTACTAGTAAACTGCAGTCCTTTGAGGATTTAGC
CAGTATTTCTACCTATGGCTTTCGAGGTGAGgtaagctaaagattcaagaa
(SEQ ID NO.: 390)
MLH1 3B
ATATTGTATGTGAAAGGTTCACTACTAGTAAACTGCAGTCCTTTGAGGATTTA
GCCAGTATTTCTACCTATGGCTTTCGAGGTGAGgtaagctaaagattcaagaaatgtgta
aaatatcctcctgtgatgacattgtctgtcatttgttagtatgtatttctcaacatagataaataaggtttggtacCGG
GCGGGGGCGGCGGGGCGGGCGCGGGGCGCGGCGGGCG
(SEQ ID NO.: 391)
MLH1 4A4
ggtgaggtgacagtgggtgacccagcagtgagtttttctttcagtctattttcttttcttccttagGCTTTGGCCA
GCATAAGCCATGTGGCTCATGTTACTATTACAACGAAAACAGCTGATGGAAA
GTGTGCATACAGgtatagtgctgacttcttttactcatatatattcaCGGGCGGGGGCGGCGG
GGCGGGCGCGGGGCGCGGCGGGCG (SEQ ID NO.: 392)
MLH1 4B2
TCATGTTACTATTACAACGAAAACAGCTGATGGAAAGTGTGCATACAGgtatagt
gctgacttcttttactcatatatattcattctgaaatgtattttttgcctaggtctcagagtaatcctgtctcaacaccagtg
ttatcCGGGCGGGGGCGGCGGGGCGGGCGCGGGGCGCGGCGGGCG
(SEQ ID NO.: 393)
MLH1 5A
CGCCCGCCGCGCCCCGCGCCCGCCCCGCCGCCCCCGCCCGgggattagtatcta
tctctctactggatattaatttgttatattttctcattagAGCAAGTTACTCAGATGGAAAACTGAAAG
(SEQ ID NO.: 394)
MLH1 5B2
CTGAAAGCCCCTCCTAAACCATGTGCTGGCAATCAAGGGACCCAGATCACG
gtaagaatggtacatgggagagtaaattgttgaagctCGGGCGGGGGCGGCGGGGCGGGC
GCGGGGCGCGGCGGGCG (SEQ ID NO.: 395)
MLH1 5C2
GGGACCCAGATCACGgtaagaatggtacatgggagagtaaattgttgaagctttgtttgtataaatattg
gaat CGGGCGGGGGCGGCGGGGCGGGCGCGGGGCGCGGCGGGCG
(SEQ ID NO.: 396)
MLH1 5D
CGCCCGCCGCGCCCCGCGCCCGCCCCGCCGCCCCCGCCCGtttgttatattttctca
ttagAGCAAGTTACTCAGATGGAAAACTGAAAGCCCCTCCTAAACCATGTGCT
GGCAATCAAGGGACCCAGATCACGgtaagaat (SEQ ID NO.: 397)
MLH1 6-5
CGCCCGCCGCGCCCCGCGCCCGCCCCGCCGCCCCCGCCCGattcactatcttaa
gacctcgcttttgccaggacatcttgggttttattttcaagtacttctatgaatttacaagaaaaatcaatcttctgttca
gGTGGAGGACCTTTTTTACAACATAGCCACGAGGAGAAAAGCTTTAAAAAAT
CCAAGTGAAGAATATGGGAAAATTTTGGAAGTTGTTGGCAGgtacagtccaaaatct
gggagtgggtctctgagatttgtcatcaaagtaatgtgttctag
(SEQ ID NO.: 398)
MLH1 7
taactaaaagggggctctgacatctagtgtgtgtttttggcaactcttttcttactcttttgtttttcttttccagGTATTC
AGTACACAATGCAGGCATTAGTTTCTCAGTTAAAAAAgtaagttcttggtttatgggggat
ggttttgttttatgaaaagaaaaaaggggatttttaatagtttgctggtggagataaggttatgatgtttcagtctcagc
catgagacaataaaCGGGCGGGGGCGGCGGGGCGGGCGCGGGGCGCGGCGG
GCG (SEQ ID NO.: 399)
MLH1 8A
CGCCCGCCGCGCCCCGCGCCCGCCCCGCCGCCCCCGCCCGgctggtggagata
aggttatgatgtttcagtctcagccatgagacaataaatccttgtgtcttctgctgtttgtttatcagCAAGGAGA
GACAGTAGCTGATGTTAGGACACTACCCAATGCCTCAACCGTGGACA
(SEQ ID NO.: 400)
MLH1 8B2 (also has 4 bp miniclamp)
GGGGGCAAGGAGAGACAGTAGCTGATGTTAGGACACTACCCAATGCCTCAA
CCGTGGACAATATTCGCTCCATCTTTGGAAATGCTGTTAGTCGgtatgtcgataac
ctatat CGGGCGGGGGCGGCGGGGCGGGCGCGGGGCGCGGCGGGCG
(SEQ ID NO.: 401)
MLH1 8C2
AAATGCTGTTAGTCGgtatgtcgataacctatataaaaaaatcttttacatttattatcttggtttatcattcca
tcacattattttggaacctttcaagaCGGGCGGGGGCGGCGGGGCGGGCGCGGGGCGC
GGCGGGCG (SEQ ID NO.: 402)
MLH1 9A3
CGCCCGCCGCGCCCCGCGCCCGCCCCGCCGCCCCCGCCCGgtaatgtttgagtttt
gagtattttcaaaagcttcagaatctcttttctaatagAGAACTGATAGAAATTGGATGTGAGGAT
AAAACCCTAGCCTTCAAAATGAATGGTTACATATCCAATGCAAACTACTCAG
TGAAGAAGTGCATCTTCTTACTCTTCATCAACCgtaagttaaaaagaaccacatgggaa
atccactcacaggaaacacccacagggaattttatgggaccatggaaaaatttctg
(SEQ ID NO.: 403)
MLH1 9B
CGCCCGCCGCGCCCCGCGCCCGCCCCGCCGCCCCCGCCCGcaaagttagtttat
gggaaggaaccttgtgtttttaaattctgattcttttgtaatgtttgagttttgagtattttcaaaagcttcagaatctcttttc
taatagAGAACTGATAGAAATTGGATGTGAGGATAAAACCCTAGCCTTCAAAAT
GAATGGTTACATATCCAATGCAAACTACTCAGTGAAGAAGTGCATCTTCTTA
CTCTTC (SEQ ID NO.: 404)
MLH1 9C
CGCCCGCCGCGCCCCGCGCCCGCCCCGCCGCCCCCGCCCGCTTCAAAAT
GAATGGTTACATATCCAATGCAAACTACTCAGTGAAGAAGTGCATCTTCTTA
CTCTTCATCAACCgtaagttaaaaagaaccacatgggaaatccactcacaggaaacacccacaggg
aat (SEQ ID NO.: 405)
MLH1 10
CGCCCGCCGCGCCCCGCGCCCGCCCCGCCGCCCCCGCCCGtgaatgtacacct
gtgacctcacccctcaggacagttttgaactggttgctttctttttattgtttagATCGTCTGGTAGAATCAA
CTTCCTTGAGAAAGCCATAGAAACAGTGTATGCAGCCTATTTGCCCAAAAA
CACACACCCATTCCTGTACCTCAGgtaatgtagcaccaaactcctcaaccaagactcacaagg
aacagatgttcta (SEQ ID NO.: 406)
MLH1 11A
CGCCCGCCGCGCCCCGCGCCCGCCCCGCCGCCCCCGCCCGttgaccactgtgtc
atctggcctcaaatcttctggccaccacatacaccatatgtgggctttttctccccctcccactatctaaggtaattgtt
ctctcttattttcctgacagTTTAGAAATCAGTCCCCAGAATGTGGATGTTAATGTGCAC
CCCACAAAGCATGAAGTTCACTTCCTGCAC (SEQ ID NO.: 407)
MLH1 11B
CGCCCGCCGCGCCCCGCGCCCGCCCCGCCGCCCCCGCCCGCAGAATGTG
GATGTTAATGTGCACCCCACAAAGCATGAAGTTCACTTCCTGCACGAGGAG
AGCATCCTGGAGCGGGTGCAGCAGCACATCGAGAGCAAGCTCCTGGGCTC
CAATTCCTCC (SEQ ID NO.: 408)
MLH1 11C4
cagcagcacatcgagagcaagctcctgggctccaattcctccaggatgtacttcacccaggtcagggcgcttct
catccagctacttctctggggcctttgaaatgtgcccggccagacgtgagagcccagatCGGGCGGGGG
CGGCGGGGCGGGCGCGGGGCGCGGCGGGCG (SEQ ID NO.: 409)
MLH1 12B
CGCCCGCCGCGCCCCGCGCCCGCCCCGCCGCCCCCGCCCGttttttttaatacagA
CTTTGCTACCAGGACTTGCTGGCCCCTCTGGGGAGATGGTTAAATCCACAA
CAAGTCTGACCTCGTCTTCTACTTCTGGAAGTAGTGATAAGGTCTATGCCCA
CCAGATGGTTCGTACAGATTCCCGGGAACAGAAGCTTGATGCATTTCTGCA
GCCTCTGAGCAAACCCCTGTCCAGTCAGCCCCAGGCCATTGTCAC
(SEQ ID NO.: 410)
MLH1 12C
CATTTCTGCAGCCTCTGAGCAAACCCCTGTCCAGTCAGCCCCAGGCCATTG
TCACAGAGGATAAGACAGATATTTCTAGTGGCAGGGCTAGGCAGCAAGATG
AGGAGATGCTTGAACTCCCAGCCCCTGCTGAAGTGGCTGCCAAAAACGGG
CGGGGGCGGCGGGGCGGGCGCGGGGCGCGGCGGGCG
(SEQ ID NO.: 411)
MLH1 12D3
AGCCCCTGCTGAAGTGGCTGCCAAAAATCAGAGCTTGGAGGGGGATACAA
CAAAGGGGACTTCAGAAATGTCAGAGAAGAGAGGACCTACTTCCAGCAACC
CCAGgtatggccttttgggaaaagtacagcctacctcctttattctgtaataaaactgccttctCGGGCGGG
GGCGGCGGGGCGGGCGCGGGGCGCGGCGGGCG (SEQ ID NO.: 412)
MLH1 12E
CGCCCGCCGCGCCCCGCGCCCGCCCCGCCGCCCCCGCCCGTGTCCAGTC
AGCCCCAGGCCATTGTCACAGAGGATAAGACAGATATTTCTAGTGGCAGGG
CTAGGCAGCAAGATGAGGAGATGCTTGAACTCCCAGCCCCTGCTGAAGTG
GCTGCCAAAAATCAGAG (SEQ ID NO.: 413)
MLH1 13A
CGCCCGCCGCGCCCCGCGCCCGCCCCGCCGCCCCCGCCCGaatttggctaagttt
aaaaacaagaataataatgatctgcacttccttttcttcattgcagAAAGAGACATCGGGAAGATTC
TGATGTGGAAATGGTGGAAGATGATTCC (SEQ ID NO.: 414)
MLH1 13B3 (also has 12 bp miniclamp)
CGCCCGCCGCGCCCCGCGCCCGCCCCGCCGCCCCCGCCCGCattgcagAAA
GAGACATCGGGAAGATTCTGATGTGGAAATGGTGGAAGATGATTCCCGAAA
GGAAATGACTGCAGCTTGTACCCCCCGGAGAAGGATCATTAACCTCACGCG
GCGGGCG (SEQ ID NO.: 415)
MLH1 13C
CGCCCGCCGCGCCCCGCGCCCGCCCCGCCGCCCCCGCCCGTGATTCCCG
AAAGGAAATGACTGCAGCTTGTACCCCCCGGAGAAGGATCATTAACCTCAC
TAGTGTTTTGAGTCTCCAGGAAGAAATTAATGAGCAGGGACATGAGGgtacgta
aacgctgtggcctg (SEQ ID NO.: 416)
MLH1 13D
CGCCCGCCGCGCCCCGCGCCCGCCCCGCCGCCCCCGCCCGATTAACCTC
ACTAGTGTTTTGAGTCTCCAGGAAGAAATTAATGAGCAGGGACATGAGGgtac
gtaaacgctgtggcctgcctgggatgcatagggcctca
(SEQ ID NO.: 417)
MLH1 14A
CGCCCGCCGCGCCCCGCGCCCGCCCCGCCGCCCCCGCCCGggtcaatgaagtg
gggttggtaggattctattacttacctgttttttggttttattttttgttttgcagTTCTCCGGGAGATGTTGCA
TAACCACTCCTTCGTGG (SEQ ID NO.: 418)
MLH1 14B
agTTCTCCGGGAGATGTTGCATAACCACTCCTTCGTGGGCTGTGTGAATCCT
CAGTGGGCCTTGGCACAGCATCAAACCAAGTTATACCTTCTCAACACCACC
AAGCTTAGgtaaatcagctgagtgtgtgaacaagcagagctactacaacaatggtccagggagcacagg
cacaaaagctaaggagagcagcatgaggtaCGGGCGGGGGCGGCGGGGCGGGCGCG
GGGCGCGGCGGGCG (SEQ ID NO.: 419)
MLH1 15
ttcagggattacttctcccattttgtcccaactggttgtatctcaagcatgaattcagcttttccttaaagtcacttcattttt
attttcagTGAAGAACTGTTCTACCAGATACTCATTTATGATTTTGCCAATTTTGG
TGTTCTCAGGTTATCGgtaagtttagatccttttcacttctgaaatttcaactgatcgtttctgaaaatagta
gctctccactaatatcttatttgtagtatgttaaatttttcCGGGCGGGGGCGGCGGGGCGGGCGC
GGGGCGCGGCGGGCG (SEQ ID NO.: 420)
MLH1 16A
CGCCCGCCGCGCCCCGCGCCCGCCCCGCCGCCCCCGCCCGgccattctgatagt
ggattcttgggaattcaggcttcatttggatgctccgttaaagcttgctccttcatgttcttgcttcttcctagGAGCC
AGCACCGCTCTTTGACC (SEQ ID NO.: 421)
MLH1 16B
GCACCGCTCTTTGACCTTGCCATGCTTGCCTTAGATAGTCCAGAGAGTGGC
TGGACAGAGGAAGATGGTCCCAAAGAAGGACTTGCTGAATACATTGTTGAG
TTTCTGAAGAAGAAGGCTGAGATGCTTGCAGACTATTTCTCTTTGGAAATTG
ATGAGgtgtgacagccattcttatacCGGGCGGGGGCGGCGGGGCGGGCGCGGGGC
GCGGCGGGCG (SEQ ID NO.: 422)
MLH1 16C2
GGCTGAGATGCTTGCAGACTATTTCTCTTTGGAAATTGATGAGgtgtgacagccat
tcttatacttctgttgtattcttcaaataaaatttccagccgggtgcggtggctcatgCGGGCGGGGGCGG
CGGGGCGGGCGCGGGGCGCGGCGGGCG (SEQ ID NO.: 423)
MLH1 17
CGCCCGCCGCGCCCCGCGCCCGCCCCGCCGCCCCCGCCCGtgtttaaactatga
cagcattatttcttgttcccttgtcctttttcctgcaagcagGAAGGGAACCTGATTGGATTACCCCT
TCTGATTGACAACTATGTGCCCCCTTTGGAGGGACTGCCTATCTTCATTCTT
CGACTAGCCACTGAGgtcagtgatcaagcagatactaagcatttcggtacatgcatgtgtgctggagg
gaaagggcaaatgaccacc (SEQ ID NO.: 424)
MLH1 18A2
CGCCCGCCGCGCCCCGCGCCCGCCCCGCCGCCCCCGCCCGtgtgatctccgttta
gaatgagaatgtttaaattcgtacctattttgaggtattgaatttctttggaccagGTGAATTGGGACGAA
GAAAAGGAATGTTTTGAAAGCCTCAGTAAAGAATGCGCTATGTTCTATTCCA
TCCGGAAGCAGTACATATCTGAGGAGTCGACCCTCTCAG
(SEQ ID NO.: 425)
MLH1 18B3 (also has 14 bp miniclamp)
CGCCCGCCGCGCCCCGCGCCCGCCCCGCCGCCCCCGCCCGTGCGCTATG
TTCTATTCCATCCGGAAGCAGTACATATCTGAGGAGTCGACCCTCTCAGGC
CAGCAGgtacagtggtgatgcacactggcaccccaggactagCGGGCGGGGGCGGC
(SEQ ID NO.: 426)
MLH1 19A
aagtctttccagacccagtgcacatcccatcagccaggacaccagtgtatgttgggatgcaaacagggaggctt
atgacatctaatgtgttttccagagtgaAGTGCCTGGCTCCATTCCAAACTCCTGGAAGTG
GACTGTGGAACACATTGTCTATAAAGCCTTGCGCTCACACATTCTGCCTCCT
AAACATTTCACAGAAGATGGAAATATCCTGCAGCTTGCTAACCTGCCTGATC
TATACACGGGCGGGGGCGGCGGGGCGGGCGCGGGGCGCGGCGGGCG
(SEQ ID NO.: 427)
MLH1 19B4
AAGGCCTTGCGCTCACACATTCTGCCTCCTAAACATTTCACAGAAGATGGAA
ATATCCTGCAGCTTGCTAACCTGCCTGATCTATACAAAGTCTTTGAGAGGTg
GTTAAatatggttattCGGGCGGGGGCGGCGGGGCGGGCGCGGGGCGCGGCG
GGCG (SEQ ID NO.: 428)
MLH1 19C (also has 7 bp miniclamp)
CGCCCGCCGCGCCCCGCGCCCGCCCCGCCGCCCCCGCCCGCAGAAGATG
GAAATATCCTGCAGCTTGCTAACCTGCCTGATCTATACAAAGTCTTTGAGAG
GTGTTAAatatggttatttatgcactgtgggatgtgttcttctttctctgtattccgatacaaagtgttgtatcaaagt
gtgatatacaCGGGCGG (SEQ ID NO.: 429)

All exons and clamps are in capital letters.

1. A method of identifying the presence or absence of a genetic marker in the human mismatch repair genes mutL homolog 1 (MLH1) and mutS homologue 2 (MSH2) of a subject comprising:
providing a DNA sample from said subject;
providing at least one primer set from TABLE A;
contacting said DNA and said at least one primer set;
generating an extension product from said at least one primer set that comprises a region of DNA that includes the location of said genetic marker;
separating said extension produce on the basis of melting behavior; and
identifying the presence or absence of said genetic marker in said subject by analyzing the melting behavior of said extension product.

2. The method of claim 1, wherein at least two primer sets from TABLE A are contacted with said DNA.

3. The method of claim 1, wherein at least three primer sets from TABLE A are contacted with said DNA.

4. The method of claim 1, wherein at least four primer sets from TABLE A are contacted with said DNA.

5. The method of claim 1, wherein at least five primer sets from TABLE A are contacted with said DNA.

6. The method of claim 1, wherein at least six primer sets from TABLE A are contacted with said DNA.

7. The method of claim 1, wherein at least seven primer sets from TABLE A are contacted with said DNA.

8. The method of claim 1, wherein at least eight primer sets from TABLE A are contacted with said DNA.

9. The method of claim 2, wherein the extension products generated from said primer sets are grouped according to TABLE D and separated on the basis of melting behavior.

10. The method of claim 4, wherein the extension products generated from said primer sets are grouped according to TABLE D and separated on the basis of melting behavior.

11. The method of claim 6, wherein the extension products generated from said primer sets are grouped according to TABLE D and separated on the basis of melting behavior.

12. The method of claim 8, wherein the extension products generated from said primer sets are grouped according to TABLE D and separated on the basis of melting behavior.

13. A method of identifying the presence or absence of a genetic marker in the human mismatch repair genes mutL homolog 1 MLH1) and mutS homologue 2 (MSH2) of a subject comprising:
providing a DNA sample from said subject;
providing at least one primer set that is any number between 1-75 nucleotides upstream or downstream of a primer set from TABLE A;
contacting said DNA and said at least one primer set;
generating an extension product from said at least one primer set that comprises a region of DNA that includes the location of said genetic marker;
separating said extension product on the basis of melting behavior; and
identifying the melting behavior of said extension product in said subject by analyzing the melting behavior of said extension product.

14. The method of claim 13, wherein at least two primer sets that are any number between 1-75 nucleotides upstream or downstream of a primer set from TABLE A are contacted with said DNA.

15. The method of claim 13, wherein at least three primer sets that are any number between 1-75 nucleotides upstream or downstream of a primer set from TABLE A are contacted with said DNA.

16. The method of claim 13, wherein at least four primer sets that are any number between 1-75 nucleotides upstream or downstream of a primer set from TABLE A are contacted with said DNA.

17. The method of claim 13, wherein at least five primer sets that are any number between 1-75 nucleotides upstream or downstream of a primer set from TABLE A are contacted with said DNA.

18. The method of claim 13, wherein at least six primer sets that are any number between 1-75 nucleotides upstream or downstream of a primer set from TABLE A are contacted with said DNA.

19. The method of claim 13, wherein at least seven primer sets that are any number between 1-75 nucleotides upstream or downstream of a primer set from TABLE A are contacted with said DNA.

20. The method of claim 13, wherein at least eight primer sets that are any number between 1-75 nucleotides upstream or downstream of a primer set from TABLE A are contacted with said DNA.

21. The method of claim 14, wherein the extension products generated from said primer sets are grouped according to TABLE D and separated on the basis of melting behavior.

22. The method of claim 16, wherein the extension products generated from said primer sets are grouped according to TABLE D and separated on the basis of melting behavior.

23. The method of claim 18, wherein the extension products generated from said primer sets are grouped according to TABLE D and separated on the basis of melting behavior.

24. The method of claim 20, wherein the extension products generated from said primer sets are grouped according to TABLE D and separated on the basis of melting behavior.

25. The method of claim 3, wherein the extension products generated from said primer sets are grouped according to TABLE D and separated on the basis of melting behavior.

26. The method of claim 5, wherein the extension products generated from said primer sets are grouped according to TABLE D and separated on the basis of melting behavior.

27. The method of claim 7, wherein the extension products generated from said primer sets are grouped according to TABLE D and separated on the basis of melting behavior.

28. The method of claim 15, wherein the extension products generated from said primer sets are grouped according to TABLE D and separated on the basis of melting behavior.

29. The method of claim 17, wherein the extension products generated from said primer sets are grouped according to TABLE D and separated on the basis of melting behavior.

30. The method of claim 19, wherein the extension products generated from said primer sets are grouped according to TABLE D and separated on the basis of melting behavior.
